(19) United States
(12) Patent Application Publication
(10) Pub. No.: US 2024/0397813 A1
(43) Pub. Date: Nov. 28, 2024

(54) ORGANIC COMPOUND, AND ORGANIC LIGHT-EMITTING DEVICE COMPRISING SAME

(71) Applicant: P&H TECH Co., Ltd, Yongin-si (KR)

(72) Inventors: Seo-Yong HYUN, Yongin-si (KR); Gwan-hee PARK, Yongin-si (KR); Jun-Yeong CHANG, Yongin-si (KR)

(73) Assignee: P&H TECH Co., Ltd, Yongin-si (KR)

(21) Appl. No.: 18/291,697

(22) PCT Filed: Sep. 27, 2021
(86) PCT No.: PCT/KR2021/013095

§ 371 Date: Jan. 24, 2024

(30) Foreign Application Priority Data
Jul. 27, 2021 (KR) 10-2021-0098356

(51) Int. Cl. H10K 85/60; C07D 203/04; C07D 207/267; C07D 207/27; C07D 211/76; C07D 401/14; C07D 405/14; C09K 11/06; H10K 50/858

(52) U.S. Cl. CPC H10K 85/654; C07D 203/04; C07D 207/267; C07D 207/27; C07D 211/76; C07D 401/14; C07D 405/14; C09K 11/06; H10K 50/858; H10K 85/626; H10K 85/636; C09K 2211/1018

(57) ABSTRACT

The present invention relates to an organic compound and an organic light emitting device comprising same, the organic compound being employed in a light efficiency improving layer, which is provided in an organic light emitting device, so as to increase the light efficiency of the device, and thus enables low-voltage driving of the device and excellent device characteristics such as excellent color purity and improved luminous efficiency to be implemented. Therefore, the present invention can be industrially and effectively used in various lighting and display devices and the like.

### TECHNICAL FIELD

The present invention relates to an organic compound that is employed as a material for a light efficiency improving layer (capping layer) provided in an organic light emitting device, and an organic light emitting device that employs the same, thus achieving greatly improved luminescent properties such as low-voltage driving of the device and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

In addition, recently, research aimed at improving the characteristics of organic light emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

### DISCLOSURE

### Technical Problem

Thus, the present invention has been made in an effort to provide a novel organic compound which may be employed in a light efficiency improving layer (capping layer) provided in an organic light emitting device to implement excellent luminescent properties such as low-voltage driving of the device and improved luminous efficiency, and an organic light emitting device including the same.

### Technical Solution

An aspect of the present invention provides an organic compound represented by Formula I below.

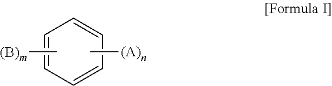

Characteristic structures of Formula I above and specific compounds A and B implemented thereby will be described below.

Another aspect of the present invention provides an organic light emitting device including a first electrode, a second electrode, and one or more organic layers arranged between the first and second electrodes, wherein the organic light emitting device further includes a light efficiency improving layer (capping layer) formed on at least one side opposite to the organic layer among the upper or lower portions of the first electrode and the second electrode, and the light efficiency improving layer includes an organic compound represented by Formula I above.

### Advantageous Effects

According to the present invention, an organic compound is employed as a material for a light efficiency improving layer provided in an organic light emitting device to implement low-voltage driving of the organic light emitting device and improved luminescent properties such as excellent luminous efficiency, color purity, etc., and thus can be effectively used in various display devices.

### BEST MODE

Hereinafter, the present invention will be described in more detail.

The present invention relates to an organic light emitting compound represented by Formula I below, which is employed as a material for a light efficiency improving layer provided in an organic light emitting device to achieve low-voltage driving of the device and luminescent properties such as excellent luminous efficiency, color purity, etc.

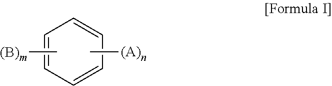

In Formula I above,
(i) A is any one selected from structures represented by Structural Formula 1 below (‘*’ indicates a linking site), n is an integer of 1 to 4, and when the n is 2 or more, a plurality of A are the same as or different from each other.

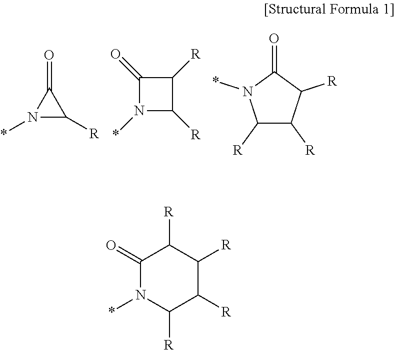

The compound represented by Formula I according to the present invention is characterized by introducing at least one structure selected from aziridinone, azetidinone, pyrrolidinone, and piperidinone in a benzene ring.

In Structural Formula 1, in each structure, R is each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and in each structure of Structural Formula 1, a plurality of R are the same as or different from each other.
(ii) B is selected from deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, a substituted or unsubstituted heteroaryl group having 2 to 30 carbon atoms, and Structural Formula 2 below, and m is an integer of 0 to 5, and when the m is each 2 or more, a plurality of B are the same as or different from each other.

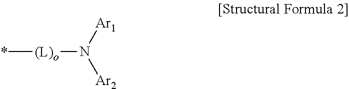

In Structural Formula 2 above,
L is a single bond or selected from a substituted or unsubstituted arylene group having 6 to 30 carbon atoms and a substituted or unsubstituted heteroarylene group having 3 to 30 carbon atoms, o is an integer of 0 to 2, and when the o is 2, a plurality of L are the same as or different from each other.
Ar₁ and Ar₂ are the same as or different from each other and are each independently selected from a substituted or unsubstituted aryl group having 6 to 30 carbon atoms and a substituted or unsubstituted heteroaryl group having 2 to 30 carbon atoms.
(iii) However, in the definition of n and m above, 3≤n+m≤6.
(iv) Meanwhile, the ‘substituted or unsubstituted’ in the definition of R, B, L, Ar₁ and Ar₂ above means substitution of R, B, L, Ar₁ and Ar₂ above with one or more substituents selected from the group consisting of deuterium, a halogen group, a cyano group, a nitro group, a hydroxy group, a silyl group, an alkyl group, an amine group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

For specific examples, the substituted aryl group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

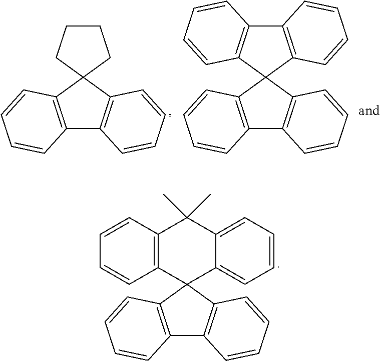

In an embodiment of the present invention, the fluorenyl groups include open structures in which one of the two cyclic organic compounds linked through one atom is cleaved, and examples thereof include

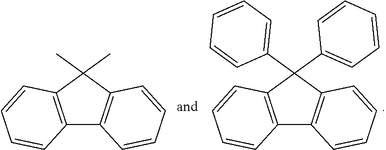

In addition, carbon atoms of the ring may be substituted with any one or more heteroatoms selected from among N, S and O, and examples thereof include

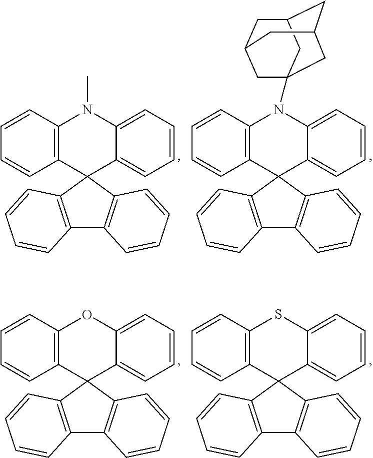

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the amine group may be —NH₂, an alkylamine group, an arylamine group, heteroaryl amino group, an arylheteroarylamine group, etc., the aryl (heteroaryl)amine group means an amine substituted with an aryl group and/or heteroaryl group, and the alkylamine group means an amine substituted with an alkyl group. Examples of the aryl (heteroaryl)amine group include a substituted or unsubstituted mono aryl (heteroaryl)amine group, a substituted or unsubstituted diaryl (heteroaryl)amine group, or a substituted or unsubstituted triaryl (heteroaryl)amine group, wherein the aryl group and the heteroaryl group in the aryl (heteroaryl)amine group are the same as the definition of the aryl group and the heteroaryl group, and the alkyl group in the alkylamine group is also the same as the definition of the alkyl group.

For example, the arylamine group includes a phenylamine group, a naphthylamine group, a biphenylamine group, an anthracenylamine group, a 3-methyl-phenylamine group, a 4-methyl-naphthylamine group, and a 2-methyl-biphenyl amine group, a 9-methyl-anthracenylamine group, a diphenyl amine group, a phenylnaphthylamine group, a ditolylamine group, a phenyltolylamine group, a triphenylamine group, etc., but is not limited thereto.

In an embodiment of the present invention, the silyl group is an unsubstituted silyl group or a silyl group substituted with an alkyl group, an aryl group, and the like, and specific examples of the silyl group may include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (CI), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound according to the present invention represented by Formula I may be used as a material for the light efficiency improving layer (capping layer) provided in the organic light emitting device due to its structural specificity.

Preferred specific examples of the organic compound represented by Formula I according to the present invention include the following compounds but are not limited thereto.

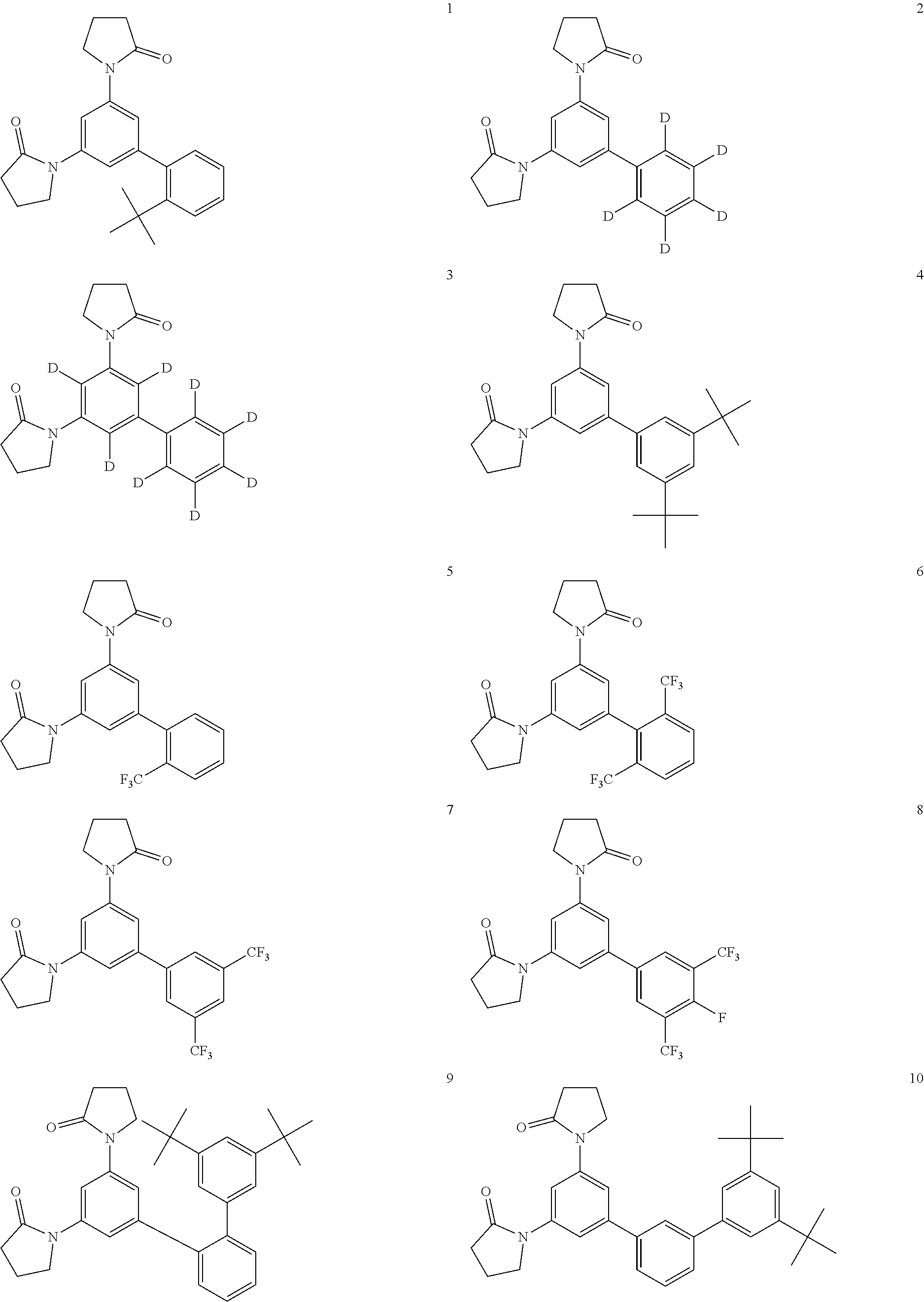

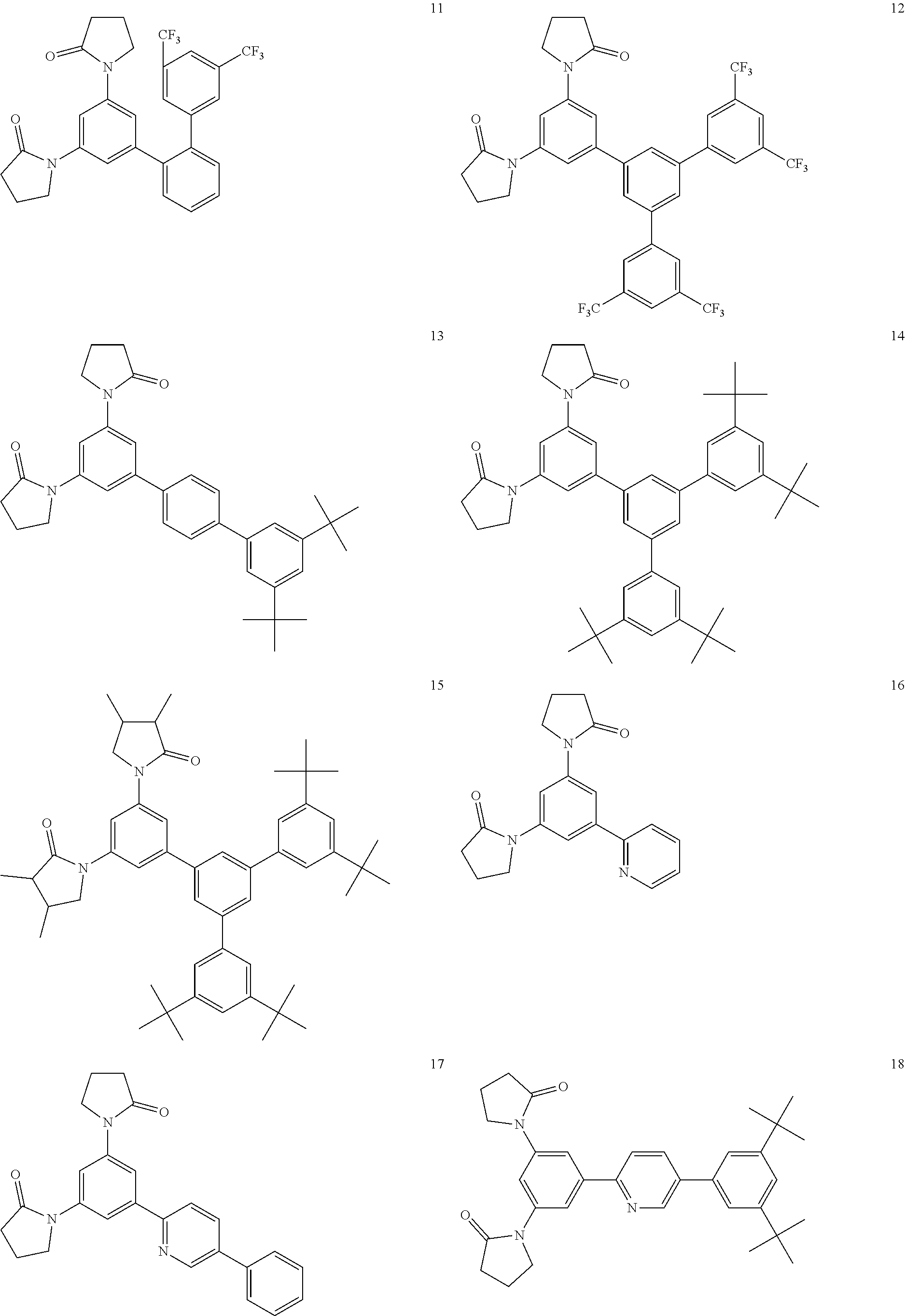

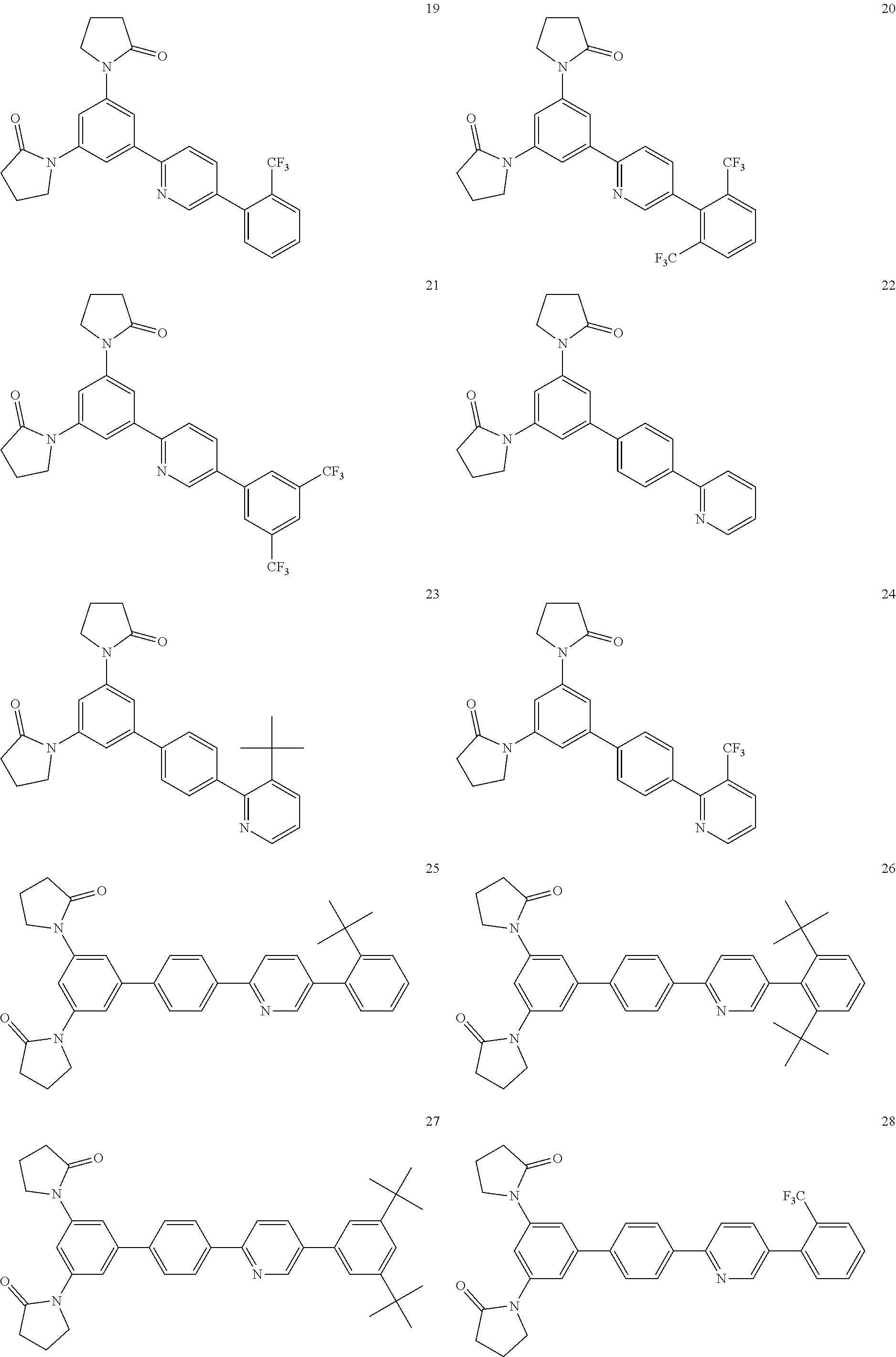

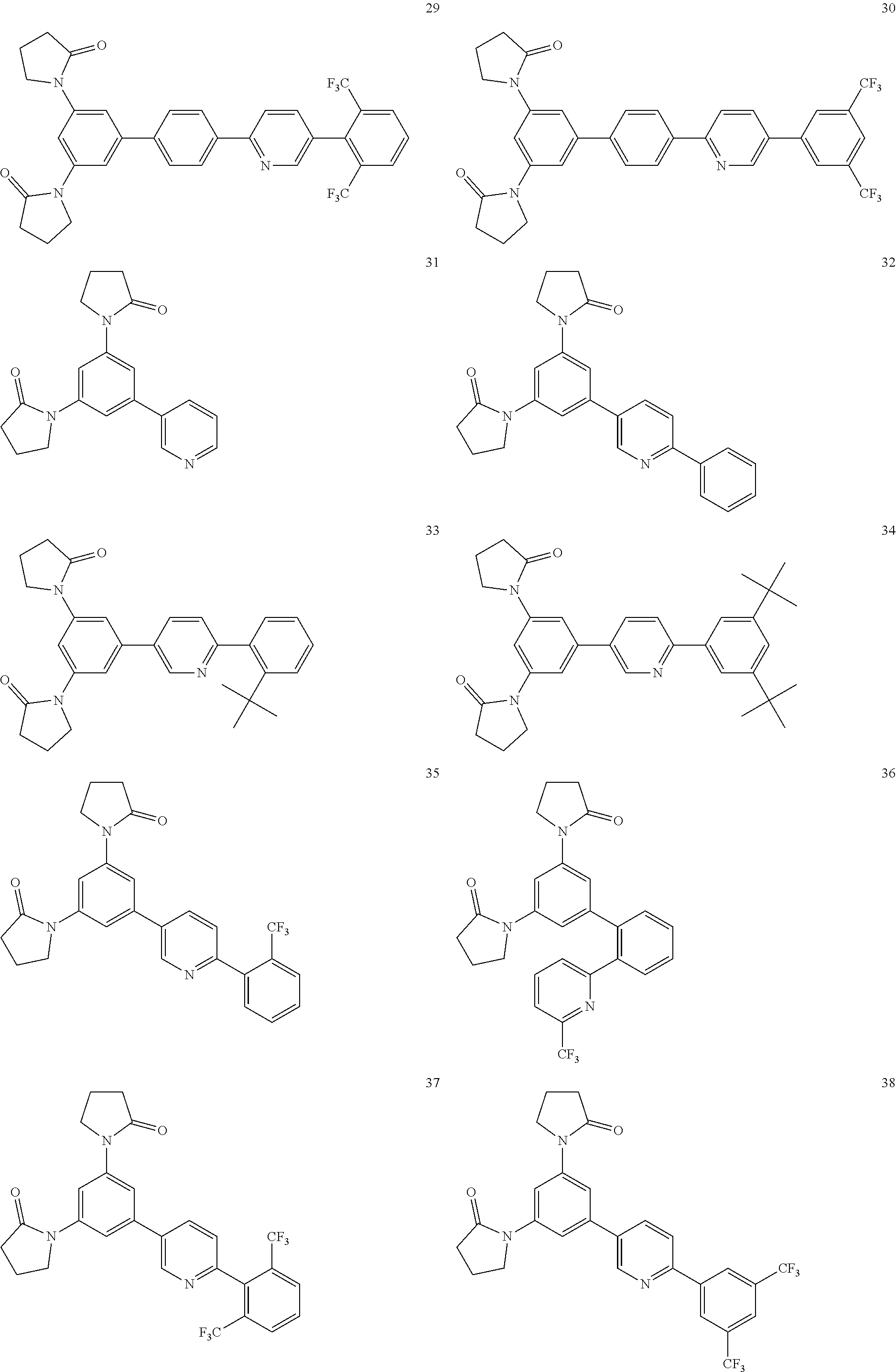

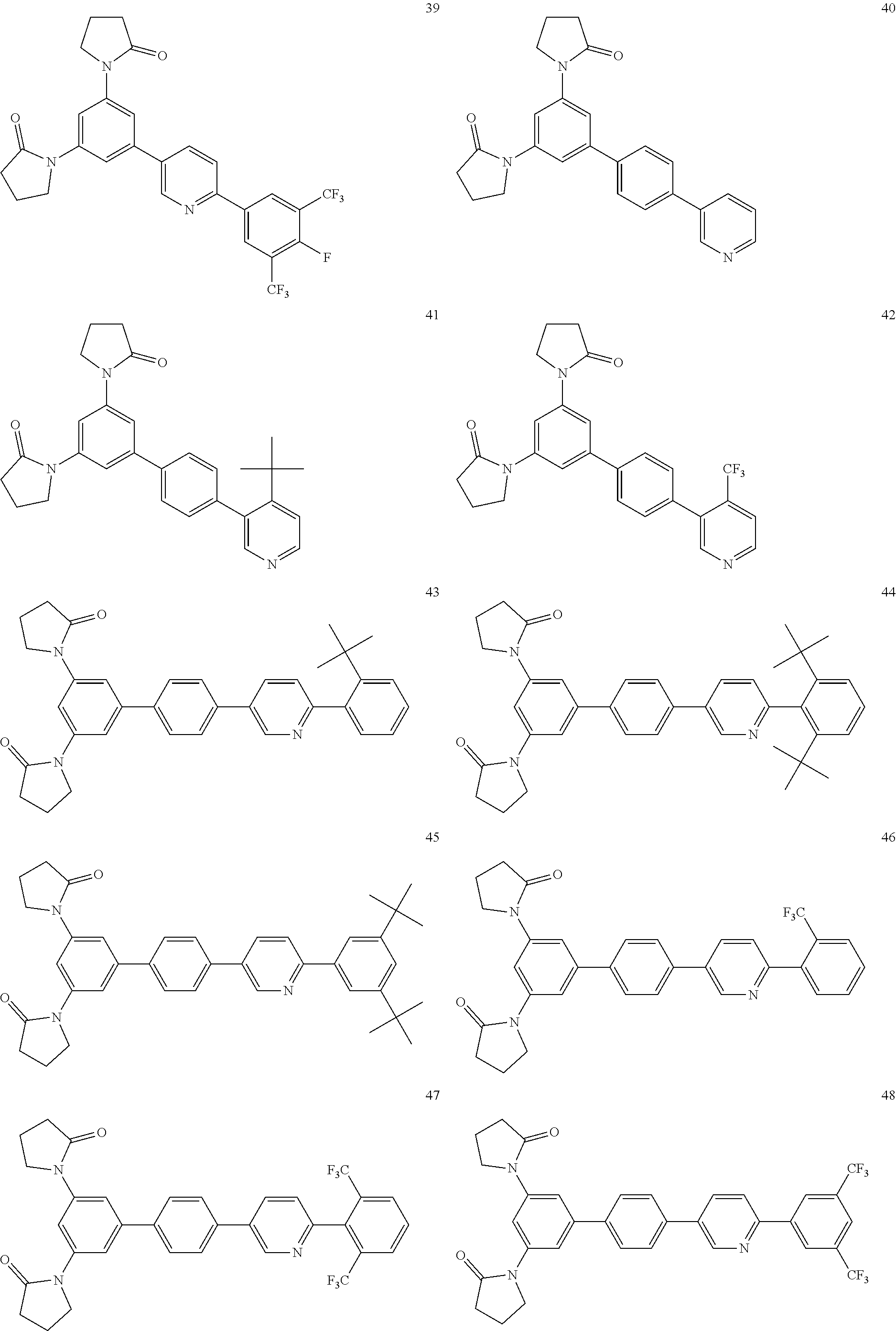

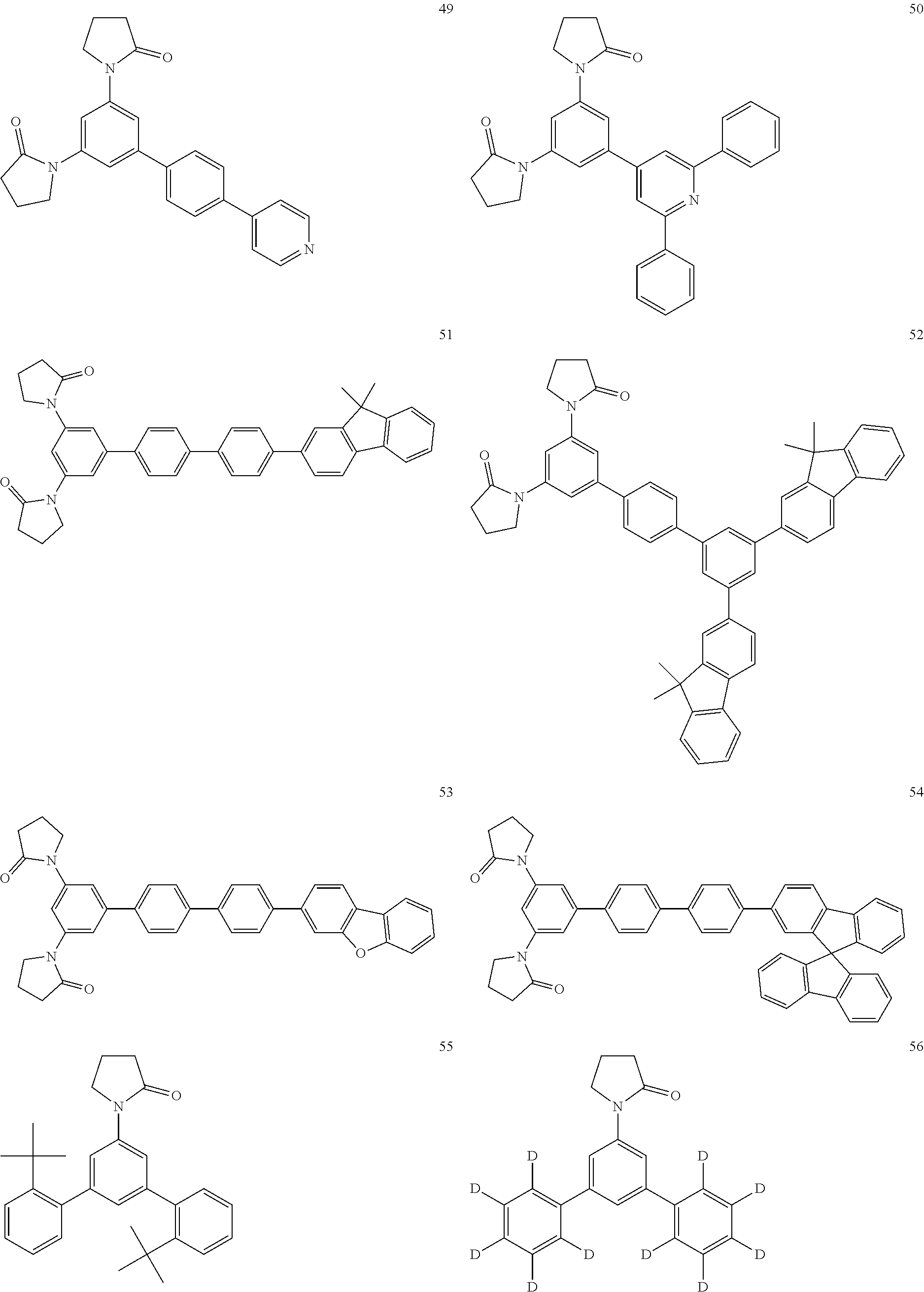

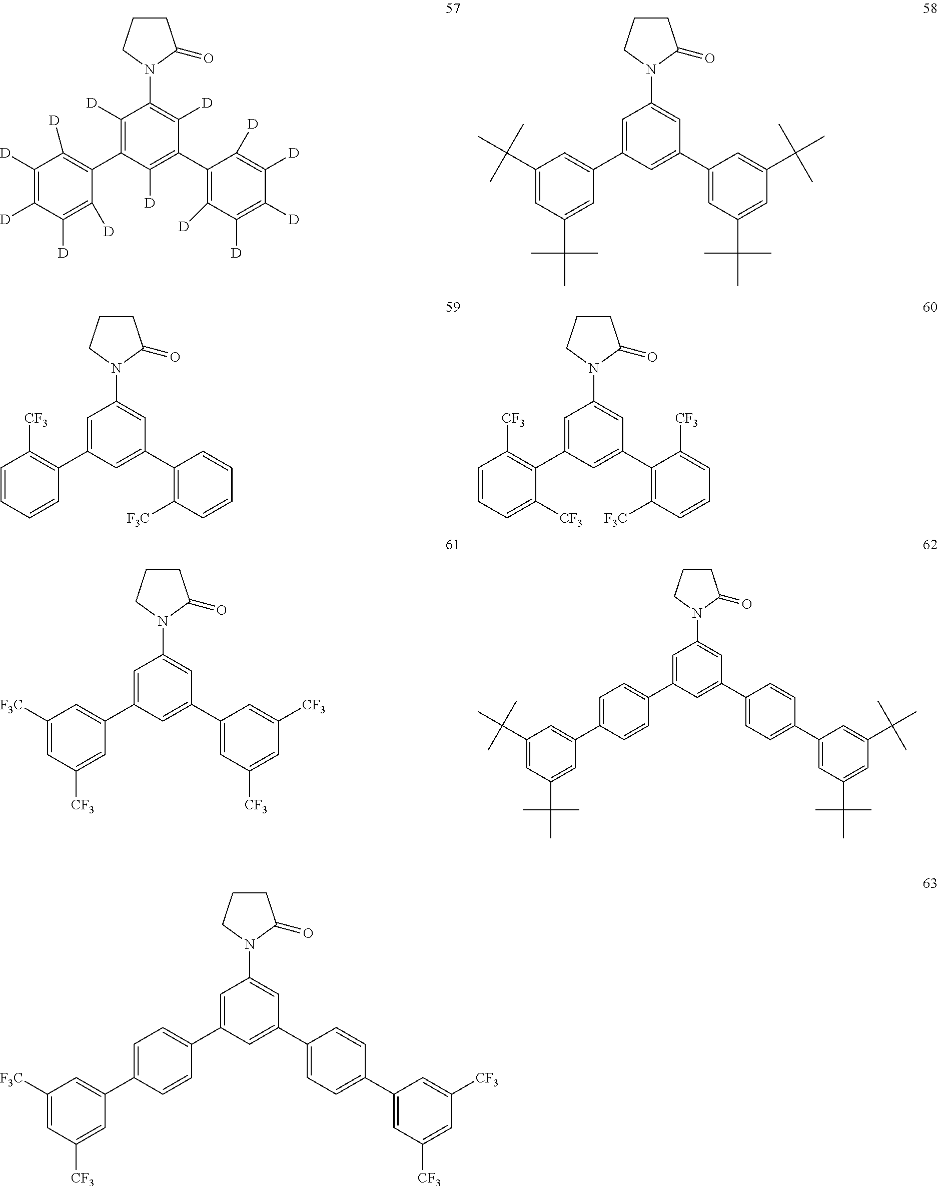

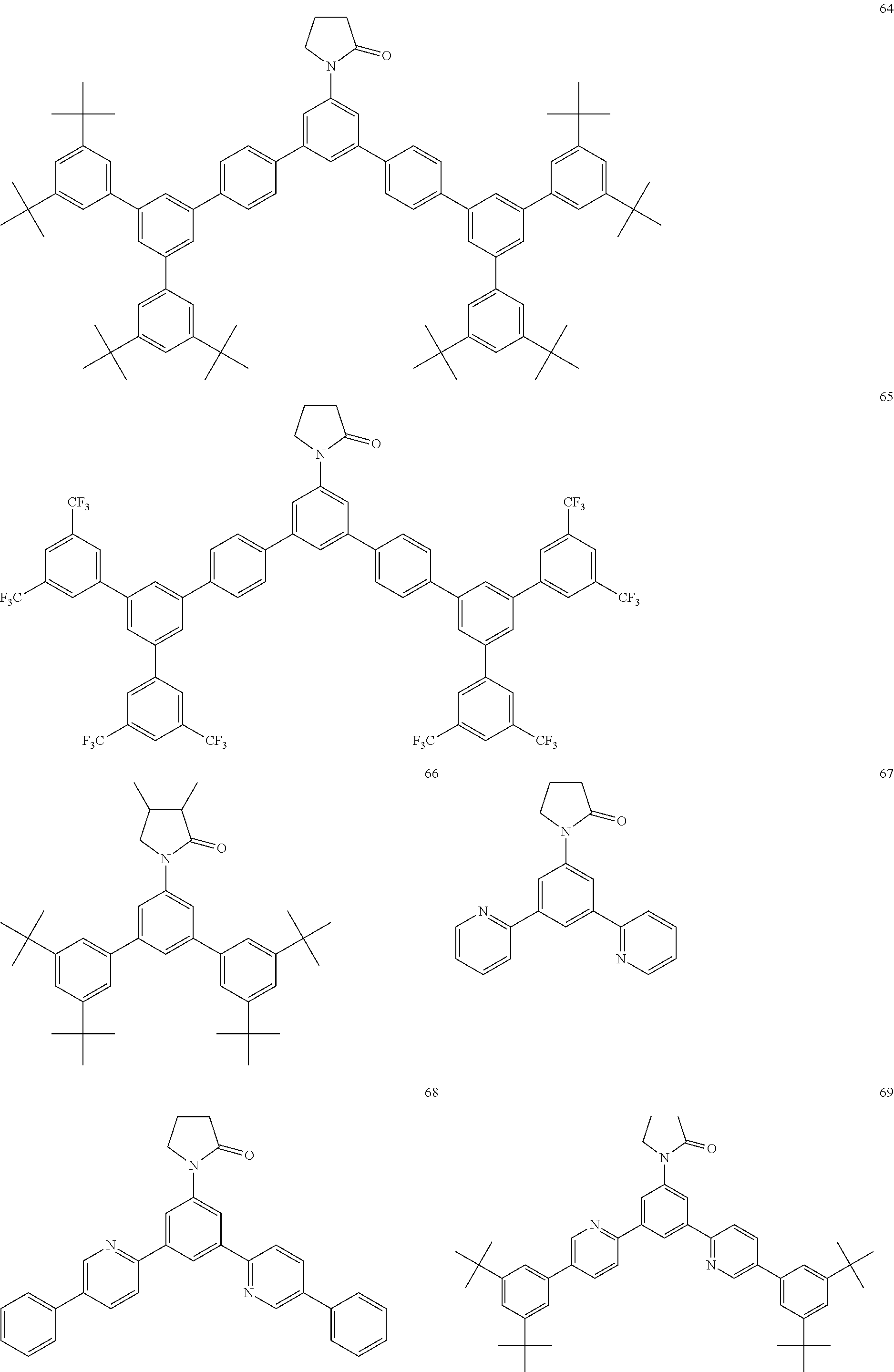

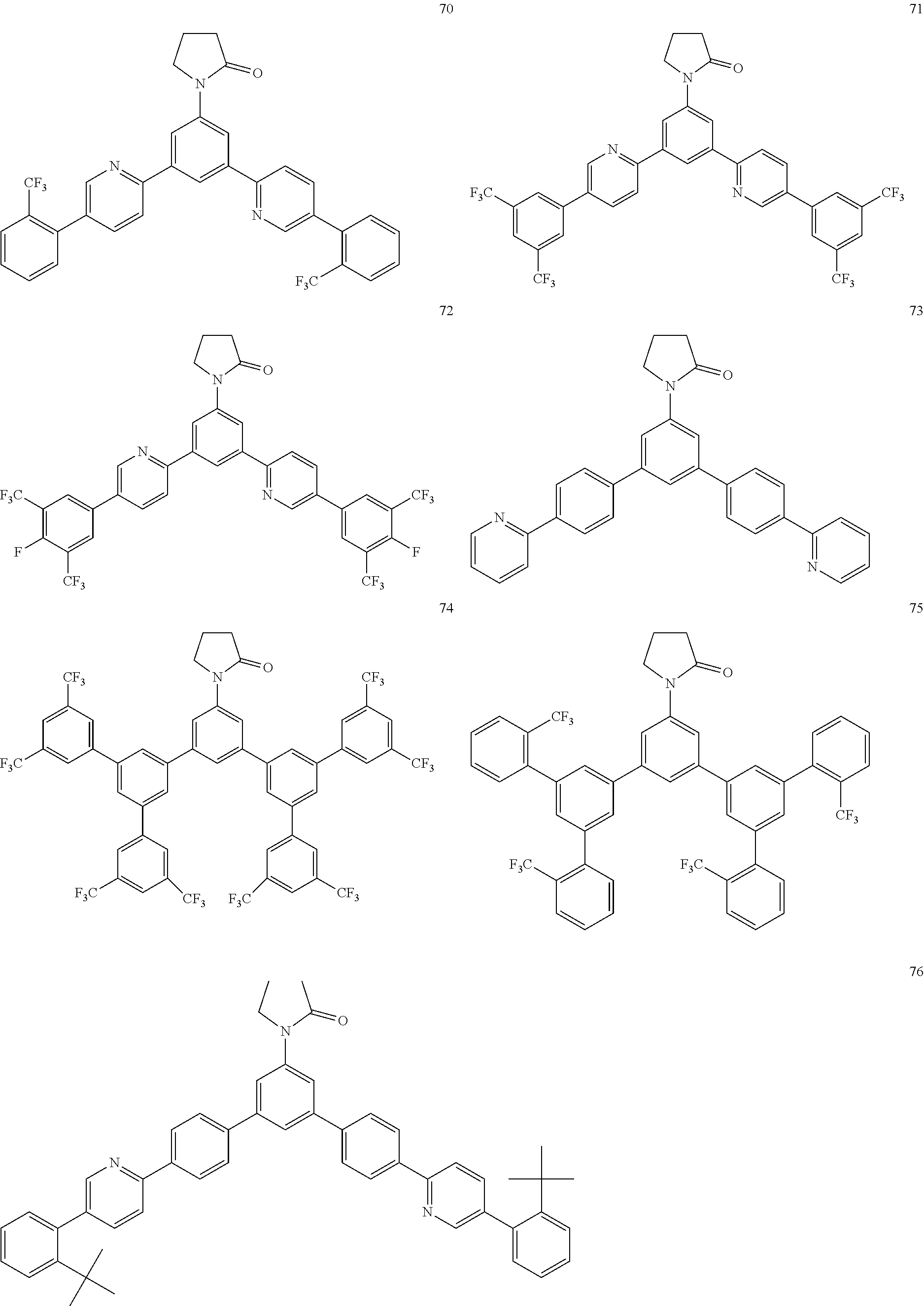

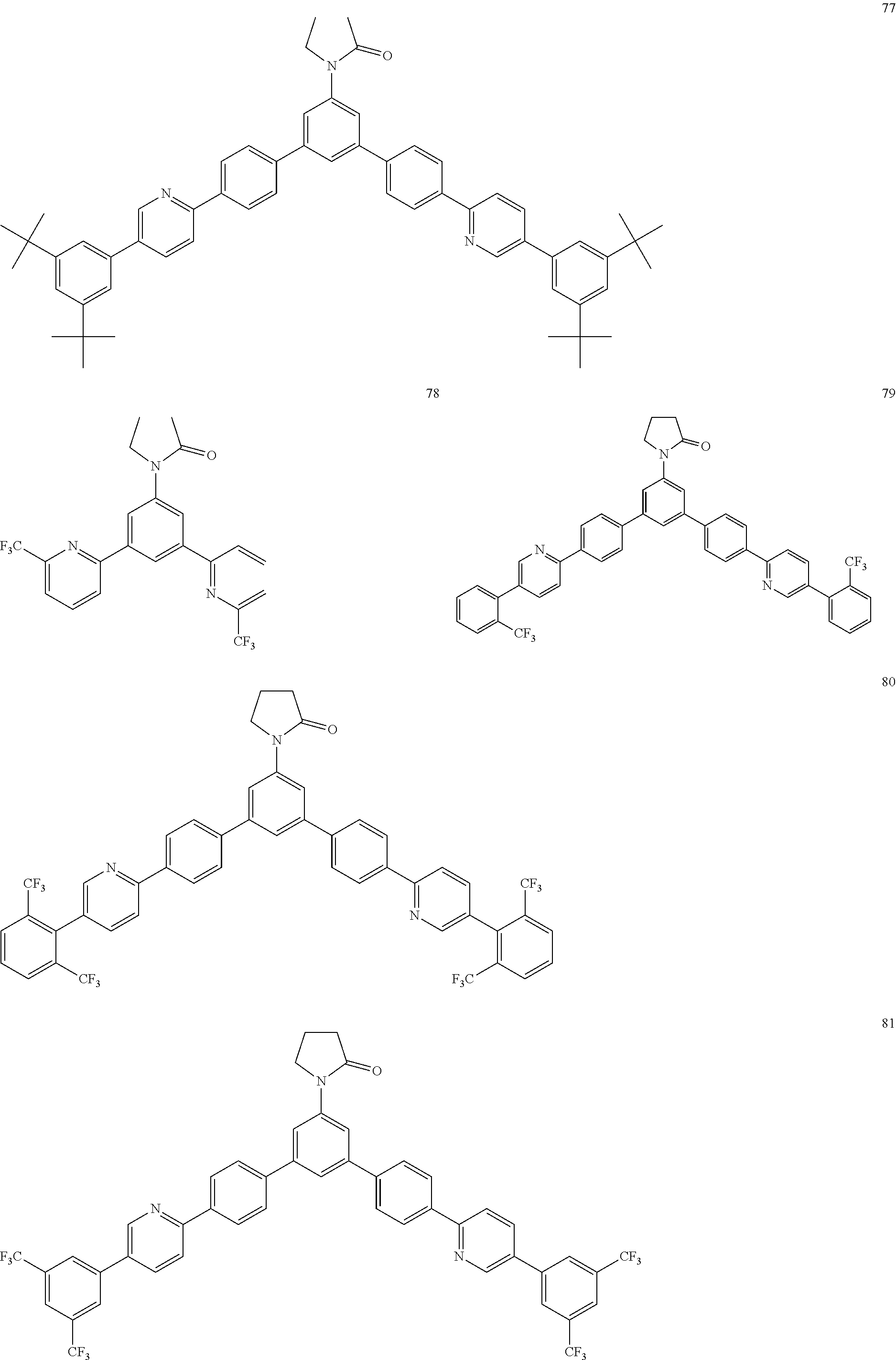

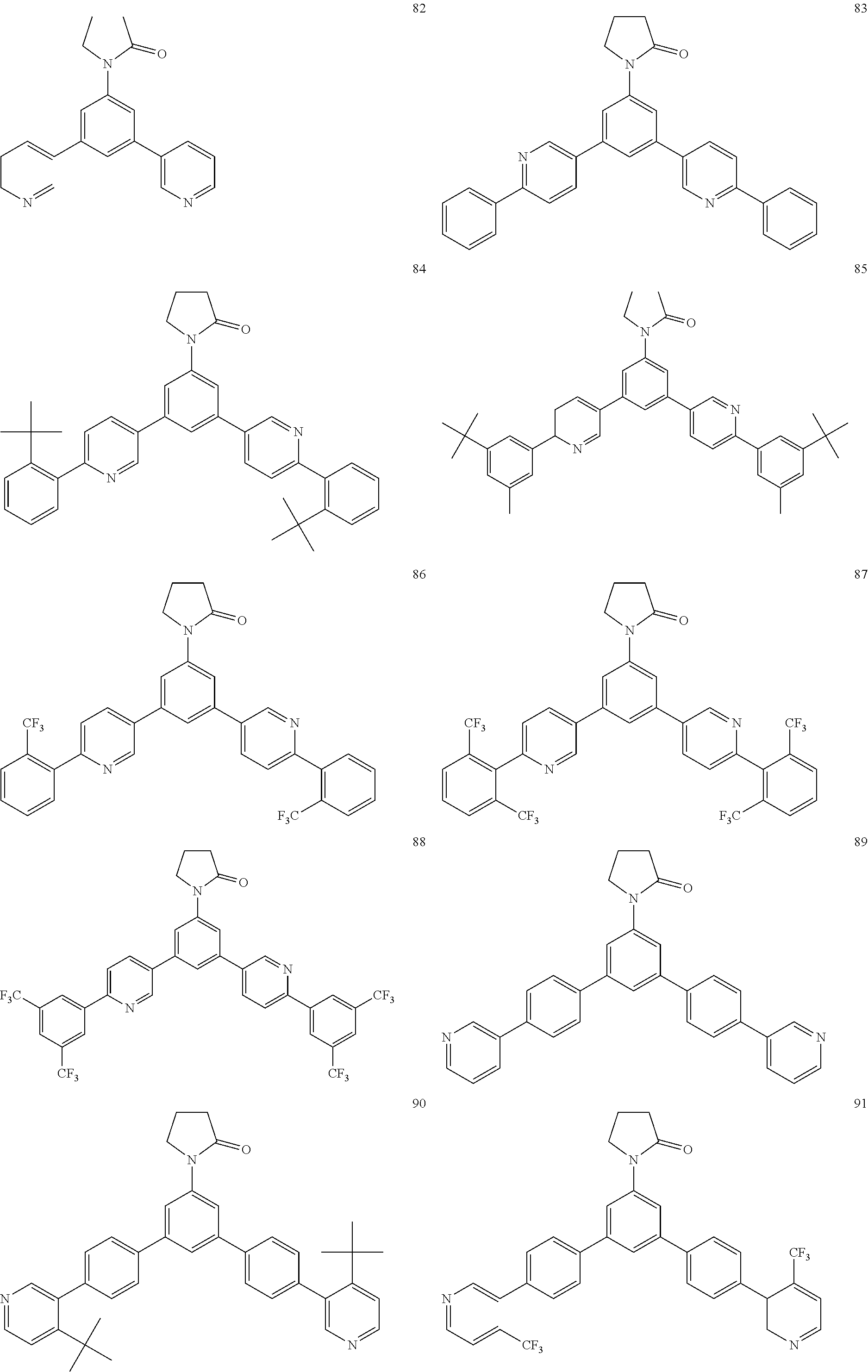

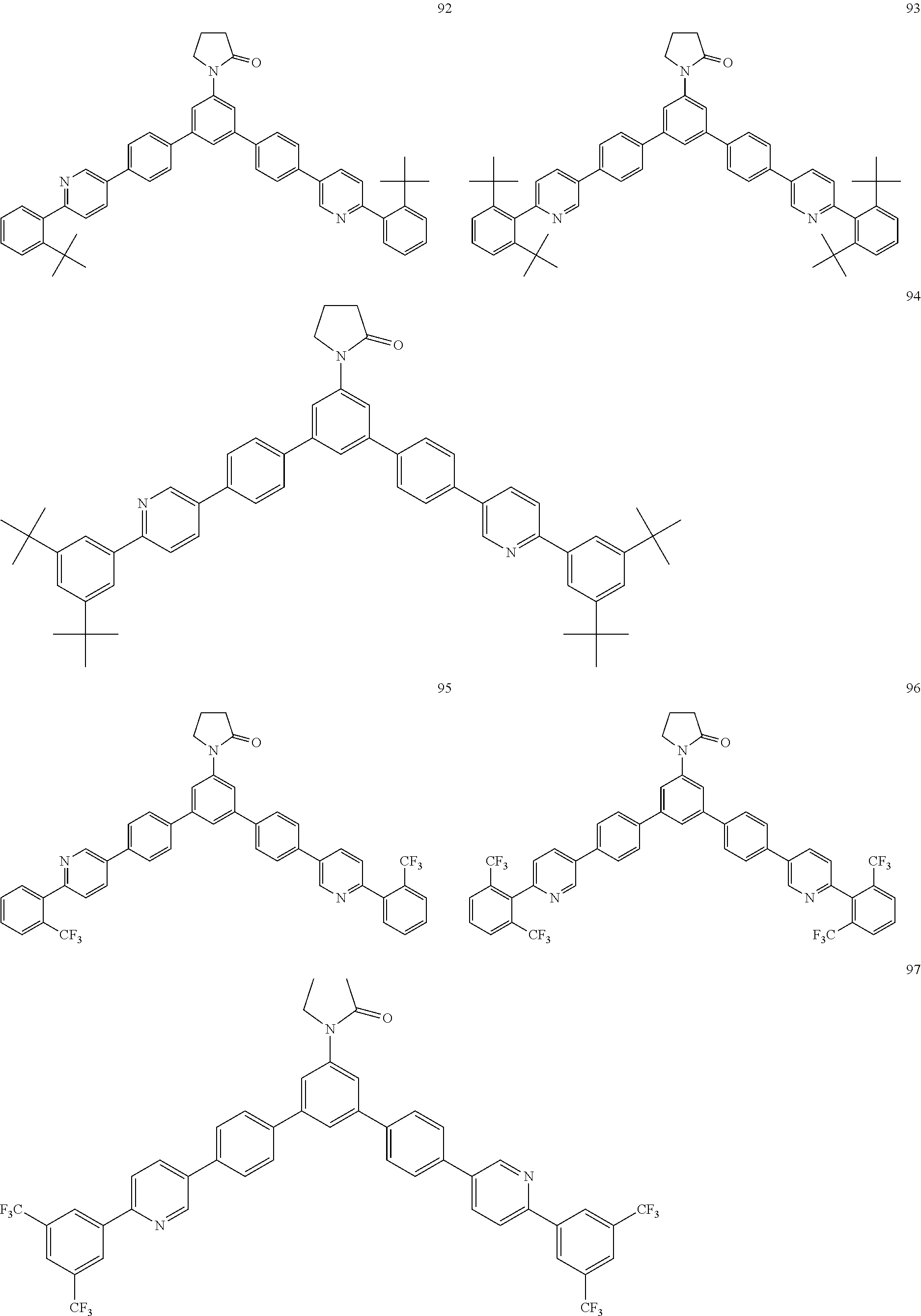

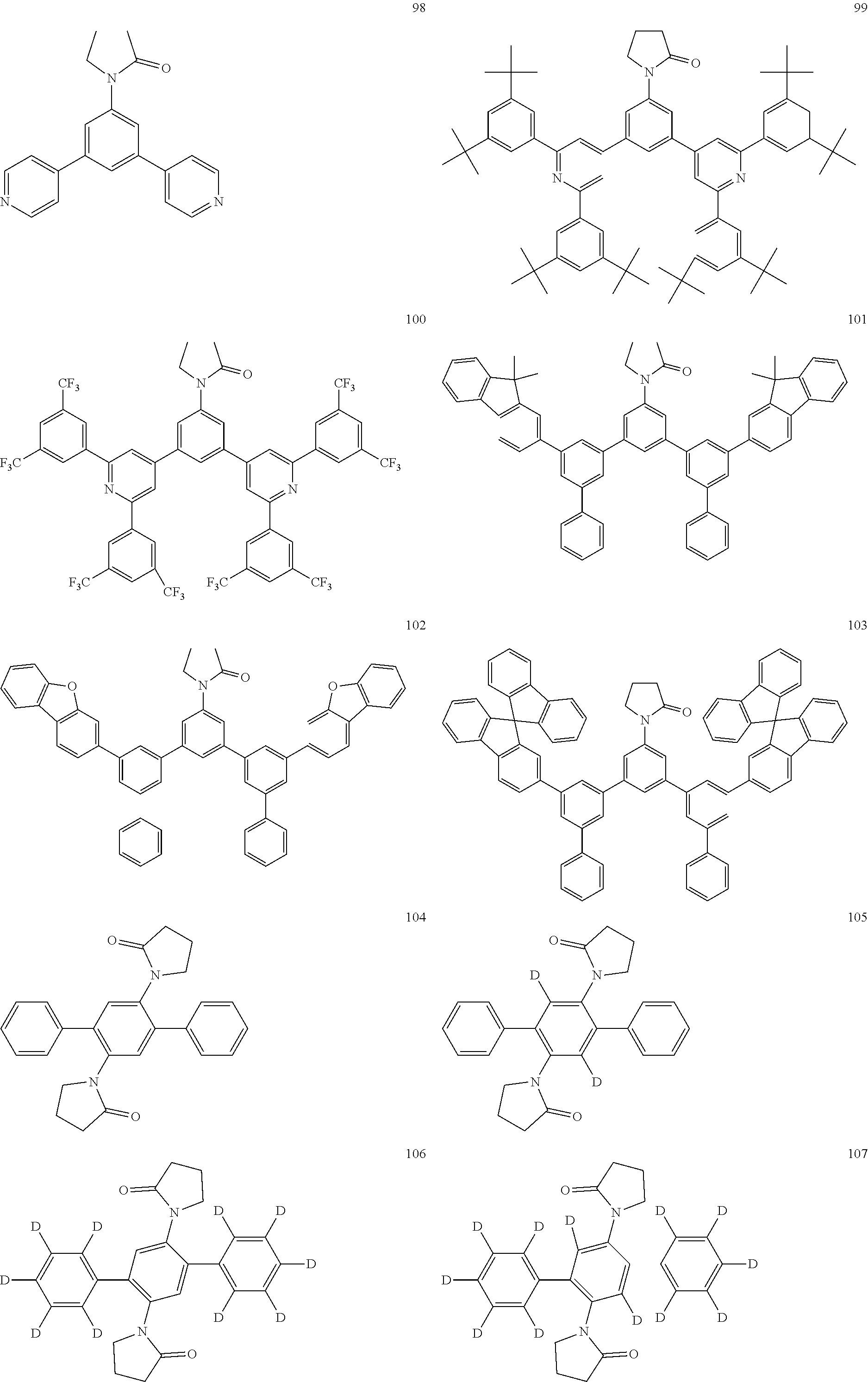

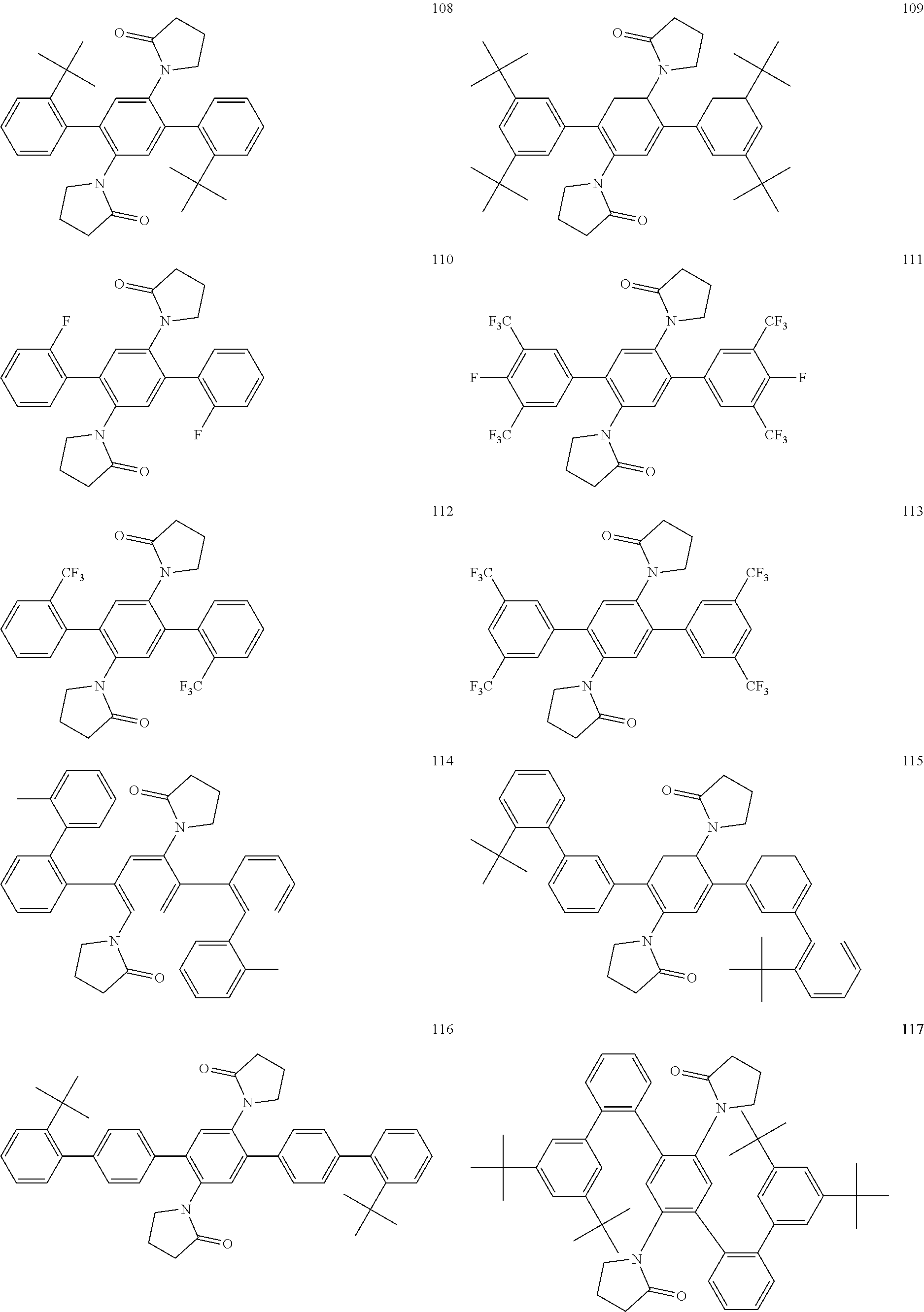

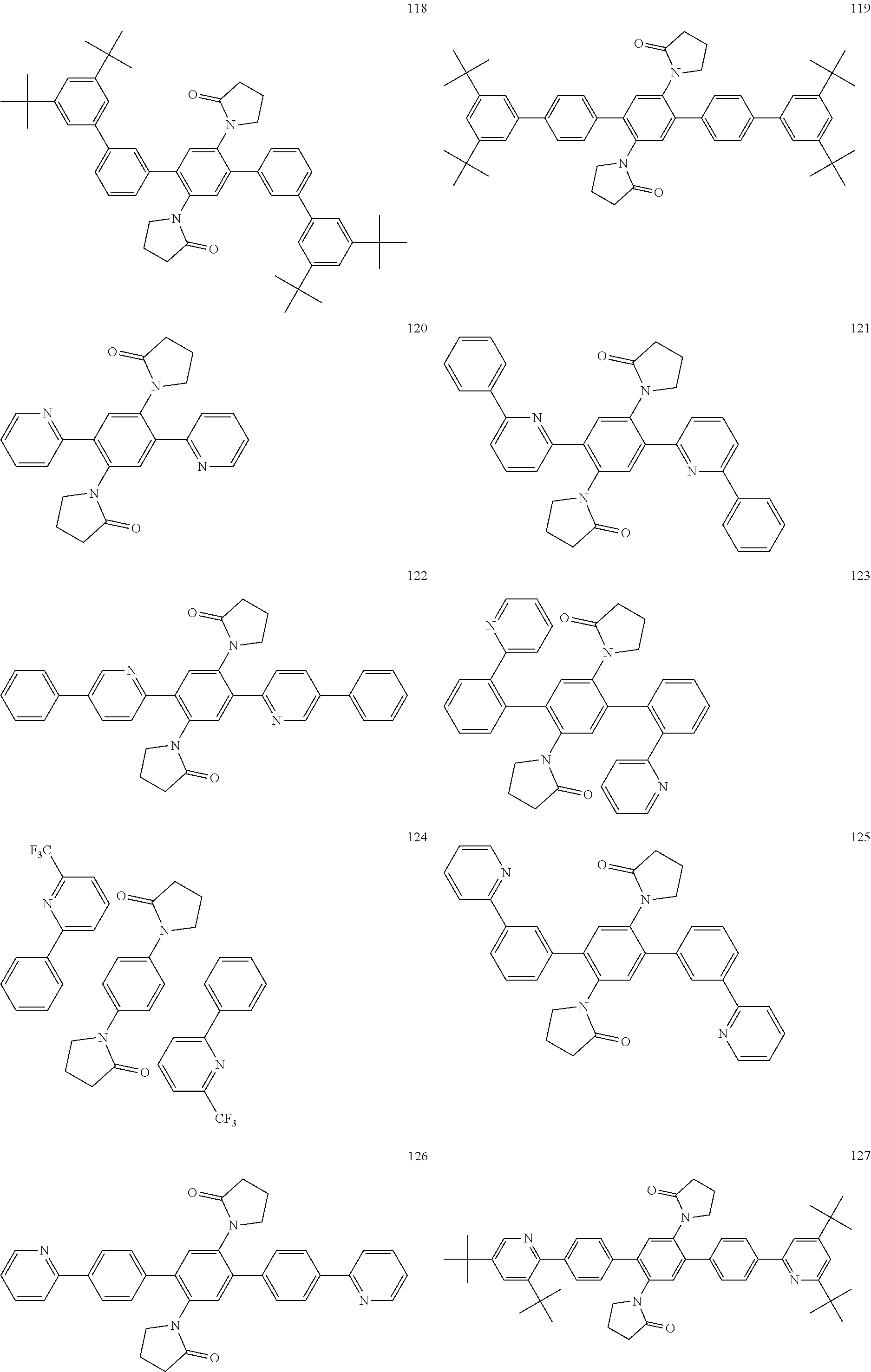

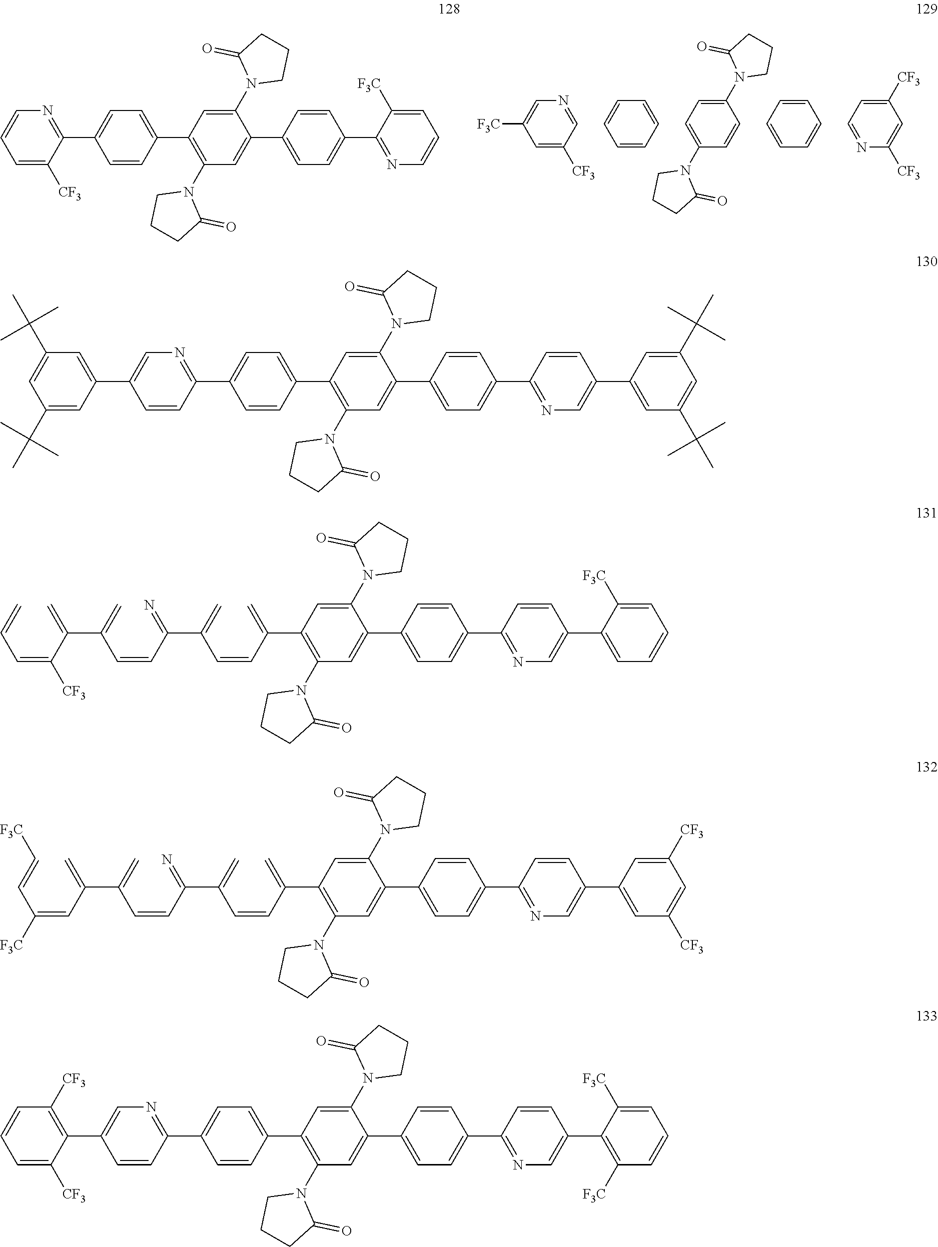

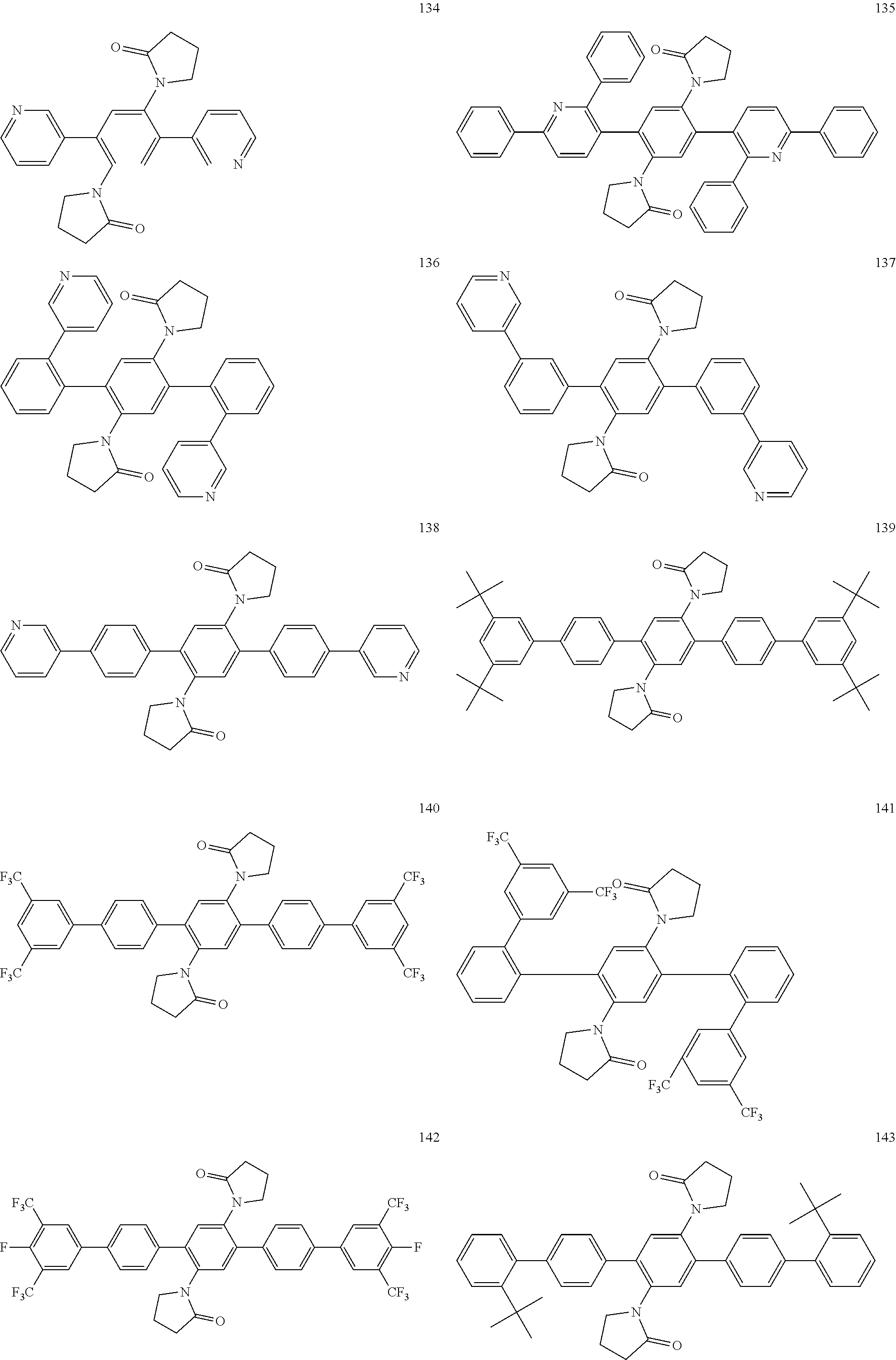

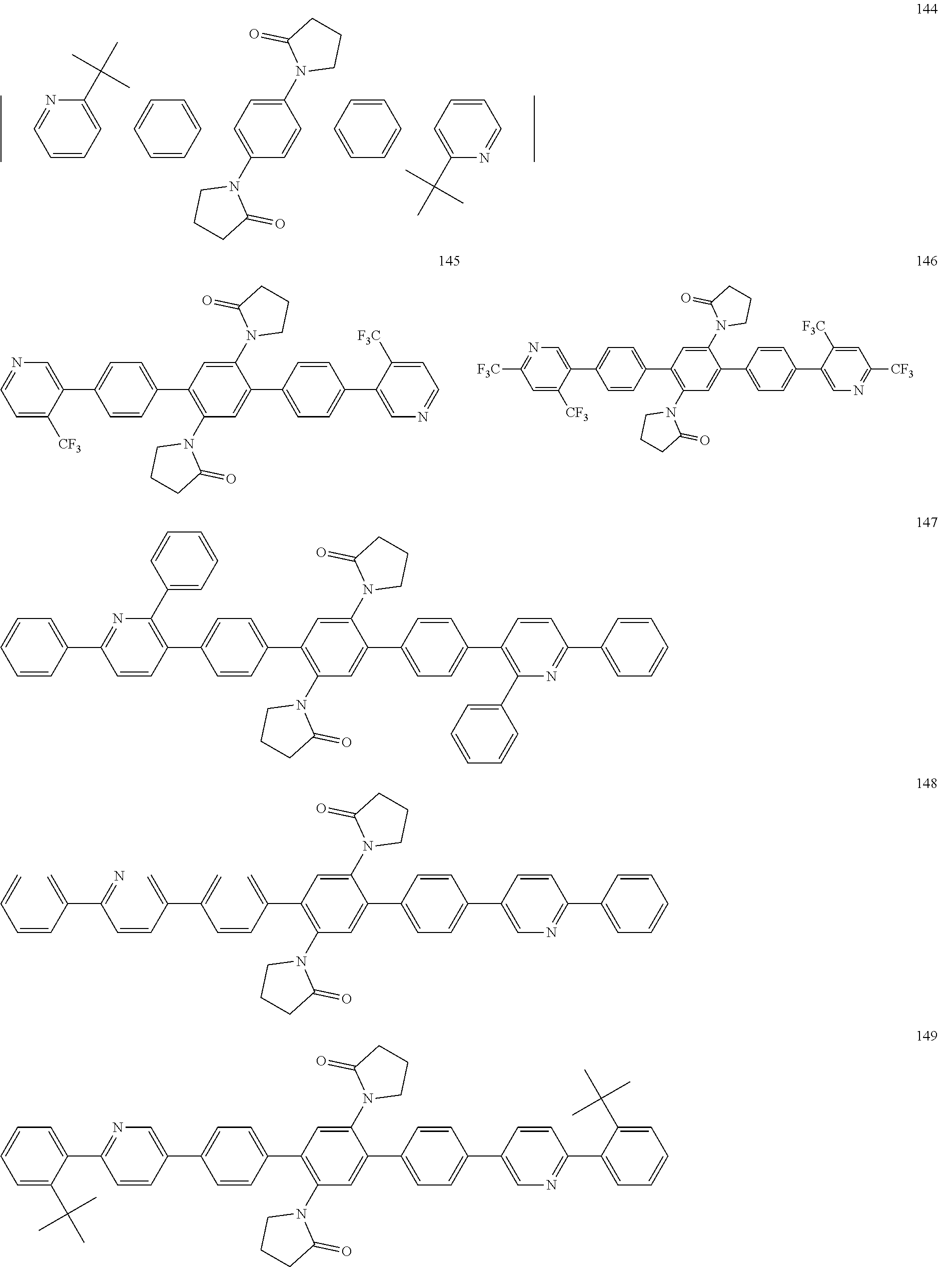

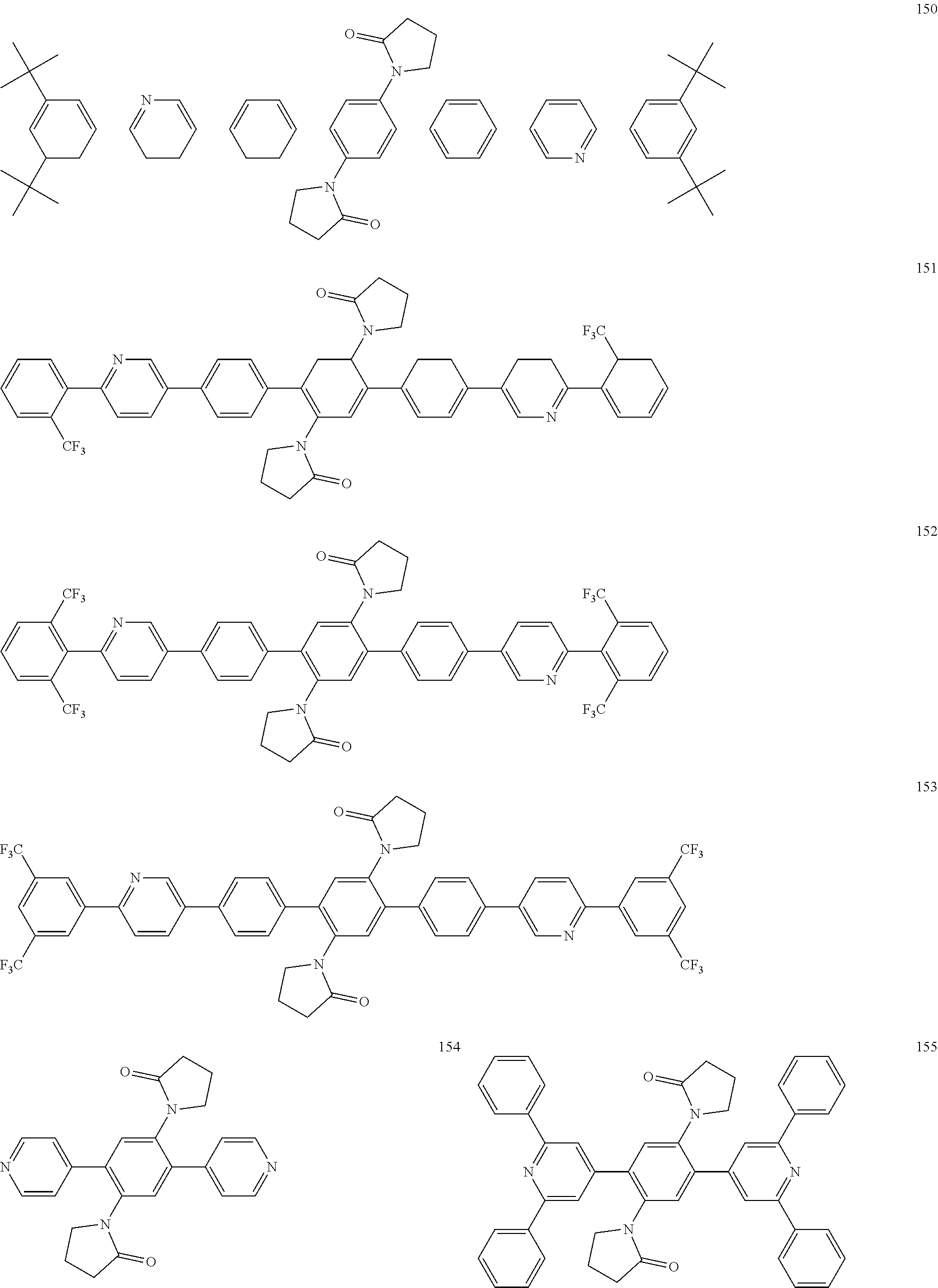

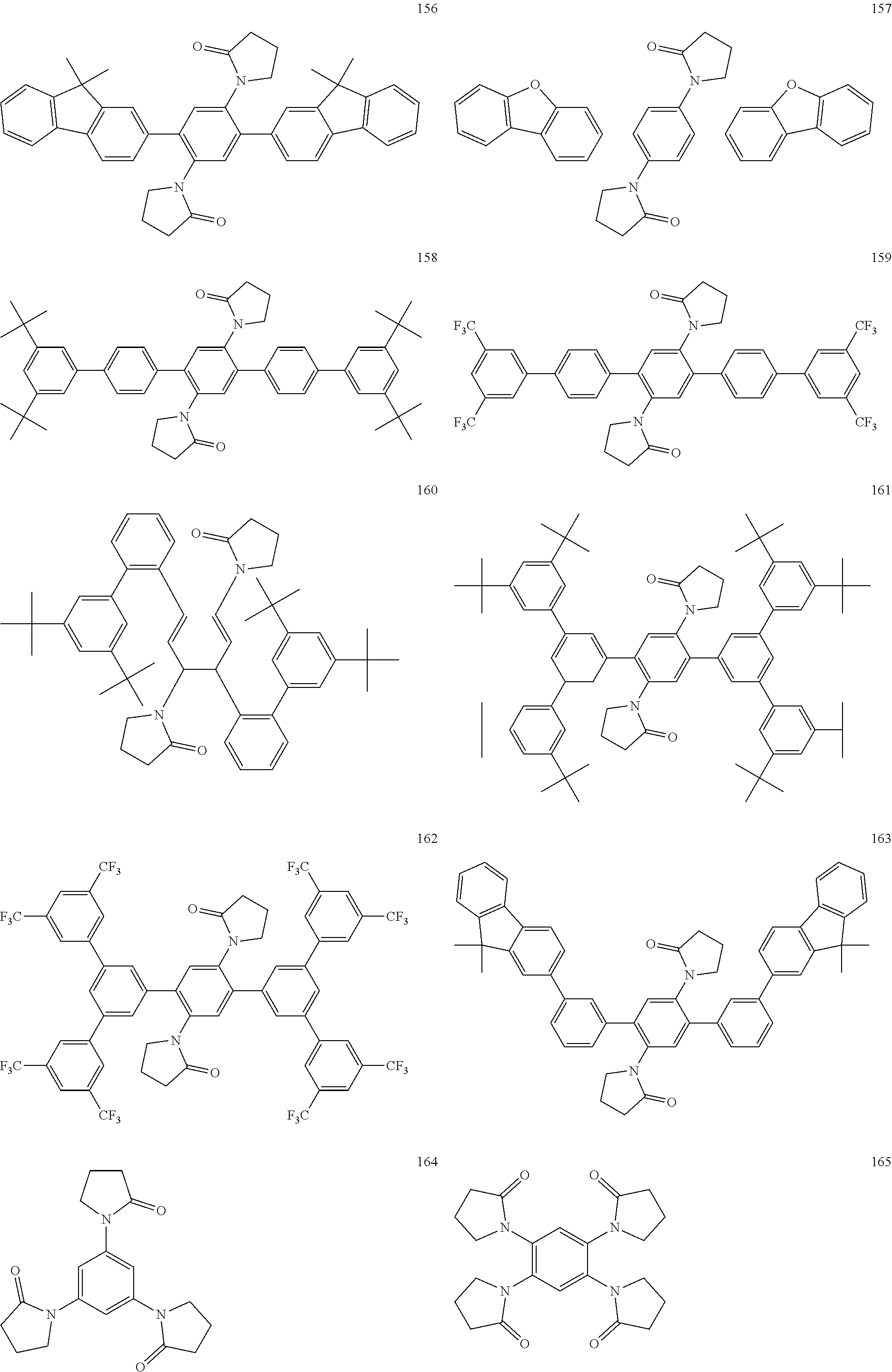

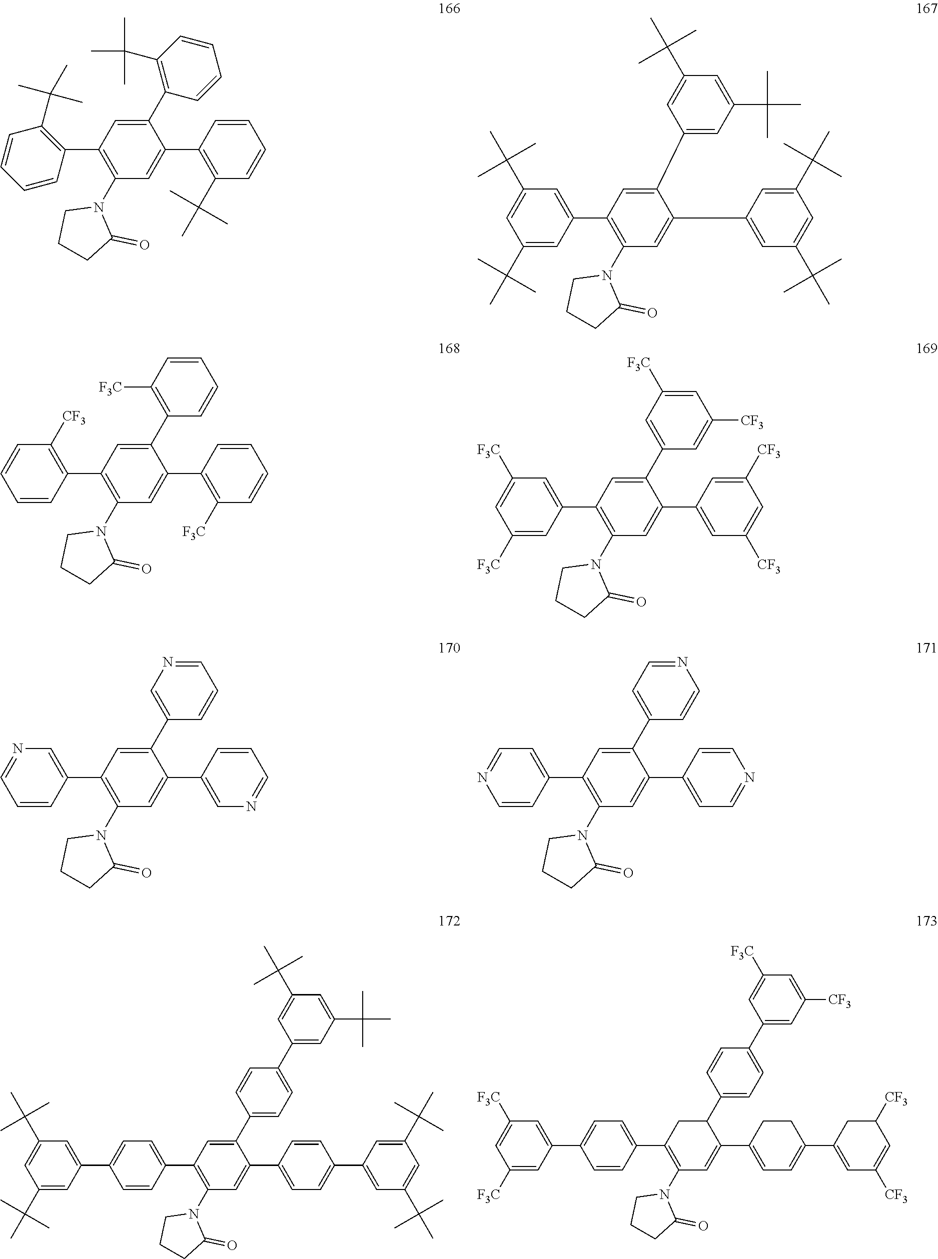

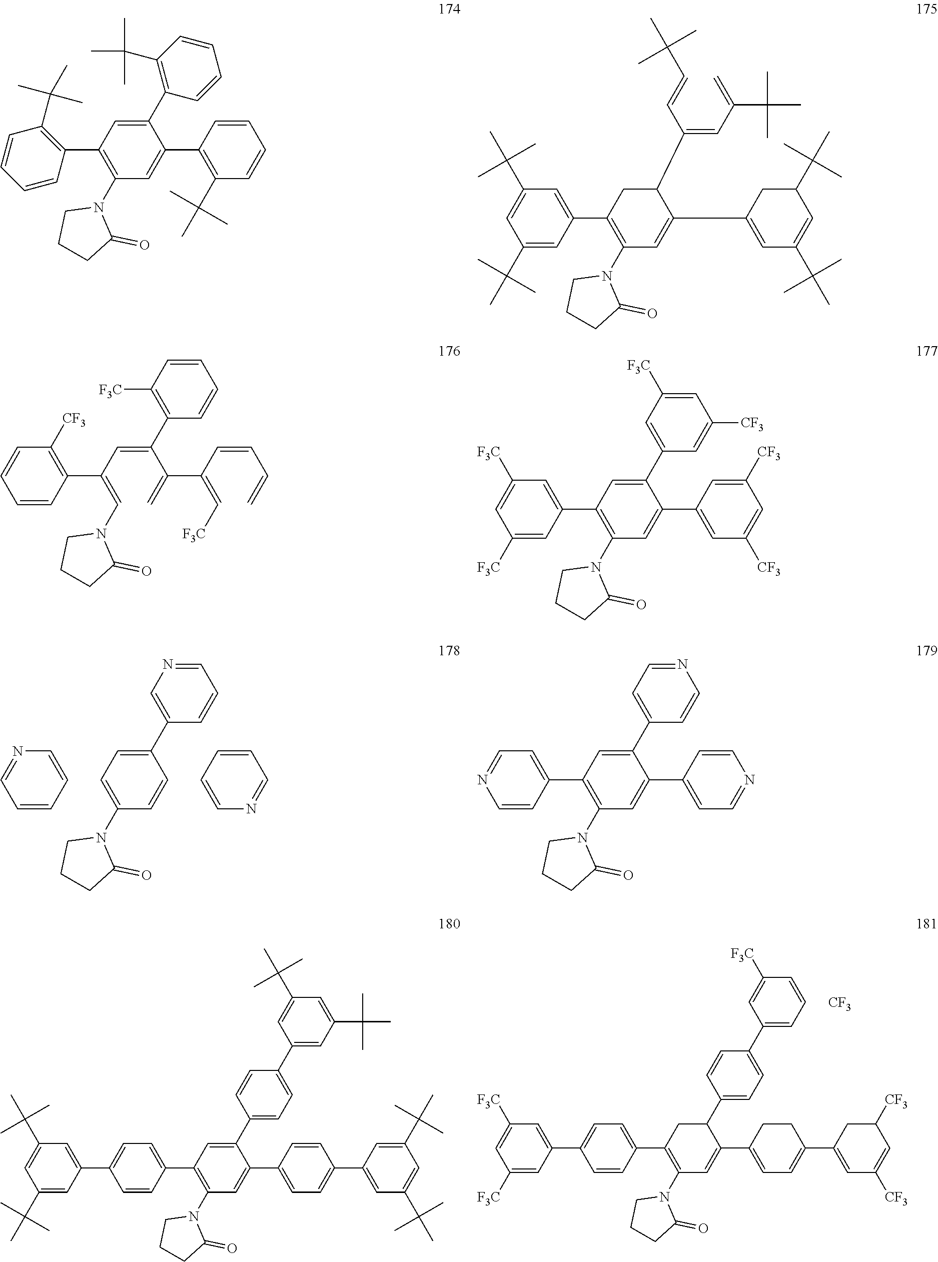

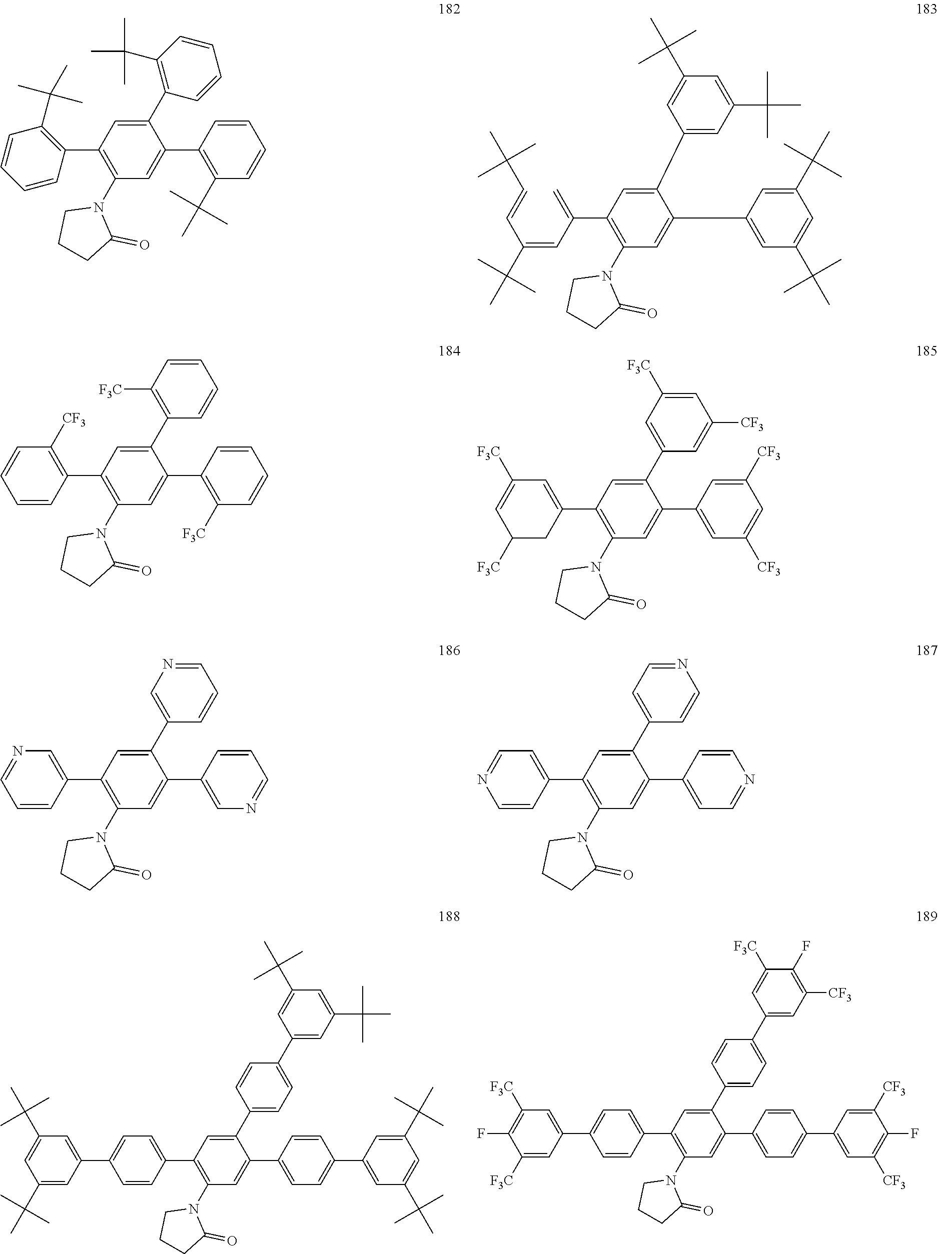

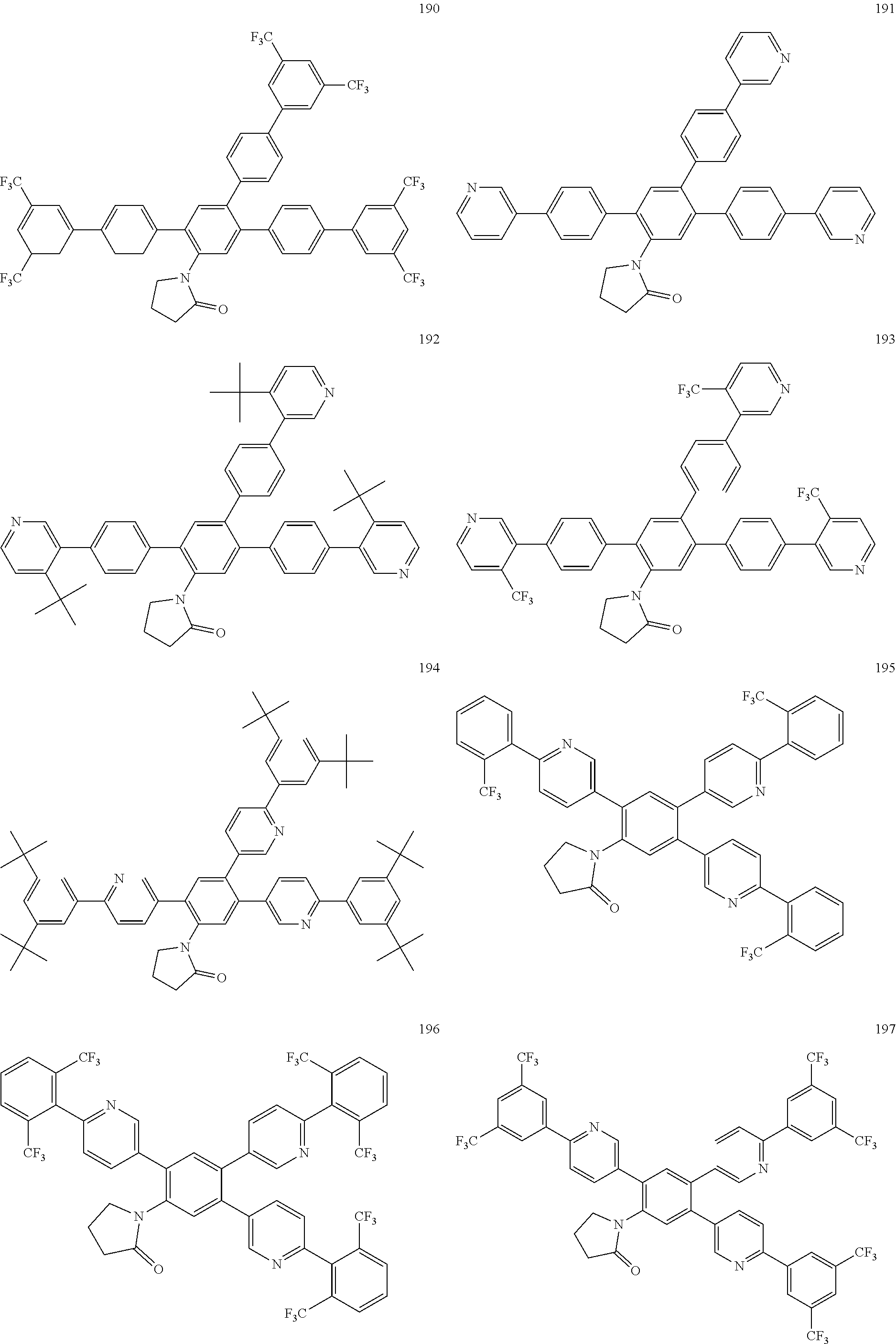

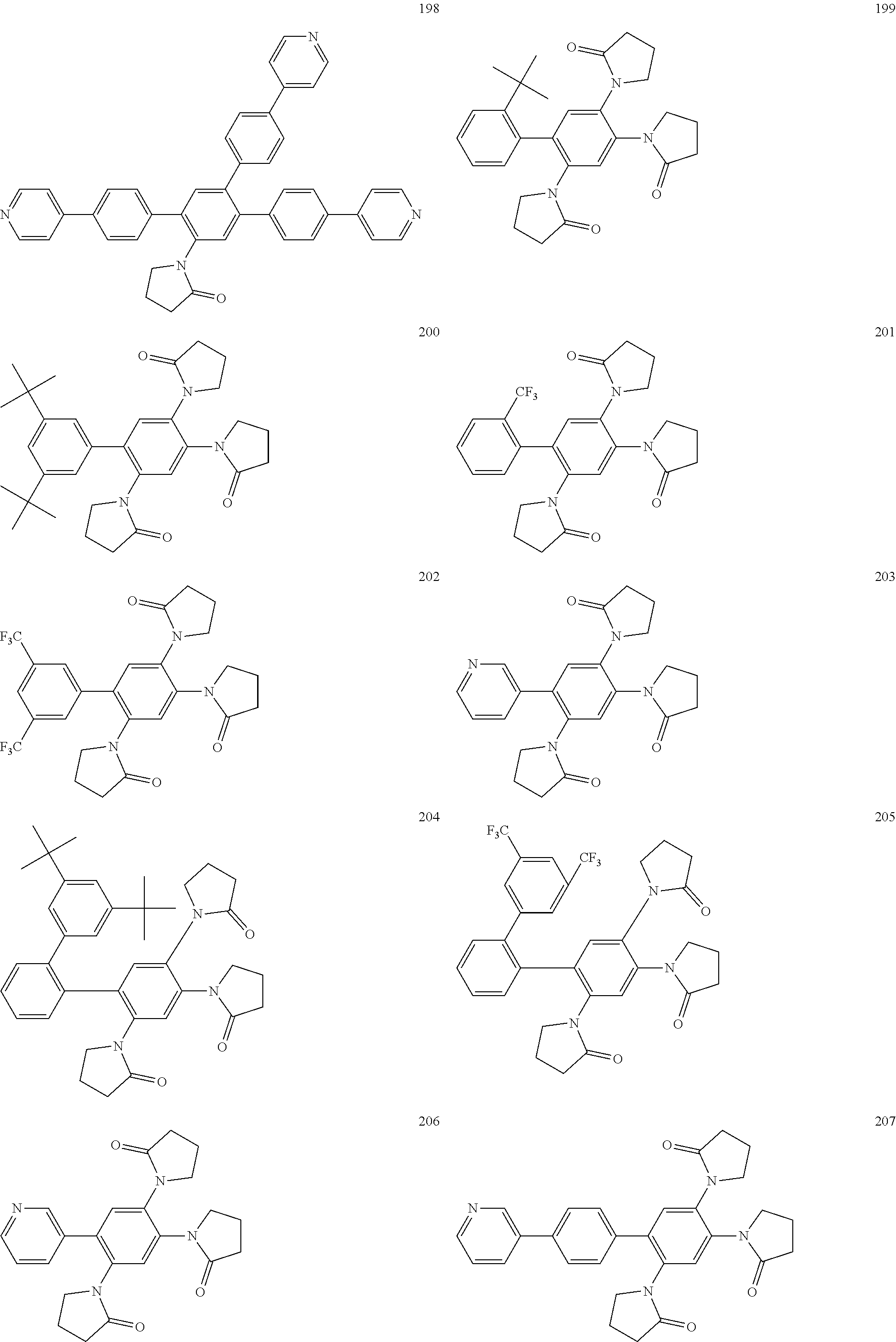

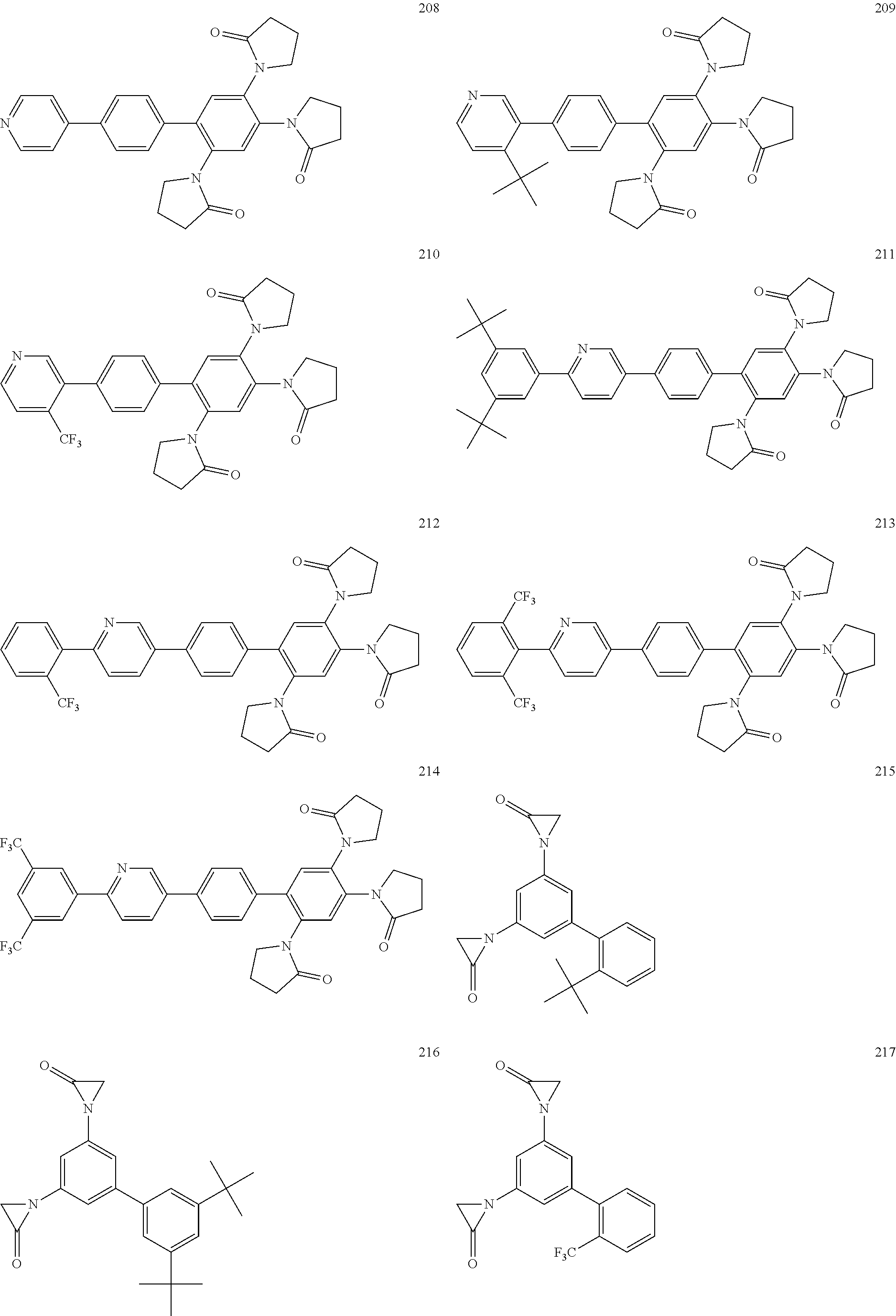

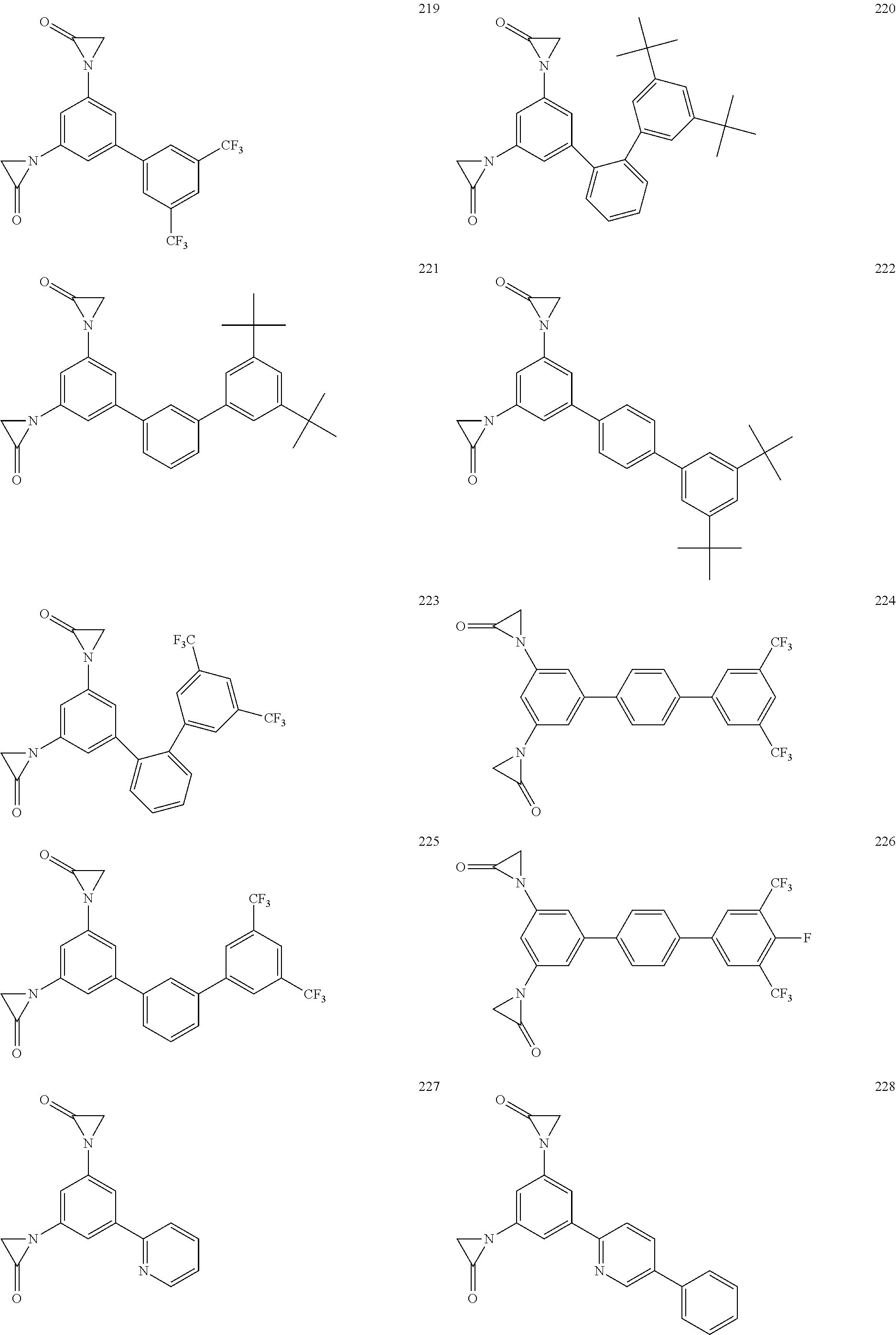

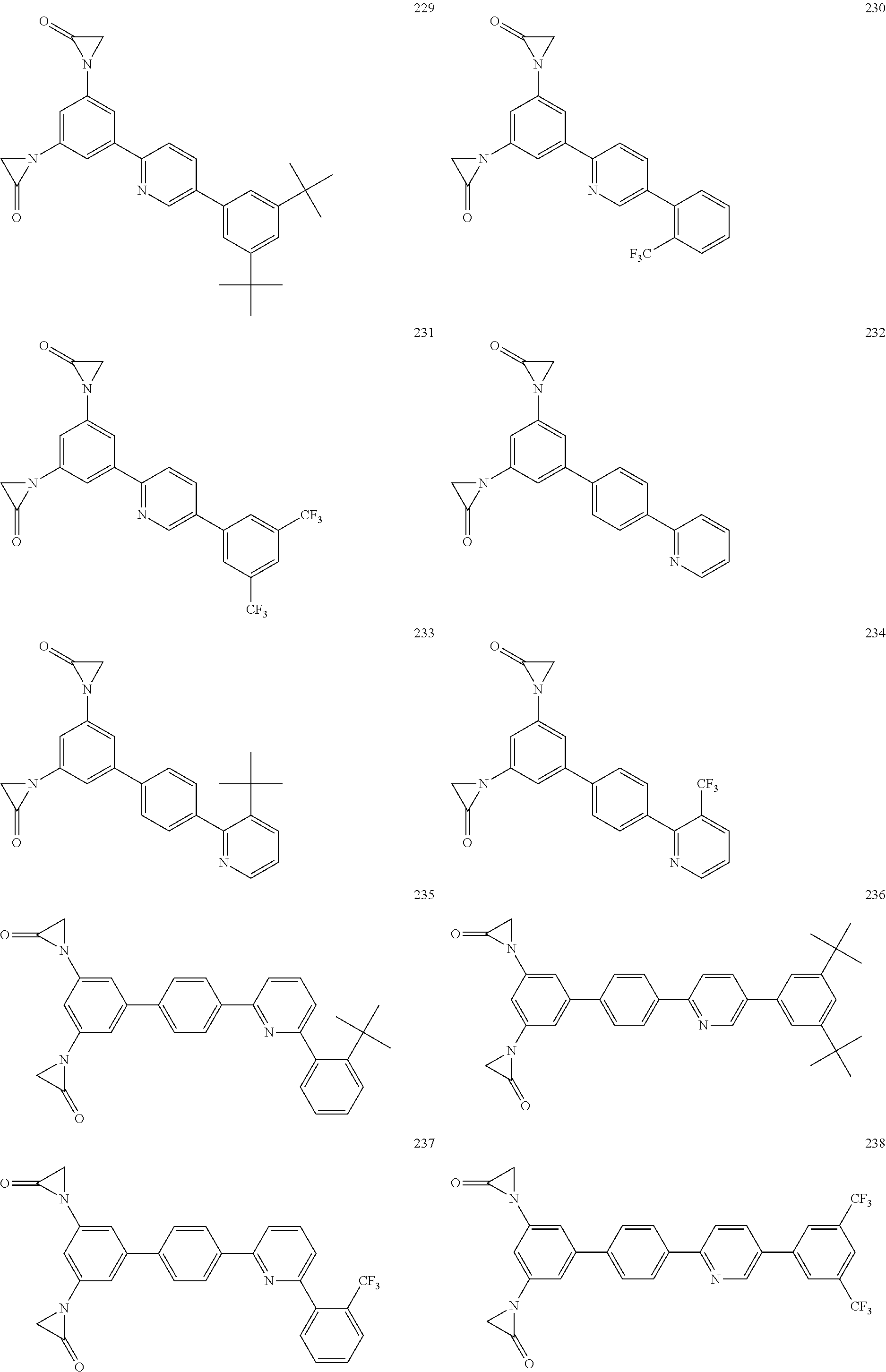

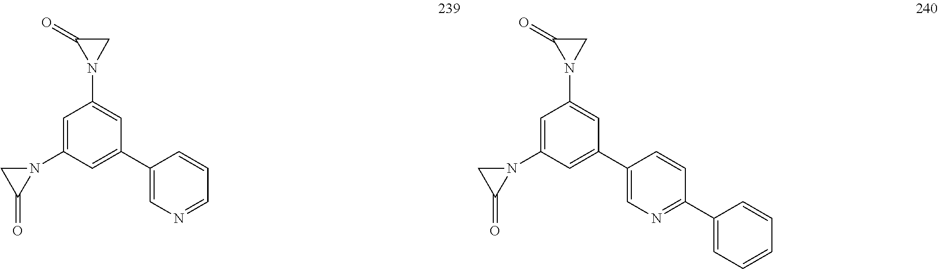 [SMILES: O=C1CN1c1cc(-c2cccnc2)cc(N2CC2=O)c1 ; O=C1CN1c1cc(-c2ccc(-c3ccccc3)nc2)cc(N2CC2=O)c1]

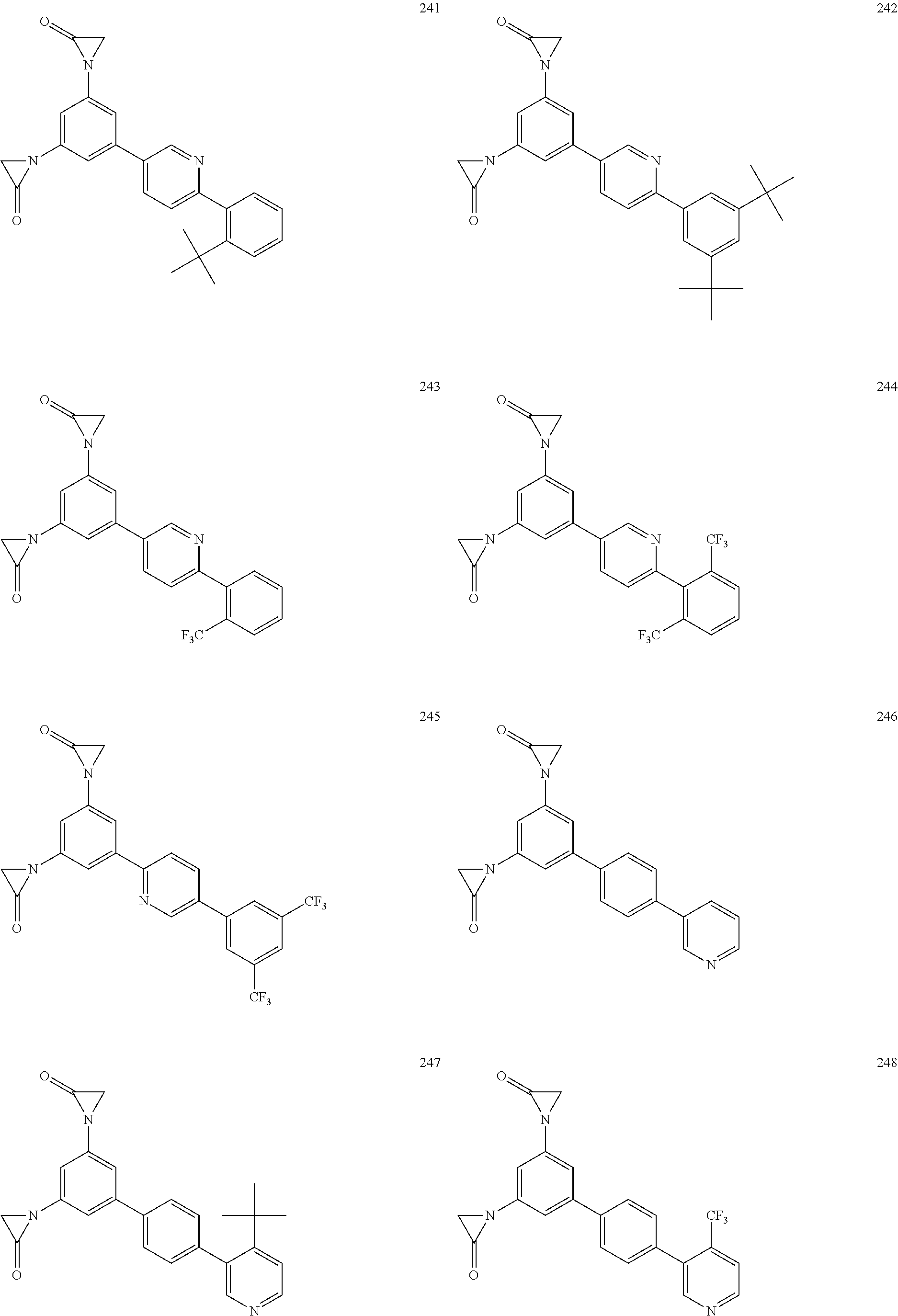

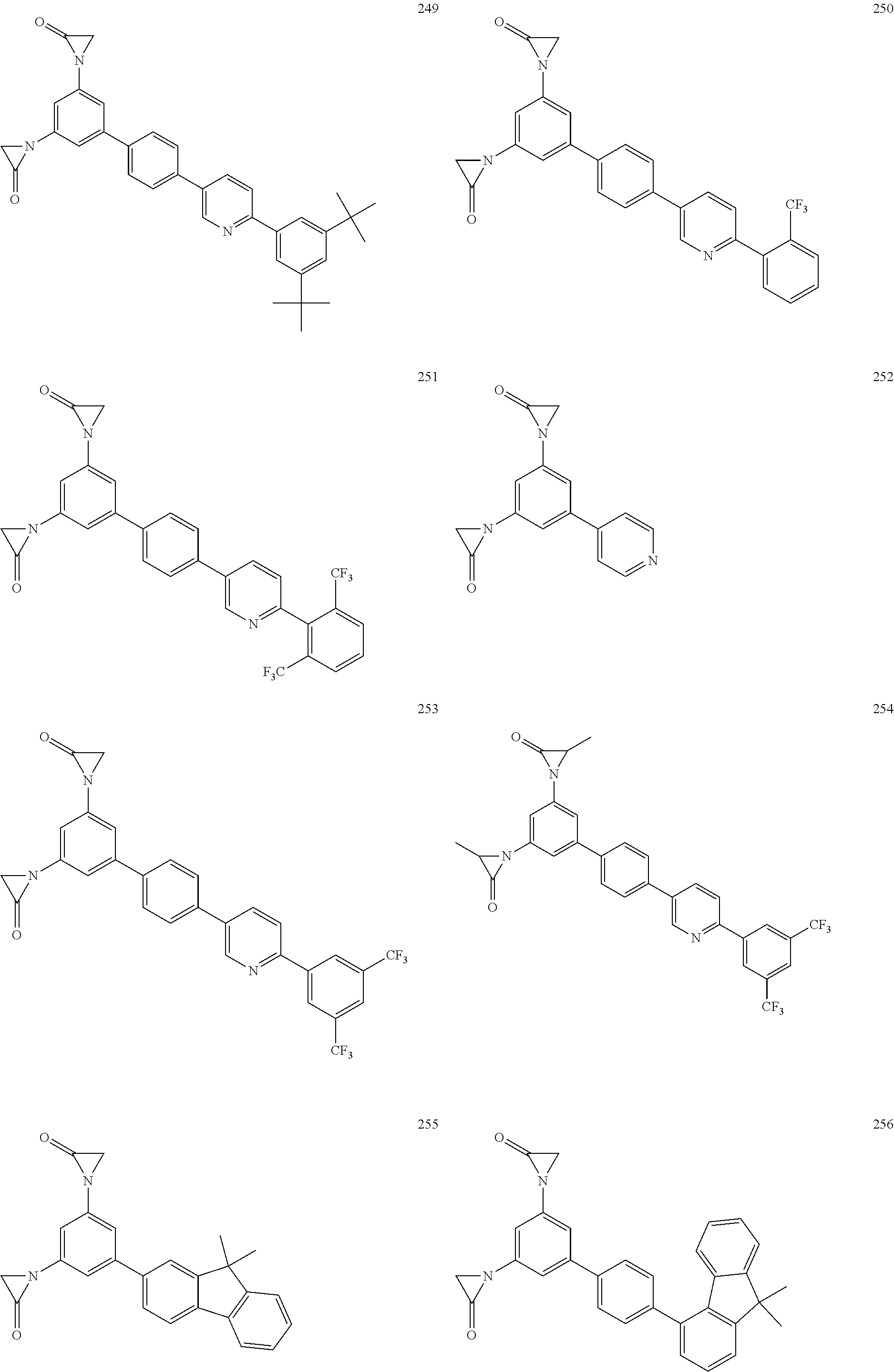

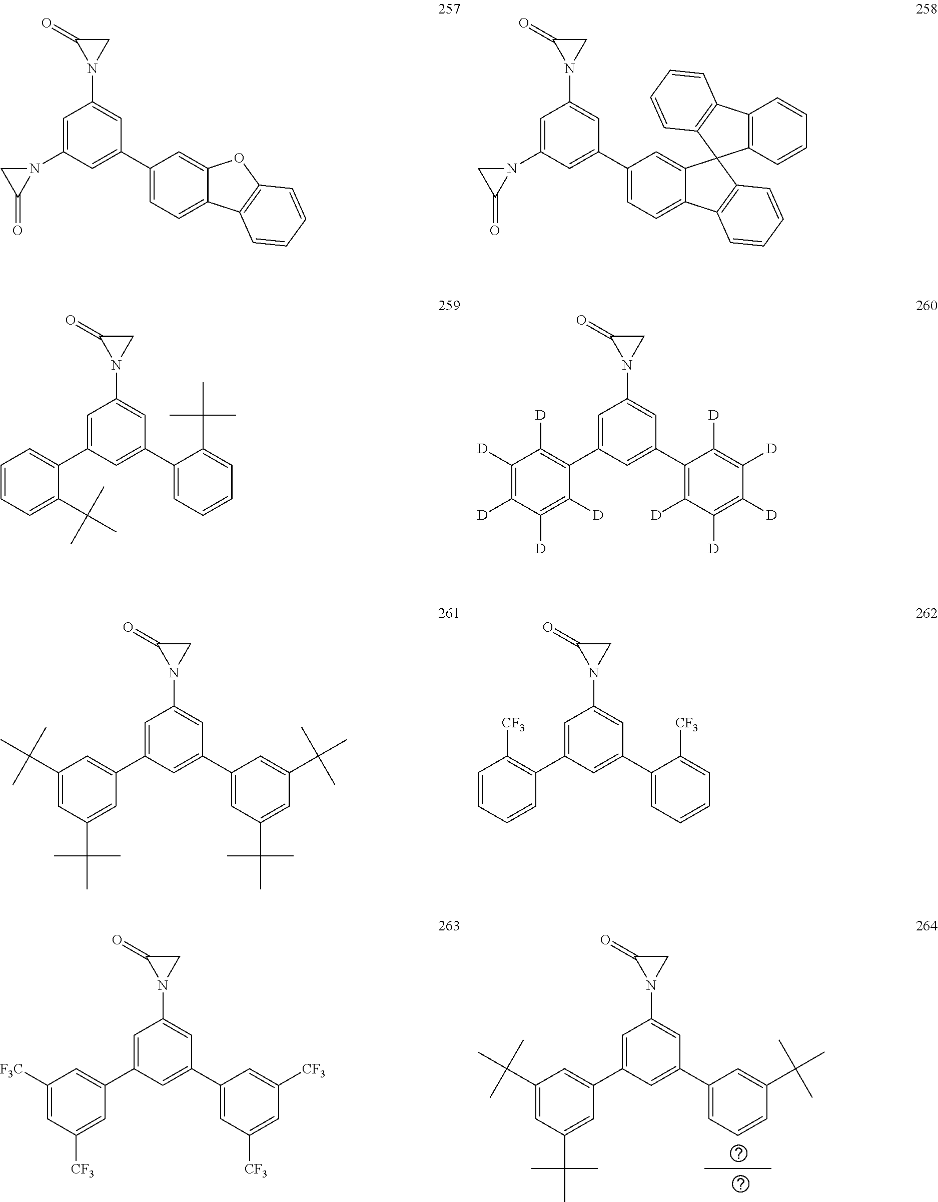

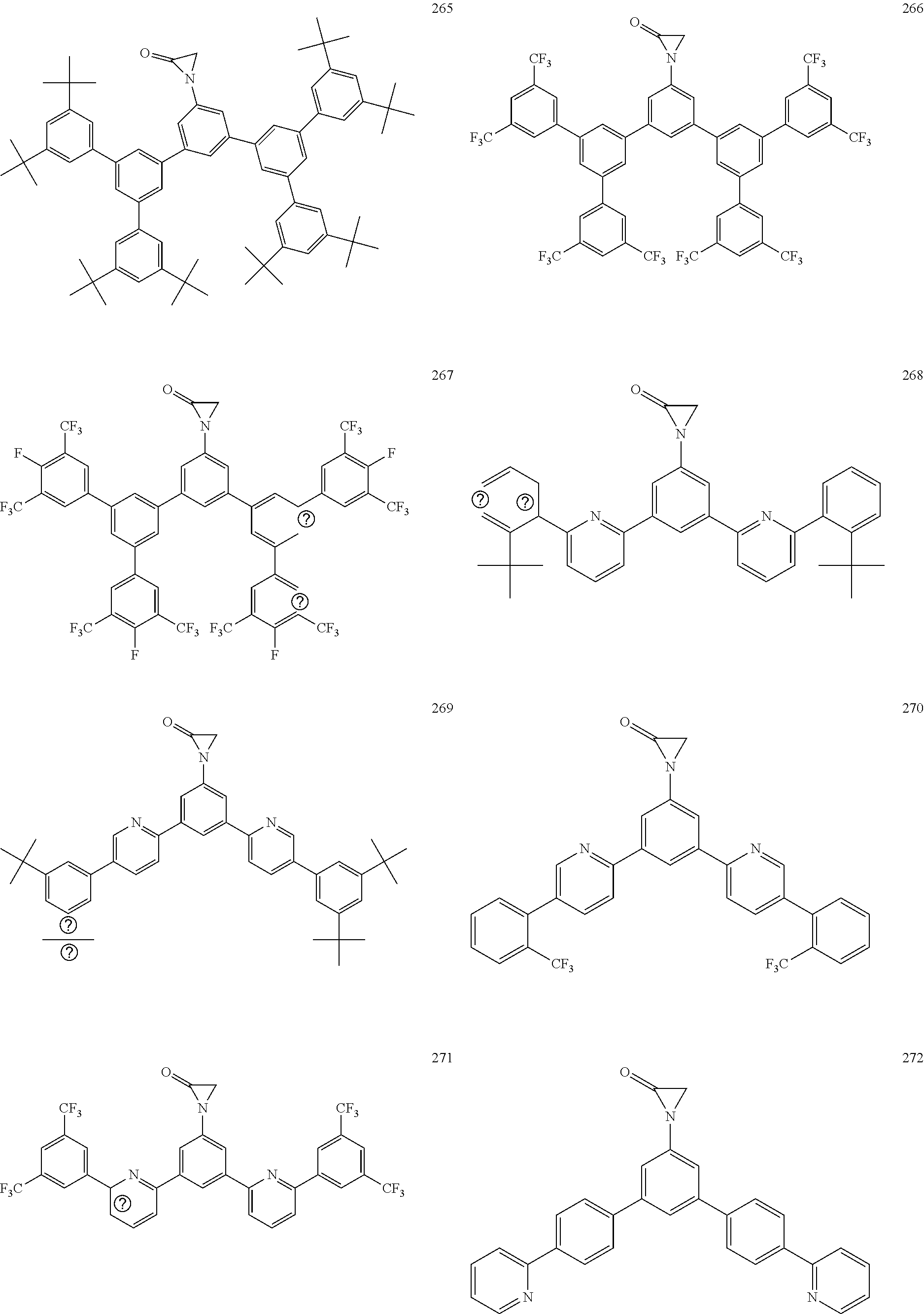

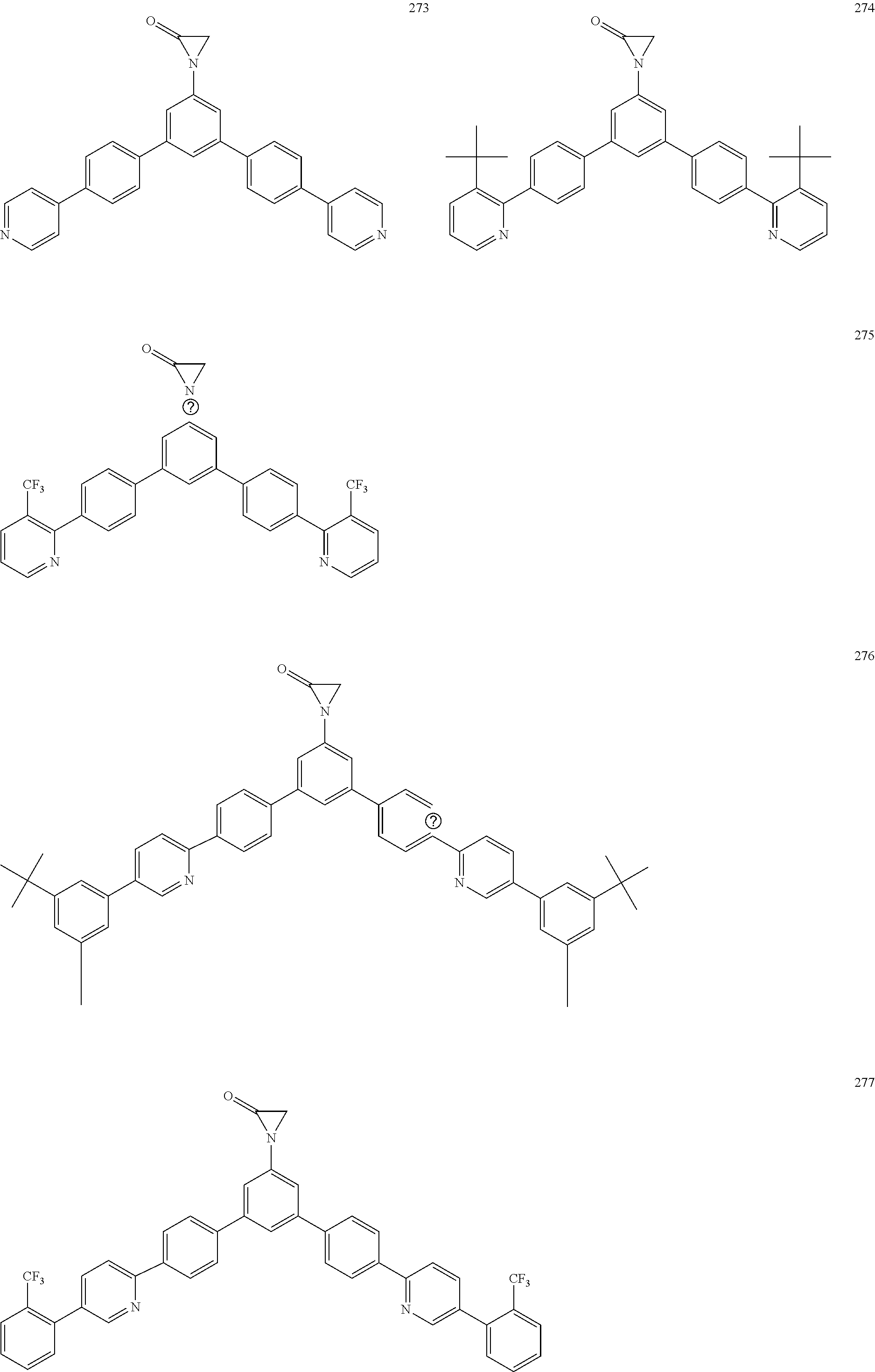

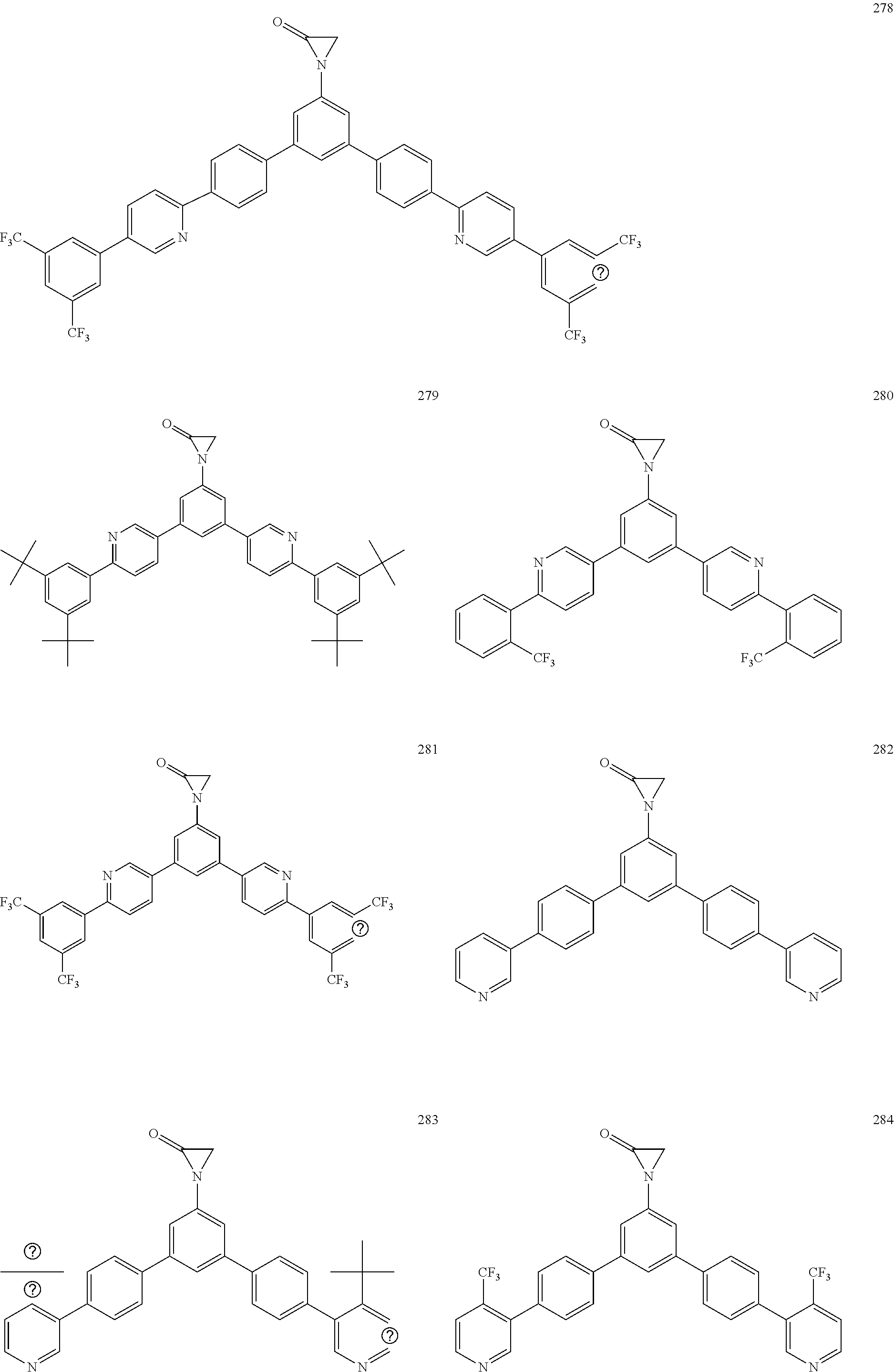

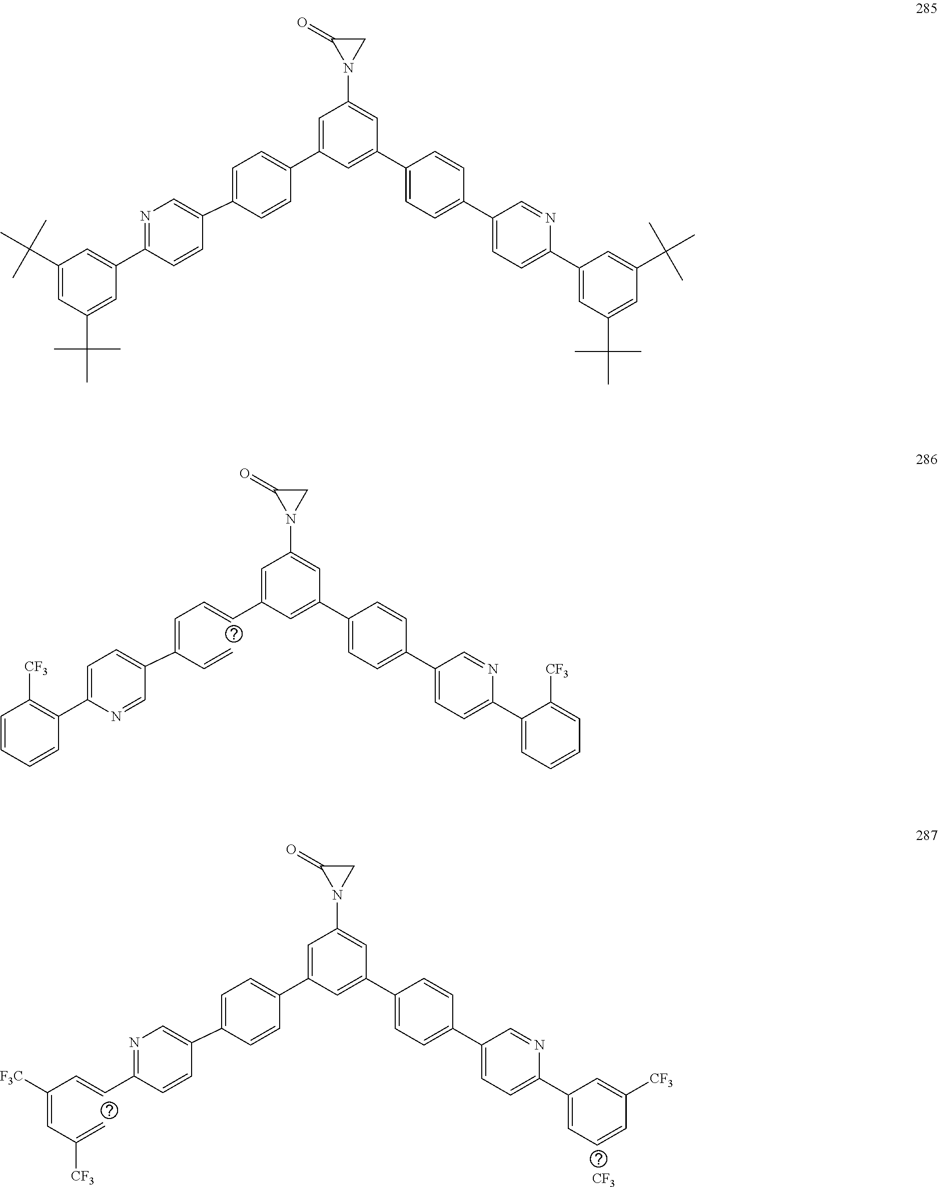

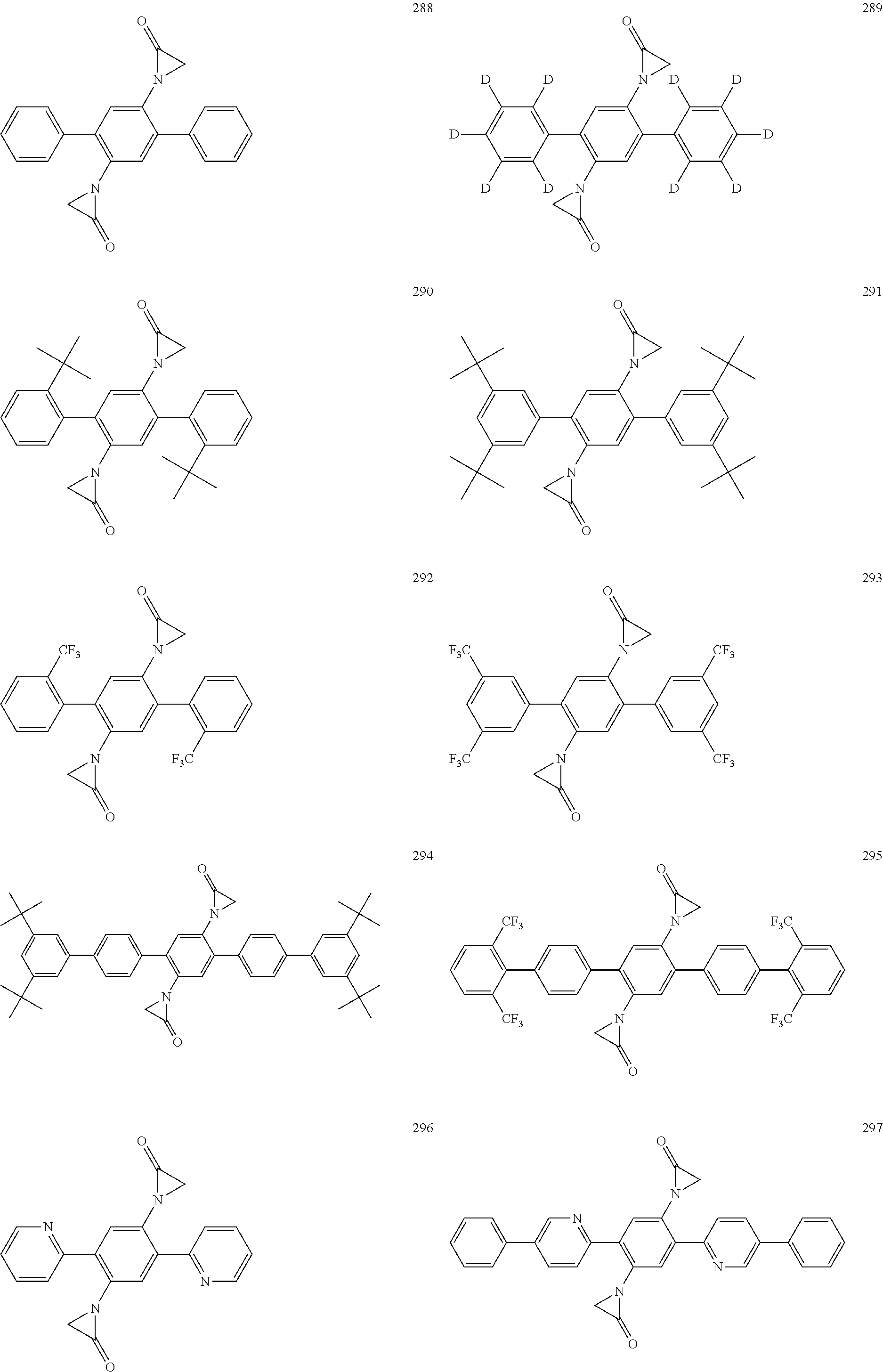

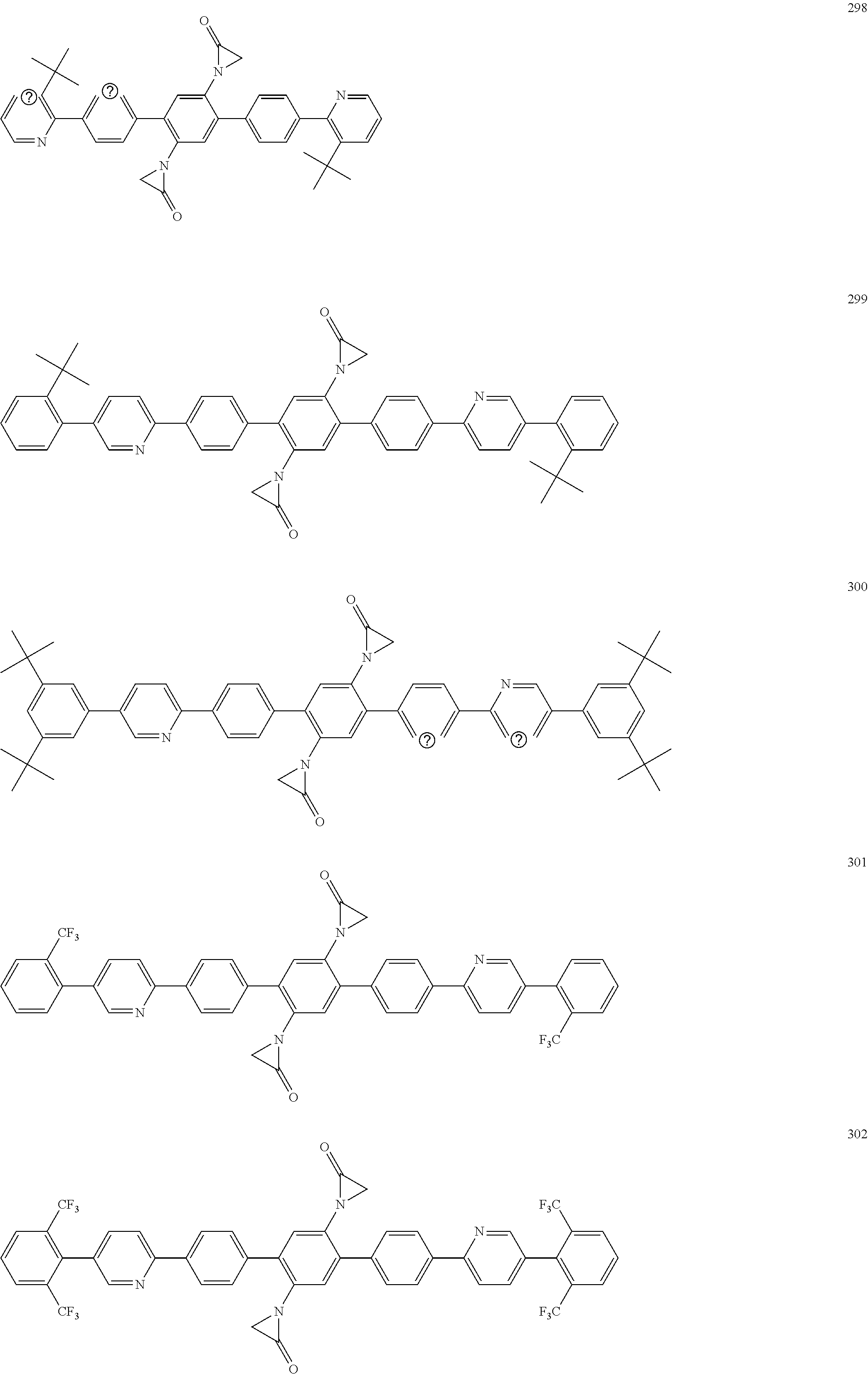

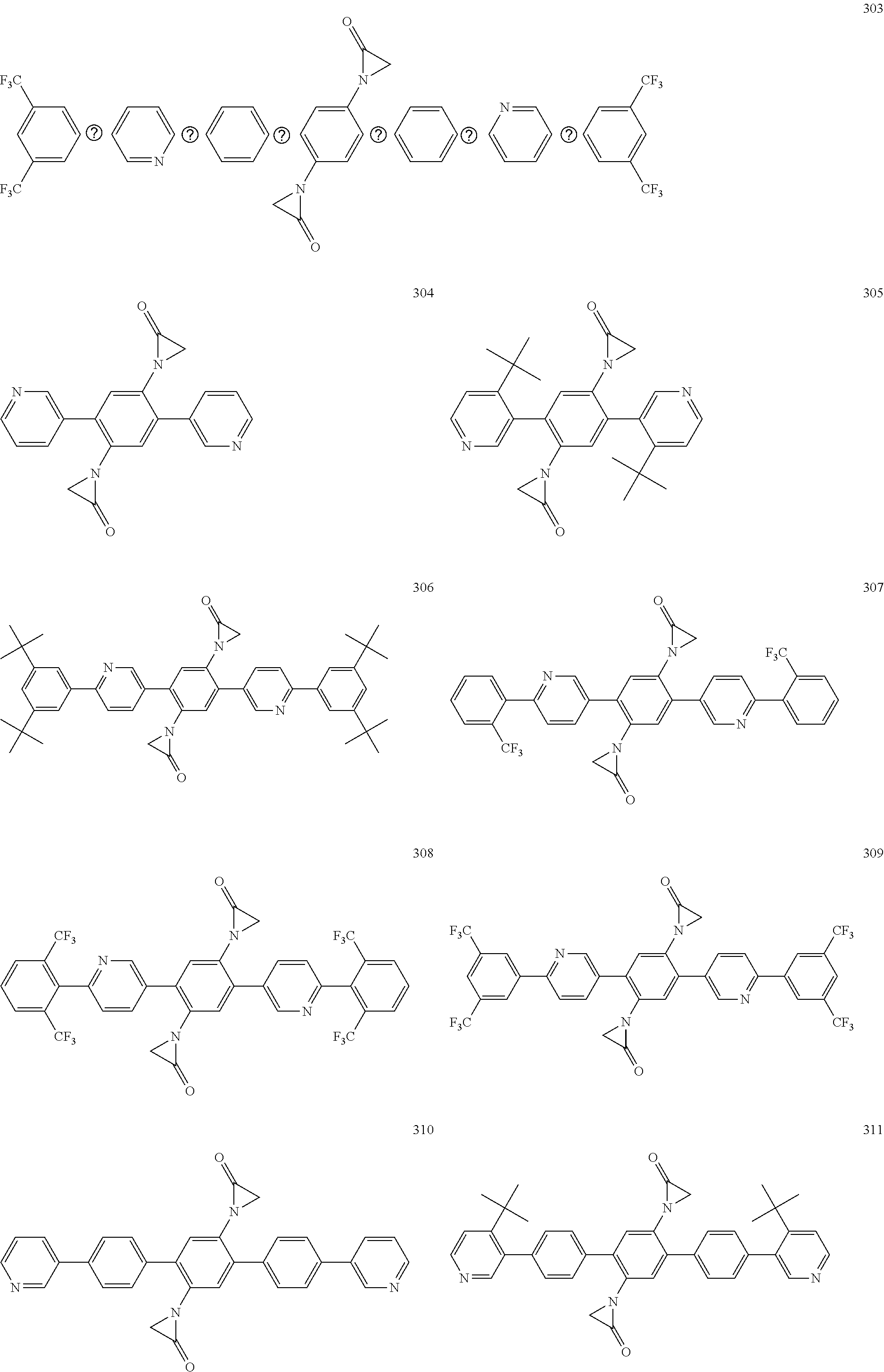

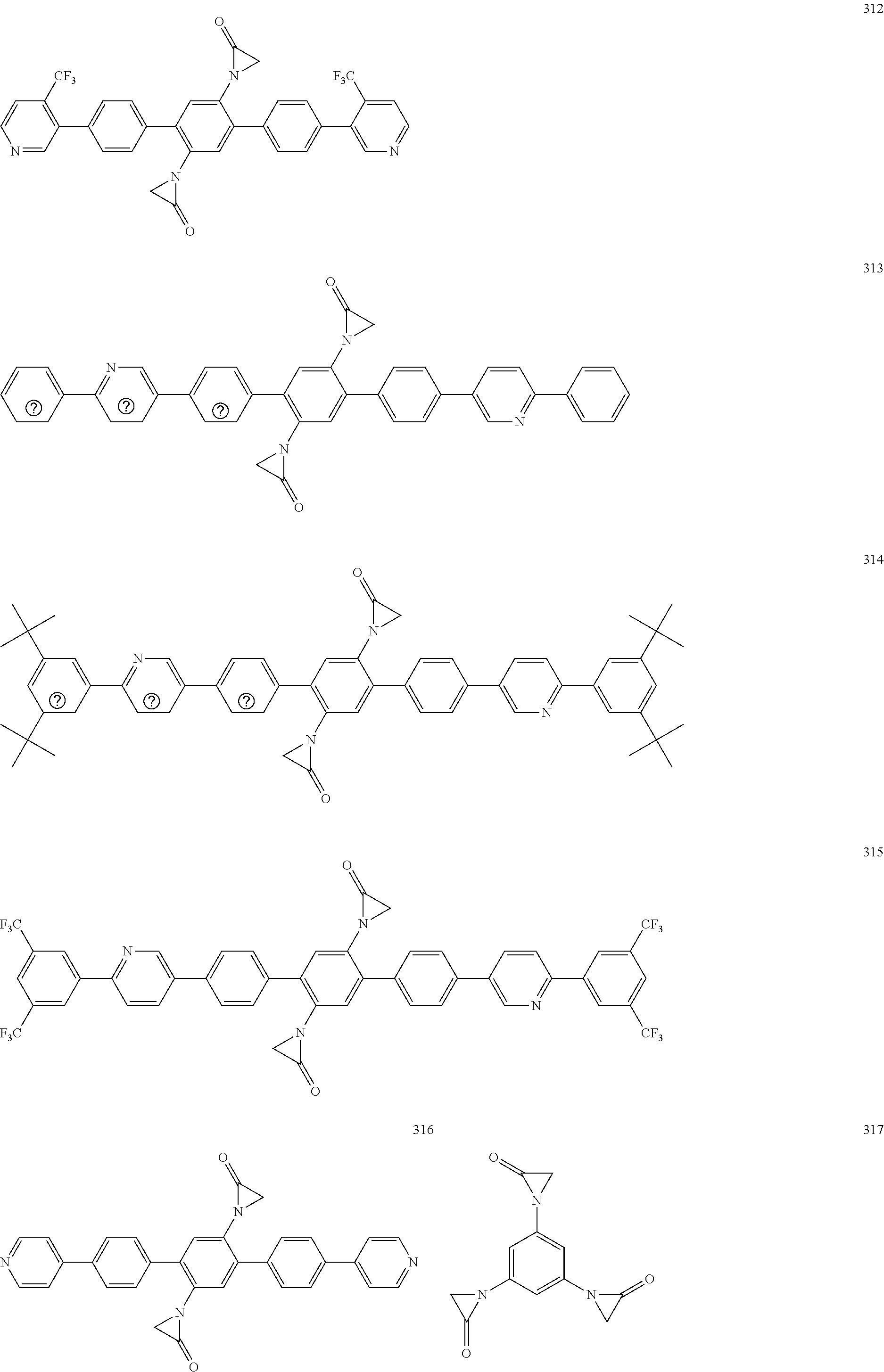

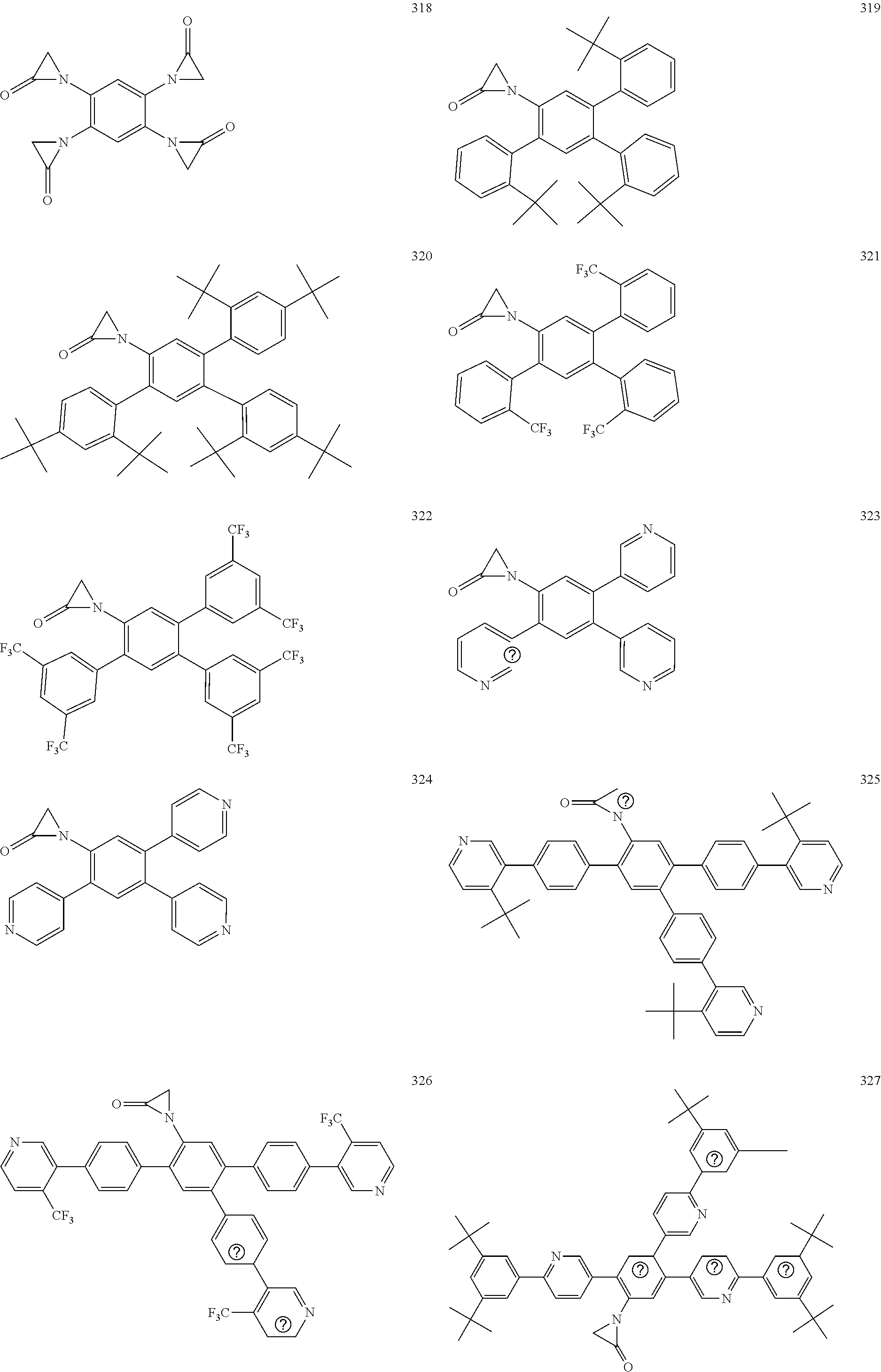

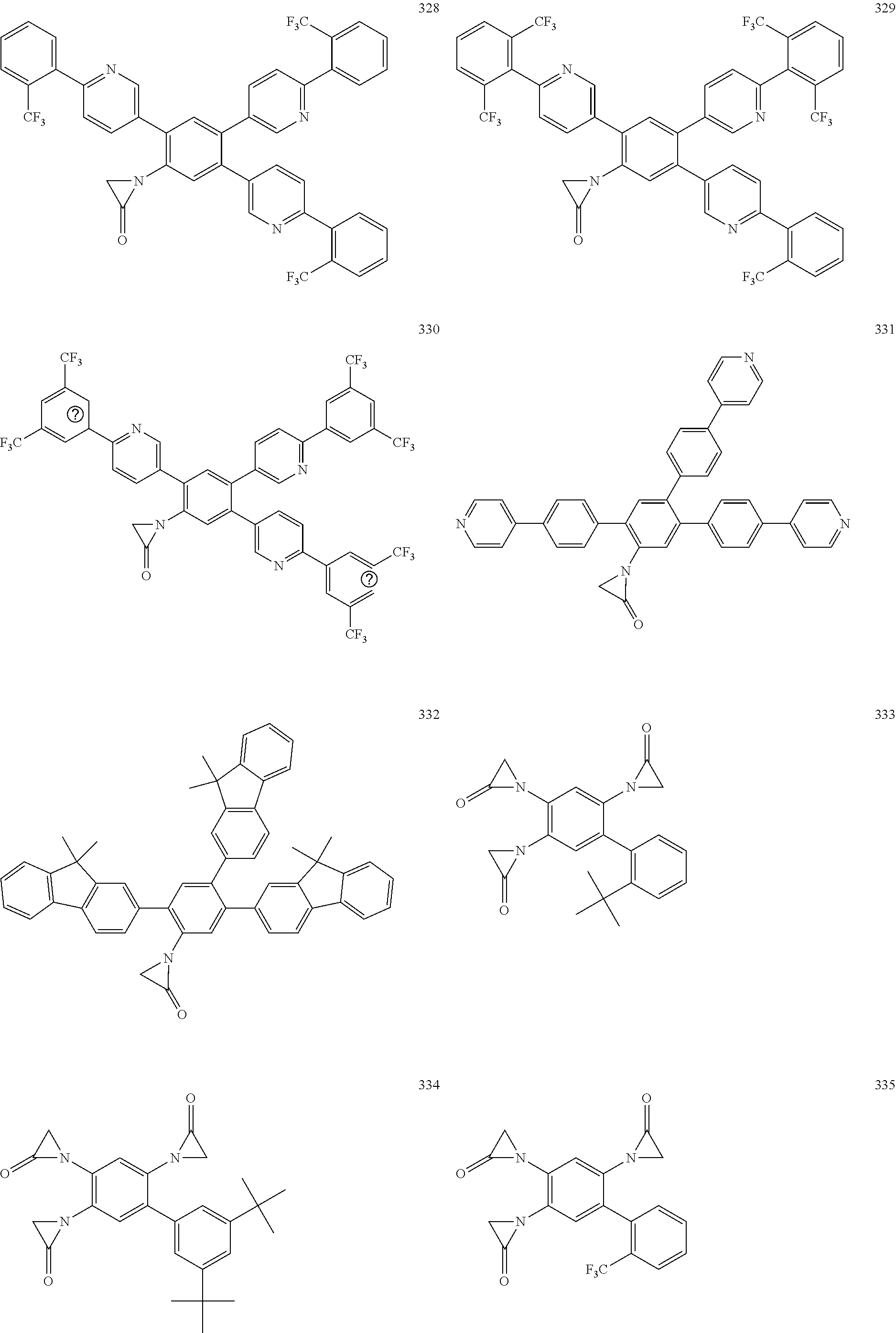

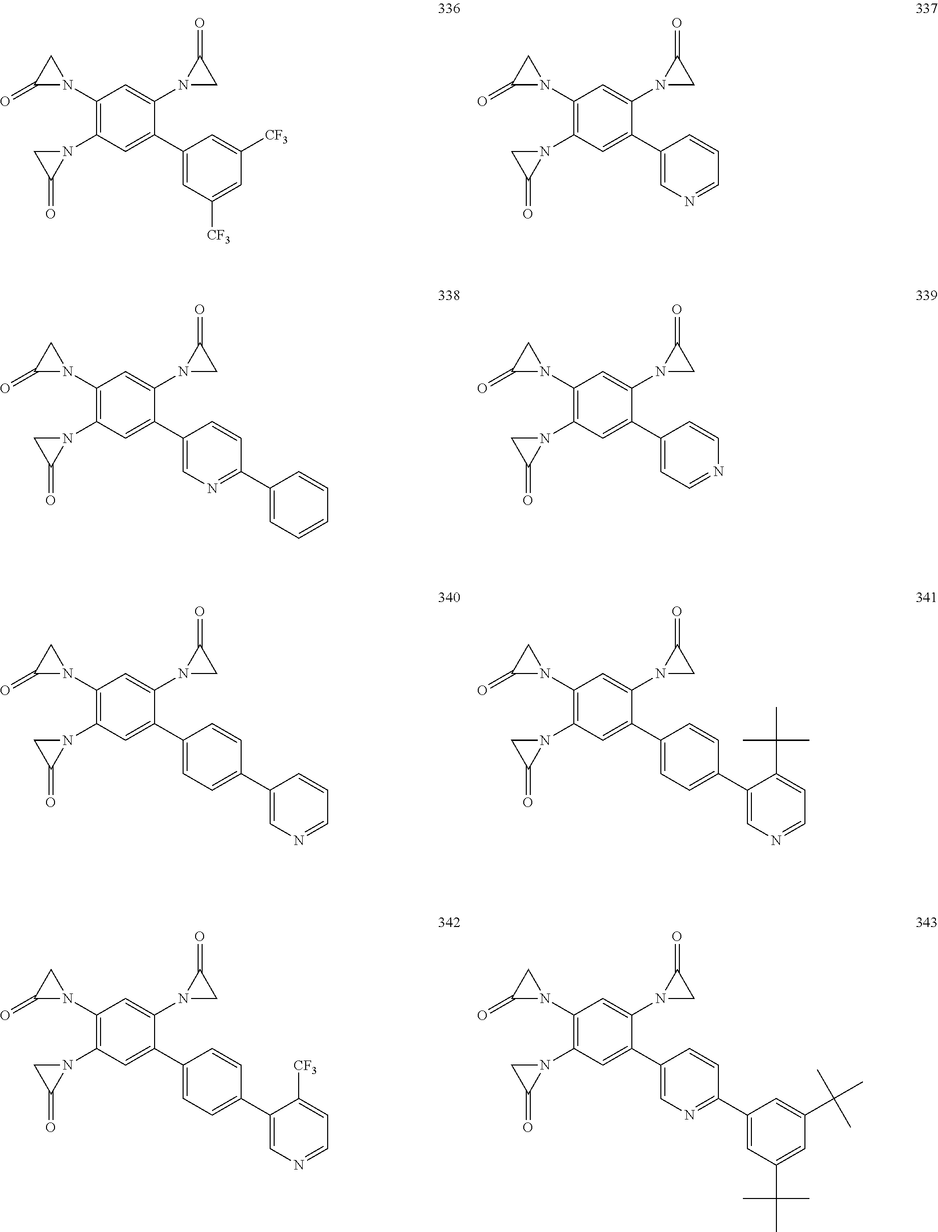

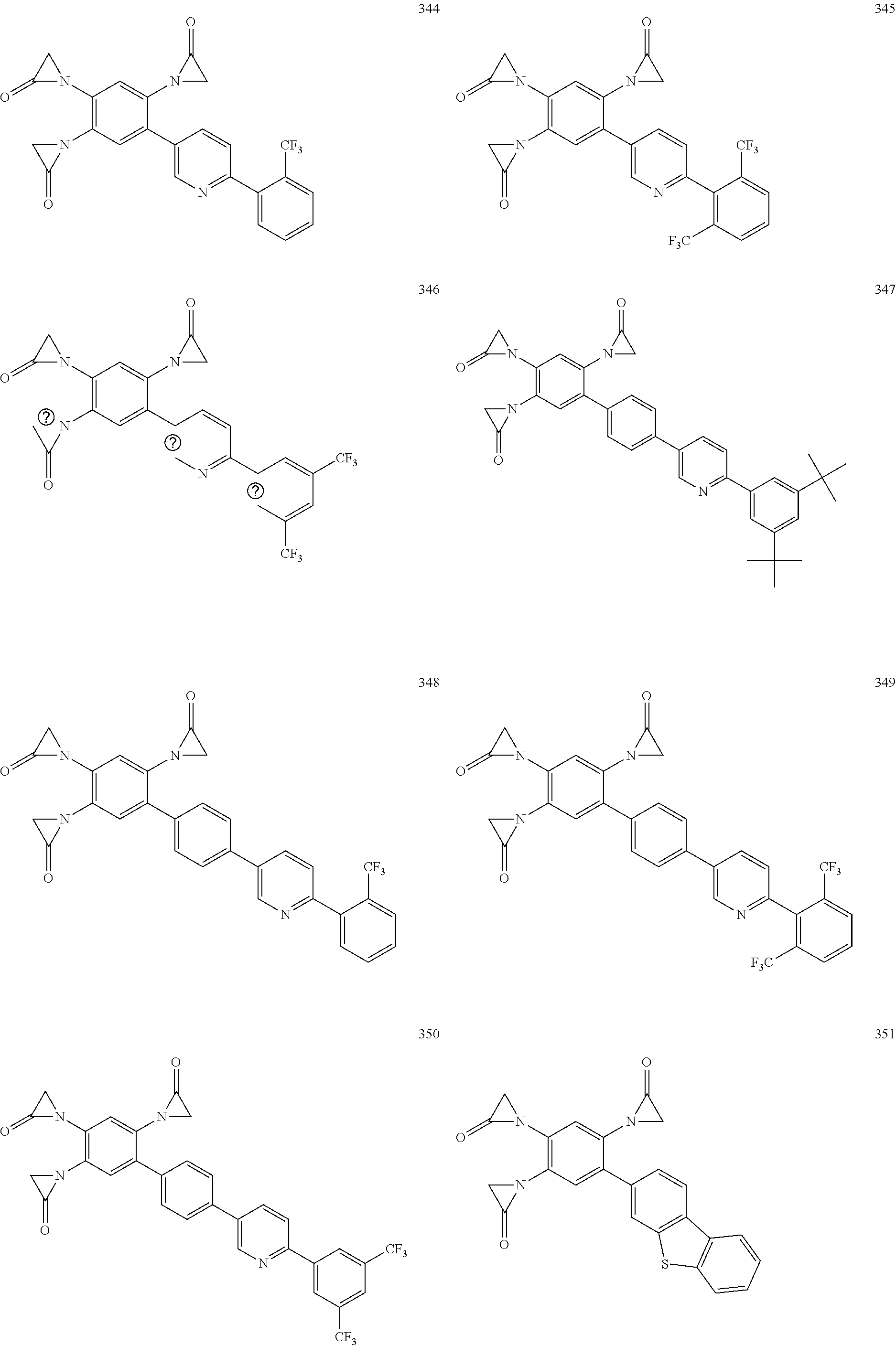

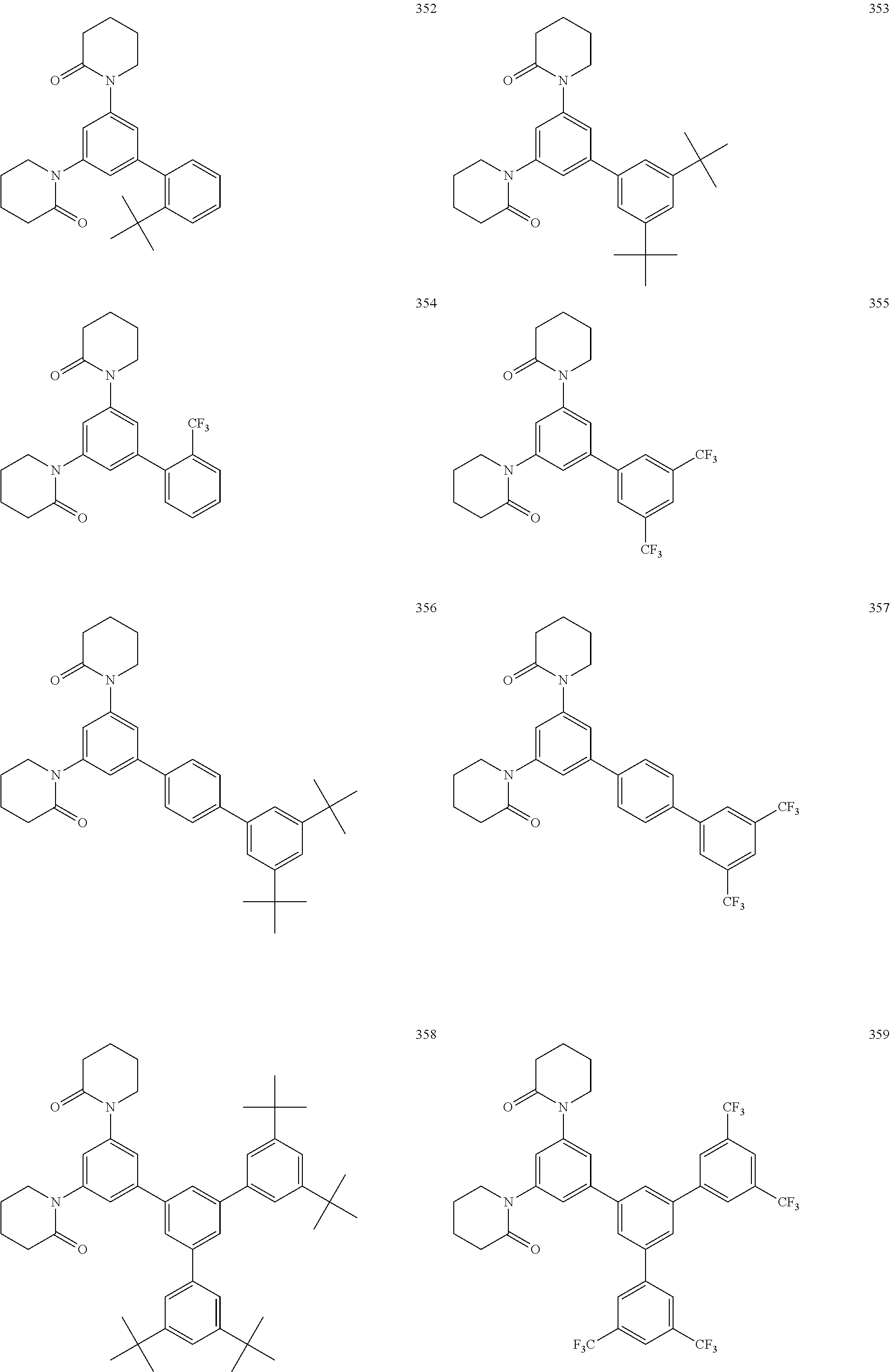

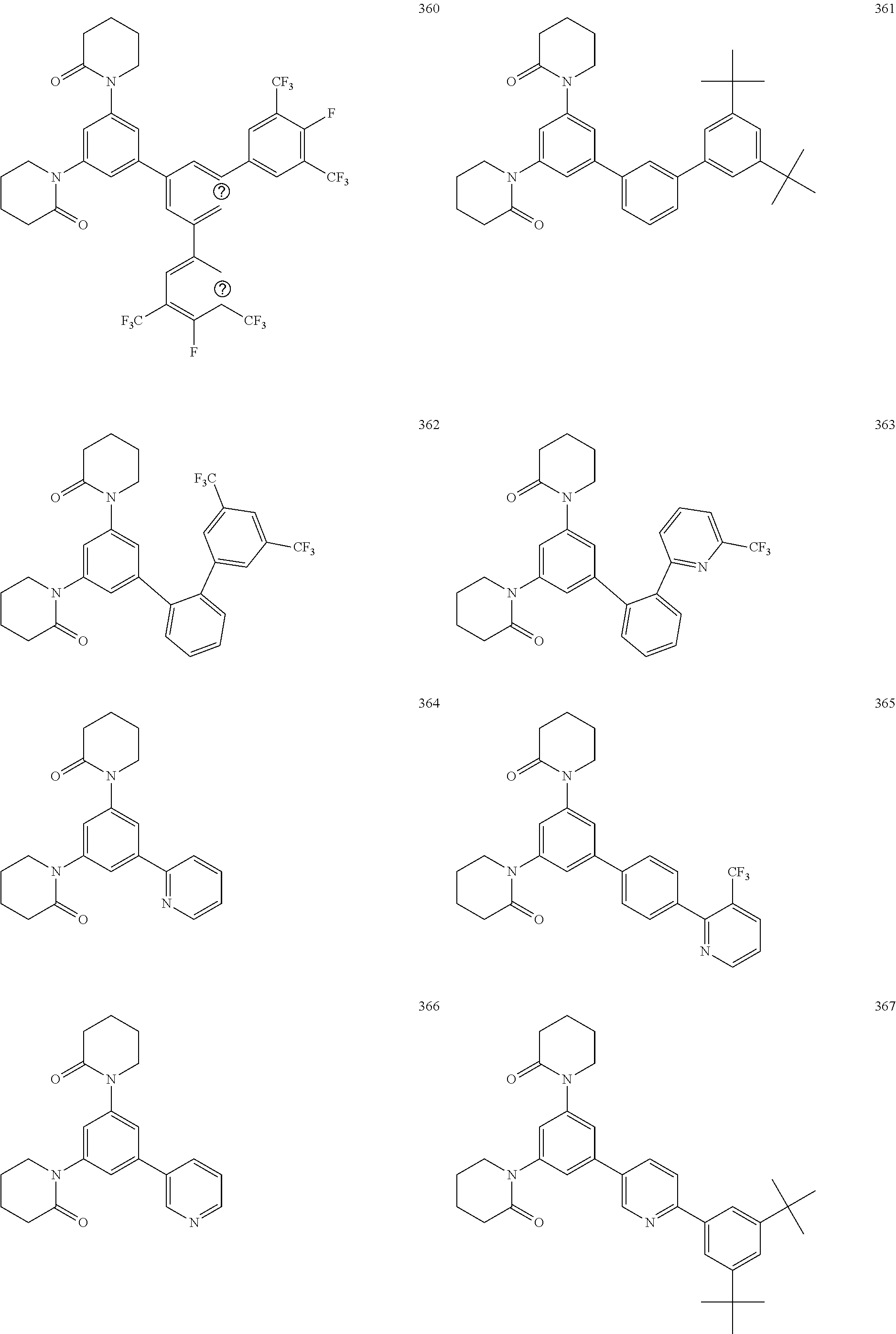

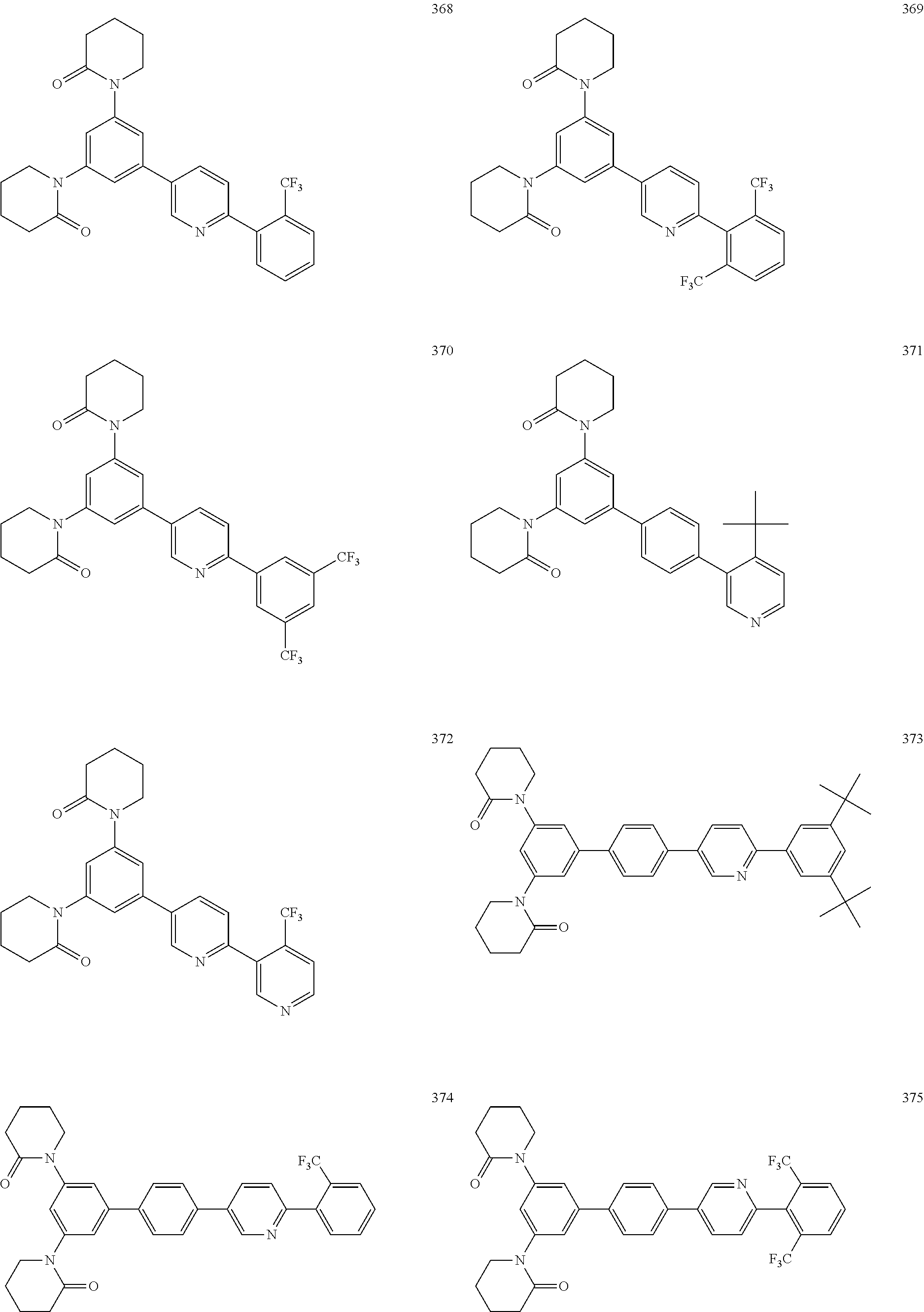

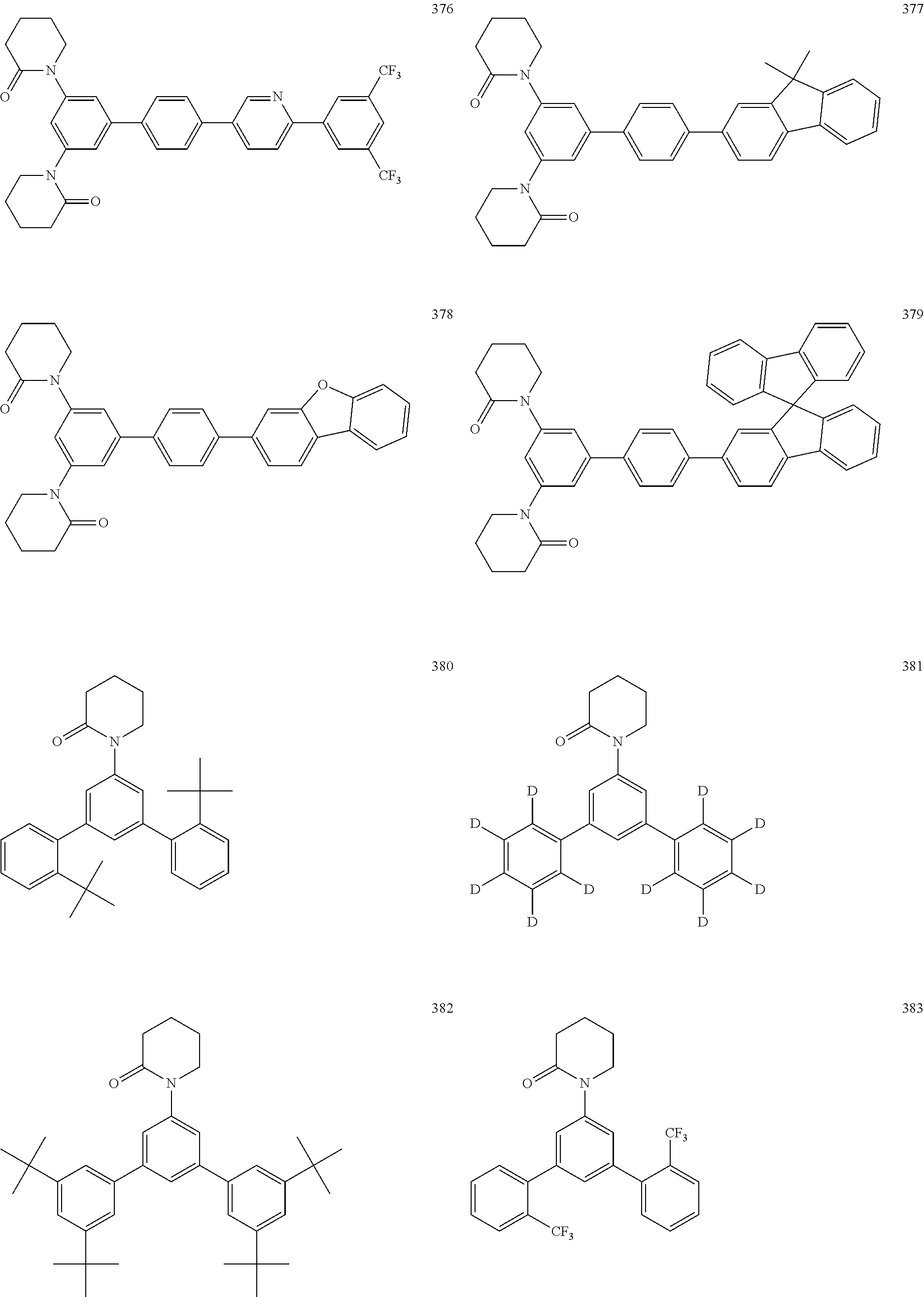

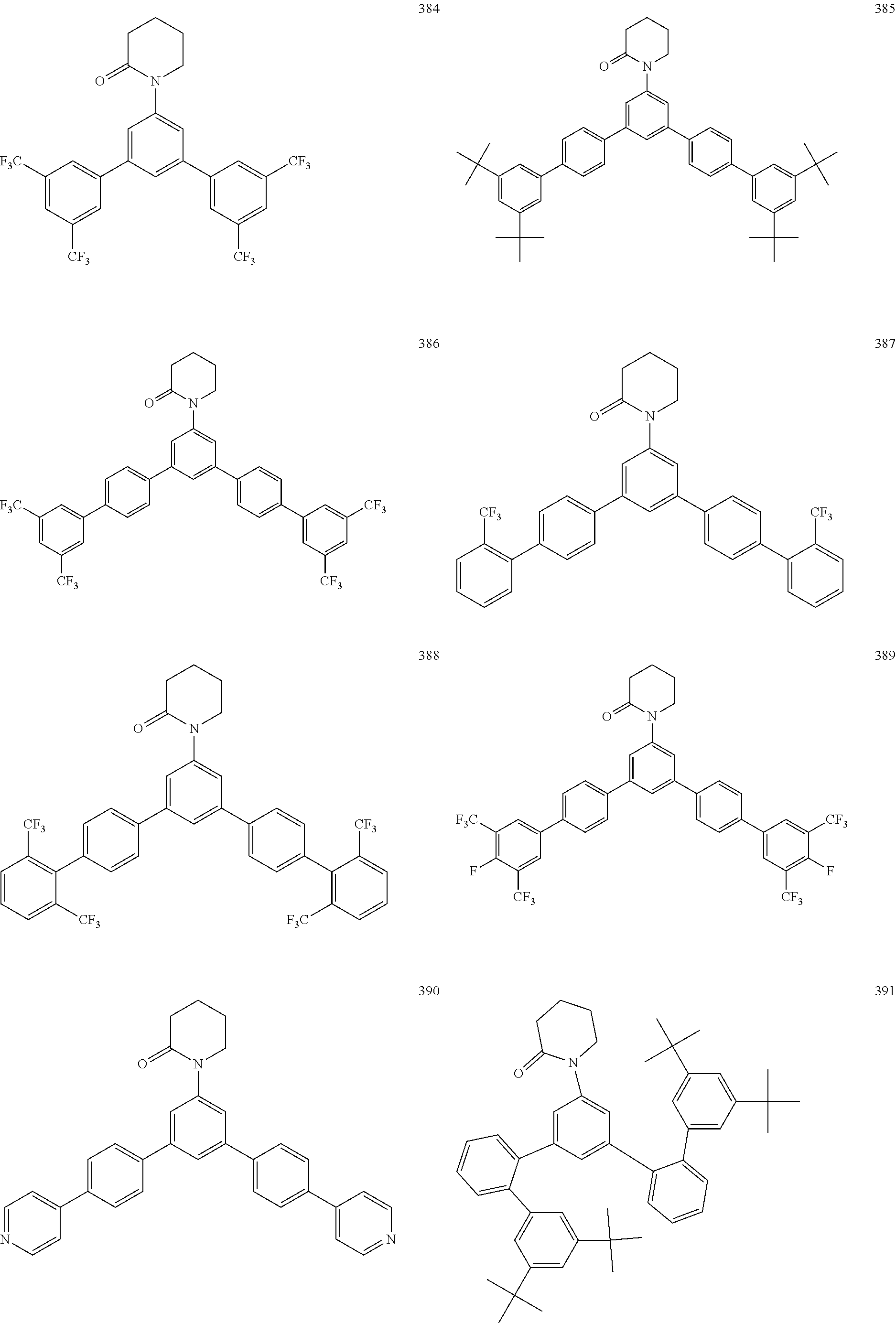

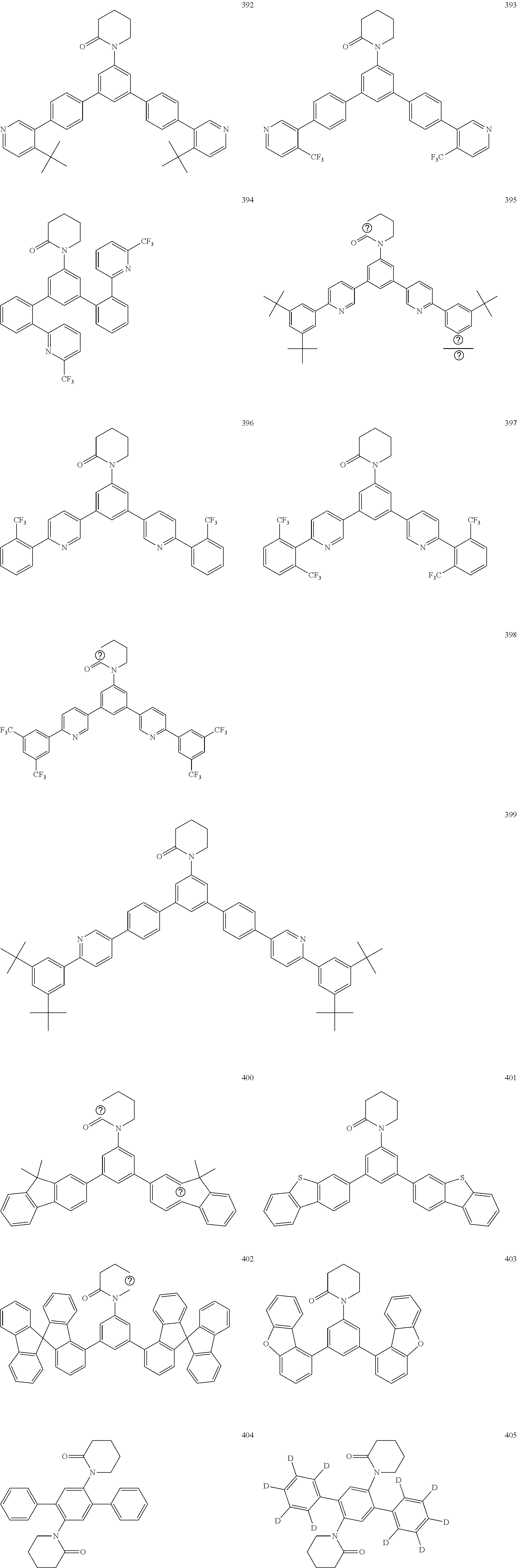

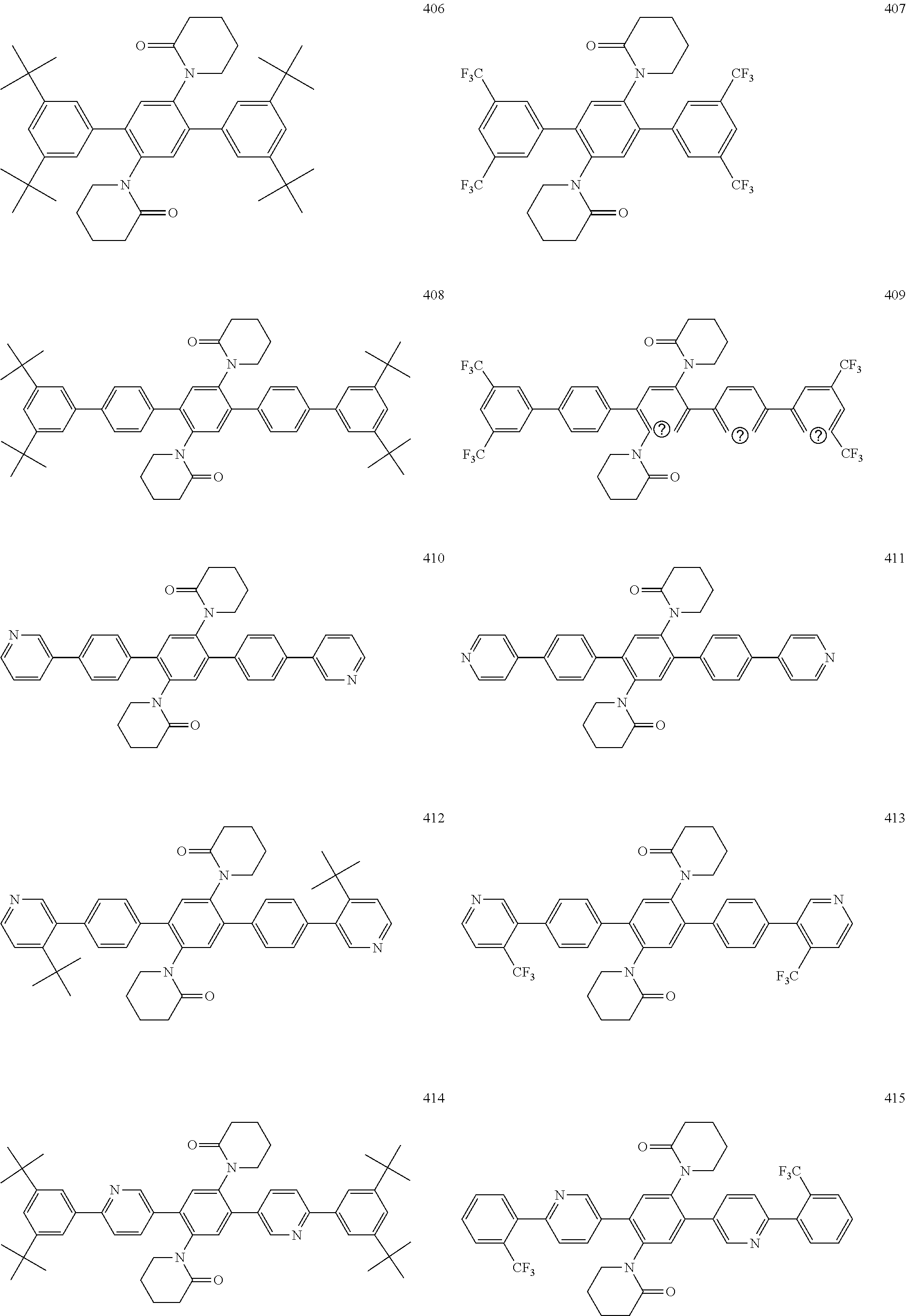

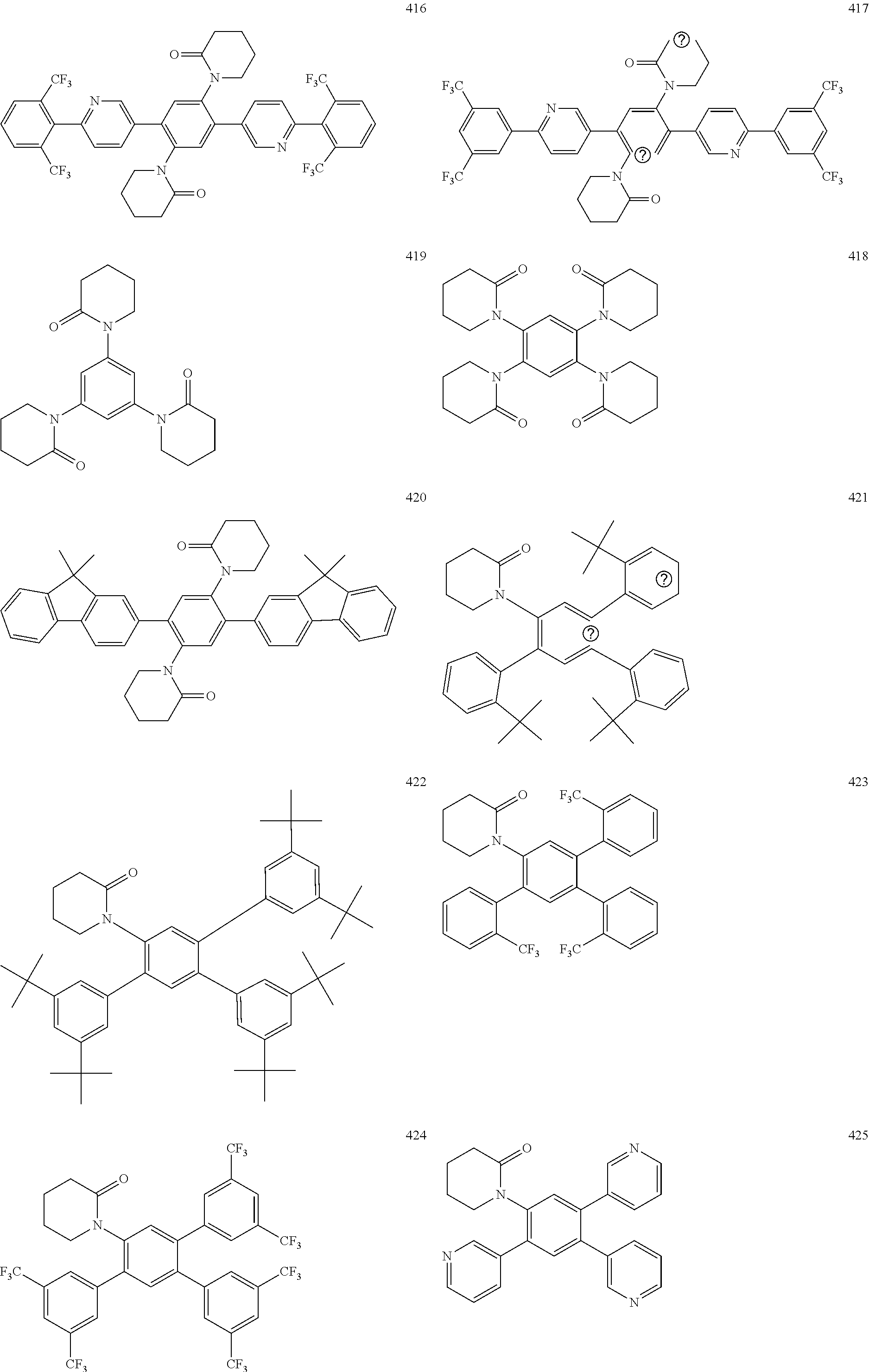

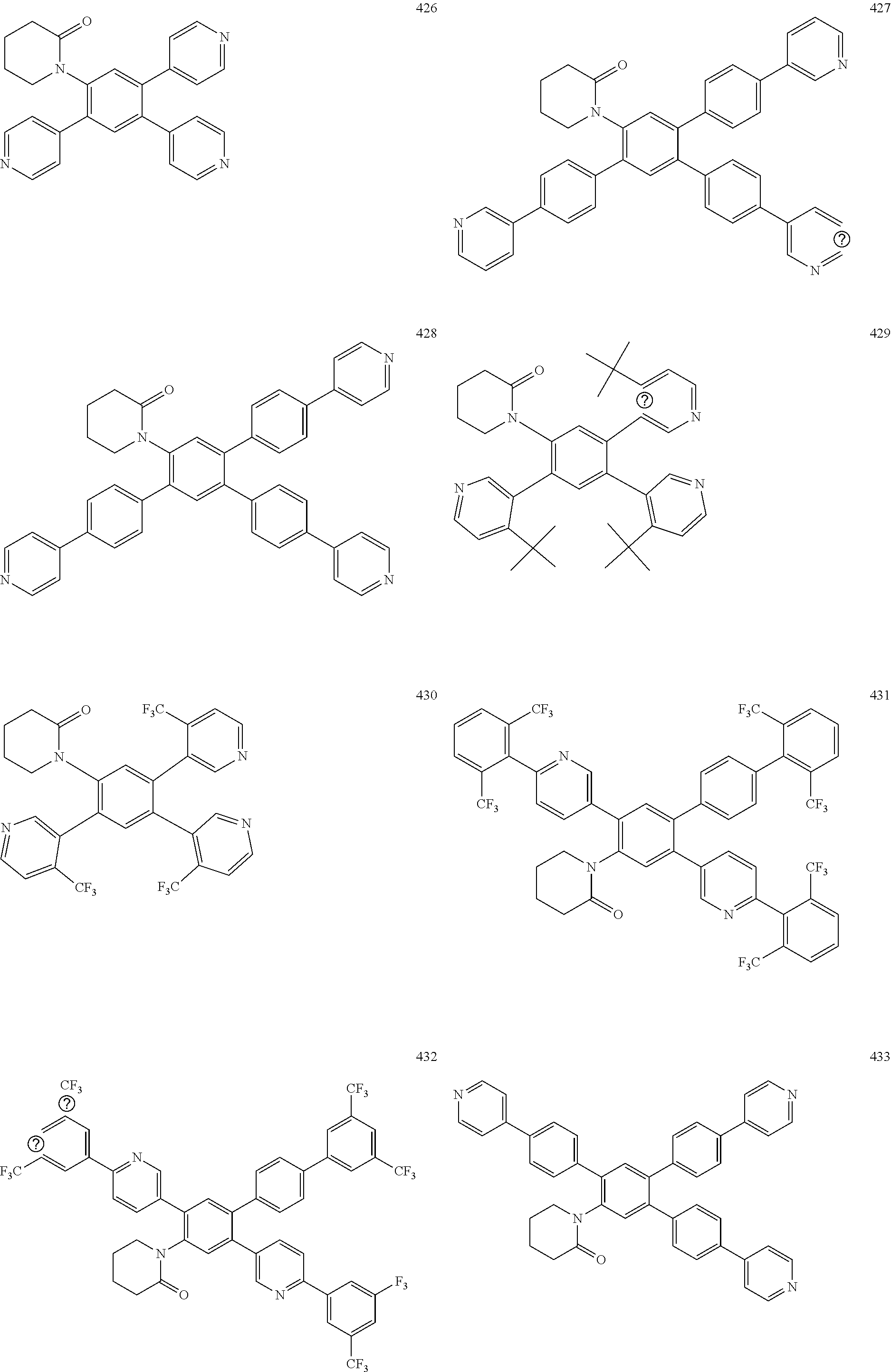

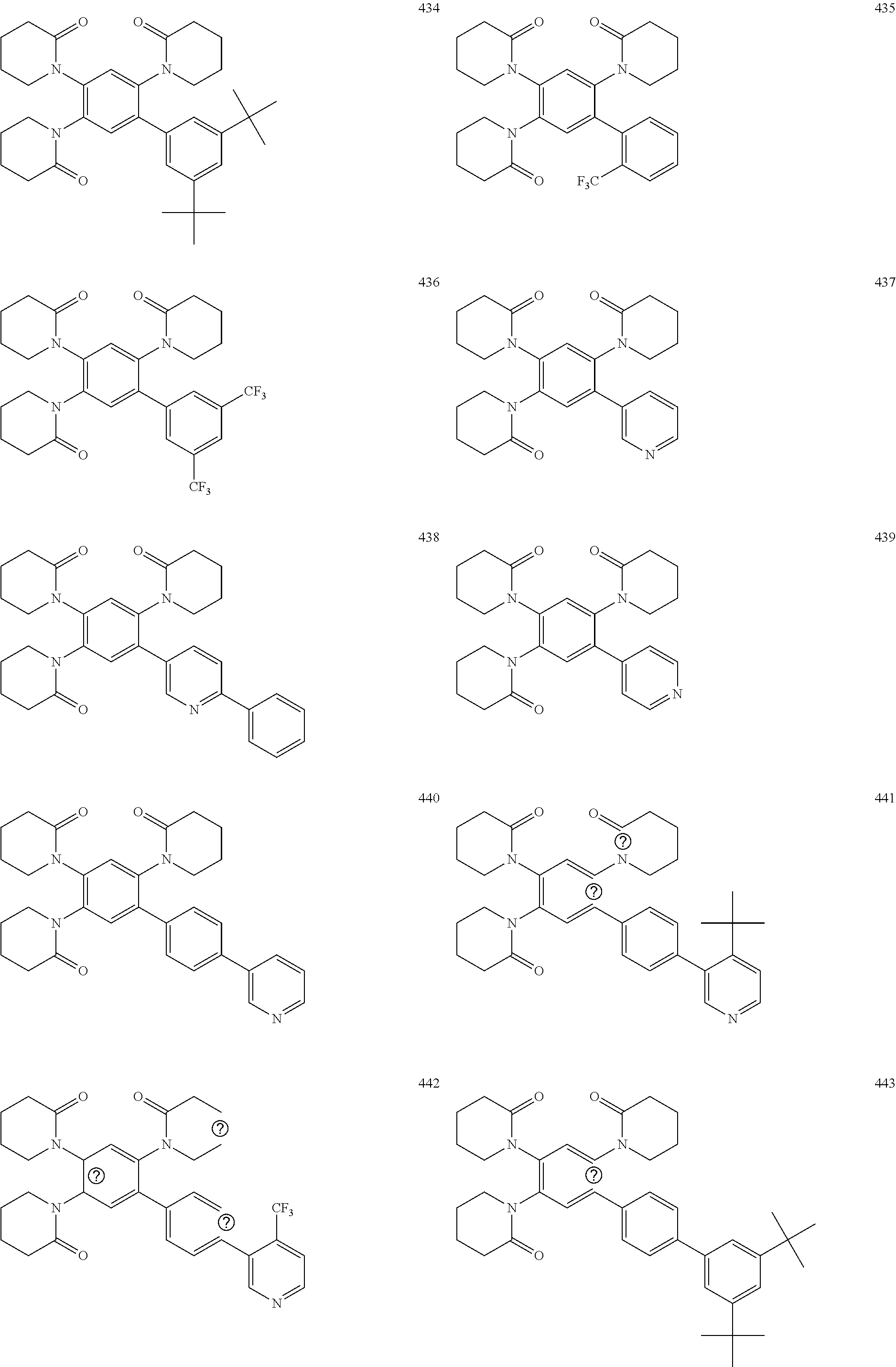

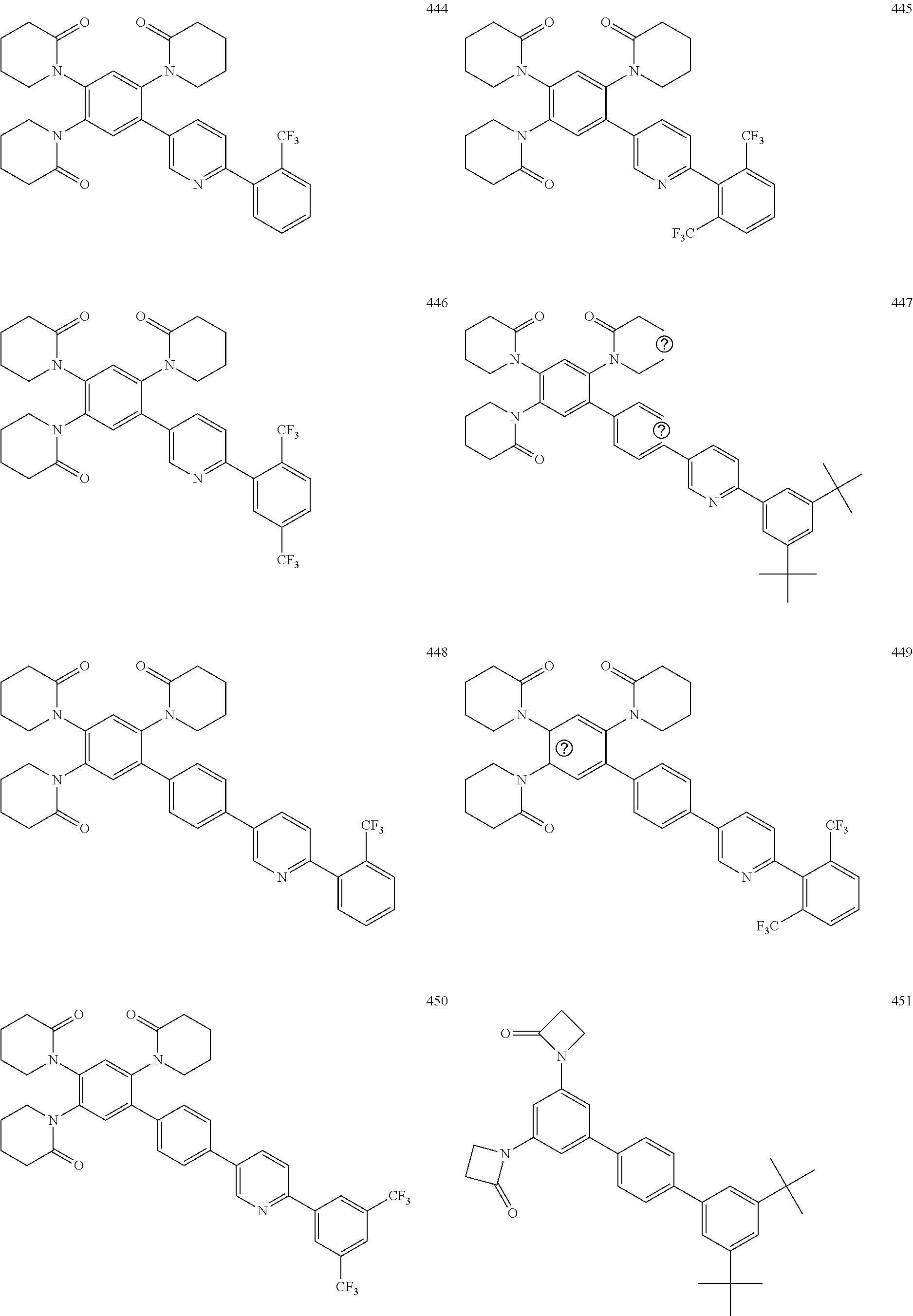

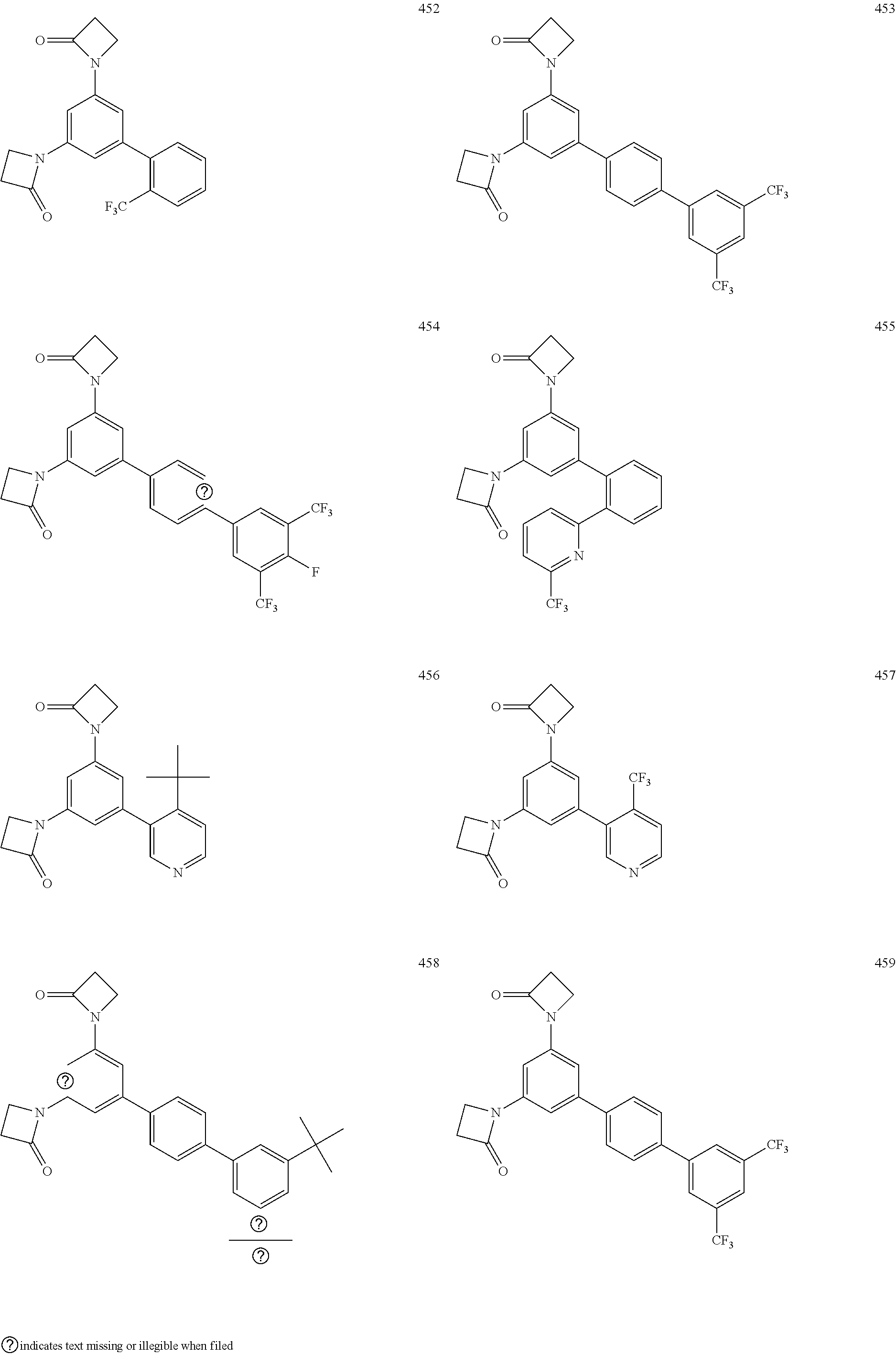

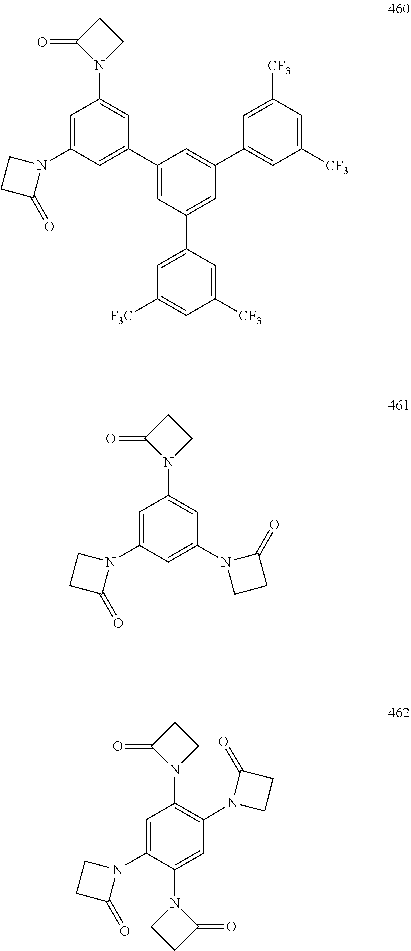 [SMILES: O=C1CCN1c1cc(-c2cc(-c3cc(C(F)(F)F)cc(C(F)(F)F)c3)cc(-c3cc(C(F)(F)F)cc(C(F)(F)F)c3)c2)cc(N2CCC2=O)c1 ; O=C1CCN1c1cc(N2CCC2=O)cc(N2CCC2=O)c1 ; O=C1CCN1c1cc(N2CCC2=O)c(N2CCC2=O)cc1N1CCC1=O]

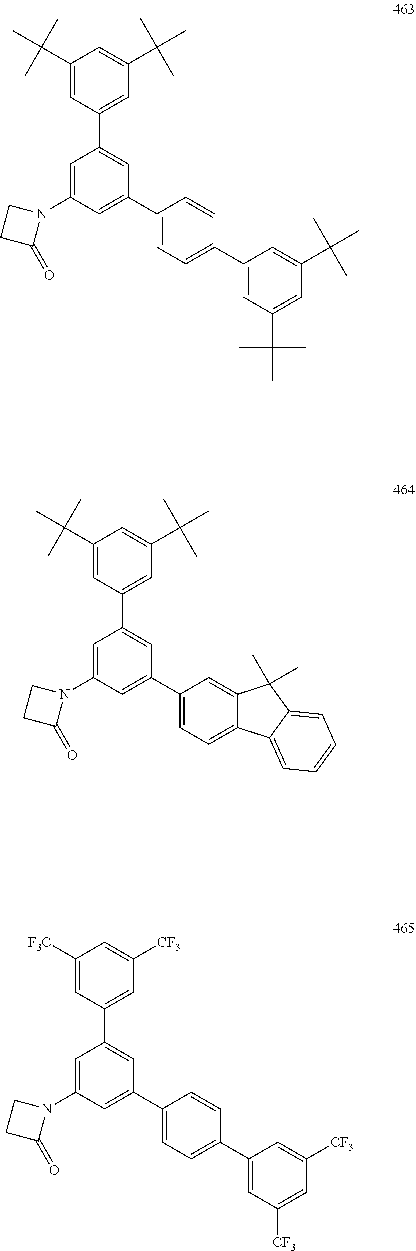

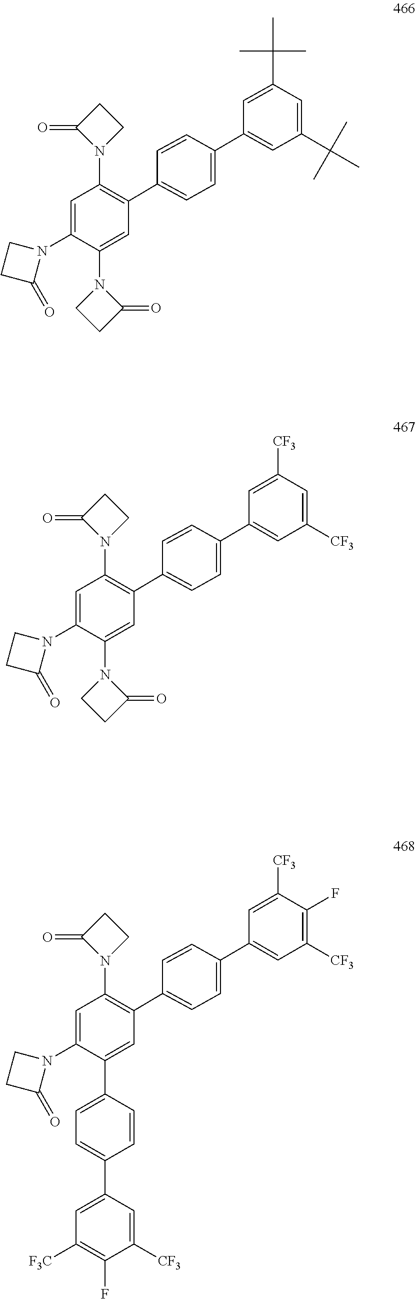 [SMILES: CC(C)(C)c1cc(-c2ccc(-c3cc(N4CCC4=O)c(N4CCC4=O)cc3N3CCC3=O)cc2)cc(C(C)(C)C)c1 ; O=C1CCN1c1cc(N2CCC2=O)c(N2CCC2=O)cc1-c1ccc(-c2cc(C(F)(F)F)cc(C(F)(F)F)c2)cc1 ; O=C1CCN1c1cc(N2CCC2=O)c(-c2ccc(-c3cc(C(F)(F)F)c(F)c(C(F)(F)F)c3)cc2)cc1-c1ccc(-c2cc(C(F)(F)F)c(F)c(C(F)(F)F)c2)cc1]

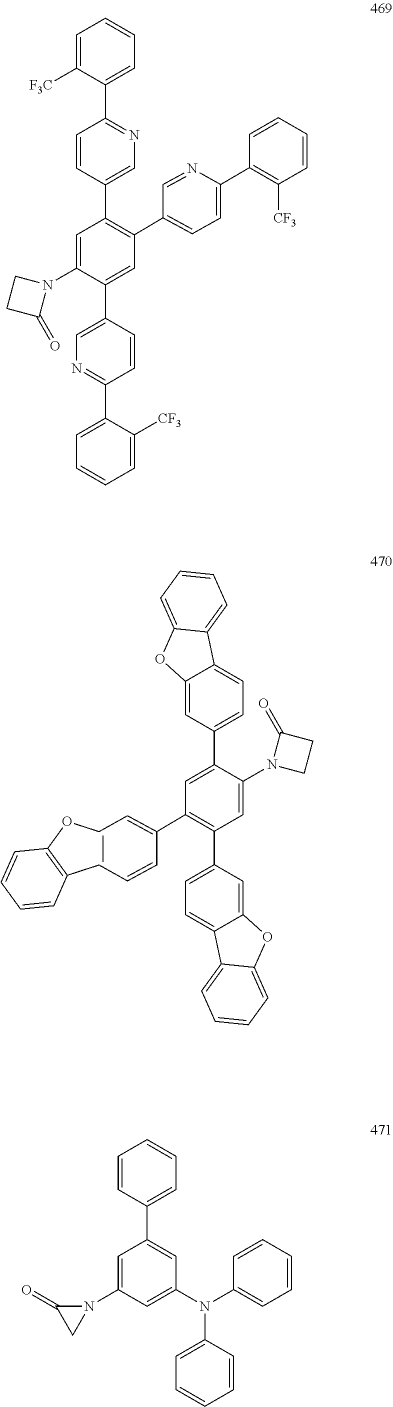

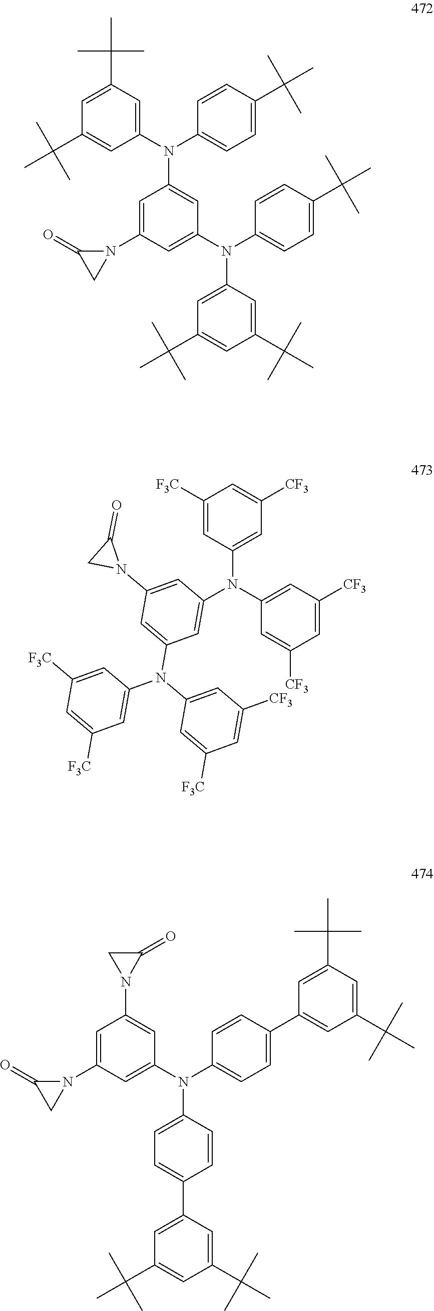 [SMILES: CC(C)(C)c1ccc(N(c2cc(N3CC3=O)cc(N(c3ccc(C(C)(C)C)cc3)c3cc(C(C)(C)C)cc(C(C)(C)C)c3)c2)c2cc(C(C)(C)C)cc(C(C)(C)C)c2)cc1 ; O=C1CN1c1cc(N(c2cc(C(F)(F)F)cc(C(F)(F)F)c2)c2cc(C(F)(F)F)cc(C(F)(F)F)c2)cc(N(c2cc(C(F)(F)F)cc(C(F)(F)F)c2)c2cc(C(F)(F)F)cc(C(F)(F)F)c2)c1 ; CC(C)(C)c1cc(-c2ccc(N(c3ccc(-c4cc(C(C)(C)C)cc(C(C)(C)C)c4)cc3)c3cc(N4CC4=O)cc(N4CC4=O)c3)cc2)cc(C(C)(C)C)c1]

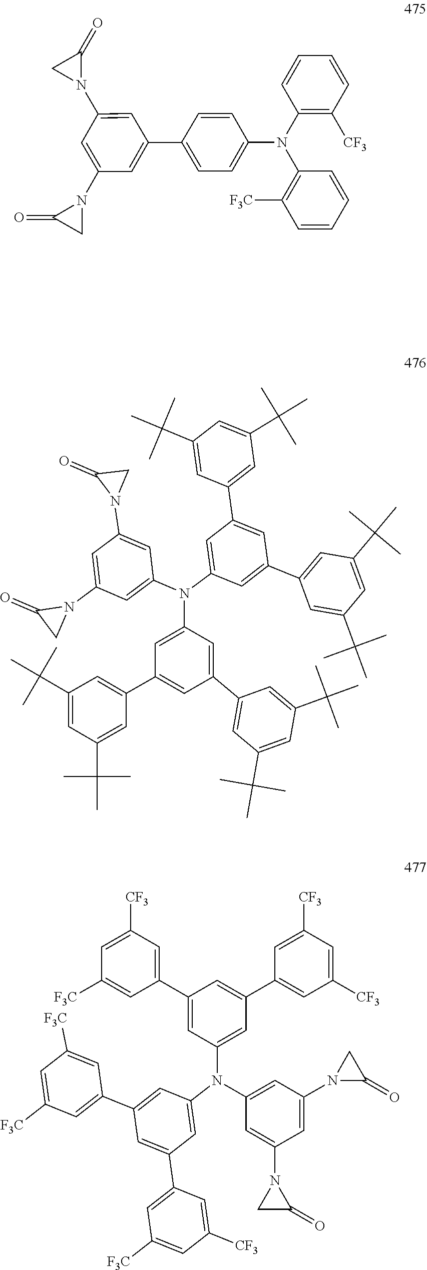 [SMILES: O=C1CN1c1cc(-c2ccc(N(c3ccccc3C(F)(F)F)c3ccccc3C(F)(F)F)cc2)cc(N2CC2=O)c1 ; CC(C)(C)c1cc(-c2cc(-c3cc(C(C)(C)C)cc(C(C)(C)C)c3)cc(N(c3cc(-c4cc(C(C)(C)C)cc(C(C)(C)C)c4)cc(-c4cc(C(C)(C)C)cc(C(C)(C)C)c4)c3)c3cc(N4CC4=O)cc(N4CC4=O)c3)c2)cc(C(C)(C)C)c1 ; O=C1CN1c1cc(N2CC2=O)cc(N(c2cc(-c3cc(C(F)(F)F)cc(C(F)(F)F)c3)cc(-c3cc(C(F)(F)F)cc(C(F)(F)F)c3)c2)c2cc(-c3cc(C(F)(F)F)cc(C(F)(F)F)c3)cc(-c3cc(C(F)(F)F)cc(C(F)(F)F)c3)c2)c1]

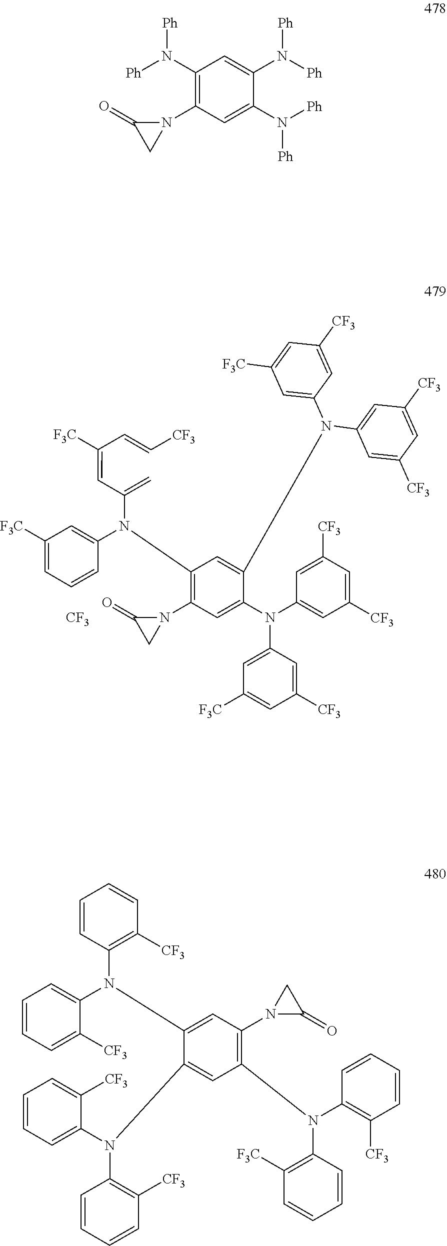

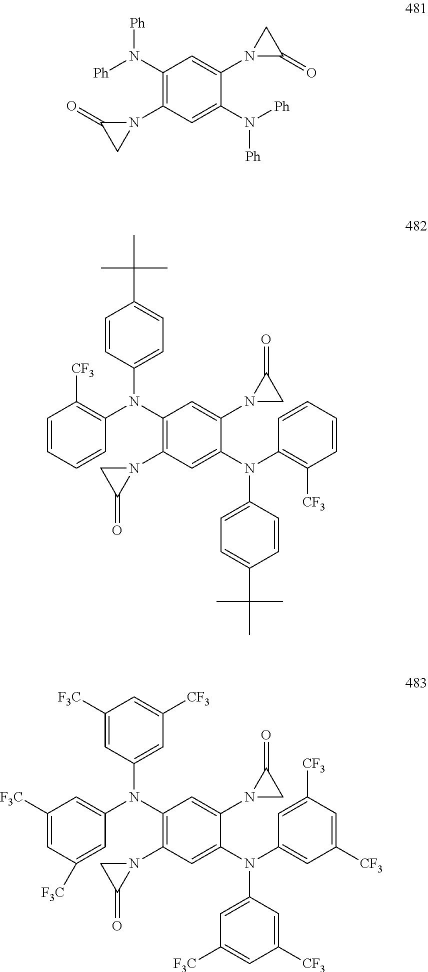 [SMILES: O=C1CN1c1cc(N(c2ccccc2)c2ccccc2)c(N2CC2=O)cc1N(c1ccccc1)c1ccccc1 ; CC(C)(C)c1ccc(N(c2cc(N3CC3=O)c(N(c3ccc(C(C)(C)C)cc3)c3ccccc3C(F)(F)F)cc2N2CC2=O)c2ccccc2C(F)(F)F)cc1 ; O=C1CN1c1cc(N(c2cc(C(F)(F)F)cc(C(F)(F)F)c2)c2cc(C(F)(F)F)cc(C(F)(F)F)c2)c(N2CC2=O)cc1N(c1cc(C(F)(F)F)cc(C(F)(F)F)c1)c1cc(C(F)(F)F)cc(C(F)(F)F)c1]

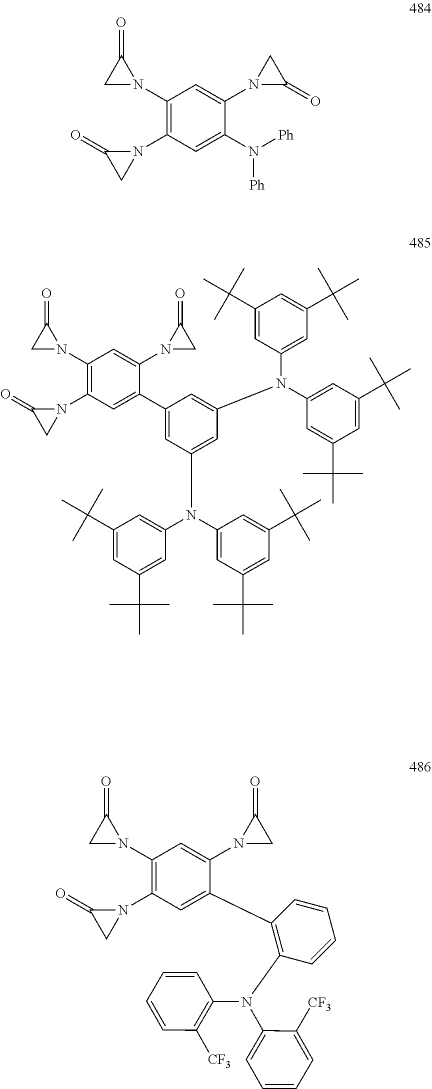 [SMILES: O=C1CN1c1cc(N2CC2=O)c(N(c2ccccc2)c2ccccc2)cc1N1CC1=O ; CC(C)(C)c1cc(N(c2cc(-c3cc(N4CC4=O)c(N4CC4=O)cc3N3CC3=O)cc(N(c3cc(C(C)(C)C)cc(C(C)(C)C)c3)c3cc(C(C)(C)C)cc(C(C)(C)C)c3)c2)c2cc(C(C)(C)C)cc(C(C)(C)C)c2)cc(C(C)(C)C)c1 ; O=C1CN1c1cc(N2CC2=O)c(N2CC2=O)cc1-c1ccccc1N(c1ccccc1C(F)(F)F)c1ccccc1C(F)(F)F]

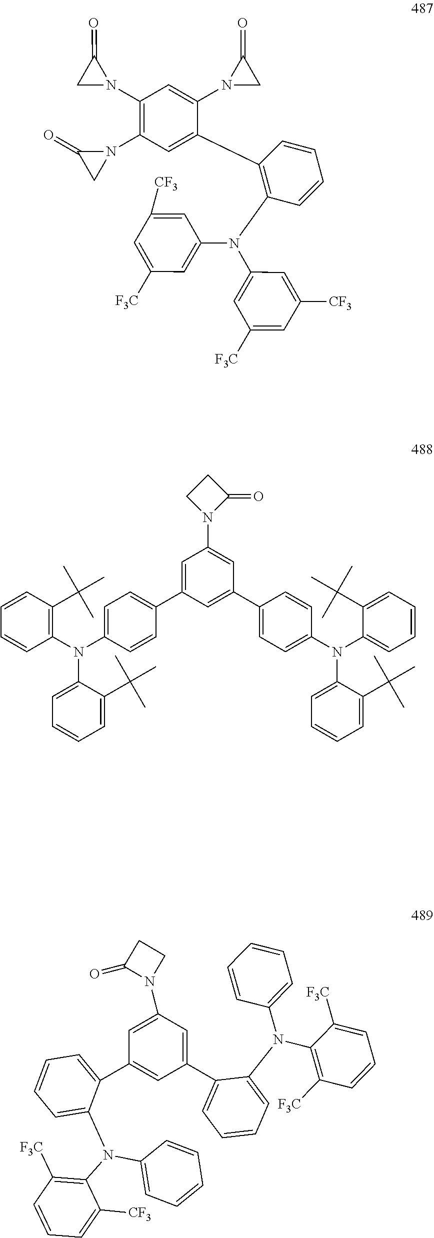 [SMILES: O=C1CN1c1cc(N2CC2=O)c(N2CC2=O)cc1-c1ccccc1N(c1cc(C(F)(F)F)cc(C(F)(F)F)c1)c1cc(C(F)(F)F)cc(C(F)(F)F)c1 ; CC(C)(C)c1ccccc1N(c1ccc(-c2cc(-c3ccc(N(c4ccccc4C(C)(C)C)c4ccccc4C(C)(C)C)cc3)cc(N3CCC3=O)c2)cc1)c1ccccc1C(C)(C)C ; O=C1CCN1c1cc(-c2ccccc2N(c2ccccc2)c2c(C(F)(F)F)cccc2C(F)(F)F)cc(-c2ccccc2N(c2ccccc2)c2c(C(F)(F)F)cccc2C(F)(F)F)c1]

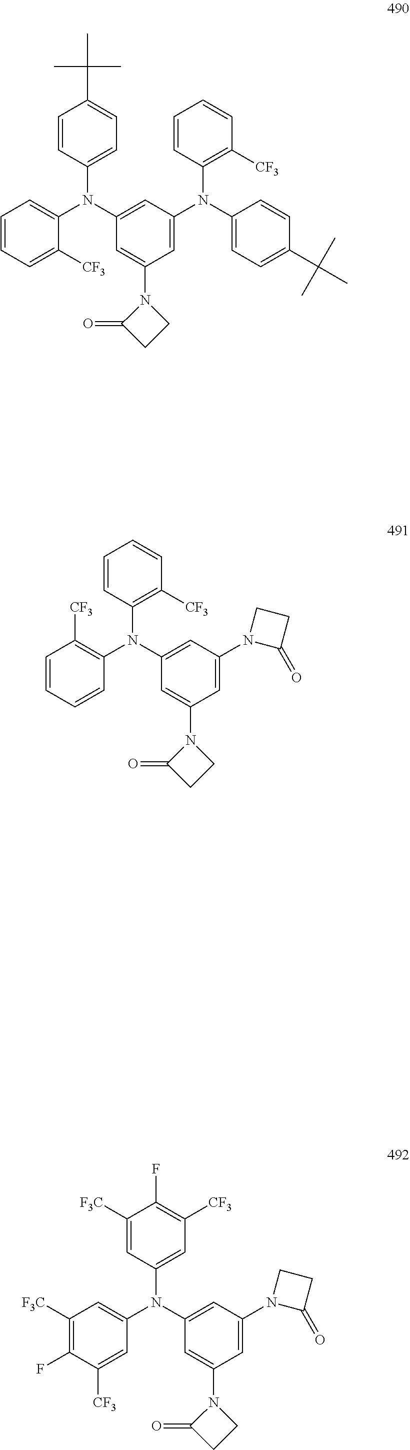 [SMILES: CC(C)(C)c1ccc(N(c2cc(N3CCC3=O)cc(N(c3ccc(C(C)(C)C)cc3)c3ccccc3C(F)(F)F)c2)c2ccccc2C(F)(F)F)cc1 ; O=C1CCN1c1cc(N2CCC2=O)cc(N(c2ccccc2C(F)(F)F)c2ccccc2C(F)(F)F)c1 ; O=C1CCN1c1cc(N2CCC2=O)cc(N(c2cc(C(F)(F)F)c(F)c(C(F)(F)F)c2)c2cc(C(F)(F)F)c(F)c(C(F)(F)F)c2)c1]

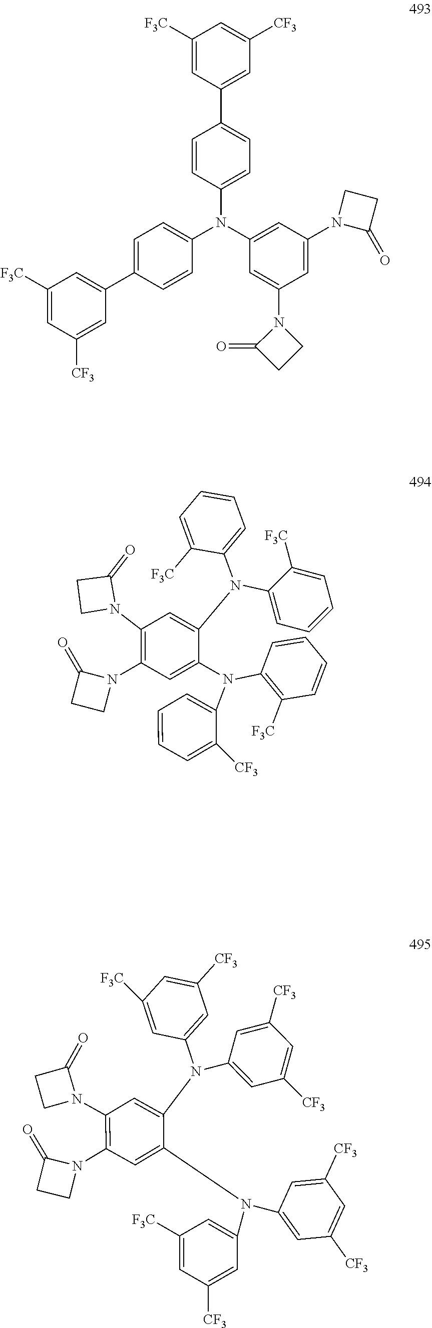 [SMILES: O=C1CCN1c1cc(N2CCC2=O)cc(N(c2ccc(-c3cc(C(F)(F)F)cc(C(F)(F)F)c3)cc2)c2ccc(-c3cc(C(F)(F)F)cc(C(F)(F)F)c3)cc2)c1 ; O=C1CCN1c1cc(N(c2ccccc2C(F)(F)F)c2ccccc2C(F)(F)F)c(N(c2ccccc2C(F)(F)F)c2ccccc2C(F)(F)F)cc1N1CCC1=O ; O=C1CCN1c1cc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)cc1N1CCC1=O]

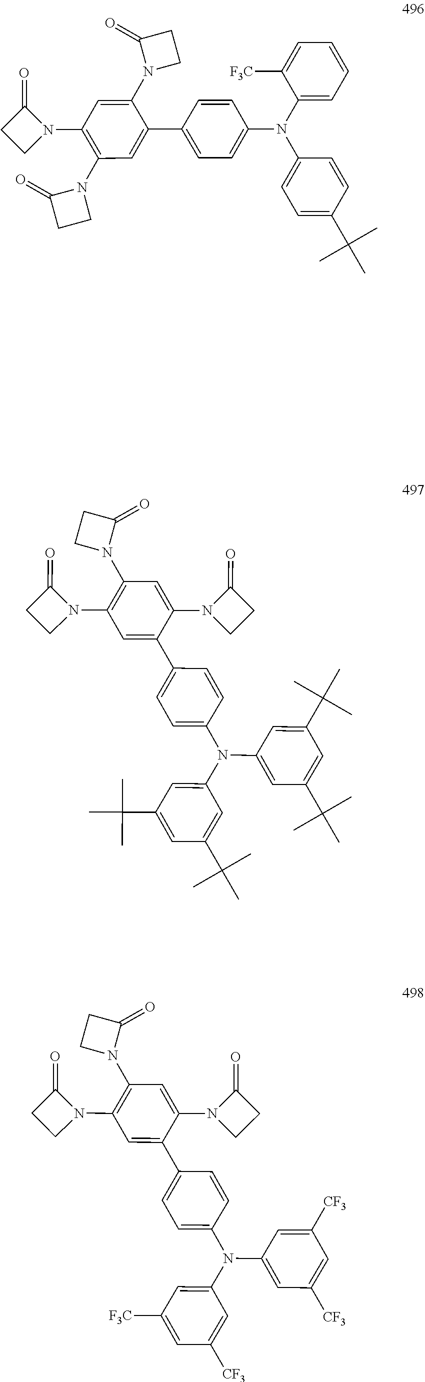 [SMILES: CC(C)(C)c1ccc(N(c2ccc(-c3cc(N4CCC4=O)c(N4CCC4=O)cc3N3CCC3=O)cc2)c2ccccc2C(F)(F)F)cc1 ; CC(C)(C)c1cc(N(c2ccc(-c3cc(N4CCC4=O)c(N4CCC4=O)cc3N3CCC3=O)cc2)c2cc(C(C)(C)C)cc(C(C)(C)C)c2)cc(C(C)(C)C)c1 ; O=C1CCN1c1cc(N2CCC2=O)c(N2CCC2=O)cc1-c1ccc(N(c2cc(C(F)(F)F)cc(C(F)(F)F)c2)c2cc(C(F)(F)F)cc(C(F)(F)F)c2)cc1]

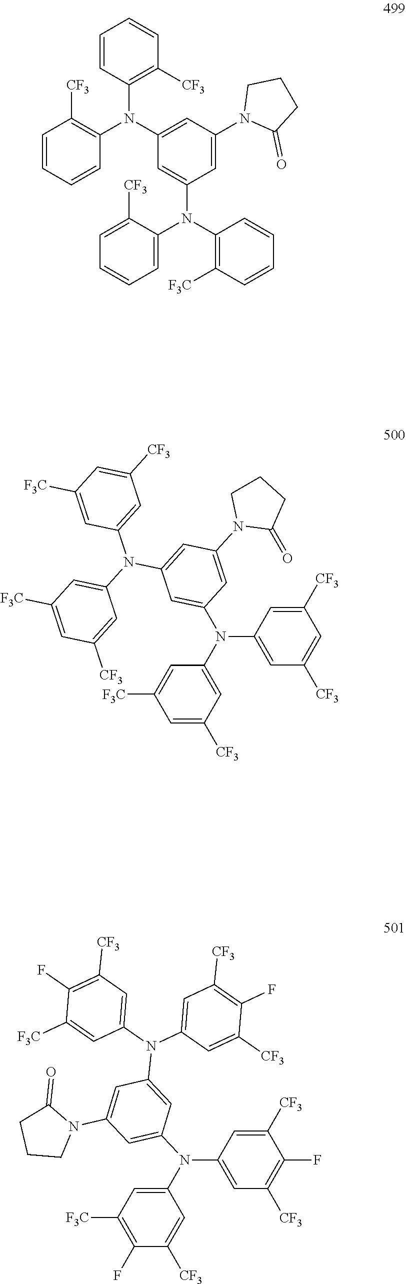 [SMILES: O=C1CCCN1c1cc(N(c2ccccc2C(F)(F)F)c2ccccc2C(F)(F)F)cc(N(c2ccccc2C(F)(F)F)c2ccccc2C(F)(F)F)c1 ; O=C1CCCN1c1cc(N(c2cc(C(F)(F)F)cc(C(F)(F)F)c2)c2cc(C(F)(F)F)cc(C(F)(F)F)c2)cc(N(c2cc(C(F)(F)F)cc(C(F)(F)F)c2)c2cc(C(F)(F)F)cc(C(F)(F)F)c2)c1 ; O=C1CCCN1c1cc(N(c2cc(C(F)(F)F)c(F)c(C(F)(F)F)c2)c2cc(C(F)(F)F)c(F)c(C(F)(F)F)c2)cc(N(c2cc(C(F)(F)F)c(F)c(C(F)(F)F)c2)c2cc(C(F)(F)F)c(F)c(C(F)(F)F)c2)c1]

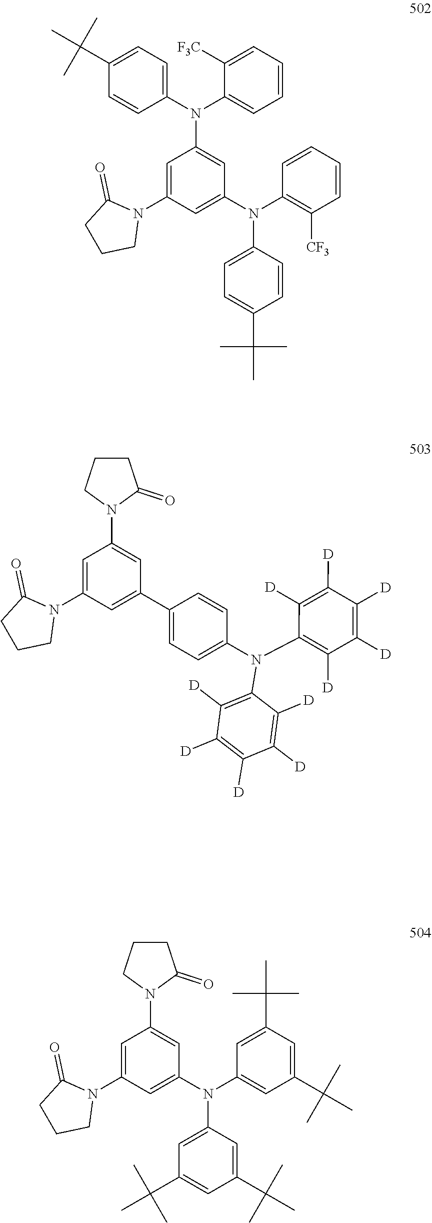 [SMILES: CC(C)(C)c1ccc(N(c2cc(N3CCCC3=O)cc(N(c3ccc(C(C)(C)C)cc3)c3ccccc3C(F)(F)F)c2)c2ccccc2C(F)(F)F)cc1 ; [2H]c1c([2H])c([2H])c(N(c2ccc(-c3cc(N4CCCC4=O)cc(N4CCCC4=O)c3)cc2)c2c([2H])c([2H])c([2H])c([2H])c2[2H])c([2H])c1[2H] ; CC(C)(C)c1cc(N(c2cc(N3CCCC3=O)cc(N3CCCC3=O)c2)c2cc(C(C)(C)C)cc(C(C)(C)C)c2)cc(C(C)(C)C)c1]

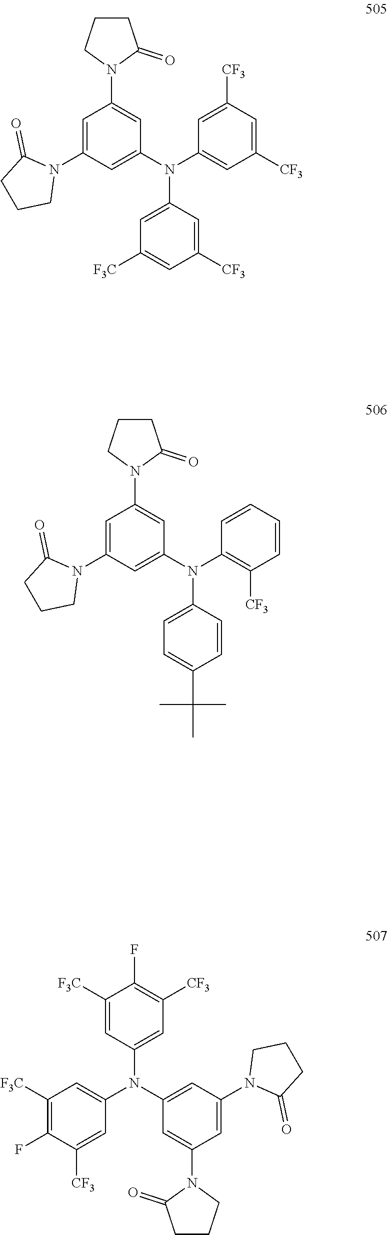 [SMILES: O=C1CCCN1c1cc(N2CCCC2=O)cc(N(c2cc(C(F)(F)F)cc(C(F)(F)F)c2)c2cc(C(F)(F)F)cc(C(F)(F)F)c2)c1 ; CC(C)(C)c1ccc(N(c2cc(N3CCCC3=O)cc(N3CCCC3=O)c2)c2ccccc2C(F)(F)F)cc1 ; O=C1CCCN1c1cc(N2CCCC2=O)cc(N(c2cc(C(F)(F)F)c(F)c(C(F)(F)F)c2)c2cc(C(F)(F)F)c(F)c(C(F)(F)F)c2)c1]

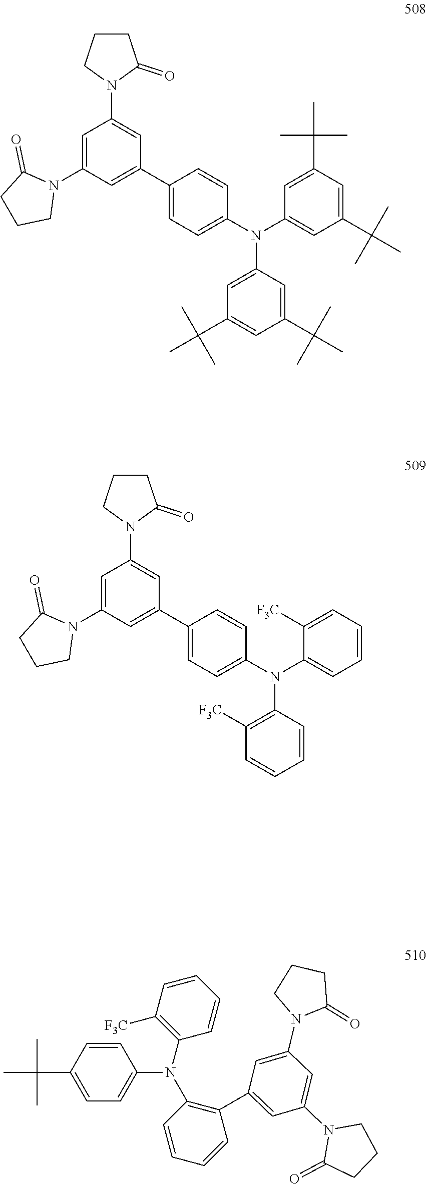

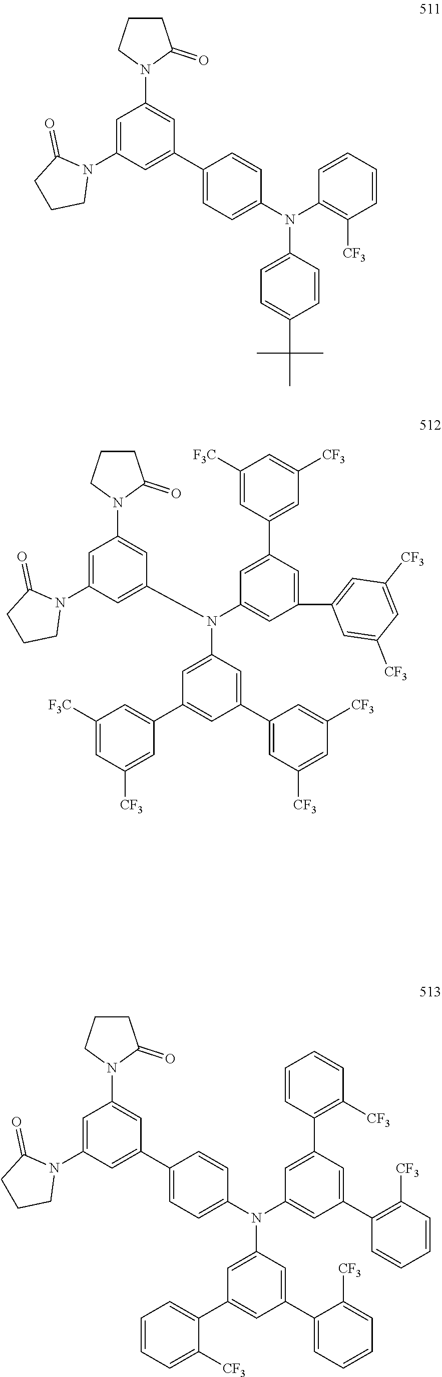 [SMILES: CC(C)(C)c1ccc(N(c2ccc(-c3cc(N4CCCC4=O)cc(N4CCCC4=O)c3)cc2)c2ccccc2C(F)(F)F)cc1 ; O=C1CCCN1c1cc(N2CCCC2=O)cc(N(c2cc(-c3cc(C(F)(F)F)cc(C(F)(F)F)c3)cc(-c3cc(C(F)(F)F)cc(C(F)(F)F)c3)c2)c2cc(-c3cc(C(F)(F)F)cc(C(F)(F)F)c3)cc(-c3cc(C(F)(F)F)cc(C(F)(F)F)c3)c2)c1 ; O=C1CCCN1c1cc(-c2ccc(N(c3cc(-c4ccccc4C(F)(F)F)cc(-c4ccccc4C(F)(F)F)c3)c3cc(-c4ccccc4C(F)(F)F)cc(-c4ccccc4C(F)(F)F)c3)cc2)cc(N2CCCC2=O)c1]

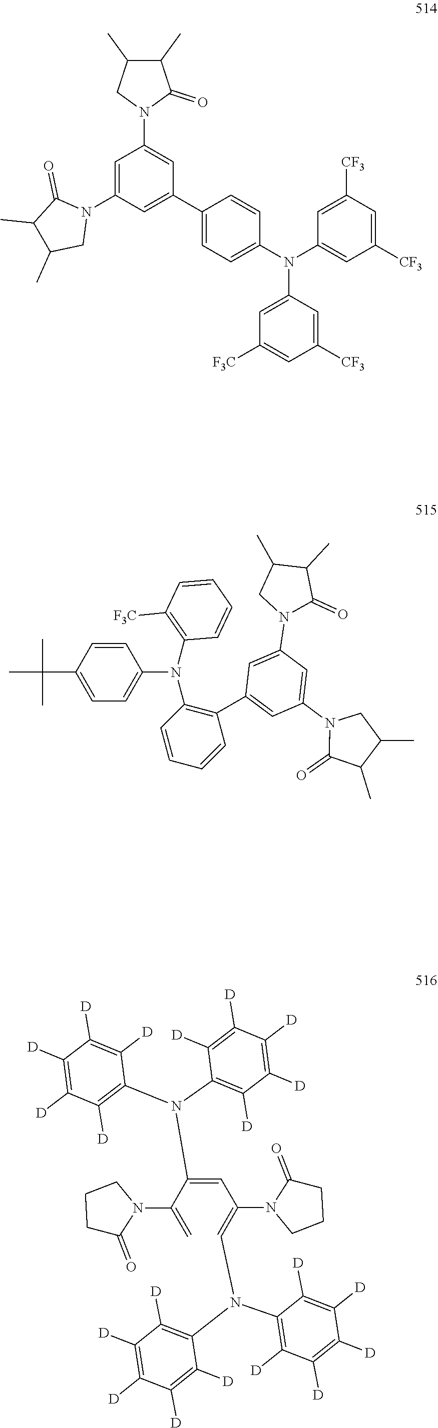 [SMILES: CC1CN(c2cc(-c3ccc(N(c4cc(C(F)(F)F)cc(C(F)(F)F)c4)c4cc(C(F)(F)F)cc(C(F)(F)F)c4)cc3)cc(N3CC(C)C(C)C3=O)c2)C(=O)C1C ; CC1CN(c2cc(-c3ccccc3N(c3ccc(C(C)(C)C)cc3)c3ccccc3C(F)(F)F)cc(N3CC(C)C(C)C3=O)c2)C(=O)C1C ; [2H]c1c([2H])c([2H])c(N(/C=C(/C=C(\C(=C)N2CCCC2=O)N(c2c([2H])c([2H])c([2H])c([2H])c2[2H])c2c([2H])c([2H])c([2H])c([2H])c2[2H])N2CCCC2=O)c2c([2H])c([2H])c([2H])c([2H])c2[2H])c([2H])c1[2H]]

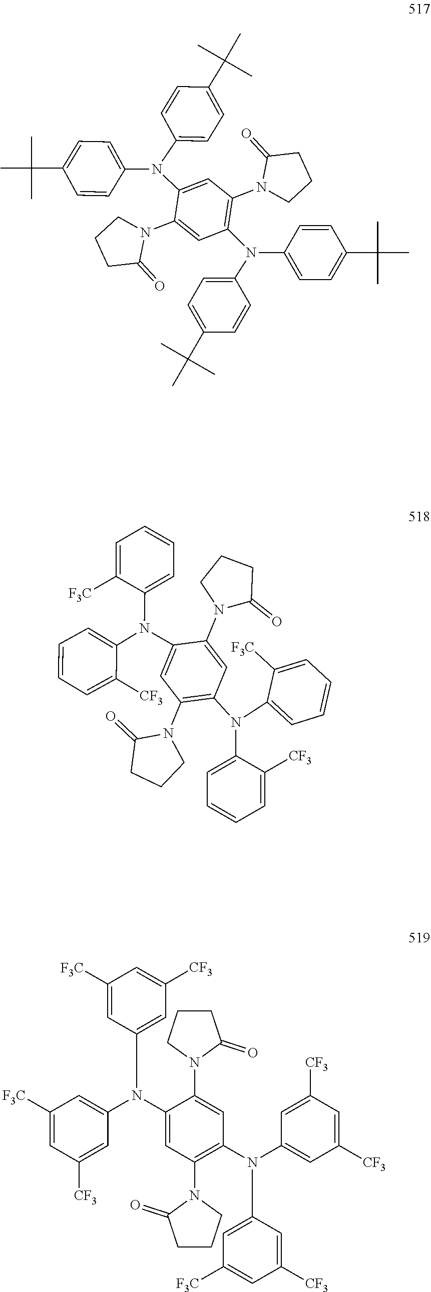 [SMILES: CC(C)(C)c1ccc(N(c2ccc(C(C)(C)C)cc2)c2cc(N3CCCC3=O)c(N(c3ccc(C(C)(C)C)cc3)c3ccc(C(C)(C)C)cc3)cc2N2CCCC2=O)cc1 ; O=C1CCCN1c1cc(N(c2ccccc2C(F)(F)F)c2ccccc2C(F)(F)F)c(N2CCCC2=O)cc1N(c1ccccc1C(F)(F)F)c1ccccc1C(F)(F)F ; O=C1CCCN1c1cc(N(c2cc(C(F)(F)F)cc(C(F)(F)F)c2)c2cc(C(F)(F)F)cc(C(F)(F)F)c2)c(N2CCCC2=O)cc1N(c1cc(C(F)(F)F)cc(C(F)(F)F)c1)c1cc(C(F)(F)F)cc(C(F)(F)F)c1]

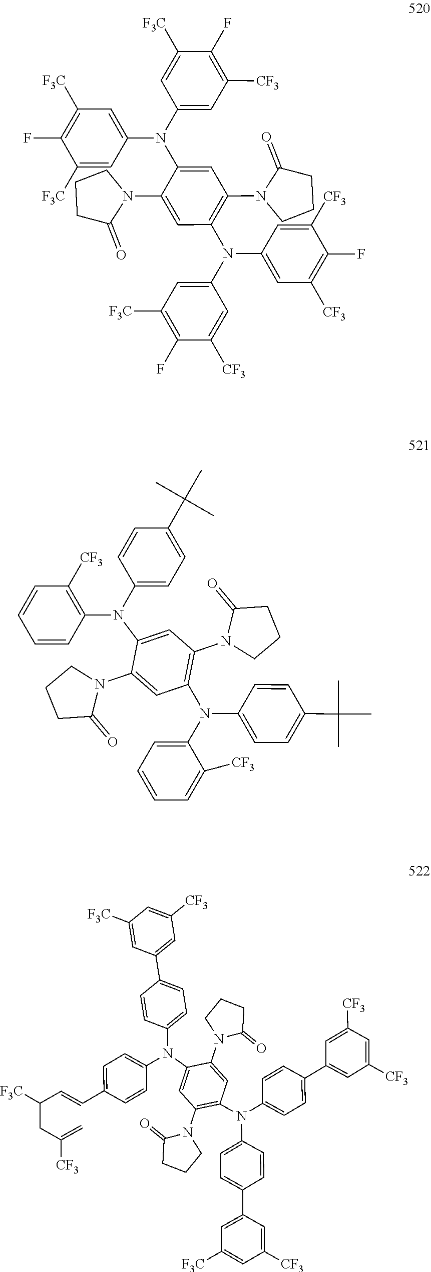

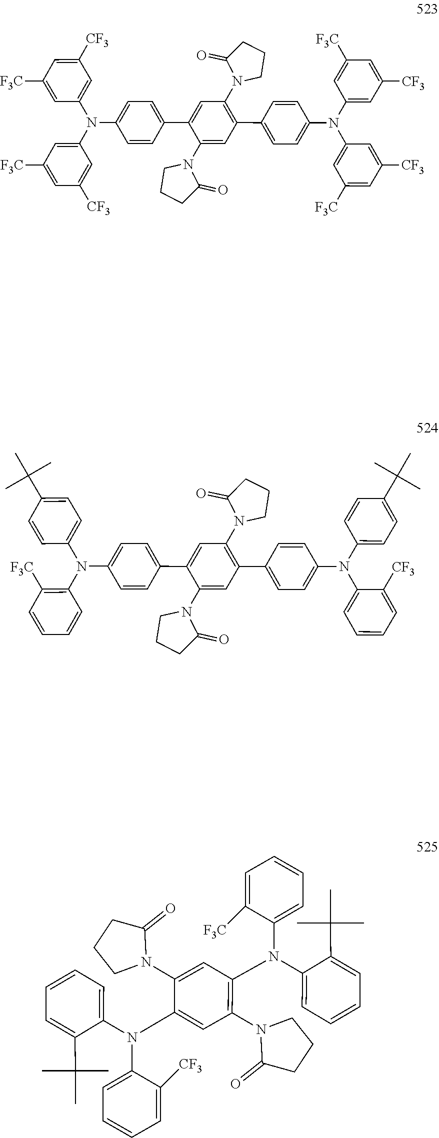

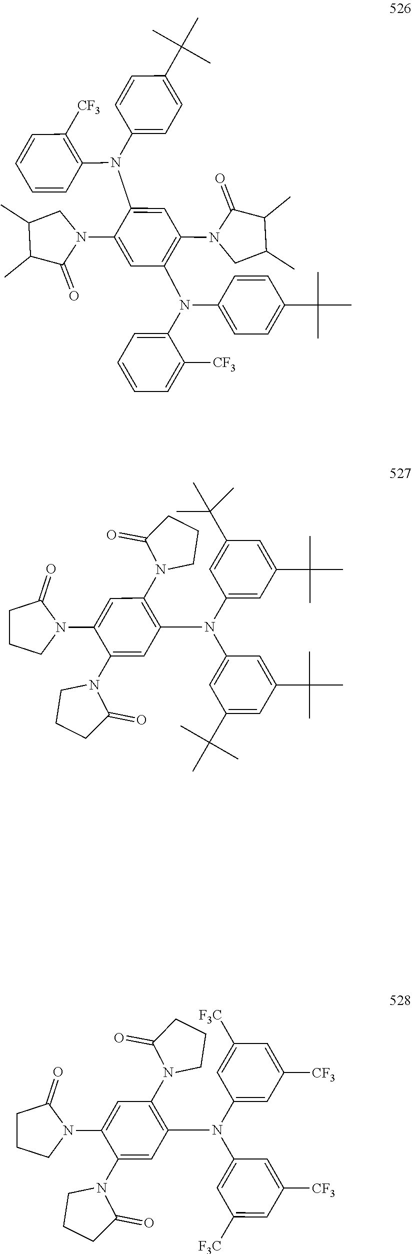

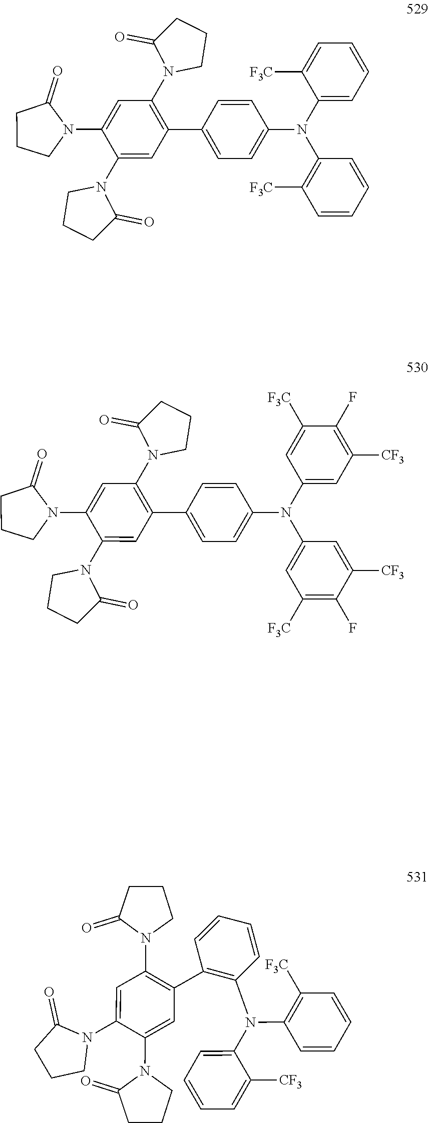 [SMILES: O=C1CCCN1c1cc(N2CCCC2=O)c(N2CCCC2=O)cc1-c1ccc(N(c2ccccc2C(F)(F)F)c2ccccc2C(F)(F)F)cc1 ; O=C1CCCN1c1cc(N2CCCC2=O)c(N2CCCC2=O)cc1-c1ccc(N(c2cc(C(F)(F)F)c(F)c(C(F)(F)F)c2)c2cc(C(F)(F)F)c(F)c(C(F)(F)F)c2)cc1 ; O=C1CCCN1c1cc(N2CCCC2=O)c(N2CCCC2=O)cc1-c1ccccc1N(c1ccccc1C(F)(F)F)c1ccccc1C(F)(F)F]

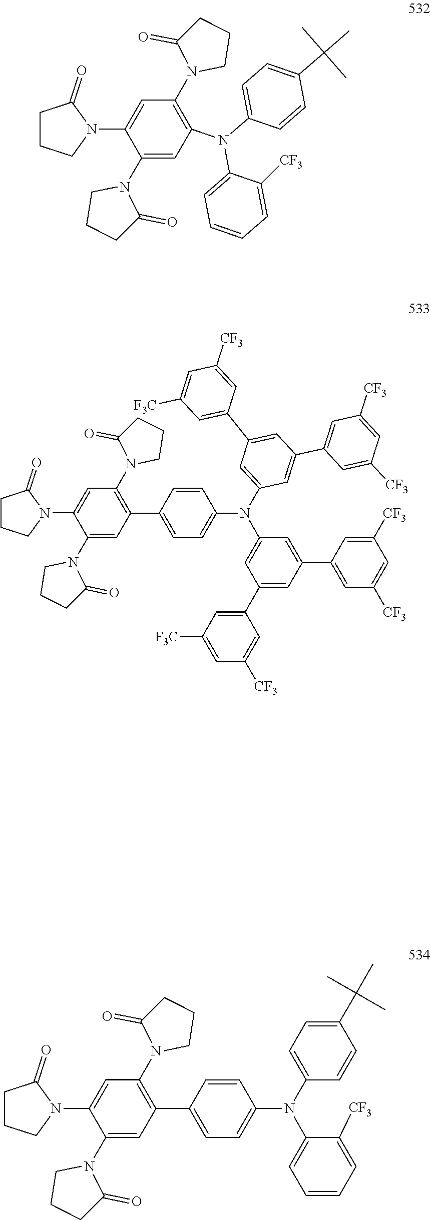 [SMILES: CC(C)(C)c1ccc(N(c2cc(N3CCCC3=O)c(N3CCCC3=O)cc2N2CCCC2=O)c2ccccc2C(F)(F)F)cc1 ; O=C1CCCN1c1cc(N2CCCC2=O)c(N2CCCC2=O)cc1-c1ccc(N(c2cc(-c3cc(C(F)(F)F)cc(C(F)(F)F)c3)cc(-c3cc(C(F)(F)F)cc(C(F)(F)F)c3)c2)c2cc(-c3cc(C(F)(F)F)cc(C(F)(F)F)c3)cc(-c3cc(C(F)(F)F)cc(C(F)(F)F)c3)c2)cc1 ; CC(C)(C)c1ccc(N(c2ccc(-c3cc(N4CCCC4=O)c(N4CCCC4=O)cc3N3CCCC3=O)cc2)c2ccccc2C(F)(F)F)cc1]

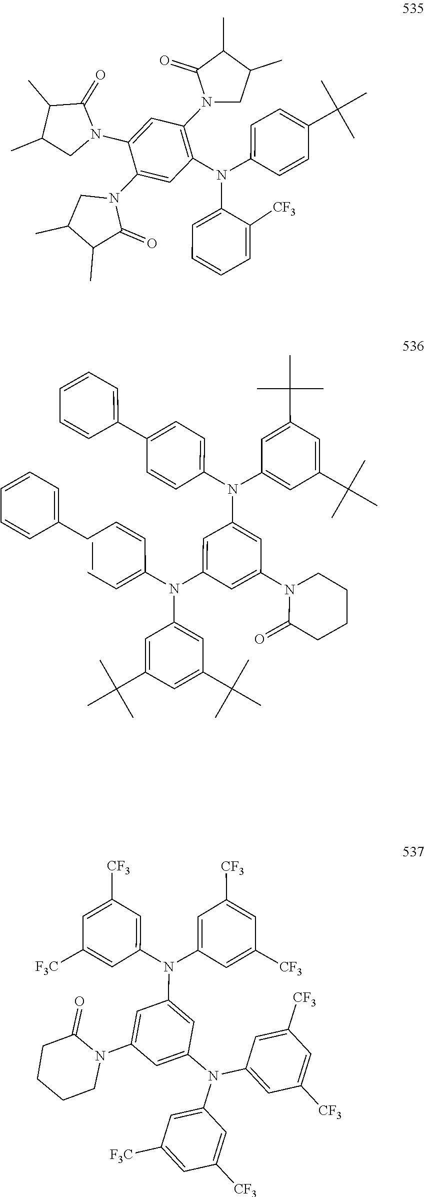

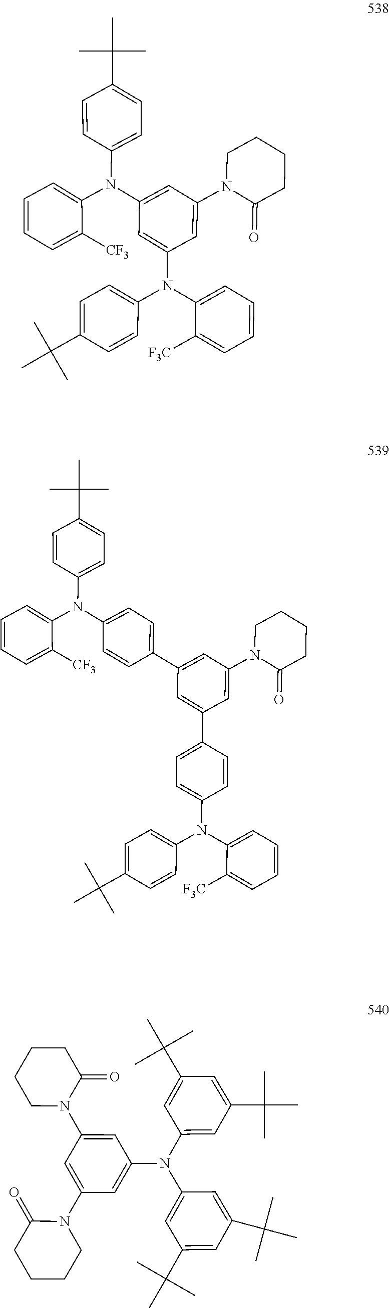 [SMILES: CC(C)(C)c1ccc(N(c2cc(N3CCCCC3=O)cc(N(c3ccc(C(C)(C)C)cc3)c3ccccc3C(F)(F)F)c2)c2ccccc2C(F)(F)F)cc1 ; CC(C)(C)c1ccc(N(c2ccc(-c3cc(-c4ccc(N(c5ccc(C(C)(C)C)cc5)c5ccccc5C(F)(F)F)cc4)cc(N4CCCCC4=O)c3)cc2)c2ccccc2C(F)(F)F)cc1 ; CC(C)(C)c1cc(N(c2cc(N3CCCCC3=O)cc(N3CCCCC3=O)c2)c2cc(C(C)(C)C)cc(C(C)(C)C)c2)cc(C(C)(C)C)c1]

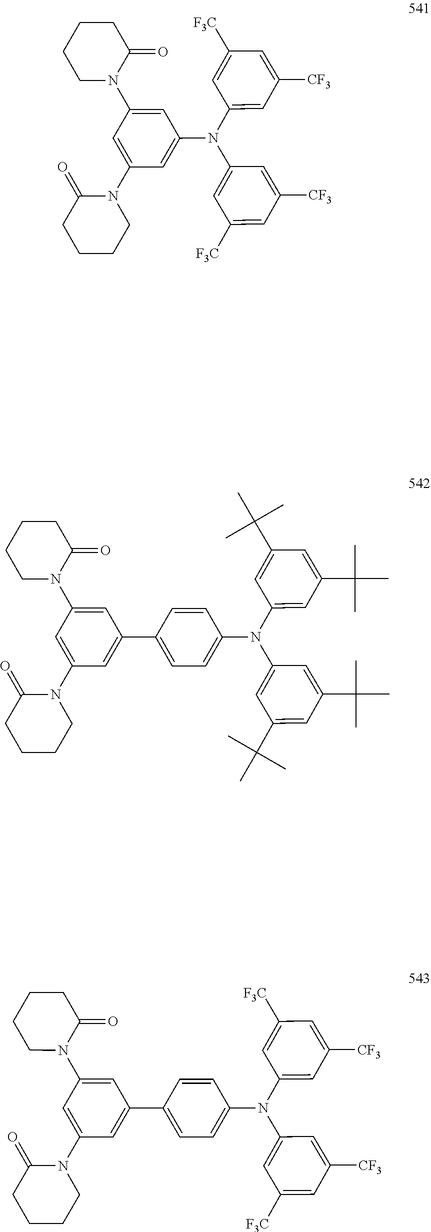 [SMILES: O=C1CCCCN1c1cc(N2CCCCC2=O)cc(N(c2cc(C(F)(F)F)cc(C(F)(F)F)c2)c2cc(C(F)(F)F)cc(C(F)(F)F)c2)c1 ; CC(C)(C)c1cc(N(c2ccc(-c3cc(N4CCCCC4=O)cc(N4CCCCC4=O)c3)cc2)c2cc(C(C)(C)C)cc(C(C)(C)C)c2)cc(C(C)(C)C)c1 ; O=C1CCCCN1c1cc(-c2ccc(N(c3cc(C(F)(F)F)cc(C(F)(F)F)c3)c3cc(C(F)(F)F)cc(C(F)(F)F)c3)cc2)cc(N2CCCCC2=O)c1]

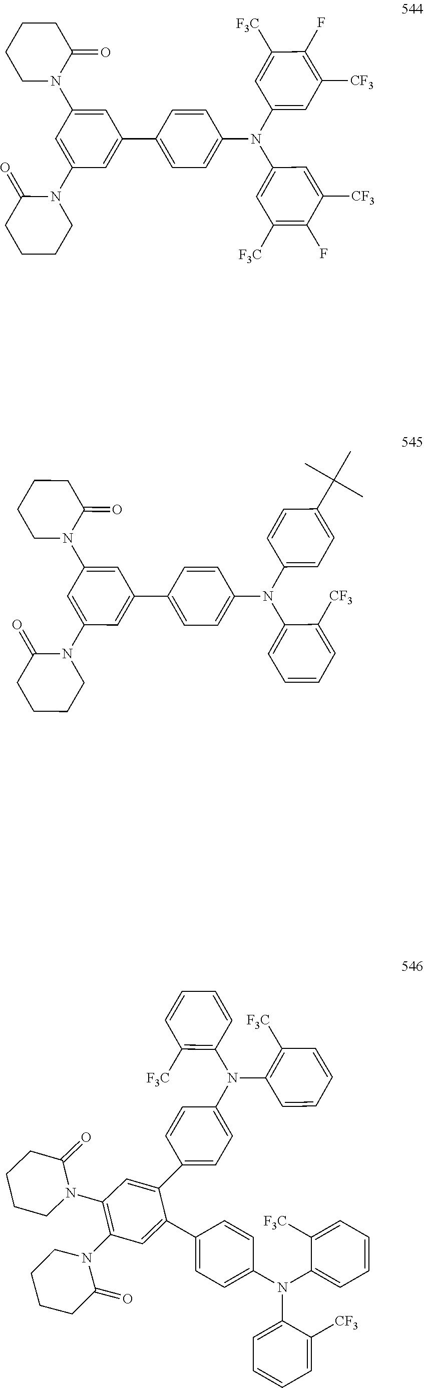 [SMILES: O=C1CCCCN1c1cc(-c2ccc(N(c3cc(C(F)(F)F)c(F)c(C(F)(F)F)c3)c3cc(C(F)(F)F)c(F)c(C(F)(F)F)c3)cc2)cc(N2CCCCC2=O)c1 ; CC(C)(C)c1ccc(N(c2ccc(-c3cc(N4CCCCC4=O)cc(N4CCCCC4=O)c3)cc2)c2ccccc2C(F)(F)F)cc1 ; O=C1CCCCN1c1cc(-c2ccc(N(c3ccccc3C(F)(F)F)c3ccccc3C(F)(F)F)cc2)c(-c2ccc(N(c3ccccc3C(F)(F)F)c3ccccc3C(F)(F)F)cc2)cc1N1CCCCC1=O]

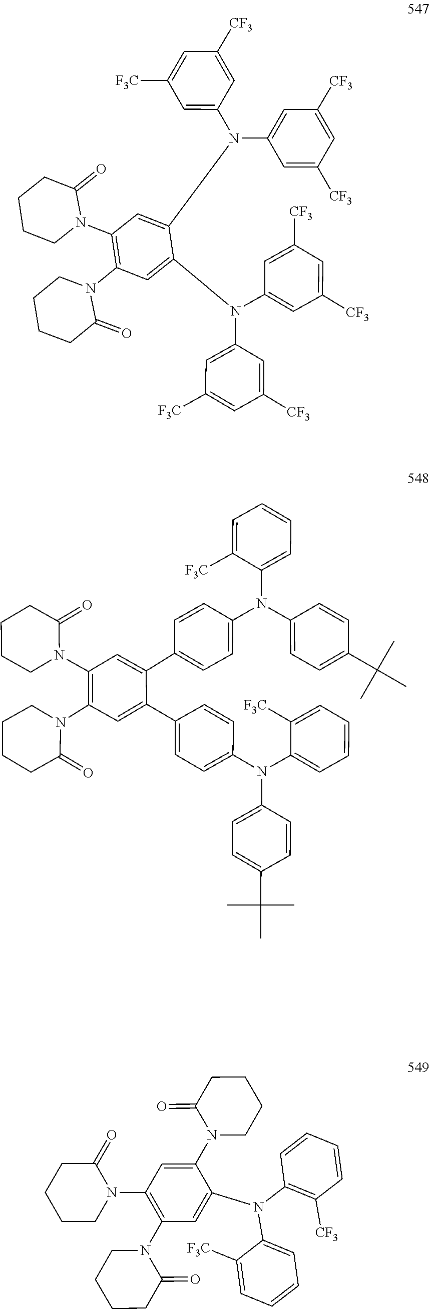 [SMILES: O=C1CCCCN1c1cc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)cc1N1CCCCC1=O ; CC(C)(C)c1ccc(N(c2ccc(-c3cc(N4CCCCC4=O)c(N4CCCCC4=O)cc3-c3ccc(N(c4ccc(C(C)(C)C)cc4)c4ccccc4C(F)(F)F)cc3)cc2)c2ccccc2C(F)(F)F)cc1 ; O=C1CCCCN1c1cc(N2CCCCC2=O)c(N(c2ccccc2C(F)(F)F)c2ccccc2C(F)(F)F)cc1N1CCCCC1=O]

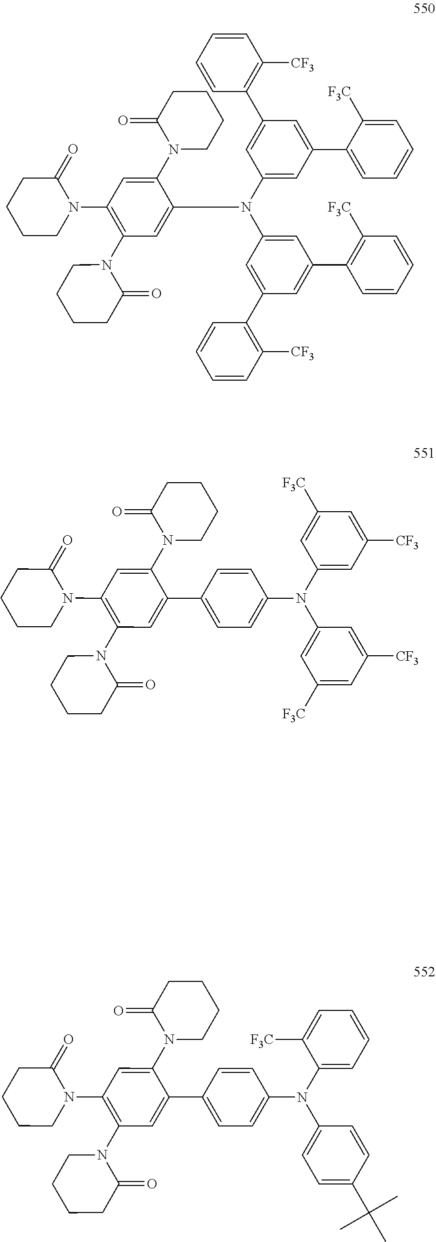 [SMILES: O=C1CCCCN1c1cc(N2CCCCC2=O)c(N(c2cc(-c3ccccc3C(F)(F)F)cc(-c3ccccc3C(F)(F)F)c2)c2cc(-c3ccccc3C(F)(F)F)cc(-c3ccccc3C(F)(F)F)c2)cc1N1CCCCC1=O ; O=C1CCCCN1c1cc(N2CCCCC2=O)c(N2CCCCC2=O)cc1-c1ccc(N(c2cc(C(F)(F)F)cc(C(F)(F)F)c2)c2cc(C(F)(F)F)cc(C(F)(F)F)c2)cc1 ; CC(C)(C)c1ccc(N(c2ccc(-c3cc(N4CCCCC4=O)c(N4CCCCC4=O)cc3N3CCCCC3=O)cc2)c2ccccc2C(F)(F)F)cc1]

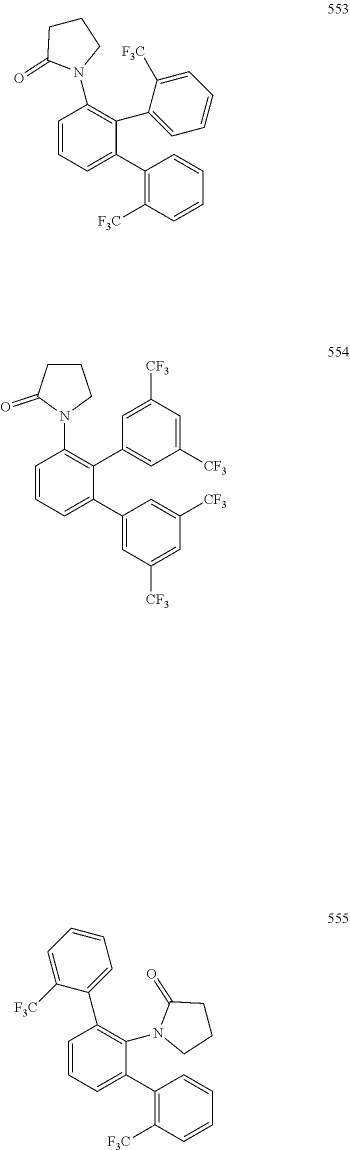 [SMILES: O=C1CCCN1c1cccc(-c2ccccc2C(F)(F)F)c1-c1ccccc1C(F)(F)F ; O=C1CCCN1c1cccc(-c2cc(C(F)(F)F)cc(C(F)(F)F)c2)c1-c1cc(C(F)(F)F)cc(C(F)(F)F)c1 ; O=C1CCCN1c1c(-c2ccccc2C(F)(F)F)cccc1-c1ccccc1C(F)(F)F]

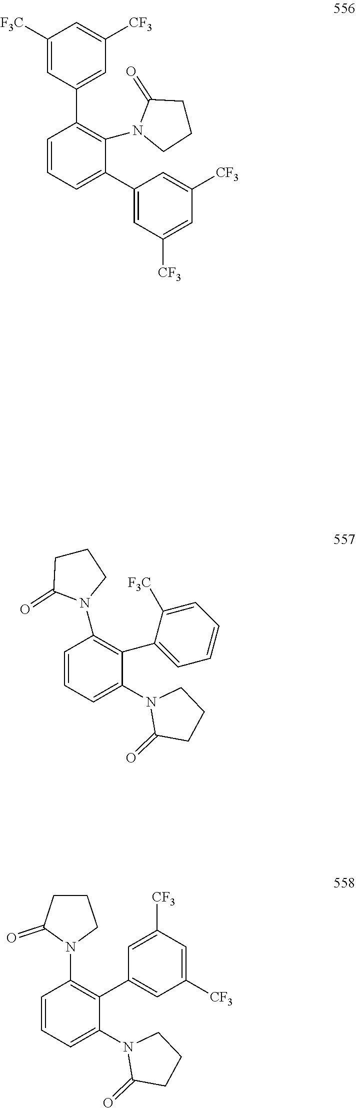 [SMILES: O=C1CCCN1c1c(-c2cc(C(F)(F)F)cc(C(F)(F)F)c2)cccc1-c1cc(C(F)(F)F)cc(C(F)(F)F)c1 ; O=C1CCCN1c1cccc(N2CCCC2=O)c1-c1ccccc1C(F)(F)F ; O=C1CCCN1c1cccc(N2CCCC2=O)c1-c1cc(C(F)(F)F)cc(C(F)(F)F)c1]

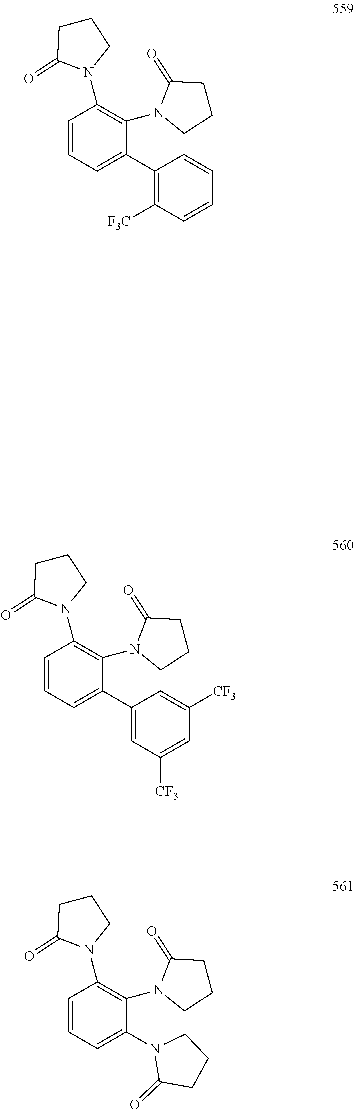 [SMILES: O=C1CCCN1c1cccc(-c2ccccc2C(F)(F)F)c1N1CCCC1=O ; O=C1CCCN1c1cccc(-c2cc(C(F)(F)F)cc(C(F)(F)F)c2)c1N1CCCC1=O ; O=C1CCCN1c1cccc(N2CCCC2=O)c1N1CCCC1=O]

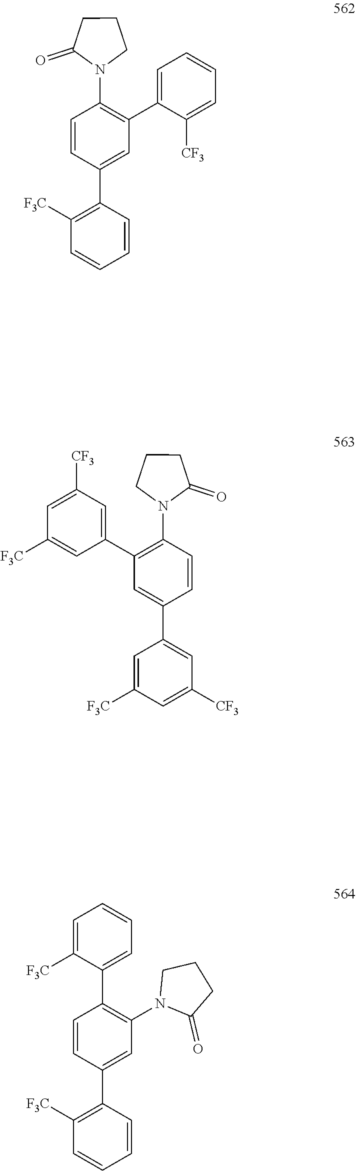 [SMILES: O=C1CCCN1c1ccc(-c2ccccc2C(F)(F)F)cc1-c1ccccc1C(F)(F)F ; O=C1CCCN1c1ccc(-c2cc(C(F)(F)F)cc(C(F)(F)F)c2)cc1-c1cc(C(F)(F)F)cc(C(F)(F)F)c1 ; O=C1CCCN1c1cc(-c2ccccc2C(F)(F)F)ccc1-c1ccccc1C(F)(F)F]

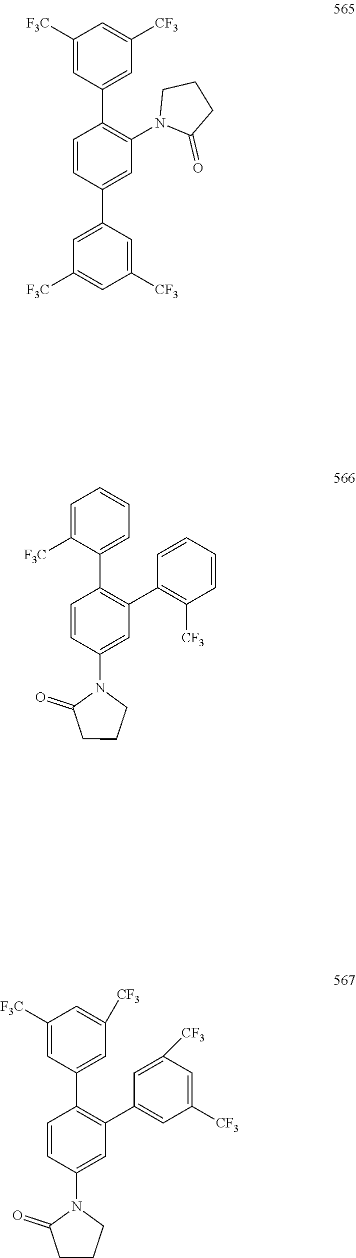 [SMILES: O=C1CCCN1c1cc(-c2cc(C(F)(F)F)cc(C(F)(F)F)c2)ccc1-c1cc(C(F)(F)F)cc(C(F)(F)F)c1 ; O=C1CCCN1c1ccc(-c2ccccc2C(F)(F)F)c(-c2ccccc2C(F)(F)F)c1 ; O=C1CCCN1c1ccc(-c2cc(C(F)(F)F)cc(C(F)(F)F)c2)c(-c2cc(C(F)(F)F)cc(C(F)(F)F)c2)c1]

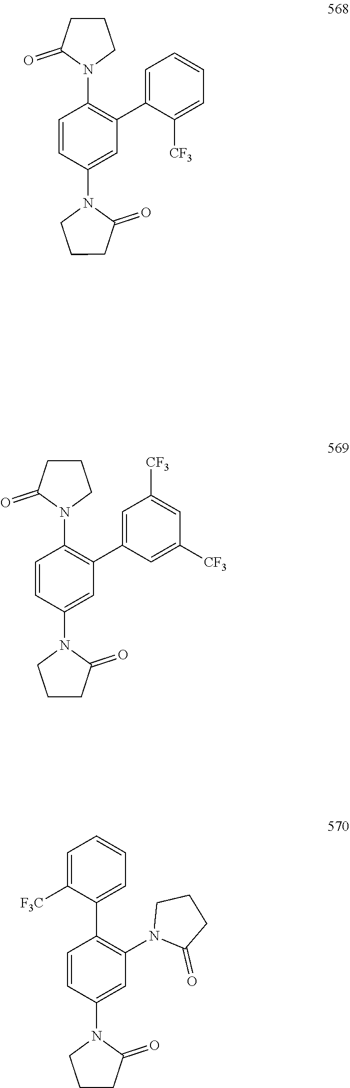 [SMILES: O=C1CCCN1c1ccc(N2CCCC2=O)c(-c2ccccc2C(F)(F)F)c1 ; O=C1CCCN1c1ccc(N2CCCC2=O)c(-c2cc(C(F)(F)F)cc(C(F)(F)F)c2)c1 ; O=C1CCCN1c1ccc(-c2ccccc2C(F)(F)F)c(N2CCCC2=O)c1]

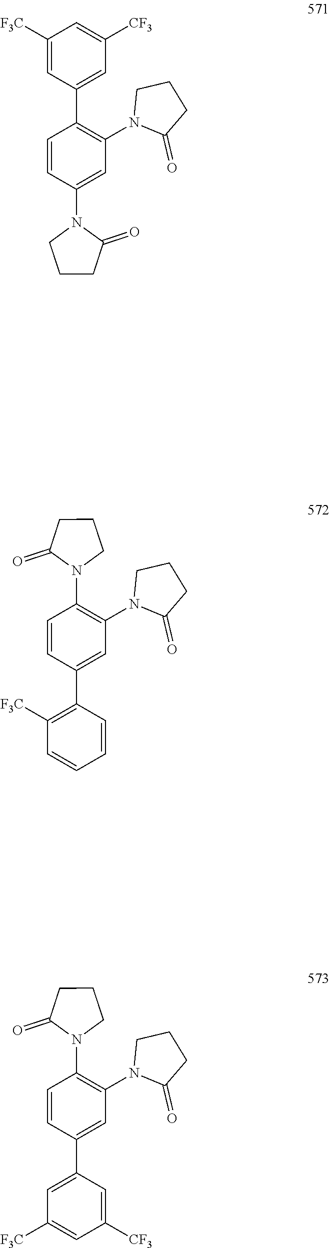 [SMILES: O=C1CCCN1c1ccc(-c2cc(C(F)(F)F)cc(C(F)(F)F)c2)c(N2CCCC2=O)c1 ; O=C1CCCN1c1ccc(-c2ccccc2C(F)(F)F)cc1N1CCCC1=O ; O=C1CCCN1c1ccc(-c2cc(C(F)(F)F)cc(C(F)(F)F)c2)cc1N1CCCC1=O]

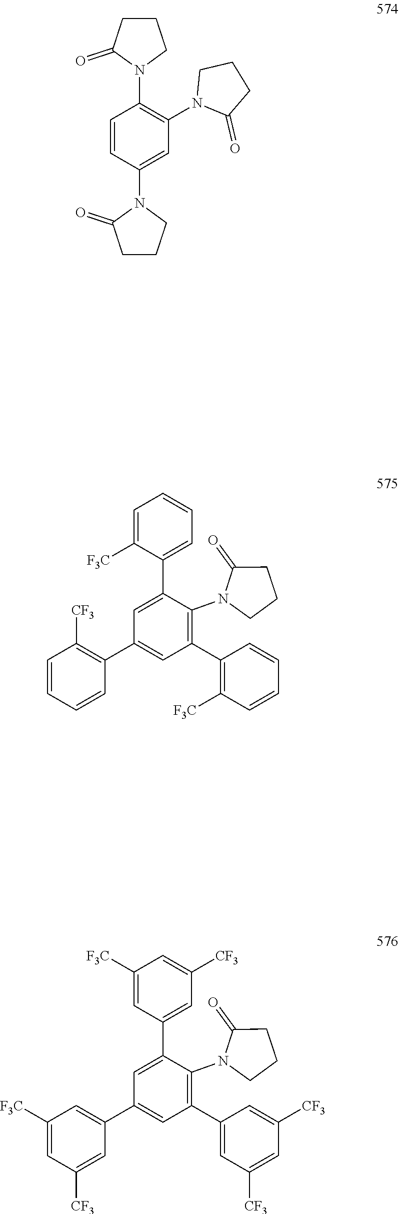 [SMILES: O=C1CCCN1c1ccc(N2CCCC2=O)c(N2CCCC2=O)c1 ; O=C1CCCN1c1c(-c2ccccc2C(F)(F)F)cc(-c2ccccc2C(F)(F)F)cc1-c1ccccc1C(F)(F)F ; O=C1CCCN1c1c(-c2cc(C(F)(F)F)cc(C(F)(F)F)c2)cc(-c2cc(C(F)(F)F)cc(C(F)(F)F)c2)cc1-c1cc(C(F)(F)F)cc(C(F)(F)F)c1]

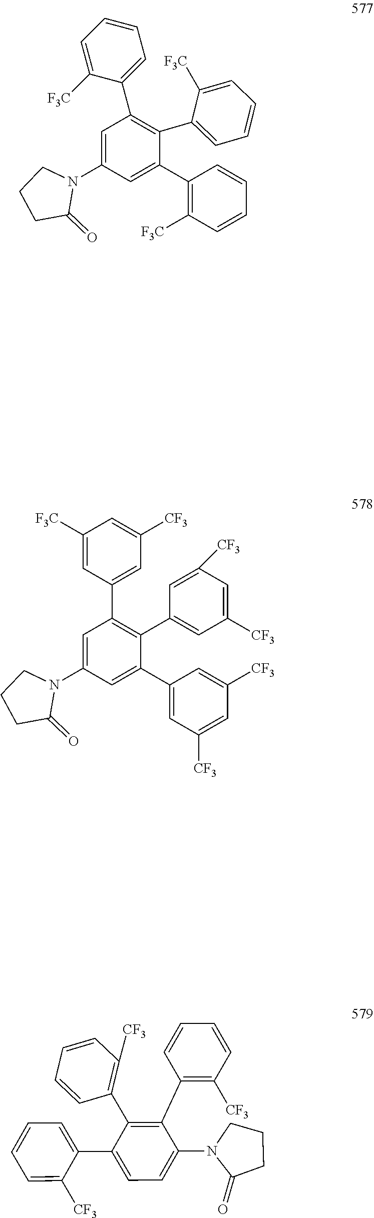 [SMILES: O=C1CCCN1c1cc(-c2ccccc2C(F)(F)F)c(-c2ccccc2C(F)(F)F)c(-c2ccccc2C(F)(F)F)c1 ; O=C1CCCN1c1cc(-c2cc(C(F)(F)F)cc(C(F)(F)F)c2)c(-c2cc(C(F)(F)F)cc(C(F)(F)F)c2)c(-c2cc(C(F)(F)F)cc(C(F)(F)F)c2)c1 ; O=C1CCCN1c1ccc(-c2ccccc2C(F)(F)F)c(-c2ccccc2C(F)(F)F)c1-c1ccccc1C(F)(F)F]

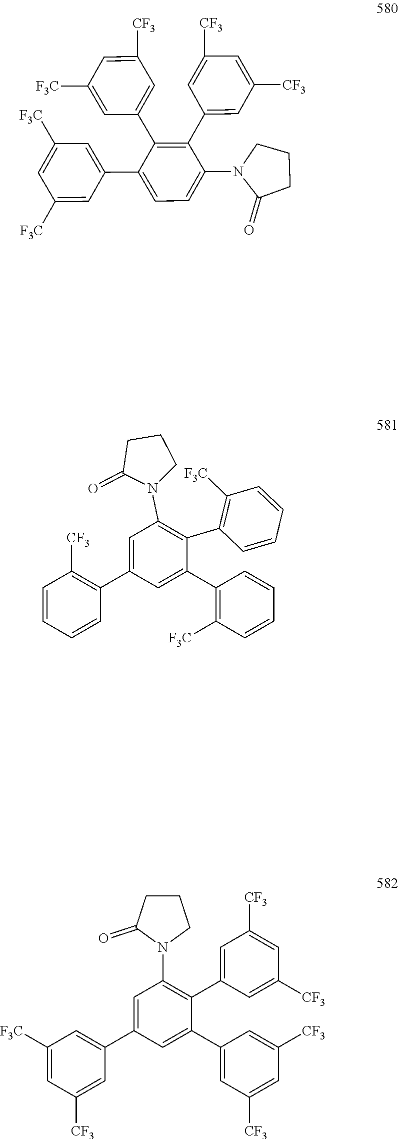 [SMILES: O=C1CCCN1c1ccc(-c2cc(C(F)(F)F)cc(C(F)(F)F)c2)c(-c2cc(C(F)(F)F)cc(C(F)(F)F)c2)c1-c1cc(C(F)(F)F)cc(C(F)(F)F)c1 ; O=C1CCCN1c1cc(-c2ccccc2C(F)(F)F)cc(-c2ccccc2C(F)(F)F)c1-c1ccccc1C(F)(F)F ; O=C1CCCN1c1cc(-c2cc(C(F)(F)F)cc(C(F)(F)F)c2)cc(-c2cc(C(F)(F)F)cc(C(F)(F)F)c2)c1-c1cc(C(F)(F)F)cc(C(F)(F)F)c1]

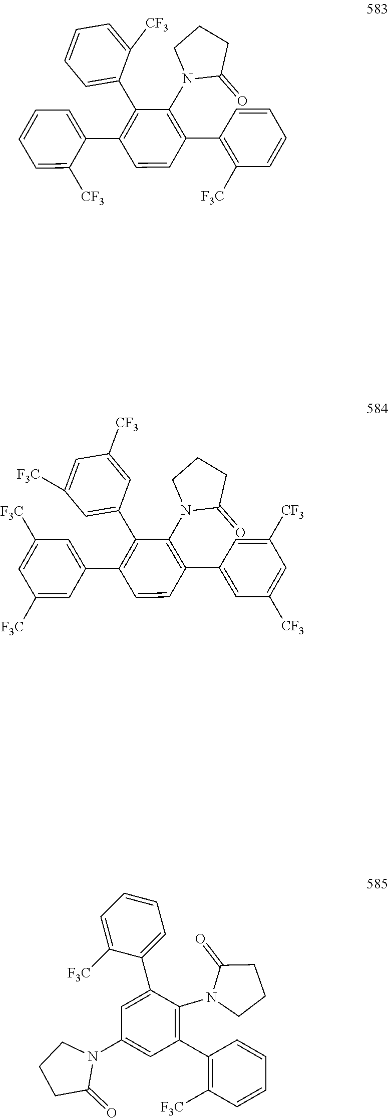 [SMILES: O=C1CCCN1c1c(-c2ccccc2C(F)(F)F)ccc(-c2ccccc2C(F)(F)F)c1-c1ccccc1C(F)(F)F ; O=C1CCCN1c1c(-c2cc(C(F)(F)F)cc(C(F)(F)F)c2)ccc(-c2cc(C(F)(F)F)cc(C(F)(F)F)c2)c1-c1cc(C(F)(F)F)cc(C(F)(F)F)c1 ; O=C1CCCN1c1cc(-c2ccccc2C(F)(F)F)c(N2CCCC2=O)c(-c2ccccc2C(F)(F)F)c1]

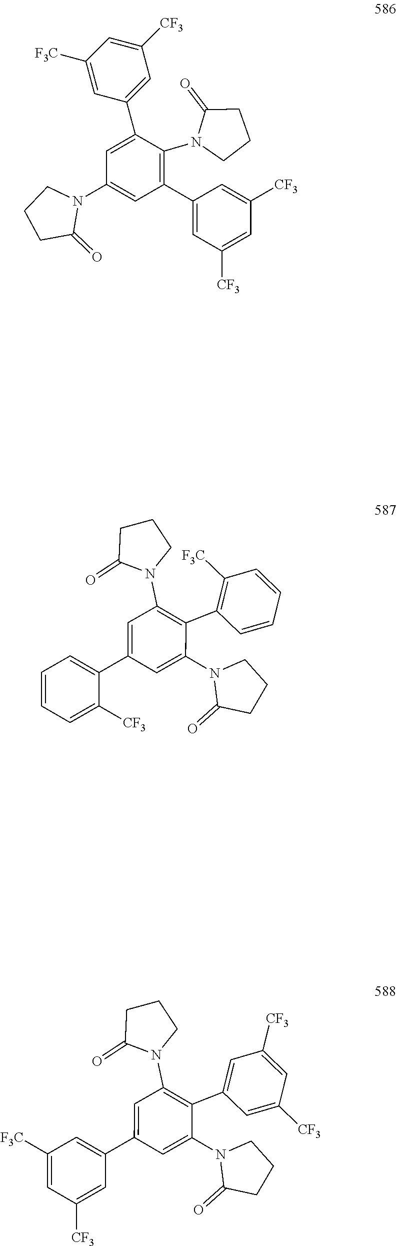 [SMILES: O=C1CCCN1c1cc(-c2cc(C(F)(F)F)cc(C(F)(F)F)c2)c(N2CCCC2=O)c(-c2cc(C(F)(F)F)cc(C(F)(F)F)c2)c1 ; O=C1CCCN1c1cc(-c2ccccc2C(F)(F)F)cc(N2CCCC2=O)c1-c1ccccc1C(F)(F)F ; O=C1CCCN1c1cc(-c2cc(C(F)(F)F)cc(C(F)(F)F)c2)cc(N2CCCC2=O)c1-c1cc(C(F)(F)F)cc(C(F)(F)F)c1]

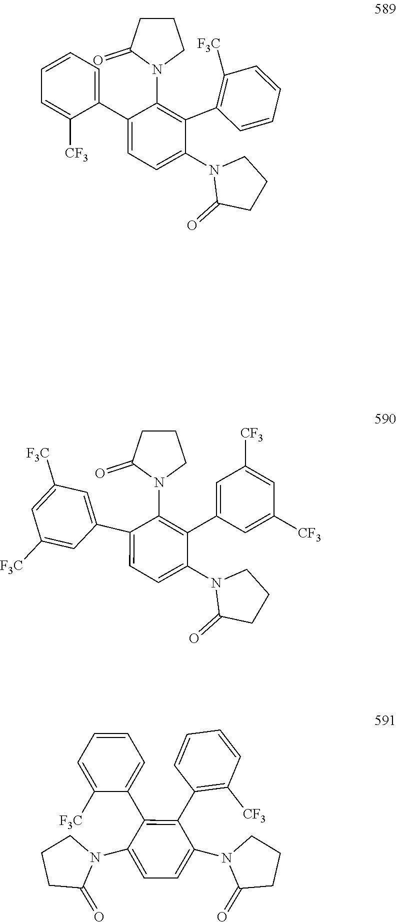 [SMILES: O=C1CCCN1c1ccc(-c2ccccc2C(F)(F)F)c(N2CCCC2=O)c1-c1ccccc1C(F)(F)F ; O=C1CCCN1c1ccc(-c2cc(C(F)(F)F)cc(C(F)(F)F)c2)c(N2CCCC2=O)c1-c1cc(C(F)(F)F)cc(C(F)(F)F)c1 ; O=C1CCCN1c1ccc(N2CCCC2=O)c(-c2ccccc2C(F)(F)F)c1-c1ccccc1C(F)(F)F]

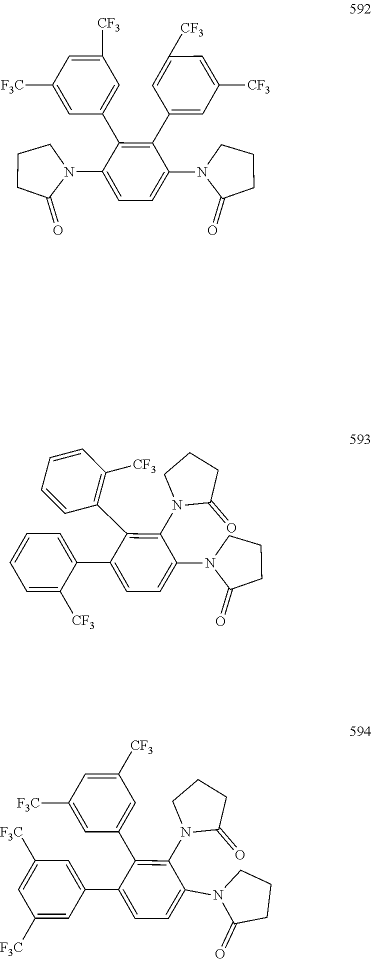 [SMILES: O=C1CCCN1c1ccc(N2CCCC2=O)c(-c2cc(C(F)(F)F)cc(C(F)(F)F)c2)c1-c1cc(C(F)(F)F)cc(C(F)(F)F)c1 ; O=C1CCCN1c1ccc(-c2ccccc2C(F)(F)F)c(-c2ccccc2C(F)(F)F)c1N1CCCC1=O ; O=C1CCCN1c1ccc(-c2cc(C(F)(F)F)cc(C(F)(F)F)c2)c(-c2cc(C(F)(F)F)cc(C(F)(F)F)c2)c1N1CCCC1=O]

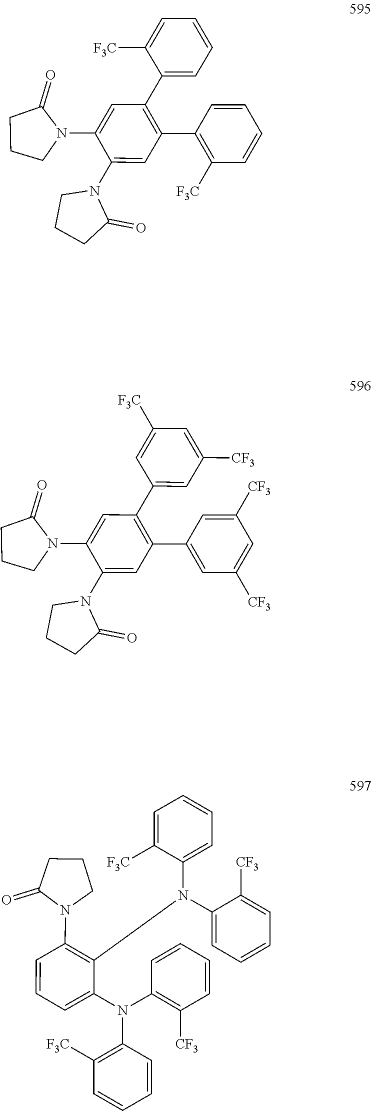 [SMILES: O=C1CCCN1c1cc(-c2ccccc2C(F)(F)F)c(-c2ccccc2C(F)(F)F)cc1N1CCCC1=O ; O=C1CCCN1c1cc(-c2cc(C(F)(F)F)cc(C(F)(F)F)c2)c(-c2cc(C(F)(F)F)cc(C(F)(F)F)c2)cc1N1CCCC1=O ; O=C1CCCN1c1cccc(N(c2ccccc2C(F)(F)F)c2ccccc2C(F)(F)F)c1N(c1ccccc1C(F)(F)F)c1ccccc1C(F)(F)F]

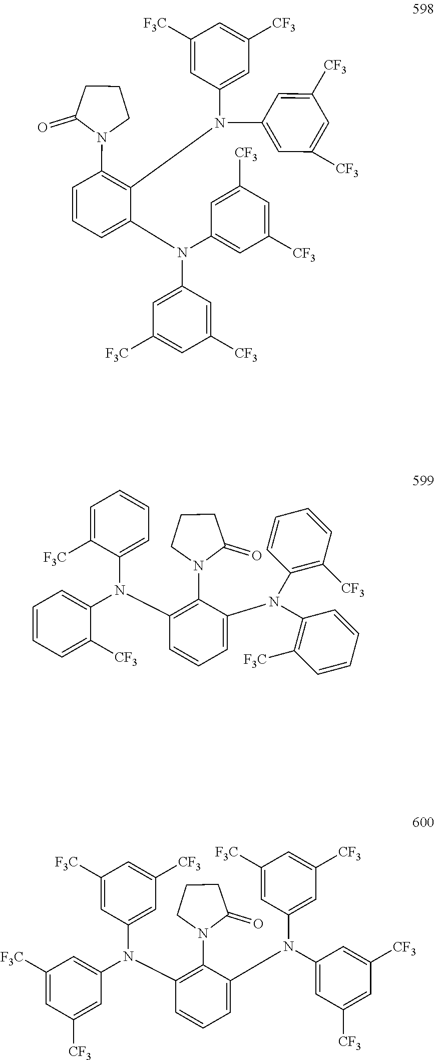 [SMILES: O=C1CCCN1c1cccc(N(c2cc(C(F)(F)F)cc(C(F)(F)F)c2)c2cc(C(F)(F)F)cc(C(F)(F)F)c2)c1N(c1cc(C(F)(F)F)cc(C(F)(F)F)c1)c1cc(C(F)(F)F)cc(C(F)(F)F)c1 ; O=C1CCCN1c1c(N(c2ccccc2C(F)(F)F)c2ccccc2C(F)(F)F)cccc1N(c1ccccc1C(F)(F)F)c1ccccc1C(F)(F)F ; O=C1CCCN1c1c(N(c2cc(C(F)(F)F)cc(C(F)(F)F)c2)c2cc(C(F)(F)F)cc(C(F)(F)F)c2)cccc1N(c1cc(C(F)(F)F)cc(C(F)(F)F)c1)c1cc(C(F)(F)F)cc(C(F)(F)F)c1]

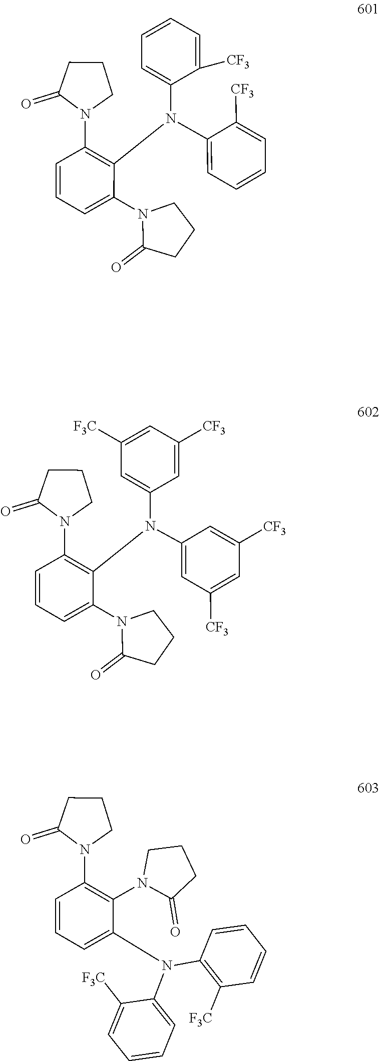 [SMILES: O=C1CCCN1c1cccc(N2CCCC2=O)c1N(c1ccccc1C(F)(F)F)c1ccccc1C(F)(F)F ; O=C1CCCN1c1cccc(N2CCCC2=O)c1N(c1cc(C(F)(F)F)cc(C(F)(F)F)c1)c1cc(C(F)(F)F)cc(C(F)(F)F)c1 ; O=C1CCCN1c1cccc(N(c2ccccc2C(F)(F)F)c2ccccc2C(F)(F)F)c1N1CCCC1=O]

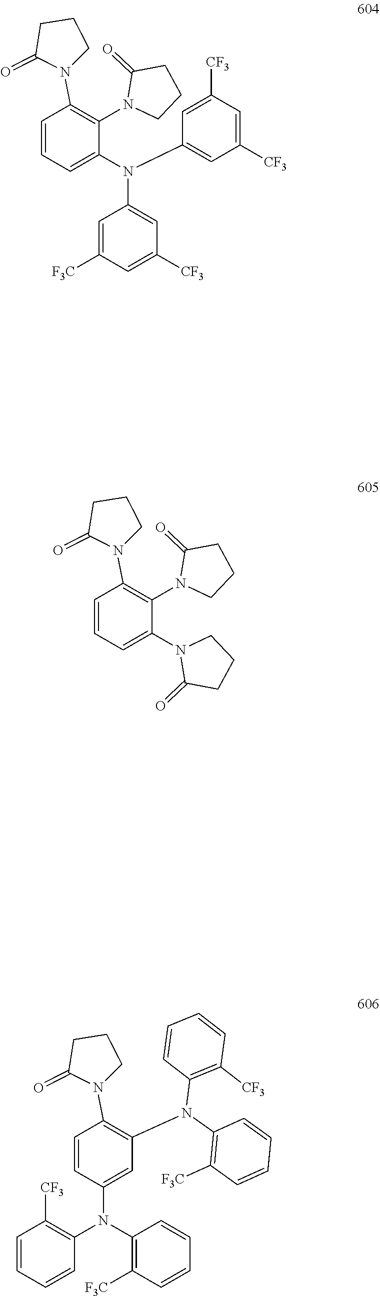 [SMILES: O=C1CCCN1c1cccc(N(c2cc(C(F)(F)F)cc(C(F)(F)F)c2)c2cc(C(F)(F)F)cc(C(F)(F)F)c2)c1N1CCCC1=O ; O=C1CCCN1c1cccc(N2CCCC2=O)c1N1CCCC1=O ; O=C1CCCN1c1ccc(N(c2ccccc2C(F)(F)F)c2ccccc2C(F)(F)F)cc1N(c1ccccc1C(F)(F)F)c1ccccc1C(F)(F)F]

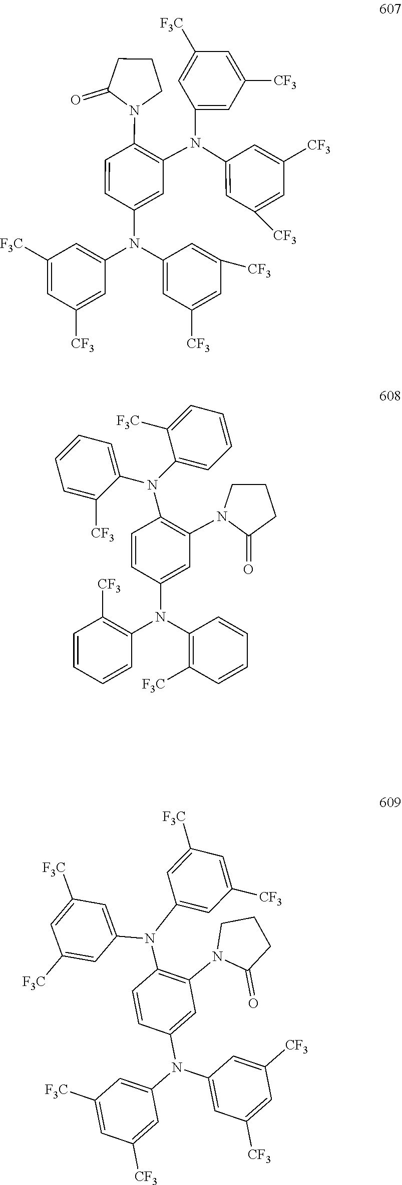 [SMILES: O=C1CCCN1c1ccc(N(c2cc(C(F)(F)F)cc(C(F)(F)F)c2)c2cc(C(F)(F)F)cc(C(F)(F)F)c2)cc1N(c1cc(C(F)(F)F)cc(C(F)(F)F)c1)c1cc(C(F)(F)F)cc(C(F)(F)F)c1 ; O=C1CCCN1c1cc(N(c2ccccc2C(F)(F)F)c2ccccc2C(F)(F)F)ccc1N(c1ccccc1C(F)(F)F)c1ccccc1C(F)(F)F ; O=C1CCCN1c1cc(N(c2cc(C(F)(F)F)cc(C(F)(F)F)c2)c2cc(C(F)(F)F)cc(C(F)(F)F)c2)ccc1N(c1cc(C(F)(F)F)cc(C(F)(F)F)c1)c1cc(C(F)(F)F)cc(C(F)(F)F)c1]

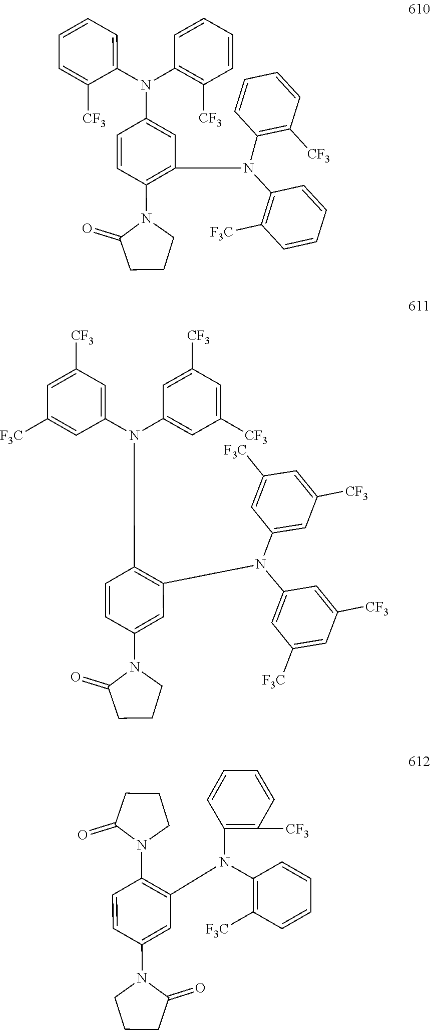 [SMILES: O=C1CCCN1c1ccc(N(c2ccccc2C(F)(F)F)c2ccccc2C(F)(F)F)cc1N(c1ccccc1C(F)(F)F)c1ccccc1C(F)(F)F ; O=C1CCCN1c1ccc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)c1 ; O=C1CCCN1c1ccc(N2CCCC2=O)c(N(c2ccccc2C(F)(F)F)c2ccccc2C(F)(F)F)c1]

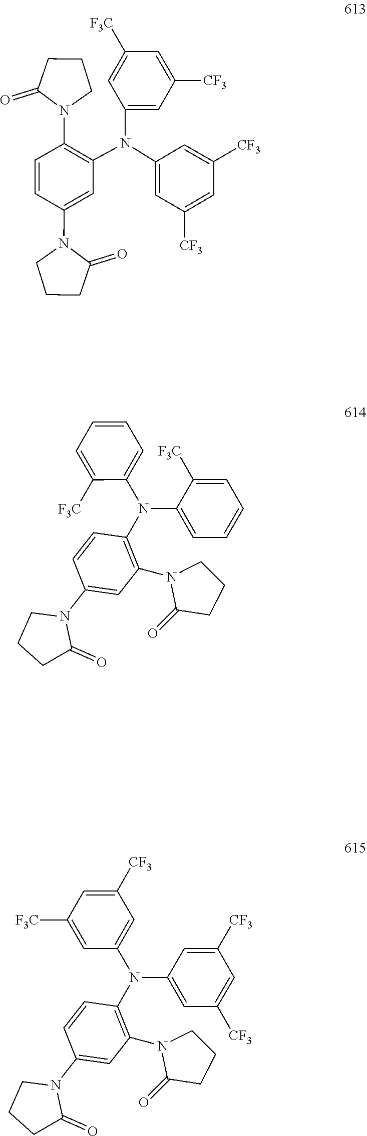 [SMILES: O=C1CCCN1c1ccc(N2CCCC2=O)c(N(c2cc(C(F)(F)F)cc(C(F)(F)F)c2)c2cc(C(F)(F)F)cc(C(F)(F)F)c2)c1 ; O=C1CCCN1c1ccc(N(c2ccccc2C(F)(F)F)c2ccccc2C(F)(F)F)c(N2CCCC2=O)c1 ; O=C1CCCN1c1ccc(N(c2cc(C(F)(F)F)cc(C(F)(F)F)c2)c2cc(C(F)(F)F)cc(C(F)(F)F)c2)c(N2CCCC2=O)c1]

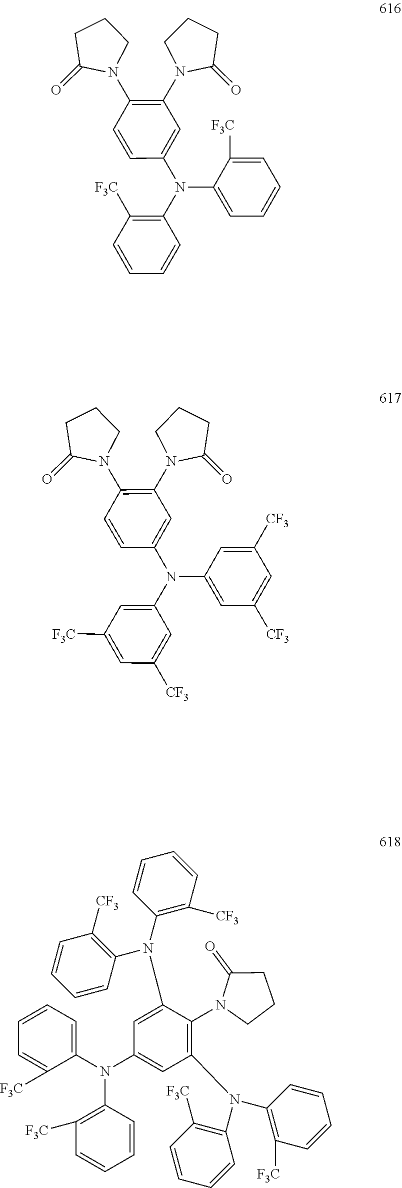 [SMILES: O=C1CCCN1c1ccc(N(c2ccccc2C(F)(F)F)c2ccccc2C(F)(F)F)cc1N1CCCC1=O ; O=C1CCCN1c1ccc(N(c2cc(C(F)(F)F)cc(C(F)(F)F)c2)c2cc(C(F)(F)F)cc(C(F)(F)F)c2)cc1N1CCCC1=O ; O=C1CCCN1c1c(N(c2ccccc2C(F)(F)F)c2ccccc2C(F)(F)F)cc(N(c2ccccc2C(F)(F)F)c2ccccc2C(F)(F)F)cc1N(c1ccccc1C(F)(F)F)c1ccccc1C(F)(F)F]

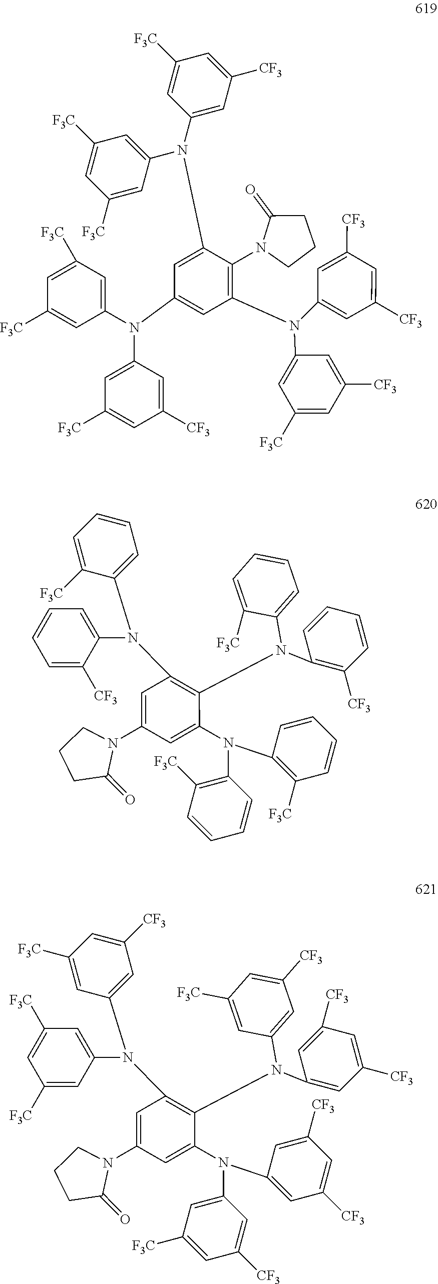

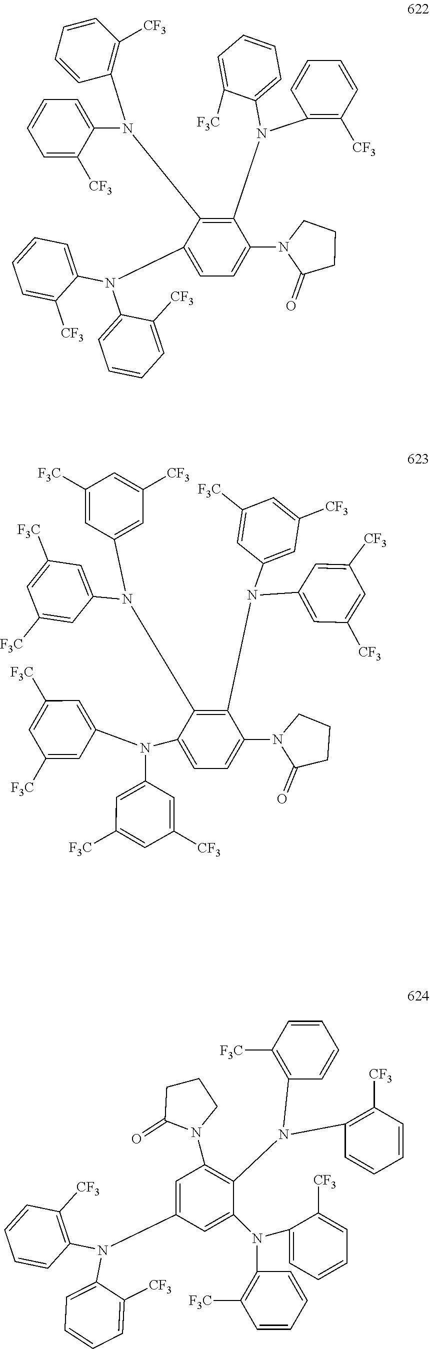 [SMILES: O=C1CCCN1c1ccc(N(c2ccccc2C(F)(F)F)c2ccccc2C(F)(F)F)c(N(c2ccccc2C(F)(F)F)c2ccccc2C(F)(F)F)c1N(c1ccccc1C(F)(F)F)c1ccccc1C(F)(F)F ; O=C1CCCN1c1ccc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)c1N(c1cc(C(F)(F)F)cc(C(F)(F)F)c1)c1cc(C(F)(F)F)cc(C(F)(F)F)c1 ; O=C1CCCN1c1cc(N(c2ccccc2C(F)(F)F)c2ccccc2C(F)(F)F)cc(N(c2ccccc2C(F)(F)F)c2ccccc2C(F)(F)F)c1N(c1ccccc1C(F)(F)F)c1ccccc1C(F)(F)F]

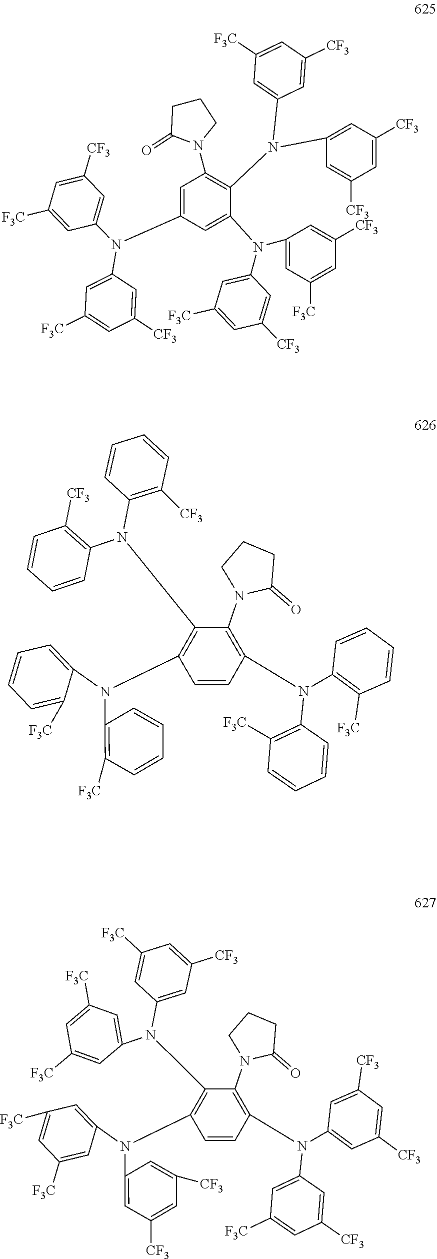

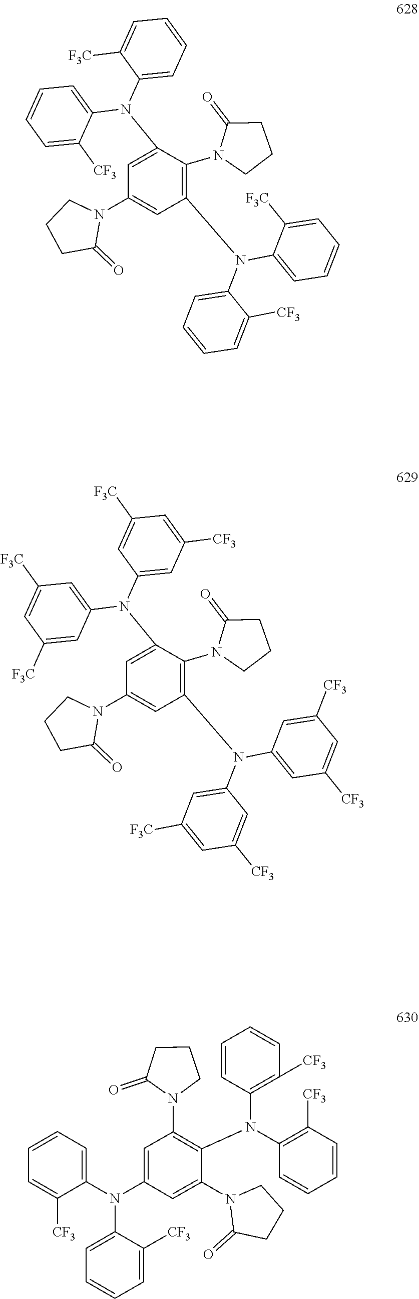 [SMILES: O=C1CCCN1c1cc(N(c2ccccc2C(F)(F)F)c2ccccc2C(F)(F)F)c(N2CCCC2=O)c(N(c2ccccc2C(F)(F)F)c2ccccc2C(F)(F)F)c1 ; O=C1CCCN1c1cc(N(c2cc(C(F)(F)F)cc(C(F)(F)F)c2)c2cc(C(F)(F)F)cc(C(F)(F)F)c2)c(N2CCCC2=O)c(N(c2cc(C(F)(F)F)cc(C(F)(F)F)c2)c2cc(C(F)(F)F)cc(C(F)(F)F)c2)c1 ; O=C1CCCN1c1cc(N(c2ccccc2C(F)(F)F)c2ccccc2C(F)(F)F)cc(N2CCCC2=O)c1N(c1ccccc1C(F)(F)F)c1ccccc1C(F)(F)F]

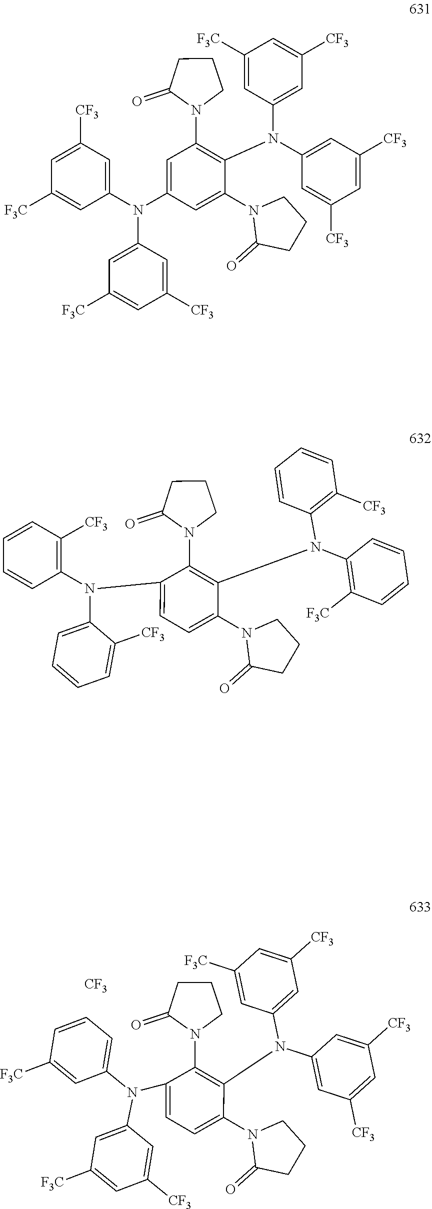 [SMILES: O=C1CCCN1c1cc(N(c2cc(C(F)(F)F)cc(C(F)(F)F)c2)c2cc(C(F)(F)F)cc(C(F)(F)F)c2)cc(N2CCCC2=O)c1N(c1cc(C(F)(F)F)cc(C(F)(F)F)c1)c1cc(C(F)(F)F)cc(C(F)(F)F)c1 ; O=C1CCCN1c1ccc(N(c2ccccc2C(F)(F)F)c2ccccc2C(F)(F)F)c(N2CCCC2=O)c1N(c1ccccc1C(F)(F)F)c1ccccc1C(F)(F)F ; O=C1CCCN1c1ccc(N(c2cccc(C(F)(F)F)c2)c2cc(C(F)(F)F)cc(C(F)(F)F)c2)c(N2CCCC2=O)c1N(c1cc(C(F)(F)F)cc(C(F)(F)F)c1)c1cc(C(F)(F)F)cc(C(F)(F)F)c1 ; "CF3"]

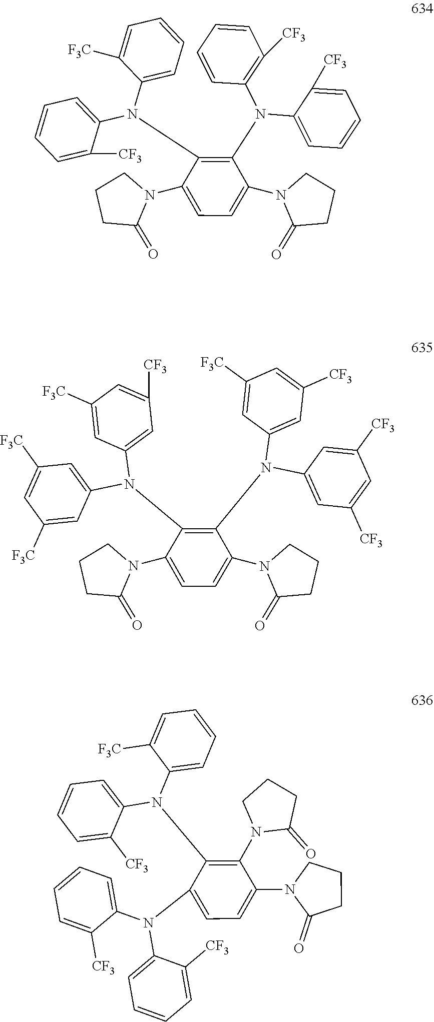 [SMILES: O=C1CCCN1c1ccc(N2CCCC2=O)c(N(c2ccccc2C(F)(F)F)c2ccccc2C(F)(F)F)c1N(c1ccccc1C(F)(F)F)c1ccccc1C(F)(F)F ; O=C1CCCN1c1ccc(N2CCCC2=O)c(N(c2cc(C(F)(F)F)cc(C(F)(F)F)c2)c2cc(C(F)(F)F)cc(C(F)(F)F)c2)c1N(c1cc(C(F)(F)F)cc(C(F)(F)F)c1)c1cc(C(F)(F)F)cc(C(F)(F)F)c1 ; O=C1CCCN1c1ccc(N(c2ccccc2C(F)(F)F)c2ccccc2C(F)(F)F)c(N(c2ccccc2C(F)(F)F)c2ccccc2C(F)(F)F)c1N1CCCC1=O]

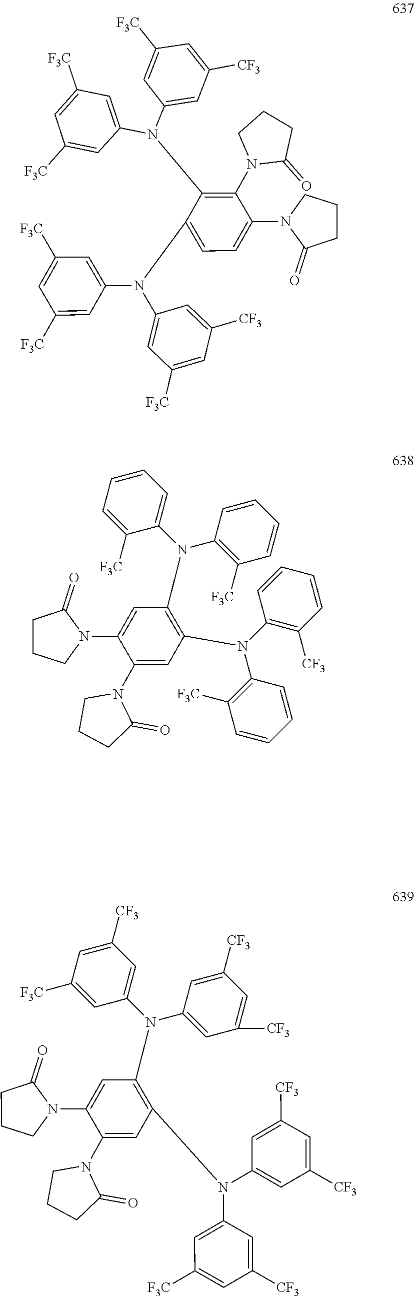 [SMILES: O=C1CCCN1c1ccc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)c1N1CCCC1=O ; O=C1CCCN1c1cc(N(c2ccccc2C(F)(F)F)c2ccccc2C(F)(F)F)c(N(c2ccccc2C(F)(F)F)c2ccccc2C(F)(F)F)cc1N1CCCC1=O ; O=C1CCCN1c1cc(N(c2cc(C(F)(F)F)cc(C(F)(F)F)c2)c2cc(C(F)(F)F)cc(C(F)(F)F)c2)c(N(c2cc(C(F)(F)F)cc(C(F)(F)F)c2)c2cc(C(F)(F)F)cc(C(F)(F)F)c2)cc1N1CCCC1=O]

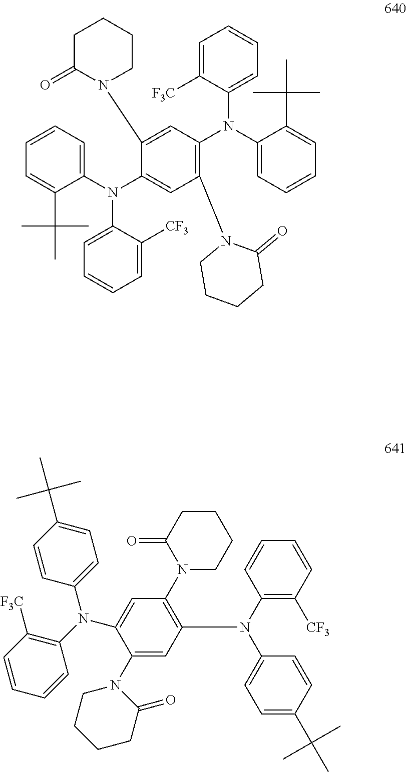 [SMILES: CC(C)(C)c1ccccc1N(c1cc(N2CCCCC2=O)c(N(c2ccccc2C(C)(C)C)c2ccccc2C(F)(F)F)cc1N1CCCCC1=O)c1ccccc1C(F)(F)F ; CC(C)(C)c1ccc(N(c2cc(N3CCCCC3=O)c(N(c3ccc(C(C)(C)C)cc3)c3ccccc3C(F)(F)F)cc2N2CCCCC2=O)c2ccccc2C(F)(F)F)cc1]

As such, the organic compound according to the present invention can synthesize organic compounds with various properties using moieties with unique properties. As a result, when the organic compound according to the present invention is applied to the light efficiency improving layer provided in the organic light emitting device, it is possible to further improve the luminescent properties such as luminous efficiency, etc. of the device.

In addition, the compound of an embodiment of the present invention may be applied to a device according to a general method for manufacturing an organic light emitting device.

An organic light emitting device according to an embodiment of the present invention may include a first electrode, a second electrode, and an organic layer arranged therebetween. The organic light emitting device may be manufactured using a general device manufacturing method and material, except that the organic compound of an embodiment of the present invention is used to form the organic layer of the device.

The organic layer of the organic light emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

In addition, the organic electroluminescent device may include a substrate, a first electrode (anode), an organic layer, a second electrode (cathode), and a light efficiency improving layer (capping layer), of which may be formed under the first electrode (bottom emission type) or on the second electrode (top emission type).

When the organic electroluminescent device is of a top emission type, light from the light emitting layer is emitted to the cathode and passes through the light efficiency improving layer (CPL) formed using the compound according to an embodiment of the present invention having a relatively high refractive index. The wavelength of the light is amplified, resulting in an increase in luminous efficiency. When the organic electroluminescent device is of a bottom emission type, the compound according to an embodiment of the present invention is employed in the light efficiency improving layer to improve the luminous efficiency of the organic electroluminescent device based on the same principle.

Preferred structures of the organic layers of the organic light emitting according to an embodiment of the present invention will be explained in more detail in the examples to be described later.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light emitting device.

### Mode for Carrying Out Invention

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 4

### (1) Preparation Example 1: Synthesis of Intermediate 4-1

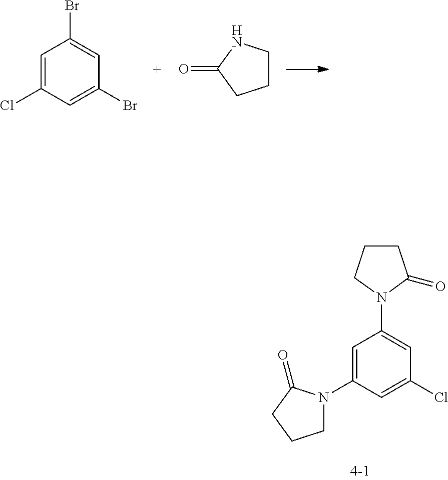 [SMILES: Clc1cc(Br)cc(Br)c1 ; O=C1CCCN1 ; O=C1CCCN1c1cc(Cl)cc(N2CCCC2=O)c1]

1,3-Dibromo-5-chlorobenzene (10.0 g, 0.037 mol), 2-pyrrolidinone (7.6 g, 0.089 mol), K₃PO₄ (47.1 g, 0.222 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then column and recrystallized to obtain 8.4 g of Intermediate 4-1 (yield 81.5%).

### (2) Preparation Example 2: Synthesis of Compound 4

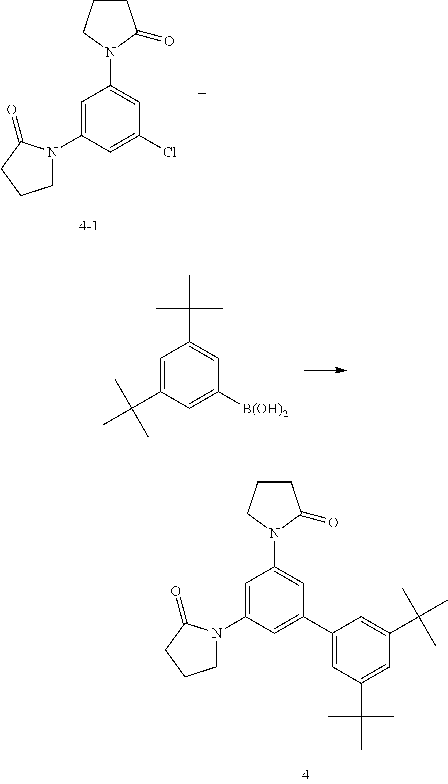 [SMILES: O=C1CCCN1c1cc(Cl)cc(N2CCCC2=O)c1 ; CC(C)(C)c1cc(B(O)O)cc(C(C)(C)C)c1 ; CC(C)(C)c1cc(-c2cc(N3CCCC3=O)cc(N3CCCC3=O)c2)cc(C(C)(C)C)c1]

Intermediate 4-1 (10.0 g, 0.036 mol), 3,5-Di-tert-butylphenylboronic Acid (10.1 g, 0.043 mol), K₂CO₃ (14.9 g, 0.108 mol), Pd(OAc)₂ (2.1 g, 0.002 mol), X-Phos (1.7 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 11.2 g of Compound 4 (yield 72.2%).

LC/MS: m/z=432[(M)⁺]

### Synthesis Example 2: Synthesis of Compound 7

### (1) Preparation Example 1: Synthesis of Compound 7

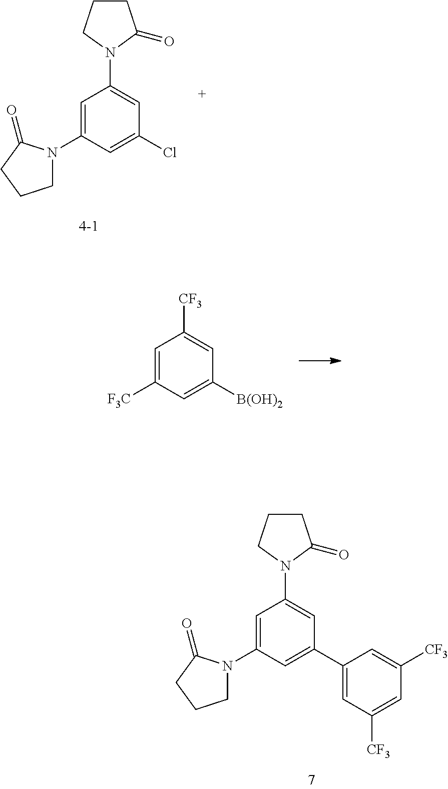 [SMILES: O=C1CCCN1c1cc(Cl)cc(N2CCCC2=O)c1 ; OB(O)c1cc(C(F)(F)F)cc(C(F)(F)F)c1 ; O=C1CCCN1c1cc(-c2cc(C(F)(F)F)cc(C(F)(F)F)c2)cc(N2CCCC2=O)c1]

Intermediate 4-1 (10.0 g, 0.036 mol), 3,5-Bis(trifluoromethyl)phenylboronic acid (11.1 g, 0.043 mol), K₂CO₃ (14.9 g, 0.108 mol), Pd(OAc)₂ (2.1 g, 0.002 mol), X-Phos (1.7 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 12.5 g of Compound 7 (yield 75.3%).

LC/MS: m/z=456[(M)⁺]

### Synthesis Example 3: Synthesis of Compound 12

### (1) Preparation Example 1: Synthesis of Compound 12

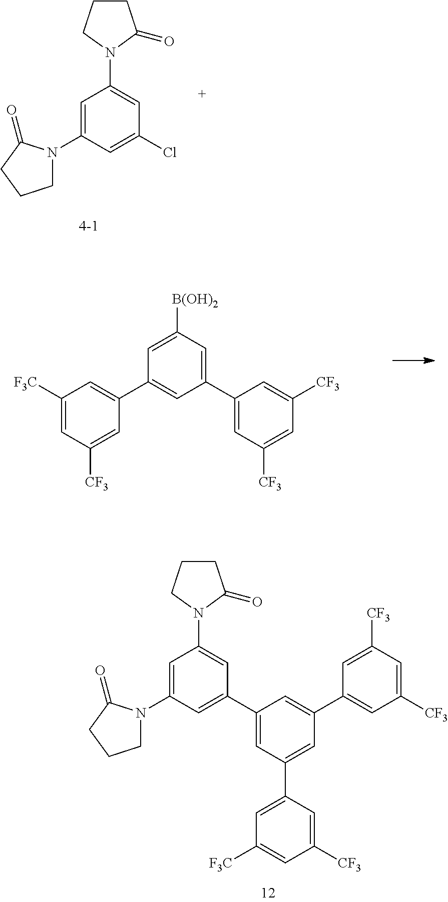 [SMILES: O=C1CCCN1c1cc(Cl)cc(N2CCCC2=O)c1 ; OB(O)c1cc(-c2cc(C(F)(F)F)cc(C(F)(F)F)c2)cc(-c2cc(C(F)(F)F)cc(C(F)(F)F)c2)c1 ; O=C1CCCN1c1cc(-c2cc(-c3cc(C(F)(F)F)cc(C(F)(F)F)c3)cc(-c3cc(C(F)(F)F)cc(C(F)(F)F)c3)c2)cc(N2CCCC2=O)c1]

Intermediate 4-1 (10.0 g, 0.036 mol), [3,5-Bis[3,5-bis(trifluoromethyl)phenyl]phenyl]boronic acid (23.5 g, 0.043 mol), K₂CO₃ (14.9 g, 0.108 mol), Pd(OAc)₂ (2.1 g, 0.002 mol), X-Phos (1.7 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 17.5 g of Compound 12 (yield 65.5%).

LC/MS: m/z=744[(M)⁺]

### Synthesis Example 4: Synthesis of Compound 22

### (1) Preparation Example 1: Synthesis of Compound 22

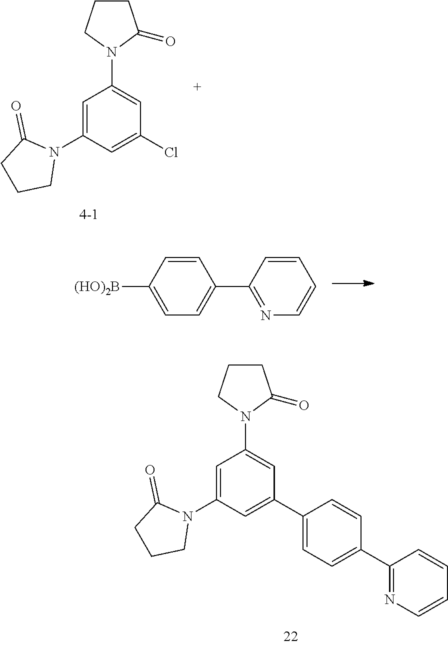 [SMILES: O=C1CCCN1c1cc(Cl)cc(N2CCCC2=O)c1 ; OB(O)c1ccc(-c2ccccn2)cc1 ; O=C1CCCN1c1cc(-c2ccc(-c3ccccn3)cc2)cc(N2CCCC2=O)c1]

Intermediate 4-1 (10.0 g, 0.036 mol), 4-(2-Pyridyl)phenyl boronic acid (8.6 g, 0.043 mol), K₂CO₃ (14.9 g, 0.108 mol), Pd(OAc)₂ (2.1 g, 0.002 mol), X-Phos (1.7 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 10.1 g of Compound 22 (yield 70.8%).

LC/MS: m/z=397[(M)⁺]

### Synthesis Example 5: Synthesis of Compound 58

### (1) Preparation Example 1: Synthesis of Intermediate 58-1

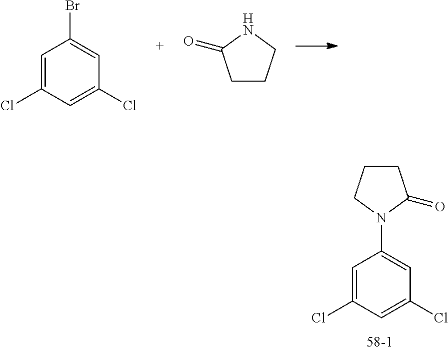 [SMILES: Clc1cc(Cl)cc(Br)c1 ; O=C1CCCN1 ; O=C1CCCN1c1cc(Cl)cc(Cl)c1]

1-Bromo-3,5-dichlorobenzene (10.0 g, 0.044 mol), 2-Pyrrolidinone (4.5 g, 0.053 mol), K₃PO₄ (28.2 g, 0.133 mol), Pd(dba)₂ (1.3 g, 0.002 mol), Xant-Phos (4.6 g, 0.008 mol), and dioxane were added and reacted while stirring under reflux for 16 hours. After completion of the reaction, the mixture was extracted, concentrated, and then column and recrystallized to obtain 7.8 g of Intermediate 58-1 (yield 76.6%).

### (2) Preparation Example 2: Synthesis of Compound 58

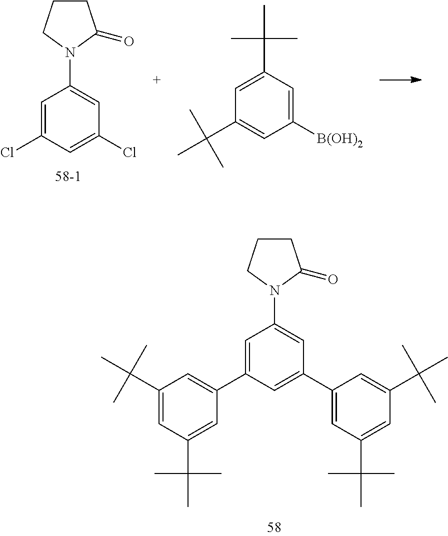 [SMILES: O=C1CCCN1c1cc(Cl)cc(Cl)c1 ; CC(C)(C)c1cc(B(O)O)cc(C(C)(C)C)c1 ; CC(C)(C)c1cc(-c2cc(-c3cc(C(C)(C)C)cc(C(C)(C)C)c3)cc(N3CCCC3=O)c2)cc(C(C)(C)C)c1]

Intermediate 58-1 (10.0 g, 0.044 mol), 3,5-Di-tert-butylphenylboronic Acid (24.4 g, 0.104 mol), K₂CO₃ (36.0 g, 0.261 mol), Pd(OAc)₂ (5.0 g, 0.004 mol), X-Phos (2.1 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 15.2 g of Compound 58 (yield 65.0%).

LC/MS: m/z=537[(M)⁺]

### Synthesis Example 6: Synthesis of Compound 74

### (1) Preparation Example 1: Synthesis of Compound 74

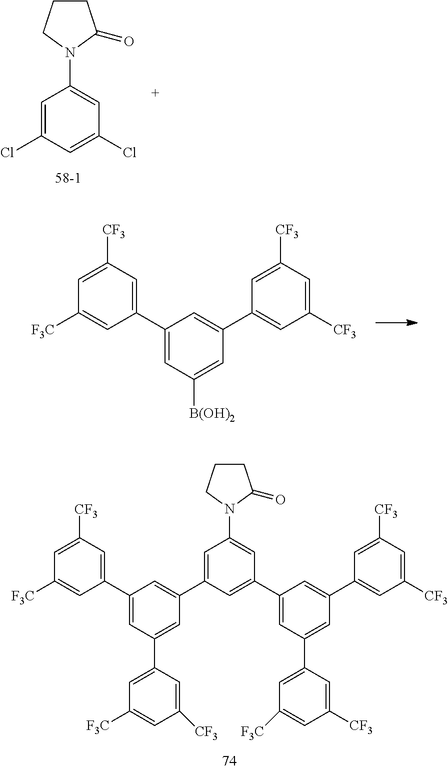

Intermediate 58-1 (10.0 g, 0.044 mol), [3,5-Bis[3,5-bis(trifluoromethyl)phenyl]phenyl]boronic acid (57.0 g, 0.104 mol), K₂CO₃ (36.0 g, 0.261 mol), Pd(OAc)₂ (5.0 g, 0.004 mol), X-Phos (2.1 g, 0.004 mol), THF 200 mL, and H₂O 50 mL were added and reacted while stirring at 90° C. for 6 hours. After completion of the reaction, the reaction mixture was extracted, concentrated, and then column and recrystallized to obtain 31.8 g of Compound 74 (yield 63.0%). LC/MS: m/z=1161[(M)⁺]

### Synthesis Example 7: Synthesis of Compound 84

### (1) Preparation Example 1: Synthesis of Intermediate 84-1

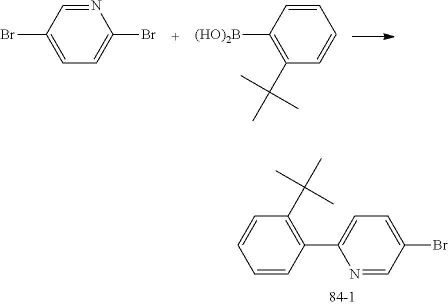 [SMILES: Brc1ccc(Br)nc1 ; CC(C)(C)c1ccccc1B(O)O ; CC(C)(C)c1ccccc1-c1ccc(Br)cn1]

2,5-Dibromopyridine (10.0 g, 0.042 mol), (2-Tert-butylphenyl) boronic acid (9.0 g, 0.051 mol), K₂CO₃ (17.5 g, 0.127 mol), and Pd(PPh₃)₄ (1.0 g, 0.0008 mol) were added with toluene 200 mL, ethanol 50 mL, and H₂O 50 mL and reacted while stirring at 100° C. for 6 hours. After completion of the reaction, the reaction mixture was extracted, concentrated, and then column and recrystallized to obtain 7.1 g of Intermediate 84-1 (yield 58.0%).

### (2) Preparation Example 2: Synthesis of Intermediate 84-2

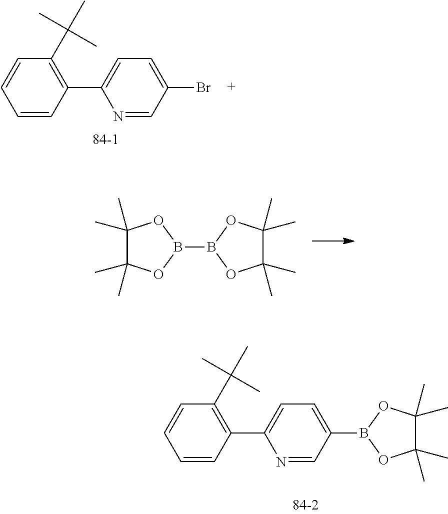

Intermediate 84-1 (10.0 g, 0.035 mol), Bis(pinacolato)diboron (10.5 g, 0.041 mol), KOAc (10.2 g, 0.103 mol), and Pd(dppf)Cl2 (1.3 g, 0.001 mol) were added with dioxane 200 mL and reacted while stirring at 100° C. for 12 hours. After completion of the reaction, the reaction mixture was extracted, concentrated, and then column and recrystallized to obtain 7.8 g of Intermediate 84-2 (yield 67.1%).

### (3) Preparation Example 3: Synthesis of Compound 84

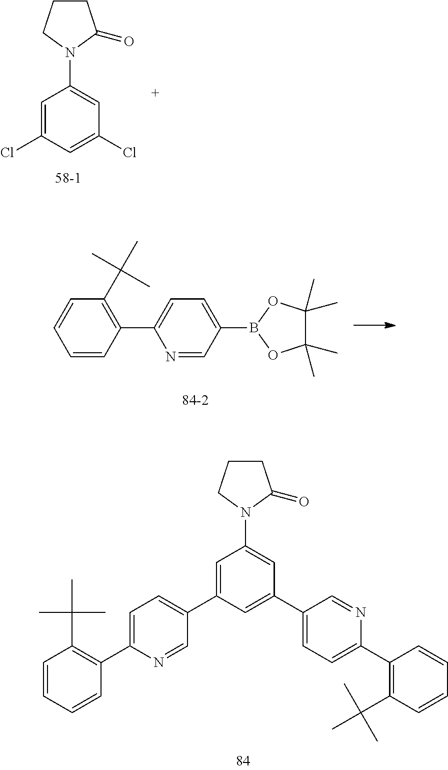

Intermediate 58-1 (10.0 g, 0.044 mol), Intermediate 84-2 (35.2 g, 0.104 mol), K₂CO₃ (36.0 g, 0.261 mol), Pd(OAc)₂ (5.0 g, 0.004 mol), X-Phos (2.1 g, 0.004 mol), THF 200 mL, and H₂O 50 mL were added and reacted while stirring at 90° C. for 6 hours.

After completion of the reaction, the reaction mixture was extracted, concentrated, and then column and recrystallized to obtain 16.2 g of Compound 84 (yield 64.3%). LC/MS: m/z=579[(M)⁺]

### Synthesis Example 8: Synthesis of Compound 101

### (1) Preparation Example 1: Synthesis of Compound 101

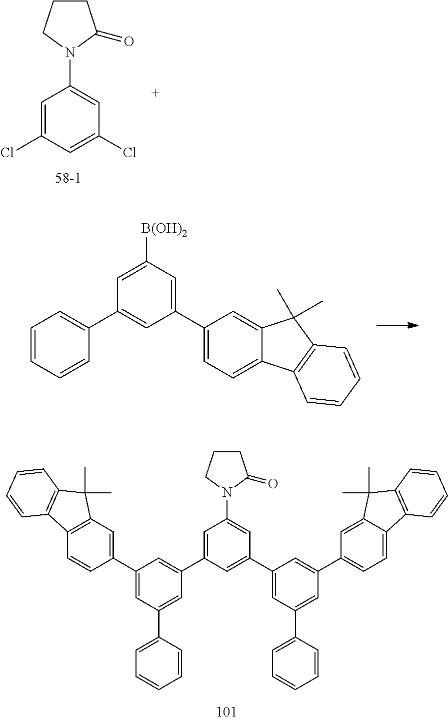 [SMILES: O=C1CCCN1c1cc(Cl)cc(Cl)c1 ; CC1(C)c2ccccc2-c2ccc(-c3cc(B(O)O)cc(-c4ccccc4)c3)cc21 ; CC1(C)c2ccccc2-c2ccc(-c3cc(-c4ccccc4)cc(-c4cc(-c5cc(-c6ccccc6)cc(-c6ccc7c(c6)C(C)(C)c6ccccc6-7)c5)cc(N5CCCC5=O)c4)c3)cc21]

Intermediate 58-1 (10.0 g, 0.044 mol), B-[5-(9,9-Dimethyl-9H-fluoren-2-yl)[1,1′-biphenyl]-3-yl]boronic acid (40.7 g, 0.104 mol), K₂CO₃ (36.0 g, 0.261 mol), Pd(OAc)₂ (5.1 g, 0.002 mol), X-Phos (2.1 g, 0.004 mol), THF 200 mL, and H₂O 50 mL were added and reacted while stirring at 90° C. for 6 hours. After completion of the reaction, the reaction mixture was extracted, concentrated, and the column and recrystallized to obtain 22.8 g of Compound 101 (yield 61.7%).

LC/MS: m/z=850[(M)⁺]

### Synthesis Example 9: Synthesis of Compound 112

### (1) Preparation Example 1: Synthesis of Intermediate 112-1

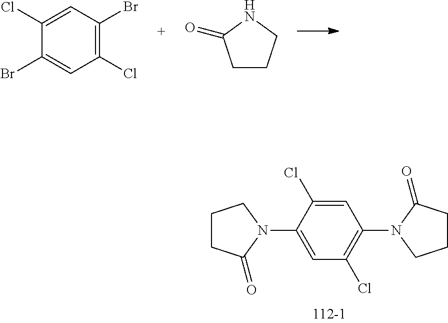 [SMILES: Clc1cc(Br)c(Cl)cc1Br ; O=C1CCCN1 ; O=C1CCCN1c1cc(Cl)c(N2CCCC2=O)cc1Cl]

1,4-Dibromo-2,5-dichlorobenzene (10.0 g, 0.033 mol), 2-Pyrrolidinone (6.7 g, 0.079 mol), K₃PO₄ (41.8 g, 0.197 mol), Pd(dba)₂ (1.9 g, 0.003 mol), Xant-Phos (13.7 g, 0.024 mol), and dioxane were added and reacted while stirring under reflux for 16 hours. After completion of the reaction, the reaction mixture was extracted, concentrated, and then column and recrystallized to obtain 6.2 g of Intermediate 112-1 (yield 60.3%).

### (2) Preparation Example 2: Synthesis of Compound 112

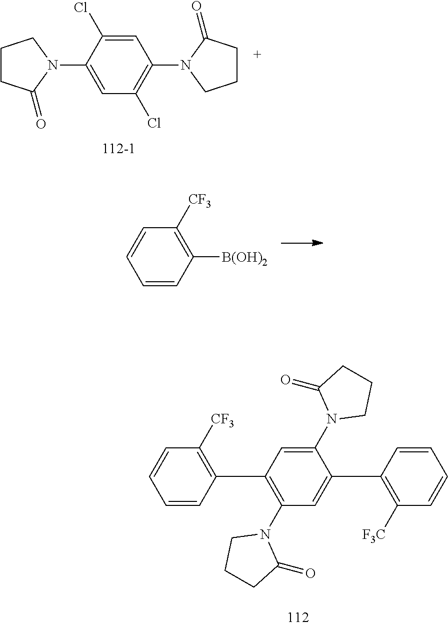 [SMILES: O=C1CCCN1c1cc(Cl)c(N2CCCC2=O)cc1Cl ; OB(O)c1ccccc1C(F)(F)F ; O=C1CCCN1c1cc(-c2ccccc2C(F)(F)F)c(N2CCCC2=O)cc1-c1ccccc1C(F)(F)F]

Intermediate 112-1 (10.0 g, 0.032 mol), 2-Trifluoromethylbenzeneboronic acid (14.6 g, 0.077 mol), K₂CO₃ (26.5 g, 0.192 mol), Pd(OAc)₂ (3.7 g, 0.003 mol), X-Phos (1.5 g, 0.003 mol), THF 200 mL, and H₂O 50 mL were added and reacted while stirring at 90° C. for 6 hours. After completion of the reaction, the reaction mixture was extracted, concentrated, and then column and recrystallized to obtain 10.7 g of Compound 112 (yield 62.9%).

LC/MS: m/z=532[(M)⁺]

### Synthesis Example 10: Synthesis of Compound 124

### (1) Preparation Example 1: Synthesis of Intermediate 124-1

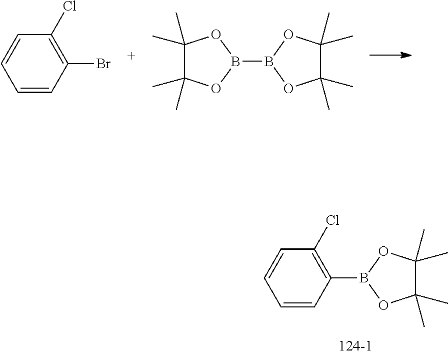

1-Bromo-2-chlorobenzene (10.0 g, 0.052 mol), Bis(pinacolato)diboron (15.9 g, 0.063 mol), KOAc (15.4 g, 0.157 mol), and Pd(dppf)Cl₂ (1.9 g, 0.003 mol) were added with dioxane 200 mL and reacted while stirring at 100° C. for 12 hours. After completion of the reaction, the reaction mixture was extracted, concentrated, and then columned to obtain 8.2 g of Intermediate 124-1 (yield 65.8%).

### (2) Preparation Example 2: Synthesis of Intermediate 124-2

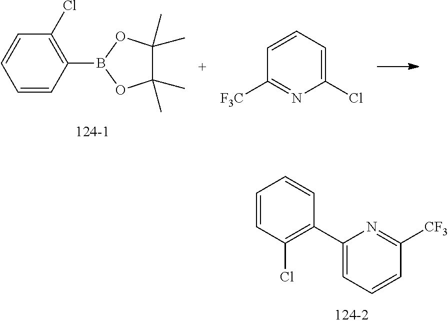 [SMILES: CC1(C)OB(c2ccccc2Cl)OC1(C)C ; FC(F)(F)c1cccc(Cl)n1 ; FC(F)(F)c1cccc(-c2ccccc2Cl)n1]

Intermediate 124-1 (10.0 g, 0.042 mol), 2-Chloro-6-(trifluoromethyl)pyridine (9.1 g, 0.050 mol), K₂CO₃ (17.4 g, 0.126 mol), and Pd(PPh₃)₄ (1.0 g, 0.0008 mol) were added with toluene 200 mL, ethanol 50 mL, and H₂O 50 mL and reacted while stirring at 100° C. for 6 hours. After completion of the reaction, the reaction mixture was extracted, concentrated, and then columned to obtain 6.6 g of Intermediate 124-2 (yield 61.1%).

### (3) Preparation Example 3: Synthesis of Intermediate 124-3

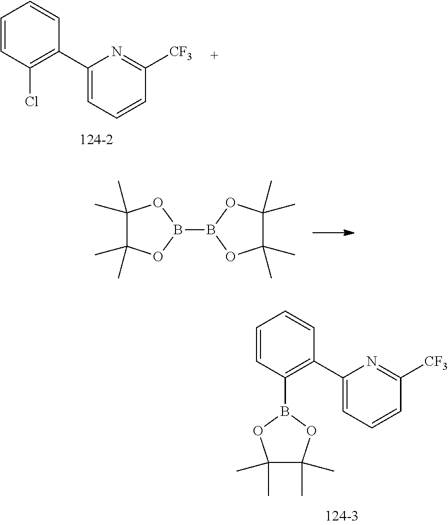

Intermediate 124-2 (10.0 g, 0.039 mol), Bis(pinacolato)diboron (11.8 g, 0.047 mol), KOAc (11.4 g, 0.116 mol), Pd(dppf)Cl₂ (1.4 g, 0.002 mol), and XPhos (5.6 g, 0.012 mol) were added with dioxane 200 mL and reacted while stirring at 100° C. for 12 hours. After completion of the reaction, the reaction mixture was extracted, concentrated, and then column and recrystallized to obtain 9.7 g of Intermediate 124-3 (yield 71.6%).

### (4) Preparation Example 4: Synthesis of Compound 124

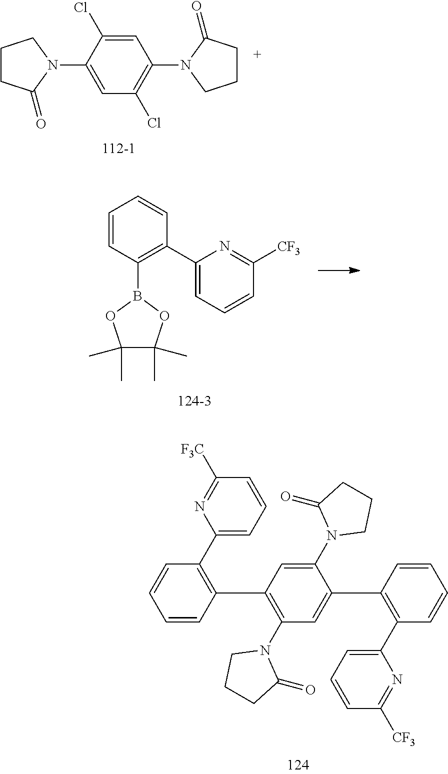 [SMILES: O=C1CCCN1c1cc(Cl)c(N2CCCC2=O)cc1Cl ; CC1(C)OB(c2ccccc2-c2cccc(C(F)(F)F)n2)OC1(C)C ; O=C1CCCN1c1cc(-c2ccccc2-c2cccc(C(F)(F)F)n2)c(N2CCCC2=O)cc1-c1ccccc1-c1cccc(C(F)(F)F)n1]

Intermediate 112-1 (10.0 g, 0.032 mol), Intermediate 124-3 (26.8 g, 0.077 mol), K₂CO₃ (22.1 g, 0.160 mol), Pd(OAc)₂ (3.7 g, 0.003 mol), X-Phos (1.5 g, 0.003 mol), THF 200 mL, and H₂O 50 mL were added and reacted while stirring at 90° C. for 6 hours. After completion of the reaction, the reaction mixture was extracted, concentrated, and then column and recrystallized to obtain 12.8 g of Compound 124 (yield 58.4%).

LC/MS: m/z=686[(M)⁺]

### Synthesis Example 11: Synthesis of Compound 136

### (1) Preparation Example 1: Synthesis of Compound 136

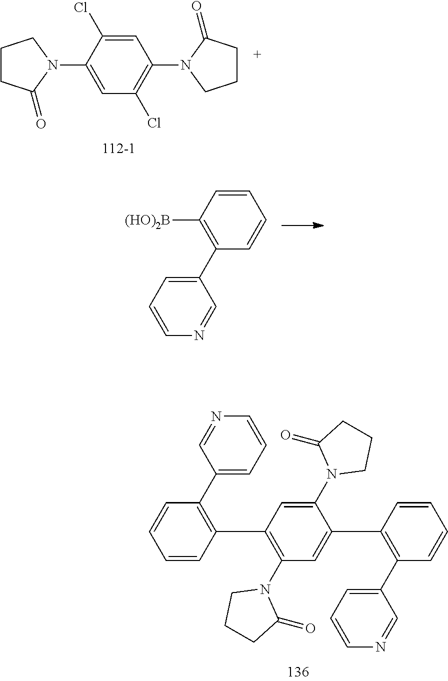 [SMILES: O=C1CCCN1c1cc(Cl)c(N2CCCC2=O)cc1Cl ; OB(O)c1ccccc1-c1cccnc1 ; O=C1CCCN1c1cc(-c2ccccc2-c2cccnc2)c(N2CCCC2=O)cc1-c1ccccc1-c1cccnc1]

Intermediate 112-1 (10.0 g, 0.032 mol), [2-(Pyridin-3-yl)phenyl]boronic acid (15.3 g, 0.077 mol), K₂CO₃ (22.1 g, 0.160 mol), Pd(OAc)₂ (3.7 g, 0.003 mol), X-Phos (1.5 g, 0.003 mol), THF 200 mL, and H₂O 50 mL were added and reacted while stirring at 90° C. for 6 hours. After completion of the reaction, the reaction mixture was extracted, concentrated, and the column and recrystallized to obtain 11.5 g of Compound 136 (yield 65.4%).

LC/MS: m/z=550[(M)⁺]

### Synthesis Example 12: Synthesis of Compound 164

### (1) Preparation Example 1: Synthesis of Compound 164

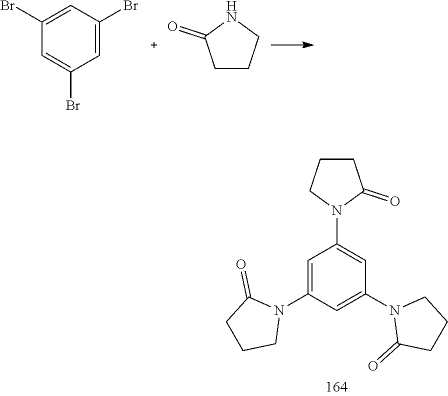 [SMILES: Brc1cc(Br)cc(Br)c1 ; O=C1CCCN1 ; O=C1CCCN1c1cc(N2CCCC2=O)cc(N2CCCC2=O)c1]

1,3,5-Tribromobenzene (10.0 g, 0.032 mol), 2-Pyrrolidinone (9.7 g, 0.114 mol), K₃PO₄ (60.7 g, 0.286 mol), Pd(dba)₂ (2.7 g, 0.005 mol), Xant-Phos (29.8 g, 0.052 mol), and dioxane were added and reacted while stirring under reflux for 16 hours. After completion of the reaction, the reaction mixture was extracted, concentrated, and then column and recrystallized to obtain 7.2 g of Compound 164 (yield 69.2%).

LC/MS: m/z=731[(M)⁺]

### Synthesis Example 13: Synthesis of Compound 165

### (1) Preparation Example 1: Synthesis of Compound 165

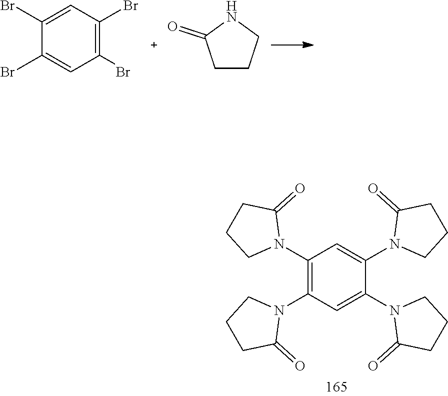 [SMILES: Brc1cc(Br)c(Br)cc1Br ; O=C1CCCN1 ; O=C1CCCN1c1cc(N2CCCC2=O)c(N2CCCC2=O)cc1N1CCCC1=O]

2,3,5,6-Tetrabromobenzene (10.0 g, 0.025 mol), 2-Pyrrolidinone (10.4 g, 0.122 mol), K₃PO₄ (64.7 g, 0.305 mol), Pd(dba)₂ (2.9 g, 0.005 mol), Xant-Phos (42.3 g, 0.073 mol), and dioxane were added and reacted while stirring under reflux for 16 hours. After completion of the reaction, the reaction mixture was extracted, concentrated, and then column and recrystallized to obtain 5.8 g of Compound 165 (yield 55.6%).

LC/MS: m/z=410[(M)⁺]

### Synthesis Example 14: Synthesis of Compound 224

### (1) Preparation Example 1: Synthesis of Intermediate 224-1

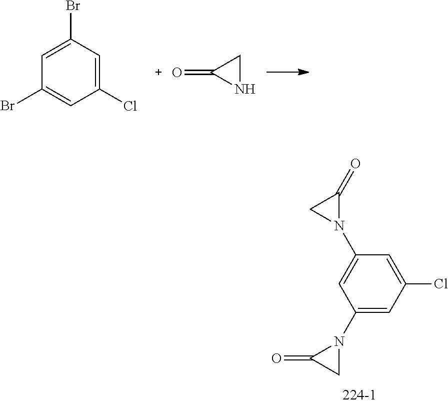

1,3-Dibromo-5-chlorobenzene (10.0 g, 0.037 mol), Aziridinone (5.1 g, 0.089 mol), K₃PO₄ (47.1 g, 0.222 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then columned to obtain 6.8 g of Intermediate 224-1 (yield 82.6%).

### (2) Preparation Example 2: Synthesis of Intermediate 224-2

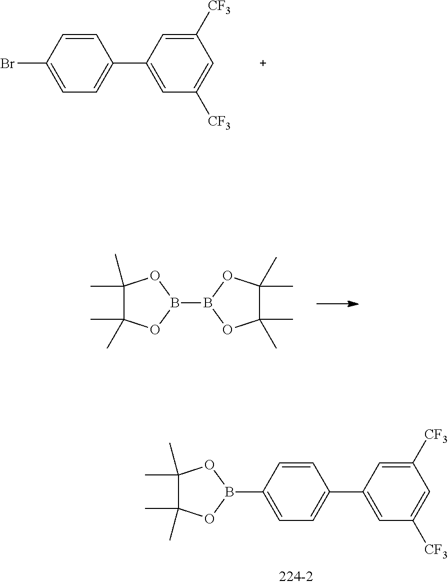

1-Bromo-3′,5′-bis(trifluoromethyl)-1,1′-biphenyl (10.0 g, 0.027 mol), Bis(pinacolato)diboron (8.3 g, 0.033 mol), KOAc (8.0 g, 0.081 mol), and Pd(dppf)Cl₂ (1.0 g, 0.001 mol) were added with dioxane 200 mL and reacted while stirring at 100° C. for 12 hours. After completion of the reaction, the reaction mixture was extracted, concentrated, and then column and recrystallized to obtain 8.1 g of Intermediate 224-2 (yield 71.8%).

### (3) Preparation Example 3: Synthesis of Compound 224

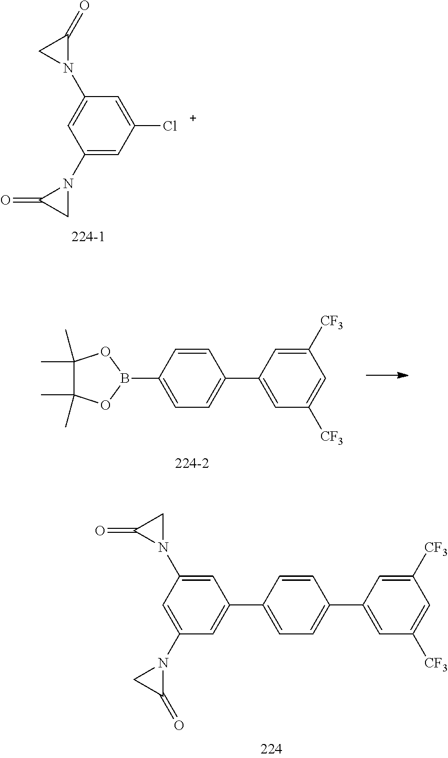 [SMILES: O=C1CN1c1cc([ClH+])cc(N2CC2=O)c1 ; CC1(C)OB(c2ccc(-c3cc(C(F)(F)F)cc(C(F)(F)F)c3)cc2)OC1(C)C ; O=C1CN1c1cc(-c2ccc(-c3cc(C(F)(F)F)cc(C(F)(F)F)c3)cc2)cc(N2CC2=O)c1]

Intermediate 224-1 (10.0 g, 0.045 mol), Intermediate 224-2 (22.4 g, 0.054 mol), K₂CO₃ (18.6 g, 0.135 mol), Pd(OAc)₂ (2.6 g, 0.002 mol), X-Phos (2.1 g, 0.005 mol), THF 200 mL, and H₂O 50 mL were added and reacted while stirring at 90° C. for 6 hours. After completion of the reaction, the reaction mixture was extracted, concentrated, and then column and recrystallized to obtain 16.2 g of Compound 224 (yield 75.7%).

LC/MS: m/z=476[(M)⁺]

### Synthesis Example 15: Synthesis of Compound 396

### (1) Preparation Example 1: Synthesis of Intermediate 396-1

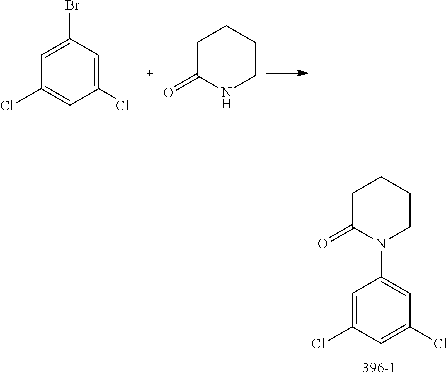

1-Bromo-3,5-dichlorobenzene (10.0 g, 0.044 mol), 2-Piperidone (5.3 g, 0.053 mol), K₃PO₄ (28.2 g, 0.133 mol), Pd(dba)₂ (1.3 g, 0.002 mol), Xant-Phos (4.6 g, 0.008 mol), and dioxane 50 mL were added and reacted while stirring under reflux for 16 hours. After completion of the reaction, the reaction mixture was extracted, concentrated, and then columned to obtain 8.5 g of Intermediate 396-1 (yield 78.7%).

### (2) Preparation Example 2: Synthesis of Intermediate 396-2

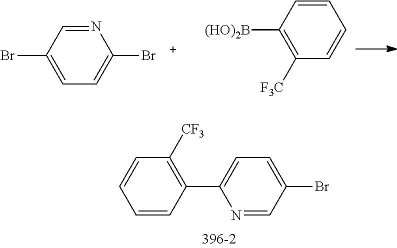 [SMILES: Brc1ccc(Br)nc1 ; OB(O)c1ccccc1C(F)(F)F ; FC(F)(F)c1ccccc1-c1ccc(Br)cn1]

2,5-Dibromopyridine (10.0 g, 0.042 mol), 2-Trifluoromethylbenzeneboronic acid (9.6 g, 0.051 mol), K₂CO₃ (17.5 g, 0.127 mol), and Pd(PPh₃)₄ (1.0 g, 0.0008 mol) were added with toluene 200 mL, ethanol 50 mL, and H₂O 50 mL and reacted while stirring at 100° C. for 6 hours. After completion of the reaction, the reaction mixture was extracted, concentrated, and then columned to obtain 9.3 g of Intermediate 396-2 (yield 72.9%).

### (3) Preparation Example 3: Synthesis of Intermediate 396-3

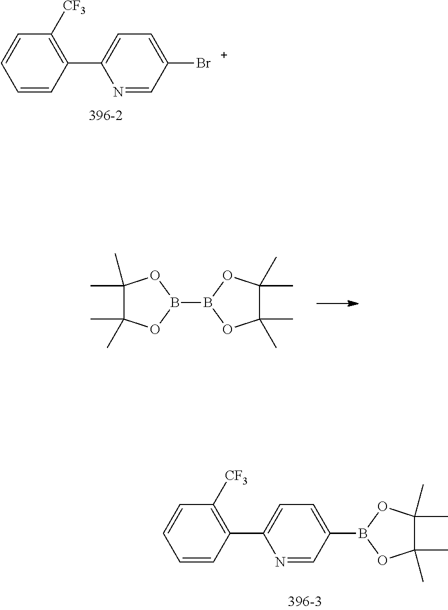

Intermediate 396-2 (10.0 g, 0.033 mol), Bis(pinacolato)diboron (10.1 g, 0.040 mol), KOAc (9.8 g, 0.099 mol), and Pd(dppf)Cl2 (1.2 g, 0.002 mol) were added with dioxane 200 mL and reacted while stirring at 100° C. for 12 hours. After completion of the reaction, the reaction mixture was extracted, concentrated, and then column and recrystallized to obtain 8.6 g of Intermediate 396-3 (yield 74.4%).

### (4) Preparation Example 4: Synthesis of Compound 396

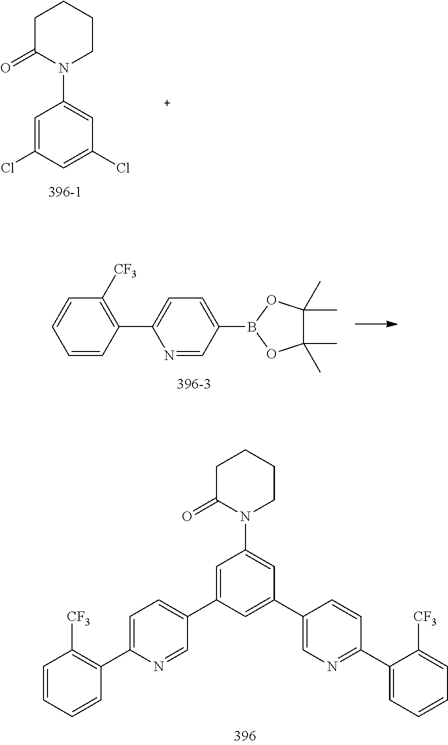 [SMILES: O=C1CCCCN1c1cc(Cl)cc(Cl)c1 ; CC1(C)OB(c2ccc(-c3ccccc3C(F)(F)F)nc2)OC1(C)C ; O=C1CCCCN1c1cc(-c2ccc(-c3ccccc3C(F)(F)F)nc2)cc(-c2ccc(-c3ccccc3C(F)(F)F)nc2)c1]

Intermediate 396-1 (10.0 g, 0.041 mol), Intermediate 396-3 (34.3 g, 0.098 mol), K₂CO₃ (28.3 g, 0.205 mol), Pd(OAc)₂ (4.7 g, 0.004 mol), X-Phos (2.0 g, 0.004 mol), THF 200 mL, and H₂O 50 mL were added and reacted while stirring at 90° C. for 6 hours. After completion of the reaction, the reaction mixture was extracted, concentrated, and the column and recrystallized to obtain 17.4 g of Compound 396 (yield 68.8%).

LC/MS: m/z=617[(M)⁺]

### Synthesis Example 16: Synthesis of Compound 422

### (1) Preparation Example 1: Synthesis of Intermediate 422-1

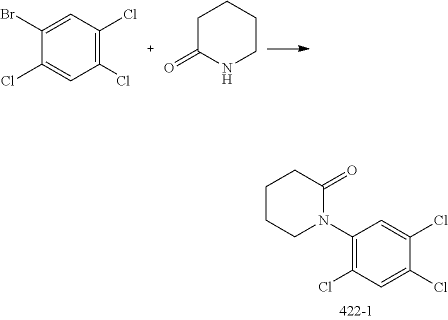

2,4,5-Trichlorobromobenzene (10.0 g, 0.038 mol), 2-Piperidone (4.6 g, 0.046 mol), K₃PO₄ (24.5 g, 0.115 mol), Pd(dba)₂ (1.1 g, 0.002 mol), Xant-Phos (4.0 g, 0.007 mol), and dioxane 50 mL were added and reacted while stirring under reflux for 16 hours. After completion of the reaction, the reaction mixture was extracted, concentrated, and then column and recrystallized to obtain 12.1 g of Intermediate 422-1 (yield 87.4%).

### (2) Preparation Example 2: Synthesis of Compound 422

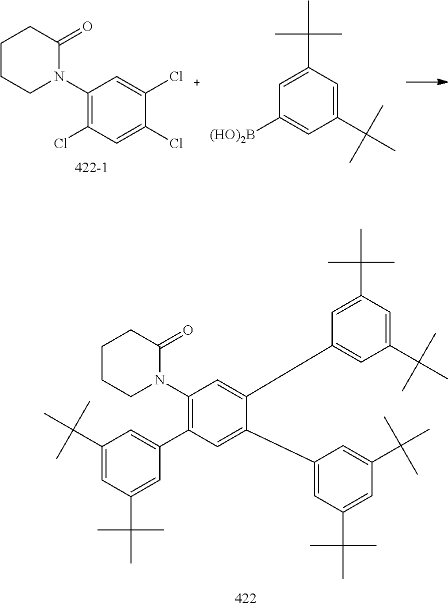 [SMILES: O=C1CCCCN1c1cc([ClH+])c(Cl)cc1Cl ; CC(C)(C)c1cc(B(O)O)cc(C(C)(C)C)c1 ; CC(C)(C)c1cc(-c2cc(-c3cc(C(C)(C)C)cc(C(C)(C)C)c3)c(N3CCCCC3=O)cc2-c2cc(C(C)(C)C)cc(C(C)(C)C)c2)cc(C(C)(C)C)c1]

Intermediate 422-2 (10.0 g, 0.036 mol), 3,5-Di-tert-butylphenylboronic Acid (30.3 g, 0.129 mol), K₂CO₃ (39.7 g, 0.287 mol), Pd(OAc)₂ (6.2 g, 0.005 mol), X-Phos (2.6 g, 0.005 mol), THF 200 mL, and H₂O 50 mL were added and reacted while stirring at 90° C. for 6 hours. After completion of the reaction, the reaction mixture was extracted, concentrated, and then column and recrystallized to obtain 14.2 g of Compound 422 (yield 53.4%).

LC/MS: m/z=740[(M)⁺]

### Synthesis Example 17: Synthesis of Compound 439

### (1) Preparation Example 1: Synthesis of Intermediate 439-1

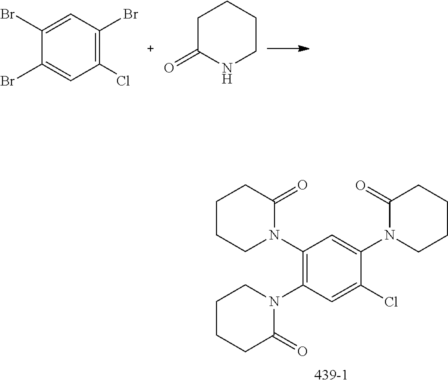

1,2,4-Tribromo-5-chlorobenzene (10.0 g, 0.029 mol), 2-Piperidone (10.2 g, 0.103 mol), K₃PO₄ (54.7 g, 0.258 mol), Pd(dba)₂ (2.5 g, 0.004 mol), Xant-Phos (26.8 g, 0.046 mol), and dioxane were added and reacted while stirring under reflux for 16 hours. After completion of the reaction, the reaction mixture was extracted, concentrated, and then column and recrystallized to obtain 9.2 g of Intermediate 439-1 (yield 79.6%).

### (2) Preparation Example 2: Synthesis of Compound 439

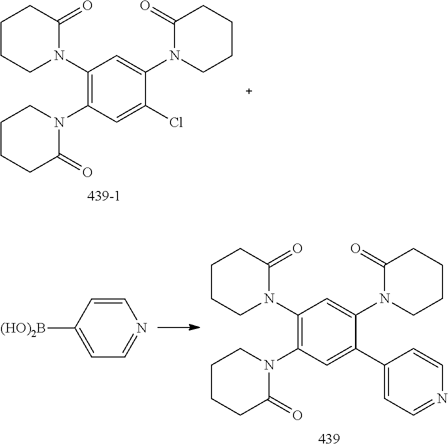 [SMILES: O=C1CCCCN1c1cc(N2CCCCC2=O)c(N2CCCCC2=O)cc1Cl ; OB(O)c1ccncc1 ; O=C1CCCCN1c1cc(N2CCCCC2=O)c(N2CCCCC2=O)cc1-c1ccncc1]

Intermediate 439-1 (10.0 g, 0.025 mol), 4-Pyridinylboronic acid (3.7 g, 0.030 mol), K₂CO₃ (10.3 g, 0.074 mol), Pd(OAc)₂ (1.4 g, 0.001 mol), X-Phos (1.2 g, 0.003 mol), THF 200 mL, and H₂O 50 mL were added and reacted while stirring at 90° C. for 6 hours. After completion of the reaction, the reaction mixture was extracted, concentrated, and then column and recrystallized to obtain 7.2 g of Compound 439 (yield 65.2%).

LC/MS: m/z=446[(M)⁺]

### Synthesis Example 18: Synthesis of Compound 470

### (1) Preparation Example 1: Synthesis of Intermediate 470-1

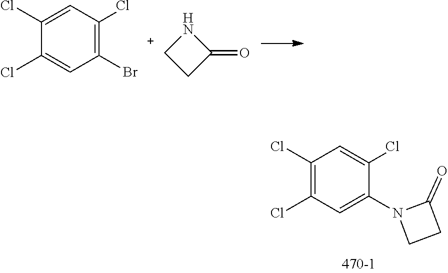

2,4,5-Trichlorobromobenzene (10.0 g, 0.038 mol), 2-Azetidinone (3.3 g, 0.046 mol), K₃PO₄ (24.5 g, 0.115 mol), Pd(dba)₂ (1.1 g, 0.002 mol), Xant-Phos (4.0 g, 0.007 mol), and dioxane 50 mL were added and reacted while stirring under reflux for 16 hours. After completion of the reaction, the reaction mixture was extracted, concentrated, and then column and recrystallized to obtain 8.6 g of Intermediate 470-1 (yield 89.4%).

### (2) Preparation Example 2: Synthesis of Compound 470

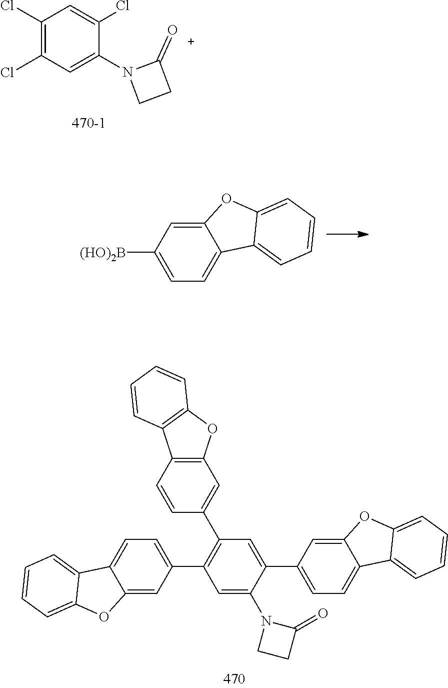 [SMILES: [OH+]=C1CCN1c1cc(Cl)c(Cl)cc1Cl ; OB(O)c1ccc2c(c1)oc1ccccc12 ; O=C1CCN1c1cc(-c2ccc3c(c2)oc2ccccc23)c(-c2ccc3c(c2)oc2ccccc23)cc1-c1ccc2c(c1)oc1ccccc12]

Intermediate 470-1 (10.0 g, 0.040 mol), 3-Dibenzofuranboronic acid (30.5 g, 0.144 mol), K₂CO₃ (49.7 g, 0.359 mol), Pd(OAc)₂ (6.9 g, 0.006 mol), X-Phos (2.9 g, 0.006 mol), THF 200 mL, and H₂O 50 mL were added and reacted while stirring at 90° C. for 6 hours. After completion of the reaction, the reaction mixture was extracted, concentrated, and the column and recrystallized to obtain 10.1 g of Compound 470 (yield 73.1%).

LC/MS: m/z=667[(M)⁺]

### Synthesis Example 19: Synthesis of Compound 502

### (1) Preparation Example 1: Synthesis of Intermediate 502-1

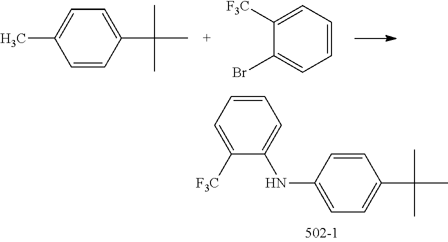

4-tert-Butylaniline (10.0 g, 0.067 mol), 1-Bromo-2-(trifluoromethyl)benzene (22.6 g, 0.101 mol), NaOtBu (12.9 g, 0.134 mol), Pd(dba)₂ (1.9 g, 3.4 mmol), and t-Bu₃P (1.4 g, 6.7 mmol) were added with Toluene 150 mL and reacted while stirring at 70° C. for 4 hours. After completion of the reaction, the reaction mixture was extracted, concentrated, and then column and recrystallized to obtain 12.8 g of Intermediate 502-1 (yield 65.1%).

### (2) Preparation Example 2: Synthesis of Compound 502

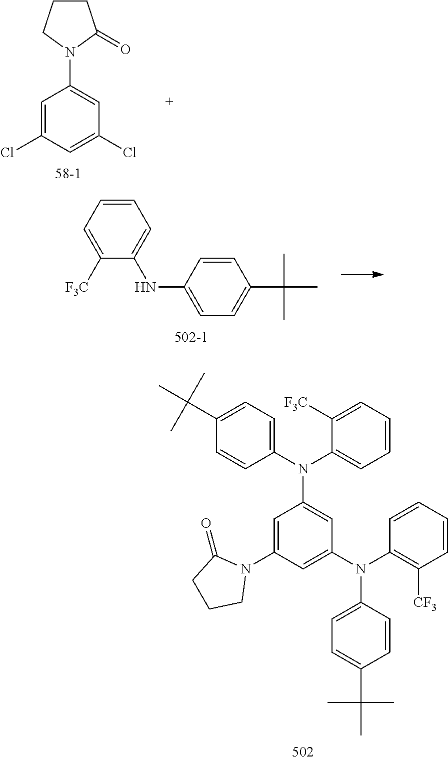 [SMILES: O=C1CCCN1c1cc(Cl)cc(Cl)c1 ; CC(C)(C)c1ccc(Nc2ccccc2C(F)(F)F)cc1 ; CC(C)(C)c1ccc(N(c2cc(N3CCCC3=O)cc(N(c3ccc(C(C)(C)C)cc3)c3ccccc3C(F)(F)F)c2)c2ccccc2C(F)(F)F)cc1]

Intermediate 58-1 (10.0 g, 0.044 mol), Intermediate 502-1 (38.3 g, 0.130 mol), NaOtBu (16.7 g, 0.174 mol), Pd(dba)₂ (2.5 g, 4.3 mmol), and t-Bu₃P (1.8 g, 8.7 mmol) were added with Toluene 150 mL and reacted while stirring at 70° C. for 4 hours. After completion of the reaction, the reaction mixture was extracted, concentrated, and then column and recrystallized to obtain 20.3 g of Compound 502 (yield 62.8%).

LC/MS: m/z=743[(M)⁺]

### Synthesis Example 20: Synthesis of Compound 518

### (1) Preparation Example 1: Synthesis of Intermediate 518-1

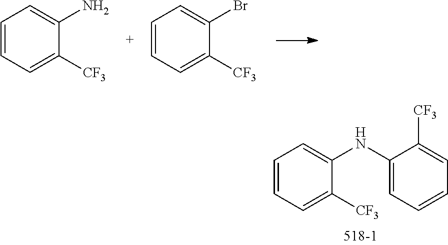 [SMILES: Nc1ccccc1C(F)(F)F ; FC(F)(F)c1ccccc1Br ; FC(F)(F)c1ccccc1Nc1ccccc1C(F)(F)F]

2-(Trifluoromethyl) aniline (10.0 g, 0.062 mol), 1-Bromo-2-(trifluoromethyl)benzene (21.0 g, 0.093 mol), NaOtBu (11.9 g, 0.124 mol), Pd(dba)₂ (1.8 g, 3.1 mmol), and t-Bu₃P (1.3 g, 6.2 mmol) were added with Toluene 150 mL and reacted while stirring at 70° C. for 4 hours. After completion of the reaction, the reaction mixture was extracted, concentrated, and then column and recrystallized to obtain 10.8 g of Intermediate 518-1 (yield 57.0%).

### (2) Preparation Example 2: Synthesis of Compound 518

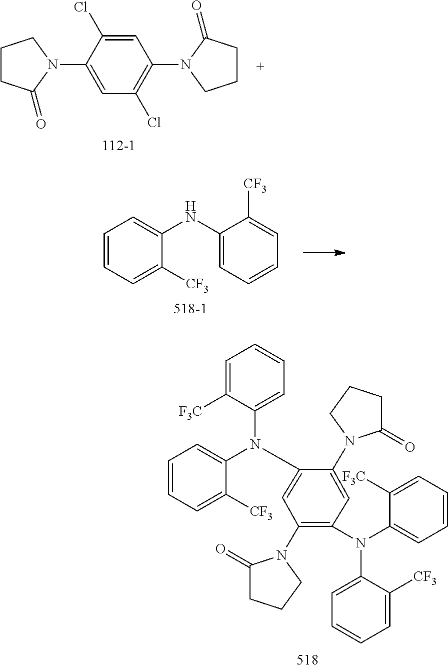 [SMILES: O=C1CCCN1c1cc(Cl)c(N2CCCC2=O)cc1Cl ; FC(F)(F)c1ccccc1Nc1ccccc1C(F)(F)F ; O=C1CCCN1c1cc(N(c2ccccc2C(F)(F)F)c2ccccc2C(F)(F)F)c(N2CCCC2=O)cc1N(c1ccccc1C(F)(F)F)c1ccccc1C(F)(F)F]

Intermediate 112-1 (10.0 g, 0.032 mol), Intermediate 518-1 (29.3 g, 0.096 mol), NaOtBu (12.3 g, 0.128 mol), Pd(dba)₂ (1.8 g, 3.2 mmol), and t-Bu₃P (1.3 g, 6.4 mmol) were added with Toluene 150 mL and reacted while stirring at 70° C. for 4 hours. After completion of the reaction, the reaction mixture was extracted, concentrated, and then column and recrystallized to obtain 13.3 g of Compound 518 (yield 49.0%).

LC/MS: m/z=850[(M)⁺]

### Synthesis Example 21: Synthesis of Compound 551

### (1) Preparation Example 1: Synthesis of Intermediate 551-1

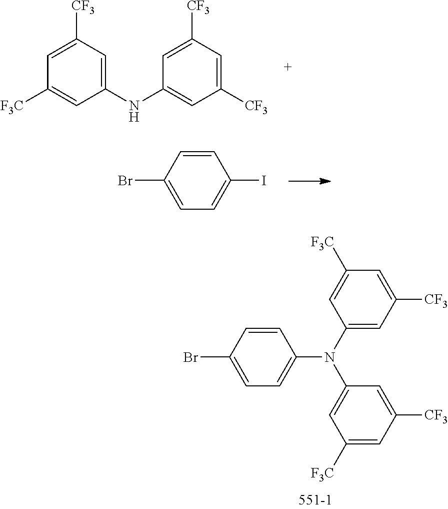 [SMILES: FC(F)(F)c1cc(Nc2cc(C(F)(F)F)cc(C(F)(F)F)c2)cc(C(F)(F)F)c1 ; Brc1ccc(I)cc1 ; FC(F)(F)c1cc(N(c2ccc(Br)cc2)c2cc(C(F)(F)F)cc(C(F)(F)F)c2)cc(C(F)(F)F)c1]

N-[3,5-Bis(trifluoromethyl)phenyl]-3,5-bis(trifluoromethyl)benzenamine (10.0 g, 0.023 mol), Bis(3,5-dimethylphenyl)amine (9.6 g, 0.034 mol), NaOtBu (4.4 g, 0.045 mol), Pd(dba)₂ (0.7 g, 1.1 mmol), and t-Bu₃P (0.5 g, 2.3 mmol) were added with Toluene 150 mL and reacted while stirring at 70° C. for 4 hours. After completion of the reaction, the reaction mixture was extracted, concentrated, and then columned to obtain 7.5 g of Intermediate 551-1 (yield 55.5%).

### (2) Preparation Example 2: Synthesis of Intermediate 551-2

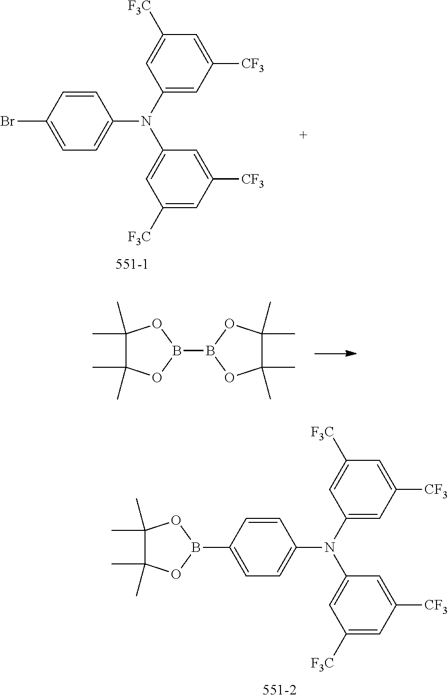

Intermediate 551-1 (10.0 g, 0.017 mol), Bis(pinacolato)diboron (5.1 g, 0.020 mol), KOAc (4.9 g, 0.050 mol), and Pd(dppf)Cl2 (0.6 g, 0.001 mol) were added with dioxane 200 mL and reacted while stirring at 100° C. for 12 hours. After completion of the reaction, the reaction mixture was extracted, concentrated, and then column and recrystallized to obtain 7.7 g of Intermediate 551-2 (yield 71.4%).

### (3) Preparation Example 3: Synthesis of Compound 551

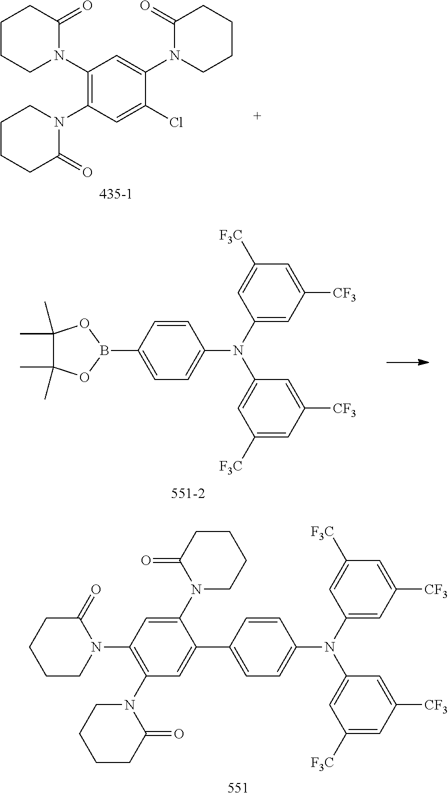 [SMILES: O=C1CCCCN1c1cc(N2CCCCC2=O)c(N2CCCCC2=O)cc1Cl ; CC1(C)OB(c2ccc(N(c3cc(C(F)(F)F)cc(C(F)(F)F)c3)c3cc(C(F)(F)F)cc(C(F)(F)F)c3)cc2)OC1(C)C ; O=C1CCCCN1c1cc(N2CCCCC2=O)c(N2CCCCC2=O)cc1-c1ccc(N(c2cc(C(F)(F)F)cc(C(F)(F)F)c2)c2cc(C(F)(F)F)cc(C(F)(F)F)c2)cc1]

Intermediate 439-1 (10.0 g, 0.025 mol), Intermediate 551-2 (19.1 g, 0.030 mol), K₂CO₃ (10.3 g, 0.074 mol), Pd(OAc)₂ (1.4 g, 0.001 mol), X-Phos (1.2 g, 0.003 mol), THF 200 mL, and H₂O 50 mL were added and reacted while stirring at 90° C. for 6 hours. After completion of the reaction, the reaction mixture was extracted, concentrated, and the column and recrystallized to obtain 13.6 g of Compound 551 (yield 62.1%).

LC/MS: m/z=884[(M)⁺]

### Synthesis Example 22: Synthesis of Compound 563

### (1) Preparation Example 1: Synthesis of Intermediate 563-1

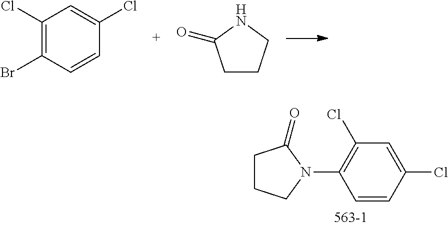 [SMILES: Clc1ccc(Br)c(Cl)c1 ; O=C1CCCN1 ; O=C1CCCN1c1ccc(Cl)cc1Cl]

2,4-Dichlorobromobenzene (10.0 g, 0.044 mol), pyrrolidin-2-one (4.5 g, 0.053 mol), K₃PO₄ (28.2 g, 0.133 mol), Pd(dba)₂ (1.3 g, 0.002 mol), Xant-Phos (4.6 g, 0.008 mol), and dioxane were added and reacted while stirring under reflux for 16 hours. After completion of the reaction, the reaction mixture was extracted, concentrated, and then column and recrystallized to obtain 8.5 g of Intermediate 563-1 (yield 83.5%).

### (2) Preparation Example 2: Synthesis of Compound 563

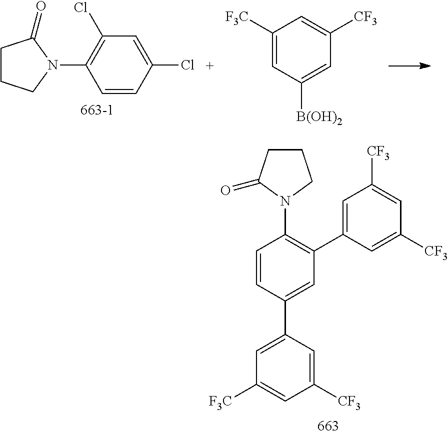

Intermediate 563-1 (10.0 g, 0.044 mol), 3,5-Bis(trifluoromethyl)phenylboronic acid (26.9 g, 0.104 mol), K₂CO₃ (36.0 g, 0.261 mol), Pd(OAc)₂ (5.0 g, 0.004 mol), X-Phos (4.1 g, 0.009 mol), THF 200 mL, and H₂O 50 mL were added and reacted while stirring at 90° C. for 6 hours. After completion of the reaction, the reaction mixture was extracted, concentrated, and the column and recrystallized to obtain 14.5 g of Compound 563 (yield 57.0%).

LC/MS: m/z=585[(M)⁺]

### Synthesis Example 23: Synthesis of Compound 575

### (1) Preparation Example 1: Synthesis of Intermediate 575-1

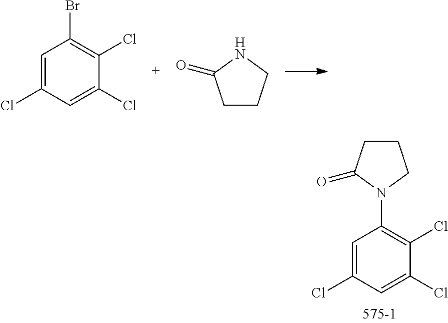 [SMILES: Clc1cc(Cl)c(Cl)c(Br)c1 ; O=C1CCCN1 ; O=C1CCCN1c1cc(Cl)cc(Cl)c1Cl]

1-Bromo-2,3,5-trichlorobenzene (10.0 g, 0.038 mol), pyrrolidin-2-one (3.9 g, 0.046 mol), K₃PO₄ (24.5 g, 0.115 mol), Pd(dba)₂ (1.1 g, 0.002 mol), Xant-Phos (4.0 g, 0.007 mol), and dioxane were added and reacted while stirring under reflux for 16 hours. After completion of the reaction, the reaction mixture was extracted, concentrated, and then column and recrystallized to obtain 7.9 g of Intermediate 575-1 (yield 77.8%).

### (2) Preparation Example 2: Synthesis of Compound 575

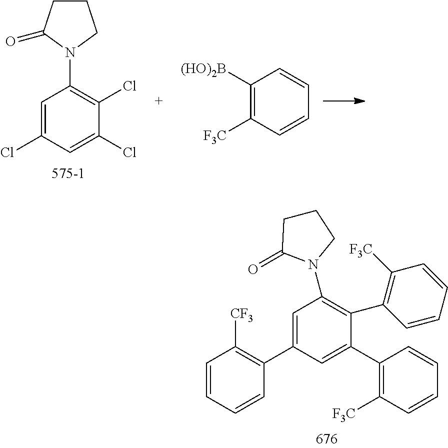 [SMILES: O=C1CCCN1c1cc(Cl)cc(Cl)c1Cl ; OB(O)c1ccccc1C(F)(F)F ; O=C1CCCN1c1cc(-c2ccccc2C(F)(F)F)cc(-c2ccccc2C(F)(F)F)c1-c1ccccc1C(F)(F)F]

Intermediate 575-1 (10.0 g, 0.038 mol), 2-(Trifluoromethyl)phenylboronic acid (25.9 g, 0.136 mol), K₂CO₃ (47.0 g, 0.340 mol), Pd(OAc)₂ (6.6 g, 0.006 mol), X-Phos (3.6 g, 0.008 mol), THF 200 mL, and H₂O 50 mL were added and reacted while stirring at 90° C. for 6 hours. After completion of the reaction, the reaction mixture was extracted, concentrated, and then column and recrystallized to obtain 12.7 g of Compound 575 (yield 56.6%).

LC/MS: m/z=593[(M)⁺]

### Synthesis Example 24: Synthesis of Compound 604

### (1) Preparation Example 1: Synthesis of Intermediate 604-1

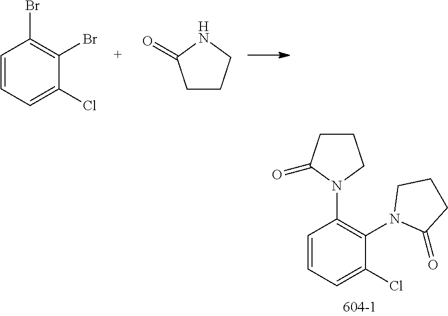 [SMILES: Clc1cccc(Br)c1Br ; O=C1CCCN1 ; O=C1CCCN1c1cccc(Cl)c1N1CCCC1=O]

1-Chloro-2,3-dibromobenzene (10.0 g, 0.037 mol), pyrrolidin-2-one (7.6 g, 0.089 mol), K₃PO₄ (47.1 g, 0.222 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then column and recrystallized to obtain 6.3 g of Intermediate 575-1 (yield 61.1%).

### (2) Preparation Example 2: Synthesis of Compound 604

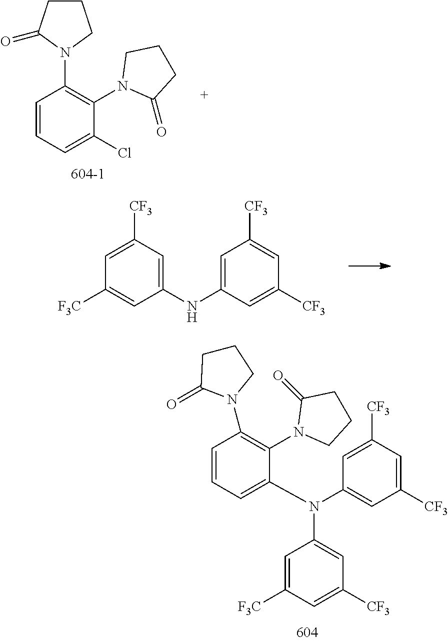

Intermediate 604-1 (10.0 g, 0.036 mol), N-[3,5-Bis(trifluoromethyl)phenyl]-3,5-bis(trifluoromethyl)benzenamine (23.7 g, 0.054 mol), NaOtBu (6.9 g, 0.072 mol), Pd(dba)₂ (1.0 g, 1.8 mmol), and t-Bu₃P (0.7 g, 3.6 mmol) were added with Toluene 150 mL and reacted while stirring at 70° C. for 4 hours. After completion of the reaction, the reaction mixture was extracted, concentrated, and then column and recrystallized to obtain 10.5 g of Compound 604 (yield 42.8%).

LC/MS: m/z=683[(M)⁺]

### Synthesis Example 25: Synthesis of Compound 629

### (1) Preparation Example 1: Synthesis of Intermediate 629-1

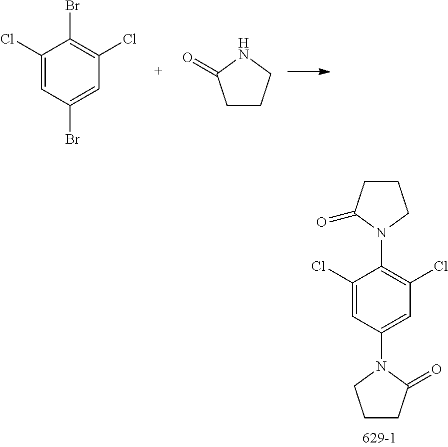 [SMILES: Clc1cc(Br)cc(Cl)c1Br ; O=C1CCCN1 ; O=C1CCCN1c1cc(Cl)c(N2CCCC2=O)c(Cl)c1]

1,4-Dibromo-2,6-dichlorobenzene (10.0 g, 0.033 mol), pyrrolidin-2-one (6.7 g, 0.079 mol), K₃PO₄ (41.8 g, 0.197 mol), Pd(dba)₂ (1.9 g, 0.003 mol), Xant-Phos (13.7 g, 0.024 mol), and dioxane were added and reacted while stirring under reflux for 16 hours. After completion of the reaction, the reaction mixture was extracted, concentrated, and then column and recrystallized to obtain 8.1 g of Intermediate 629-1 (yield 78.8%).

### (2) Preparation Example 2: Synthesis of Compound 629

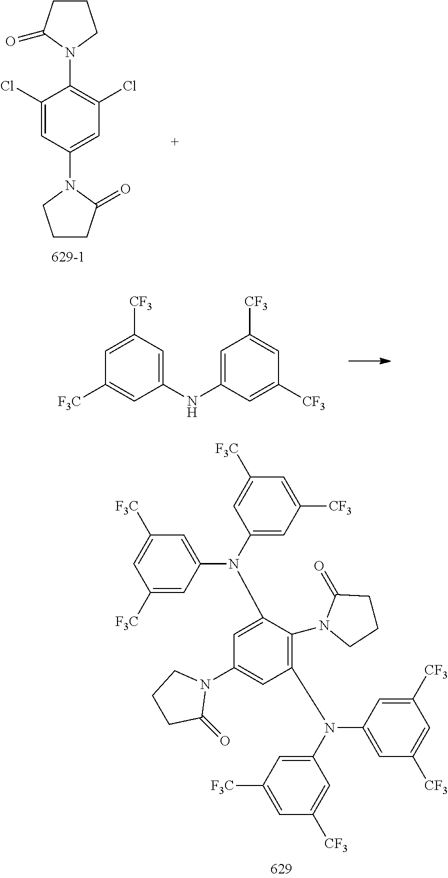 [SMILES: O=C1CCCN1c1cc(Cl)c(N2CCCC2=O)c(Cl)c1 ; FC(F)(F)c1cc(Nc2cc(C(F)(F)F)cc(C(F)(F)F)c2)cc(C(F)(F)F)c1 ; O=C1CCCN1c1cc(N(c2cc(C(F)(F)F)cc(C(F)(F)F)c2)c2cc(C(F)(F)F)cc(C(F)(F)F)c2)c(N2CCCC2=O)c(N(c2cc(C(F)(F)F)cc(C(F)(F)F)c2)c2cc(C(F)(F)F)cc(C(F)(F)F)c2)c1]

Intermediate 629-1 (10.0 g, 0.032 mol), N-[3,5-Bis(trifluoromethyl)phenyl]-3,5-bis(trifluoromethyl)benzenamine (42.3 g, 0.096 mol), NaOtBu (12.3 g, 0.128 mol), Pd(dba)₂ (1.8 g, 3.2 mmol), and t-Bu₃P (1.3 g, 6.4 mmol) were added with Toluene 150 mL and reacted while stirring at 70° C. for 4 hours. After completion of the reaction, the reaction mixture was extracted, concentrated, and then column and recrystallized to obtain 15.4 g of Compound 629 (yield 43.0%).

LC/MS: m/z=1122[(M)⁺]

### Device Example (CPL)

In Example according to the present invention, an Ag-containing ITO glass substrate having dimensions of 25 mm×25 mm×0.7 mm was used as an anode, patterned to have a light emitting area of 2 mm×2 mm, followed by cleaning. After the patterned ITO substrate was mounted in a vacuum chamber, organic materials and metals were deposited in the following structure on the substrate at a process pressure of 1×10⁻⁶ torr or more.

### Device Examples 1 to 111

The compound implemented according to the present invention was employed in the light efficiency improving layer to fabricate an organic light emitting device having the following device structure, and then the luminescent and driving properties according to the compound implemented according to the present invention were measured.

Ag:ITO/hole injecting layer (HAT-CN, 5 nm)/hole transport layer (α-NPB, 100 nm)/electron blocking layer (TCTA, 10 nm)/light emitting layer (20 nm)/electron transport layer (201:Liq, 30 nm)/LiF (1 nm)/Mg:Ag (15 nm)/light efficiency improving layer (70 nm)

On a glass substrate, HAT-CN was formed into a 5 nm-thick film to form a hole injecting layer on an Ag-containing ITO transparent electrode. Thereafter, α-NPB was formed into a 100 nm-thick film to form a hole transport layer, and then TCTA was formed into a 10 nm-thick film to form an electron blocking layer. Thereafter, a light emitting layer was formed by co-depositing BH1 as a host compound and BD1 as a dopant compound to a thickness of 20 nm. Thereafter, a 30 nm-thick electron transport layer (doped with Liq 50% of the following Compound 201) was deposited, and then LiF was formed into a 1 nm-thick film to form an electron injection layer. Thereafter, Mg:Ag was formed into a 15 nm-thick film at a ratio of 1:9 to form a cathode. In addition, the light efficiency improving layer (capping layer) was formed to a 70 nm-thick film using the compounds according to the present invention shown in Table 1 below to fabricate an organic light emitting device.

### Device Comparative Example 1

An organic light emitting device for Device Comparative Example 1 was fabricated in the same method, except for not using the light efficiency improving layer in the device structure of Example above.

### Device Comparative Example 2

An organic light emitting device for Device Comparative Example 2 was fabricated in the same method, except for using Alq₃ instead of the compound according to the present invention as the light efficiency improving layer compound in the device structure of Example above.

### Experimental Example 1: Luminescent Properties of Device Examples 1 to 111

The driving voltages, current efficiencies, and color coordinates of the organic light emitting devices fabricated in Examples and Comparative Examples above were measured using a source meter (Model 237, Keithley) and a spectroradiometer (PR-650, Photo Research). The result values at 1,000 nits were shown in Table 1 below.

TABLE 1

| | Light efficiency | | | | |
|---|---|---|---|---|---|
| Example | improving layer | V | cd/A | CIEx | CIEy |
| 1 | Formula 2 | 3.39 | 8.73 | 0.1393 | 0.0513 |
| 2 | Formula 4 | 3.25 | 8.92 | 0.1385 | 0.0566 |
| 3 | Formula 5 | 3.31 | 8.83 | 0.1368 | 0.0573 |
| 4 | Formula 6 | 3.64 | 8.23 | 0.1405 | 0.0449 |
| 5 | Formula 7 | 3.45 | 8.69 | 0.1398 | 0.0506 |
| 6 | Formula 8 | 3.29 | 8.95 | 0.1386 | 0.0551 |
| 7 | Formula 11 | 3.46 | 8.66 | 0.1357 | 0.0503 |
| 8 | Formula 12 | 3.35 | 8.70 | 0.1399 | 0.0500 |
| 9 | Formula 19 | 3.29 | 8.97 | 0.1405 | 0.0493 |
| 10 | Formula 21 | 3.64 | 8.35 | 0.1383 | 0.0458 |
| 11 | Formula 22 | 3.38 | 8.83 | 0.1388 | 0.0522 |
| 12 | Formula 24 | 3.49 | 8.59 | 0.1361 | 0.0491 |
| 13 | Formula 28 | 3.32 | 8.76 | 0.1395 | 0.0523 |
| 14 | Formula 29 | 3.45 | 8.60 | 0.1410 | 0.0485 |
| 15 | Formula 30 | 3.38 | 8.75 | 0.1423 | 0.0473 |
| 16 | Formula 38 | 3.69 | 8.33 | 0.1376 | 0.0463 |
| 17 | Formula 47 | 3.48 | 8.69 | 0.1385 | 0.0508 |
| 18 | Formula 48 | 3.60 | 8.36 | 0.1397 | 0.0473 |
| 19 | Formula 51 | 3.34 | 8.92 | 0.1382 | 0.0574 |
| 20 | Formula 58 | 3.49 | 8.66 | 0.1408 | 0.0509 |
| 21 | Formula 59 | 3.63 | 8.25 | 0.1391 | 0.0483 |
| 22 | Formula 61 | 3.56 | 8.43 | 0.1387 | 0.0453 |
| 23 | Formula 63 | 3.31 | 8.89 | 0.1356 | 0.0557 |
| 24 | Formula 66 | 3.33 | 8.83 | 0.1418 | 0.0492 |
| 25 | Formula 74 | 3.64 | 8.66 | 0.1394 | 0.0501 |
| 26 | Formula 75 | 3.32 | 8.78 | 0.1402 | 0.0503 |
| 27 | Formula 84 | 3.45 | 8.53 | 0.1364 | 0.0514 |
| 28 | Formula 109 | 3.60 | 8.35 | 0.1395 | 0.0499 |
| 29 | Formula 101 | 3.27 | 8.92 | 0.1387 | 0.0533 |
| 30 | Formula 111 | 3.32 | 8.88 | 0.1353 | 0.0596 |
| 31 | Formula 112 | 3.49 | 8.55 | 0.1402 | 0.0493 |
| 32 | Formula 113 | 3.36 | 8.85 | 0.1387 | 0.0550 |
| 33 | Formula 123 | 3.28 | 8.86 | 0.1382 | 0.0581 |
| 34 | Formula 124 | 3.39 | 8.41 | 0.1394 | 0.0510 |
| 35 | Formula 129 | 3.22 | 8.67 | 0.1366 | 0.0586 |
| 36 | Formula 136 | 3.58 | 8.13 | 0.1384 | 0.0531 |
| 37 | Formula 141 | 3.62 | 8.26 | 0.1375 | 0.0518 |
| 38 | Formula 153 | 3.27 | 8.71 | 0.1392 | 0.0504 |
| 39 | Formula 162 | 3.38 | 8.48 | 0.1358 | 0.0520 |
| 40 | Formula 164 | 3.28 | 8.69 | 0.1412 | 0.0461 |
| 41 | Formula 165 | 3.32 | 8.50 | 0.1422 | 0.0479 |
| 42 | Formula 173 | 3.28 | 8.69 | 0.1417 | 0.0472 |
| 43 | Formula 177 | 3.55 | 8.26 | 0.1381 | 0.0461 |
| 44 | Formula 185 | 3.28 | 8.75 | 0.1386 | 0.0581 |
| 45 | Formula 198 | 3.57 | 8.26 | 0.1412 | 0.0450 |
| 46 | Formula 200 | 3.40 | 8.31 | 0.1395 | 0.0486 |
| 47 | Formula 203 | 3.36 | 8.59 | 0.1351 | 0.0548 |
| 48 | Formula 218 | 3.58 | 8.17 | 0.1379 | 0.0536 |
| 49 | Formula 222 | 3.41 | 8.33 | 0.1398 | 0.0467 |
| 50 | Formula 224 | 3.28 | 8.78 | 0.1402 | 0.0482 |
| 51 | Formula 237 | 3.74 | 8.73 | 0.1370 | 0.0501 |
| 52 | Formula 254 | 3.63 | 8.97 | 0.1413 | 0.0586 |
| 53 | Formula 266 | 3.74 | 8.82 | 0.1395 | 0.0517 |
| 54 | Formula 281 | 3.69 | 8.33 | 0.1411 | 0.0483 |
| 55 | Formula 293 | 3.40 | 8.65 | 0.1385 | 0.0526 |
| 56 | Formula 300 | 3.37 | 8.71 | 0.1425 | 0.0473 |
| 57 | Formula 313 | 3.52 | 8.43 | 0.1382 | 0.0529 |
| 58 | Formula 321 | 3.20 | 8.62 | 0.1373 | 0.0554 |
| 59 | Formula 355 | 3.38 | 8.55 | 0.1395 | 0.0535 |
| 60 | Formula 357 | 3.26 | 8.69 | 0.1376 | 0.0581 |
| 61 | Formula 359 | 3.53 | 8.26 | 0.1372 | 0.0528 |
| 62 | Formula 371 | 3.38 | 8.51 | 0.1366 | 0.0511 |
| 63 | Formula 396 | 3.51 | 8.25 | 0.1383 | 0.0486 |
| 64 | Formula 405 | 3.20 | 8.63 | 0.1386 | 0.0582 |
| 65 | Formula 412 | 3.52 | 8.80 | 0.1394 | 0.0509 |
| 66 | Formula 418 | 3.66 | 8.77 | 0.1357 | 0.0535 |
| 67 | Formula 419 | 3.53 | 8.85 | 0.1389 | 0.0529 |
| 68 | Formula 425 | 3.81 | 8.45 | 0.1392 | 0.0477 |
| 69 | Formula 433 | 3.63 | 8.73 | 0.1351 | 0.0510 |
| 70 | Formula 436 | 3.87 | 8.41 | 0.1366 | 0.0535 |
| 71 | Formula 439 | 3.52 | 9.06 | 0.1376 | 0.0588 |
| 72 | Formula 452 | 3.63 | 8.65 | 0.1354 | 0.0519 |
| 73 | Formula 470 | 3.57 | 8.80 | 0.1371 | 0.0586 |
| 74 | Formula 483 | 3.65 | 8.65 | 0.1356 | 0.0527 |
| 75 | Formula 486 | 3.53 | 8.93 | 0.1389 | 0.0519 |
| 76 | Formula 491 | 3.68 | 8.74 | 0.1355 | 0.0515 |
| 77 | Formula 495 | 3.59 | 8.85 | 0.1368 | 0.0592 |
| 78 | Formula 498 | 3.43 | 9.08 | 0.1368 | 0.0548 |
| 79 | Formula 502 | 3.86 | 8.42 | 0.1381 | 0.0521 |
| 80 | Formula 505 | 3.53 | 8.95 | 0.1378 | 0.0580 |
| 81 | Formula 506 | 3.85 | 8.43 | 0.1391 | 0.0490 |
| 82 | Formula 521 | 3.61 | 8.76 | 0.1377 | 0.0536 |
| 83 | Formula 525 | 3.57 | 8.85 | 0.1386 | 0.0521 |
| 84 | Formula 528 | 3.73 | 8.51 | 0.1378 | 0.0539 |
| 85 | Formula 541 | 3.66 | 8.73 | 0.1392 | 0.0500 |
| 86 | Formula 546 | 3.83 | 8.35 | 0.1401 | 0.0497 |
| 87 | Formula 551 | 3.70 | 8.51 | 0.1379 | 0.0529 |
| 88 | Formula 554 | 3.43 | 9.17 | 0.1398 | 0.0505 |
| 89 | Formula 561 | 3.54 | 9.13 | 0.1410 | 0.0481 |
| 90 | Formula 563 | 3.68 | 9.08 | 0.1382 | 0.0528 |
| 91 | Formula 572 | 3.70 | 8.75 | 0.1376 | 0.0535 |
| 92 | Formula 575 | 3.64 | 9.07 | 0.1352 | 0.0561 |
| 93 | Formula 580 | 3.71 | 8.88 | 0.1375 | 0.0516 |
| 94 | Formula 583 | 3.64 | 8.92 | 0.1366 | 0.0592 |
| 95 | Formula 588 | 3.58 | 9.18 | 0.1352 | 0.0548 |
| 96 | Formula 592 | 3.97 | 8.55 | 0.1401 | 0.0467 |
| 97 | Formula 594 | 3.64 | 9.09 | 0.1353 | 0.0581 |
| 98 | Formula 599 | 3.92 | 8.56 | 0.1416 | 0.0453 |
| 99 | Formula 600 | 3.60 | 9.01 | 0.1352 | 0.0584 |
| 100 | Formula 604 | 3.96 | 8.52 | 0.1405 | 0.0477 |
| 101 | Formula 612 | 3.75 | 8.87 | 0.1371 | 0.0511 |
| 102 | Formula 621 | 3.93 | 8.59 | 0.1420 | 0.0482 |
| 103 | Formula 628 | 3.61 | 9.12 | 0.1352 | 0.0585 |
| 104 | Formula 629 | 3.72 | 8.71 | 0.1370 | 0.0511 |
| 105 | Formula 630 | 3.64 | 8.98 | 0.1386 | 0.0586 |
| 106 | Formula 633 | 3.50 | 8.96 | 0.1366 | 0.0574 |
| 107 | Formula 634 | 3.68 | 8.99 | 0.1352 | 0.0583 |
| 108 | Formula 636 | 3.94 | 8.42 | 0.1421 | 0.0451 |
| 109 | Formula 637 | 3.73 | 8.87 | 0.1365 | 0.0518 |
| 110 | Formula 638 | 3.56 | 9.12 | 0.1356 | 0.0562 |
| 111 | Formula 639 | 3.62 | 8.95 | 0.1351 | 0.0589 |
| Comparative | Not used | 4.68 | 7.03 | 0.1502 | 0.1412 |
| Example 1 | | | | | |
| Comparative | Alq₃ | 4.33 | 7.84 | 0.1471 | 0.0583 |
| Example 2 | | | | | |

As described in the results shown in Table 1 above, it was confirmed that when the compound according to the present invention was applied to a device as a light efficiency improving layer, the device had reduced driving voltage and improved current efficiency compared to a conventional device without a light efficiency improving layer and a conventional device using a compound used as a light efficiency improving layer material (Comparative Examples 1 and 2).

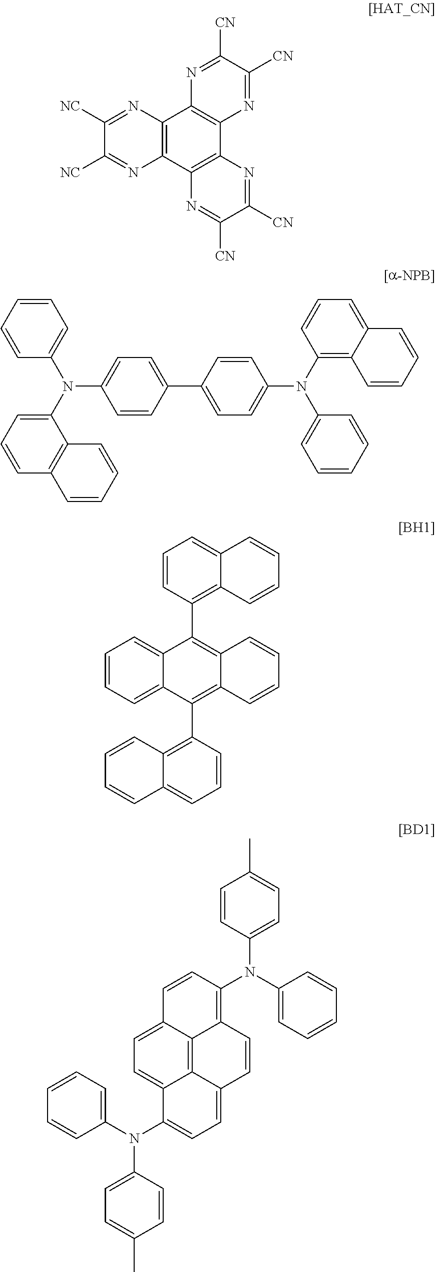

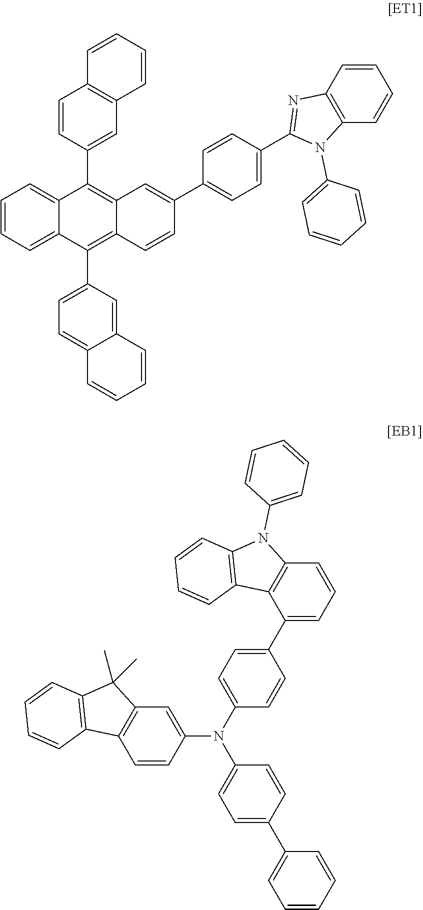 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21]

### INDUSTRIAL APPLICABILITY

According to the present invention, an organic compound is employed as a material for a light efficiency improving layer provided in an organic light emitting 5 device to achieve low-voltage driving and improved luminescent properties such as excellent luminous efficiency, color purity, etc. by increasing the light efficiency of the organic light emitting device, and thus the present invention can be effectively used industrially in various lighting and display devices.

1. A compound represented by Formula I below: 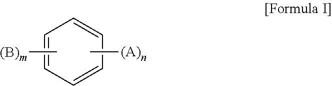
In Formula I above,
A is any one selected from structures represented by Structural Formula 1 below (‘*’ indicates a linking site), 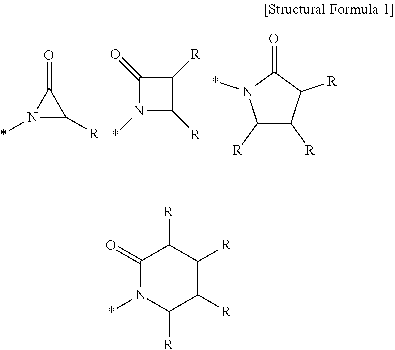
In Structural Formula 1 above,
in each structure, R is each independently any one selected from hydrogen, deuterium, cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and
in each structure of Structural Formula 1, a plurality of R are the same as or different from each other,
B is any one selected from deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, a substituted or unsubstituted heteroaryl group having 2 to 30 carbon atoms, and Structural Formula 2 below, 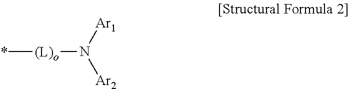
In Structural Formula 2 above,
L is a single bond or any one selected from a substituted or unsubstituted arylene group having 6 to 30 carbon atoms and a substituted or unsubstituted heteroarylene group having 3 to 30 carbon atoms,
o is an integer of 0 to 2, and when the o is 2, a plurality of L are the same as or different from each other,
Ar₁ and Ar₂ are the same as or different from each other and are each independently any one selected from a substituted or unsubstituted aryl group having 6 to 30 carbon atoms and a substituted or unsubstituted heteroaryl group having 2 to 30 carbon atoms, and
n is an integer of 1 to 4, m is an integer of 0 to 5, and when the n and m are each 2 or more, a plurality of A and B are the same as or different from each other (however, 3≤n+m≤6).

2. The compound of claim 1, wherein the ‘substituted or unsubstituted’ in the definition of R, B, L, Ar₁ and Ar₂ above means substitution of R, B, L, Ar₁ and Ar₂ above with one or at least two substituents selected from the group consisting of deuterium, a halogen group, a cyano group, a nitro group, a hydroxy group, a silyl group, an alkyl group, an amine group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

3. The compound of claim 1, wherein Formula 1 above is selected from Compounds 1 to 641 below:

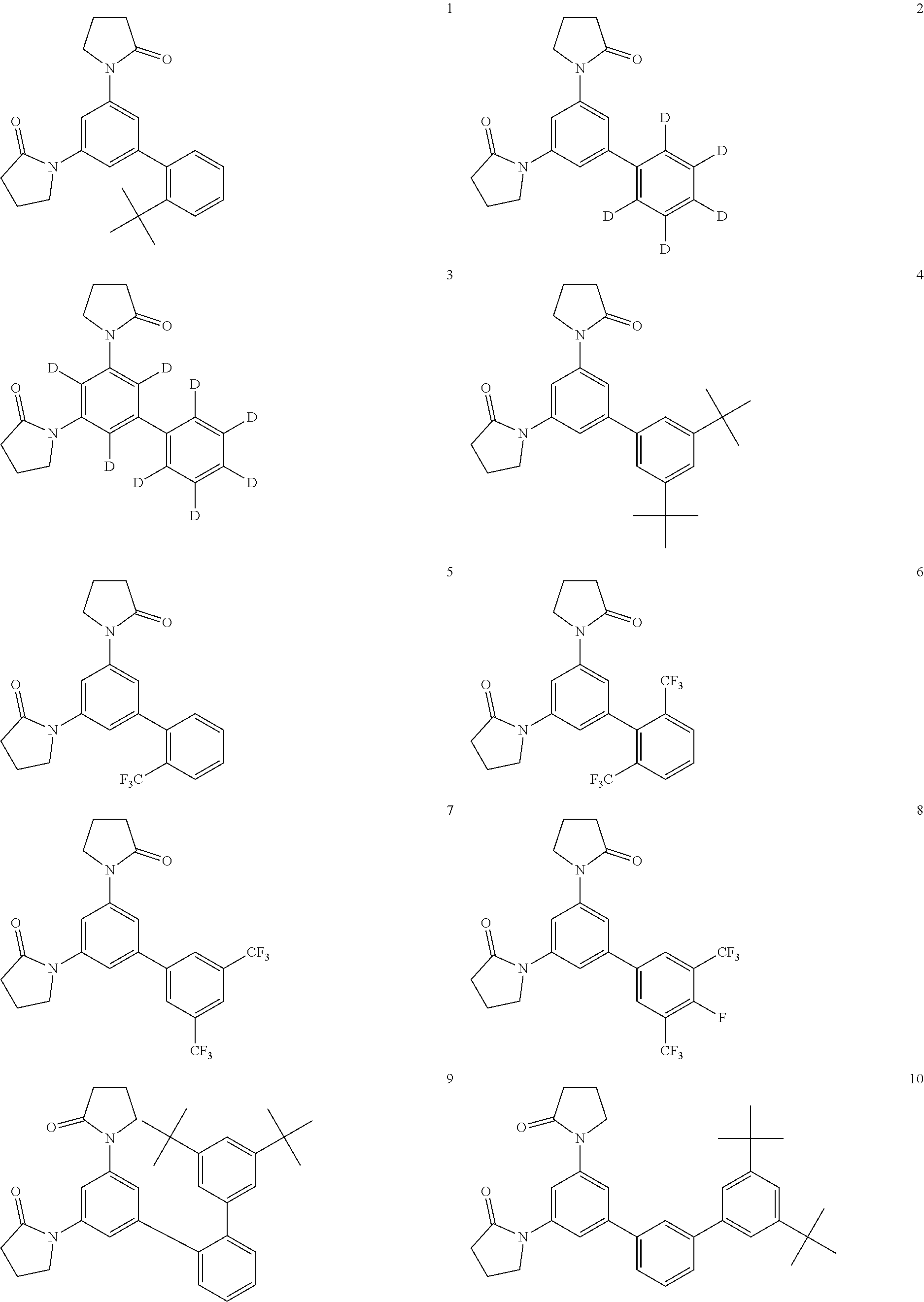

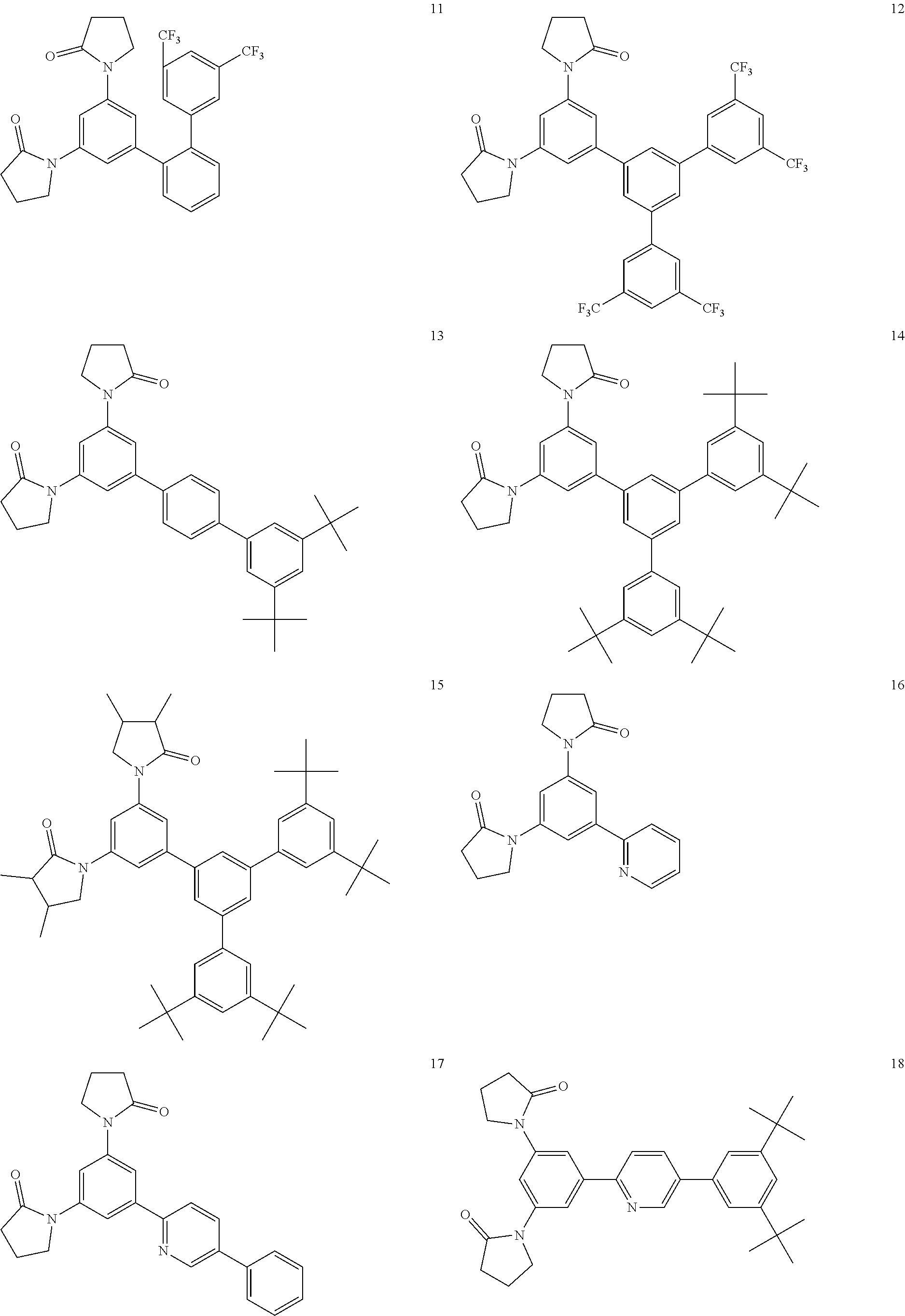

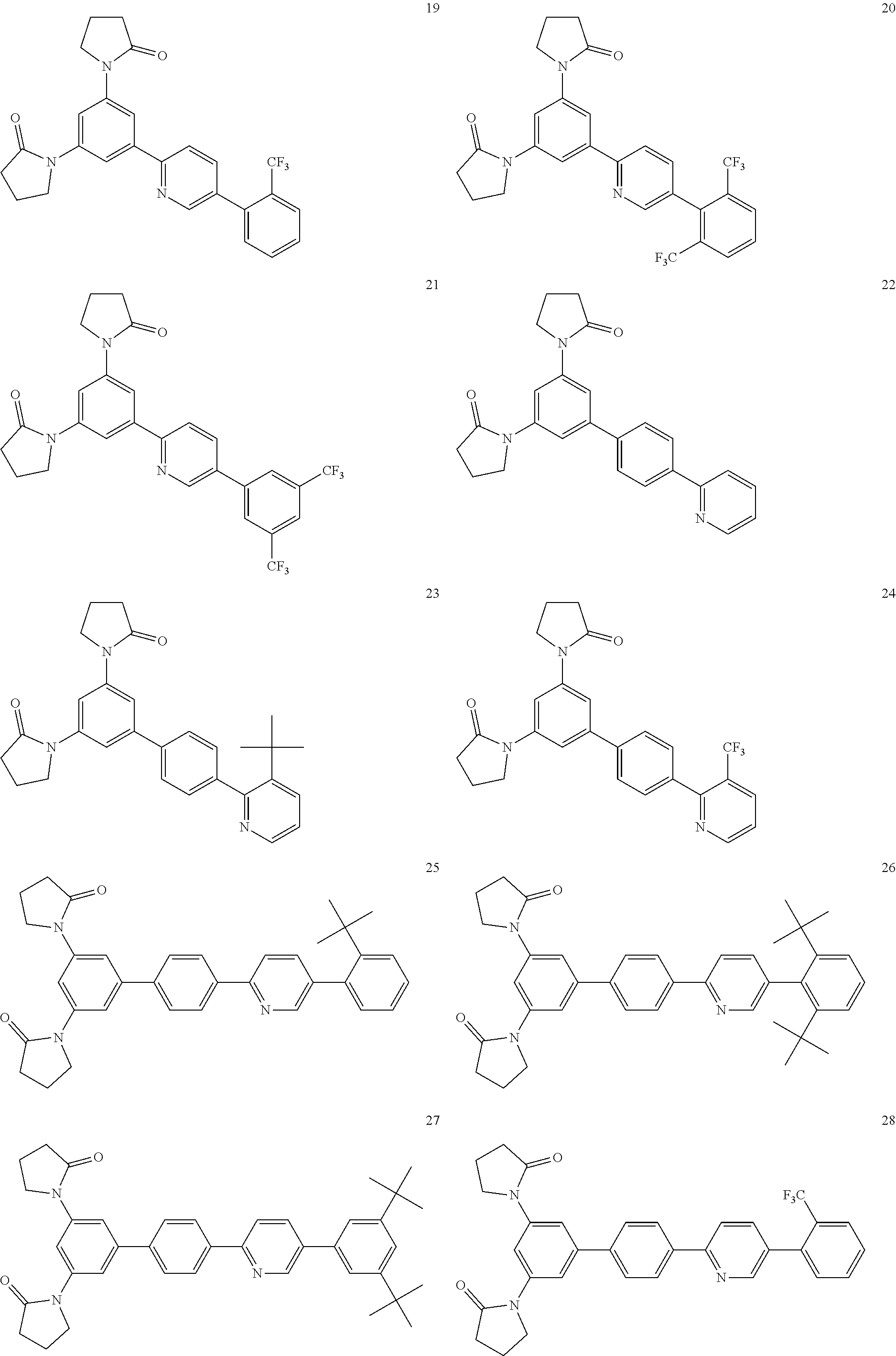

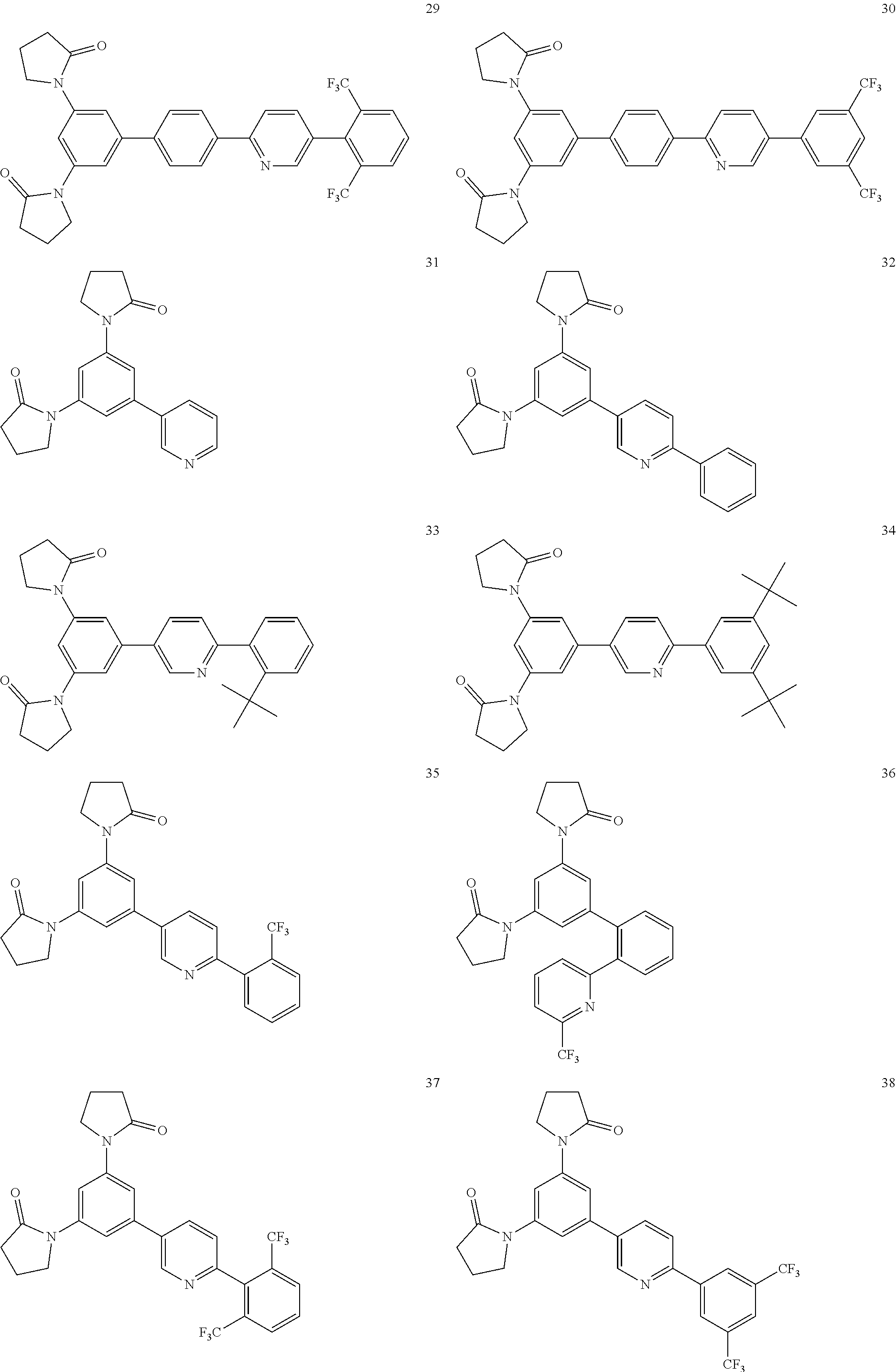

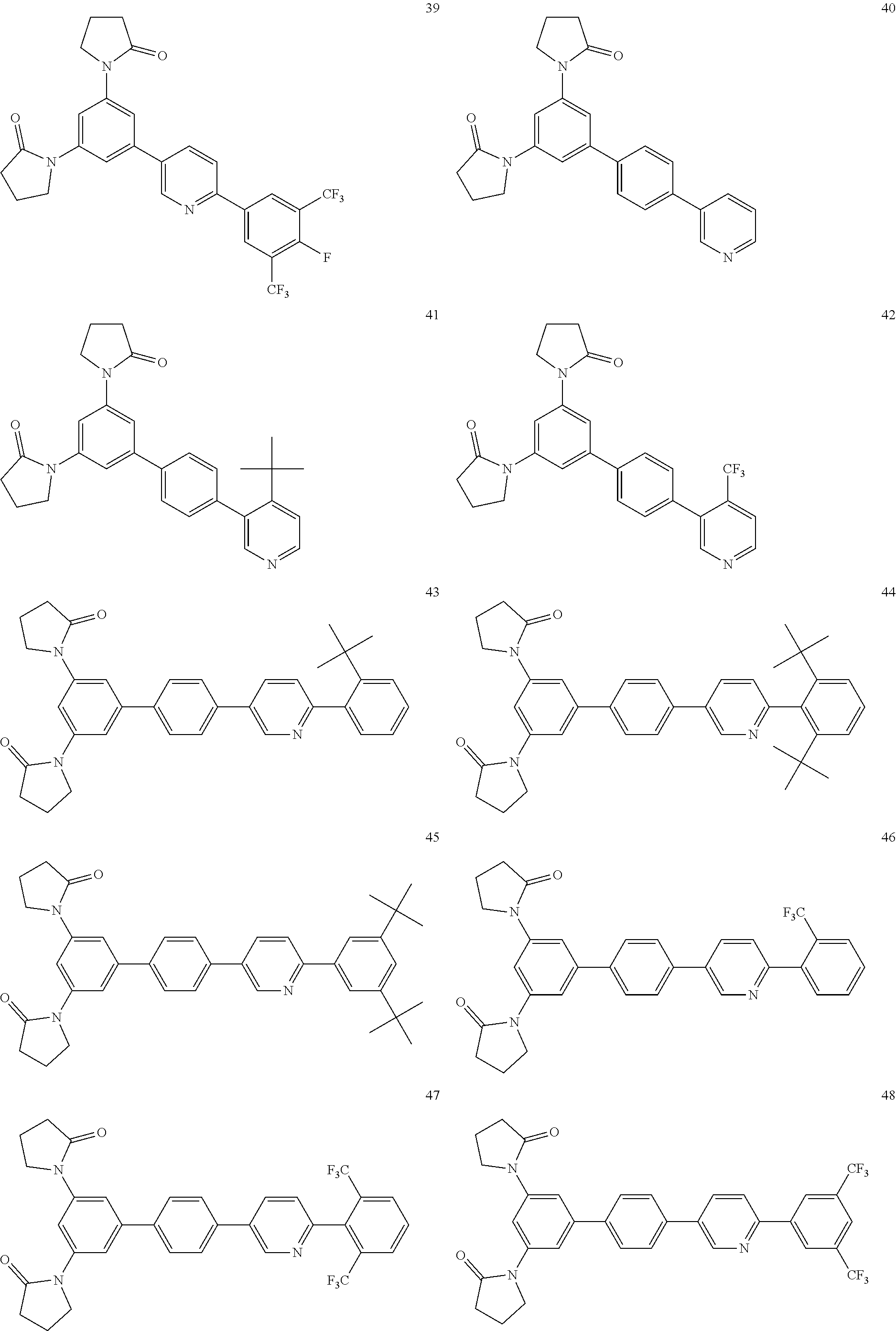

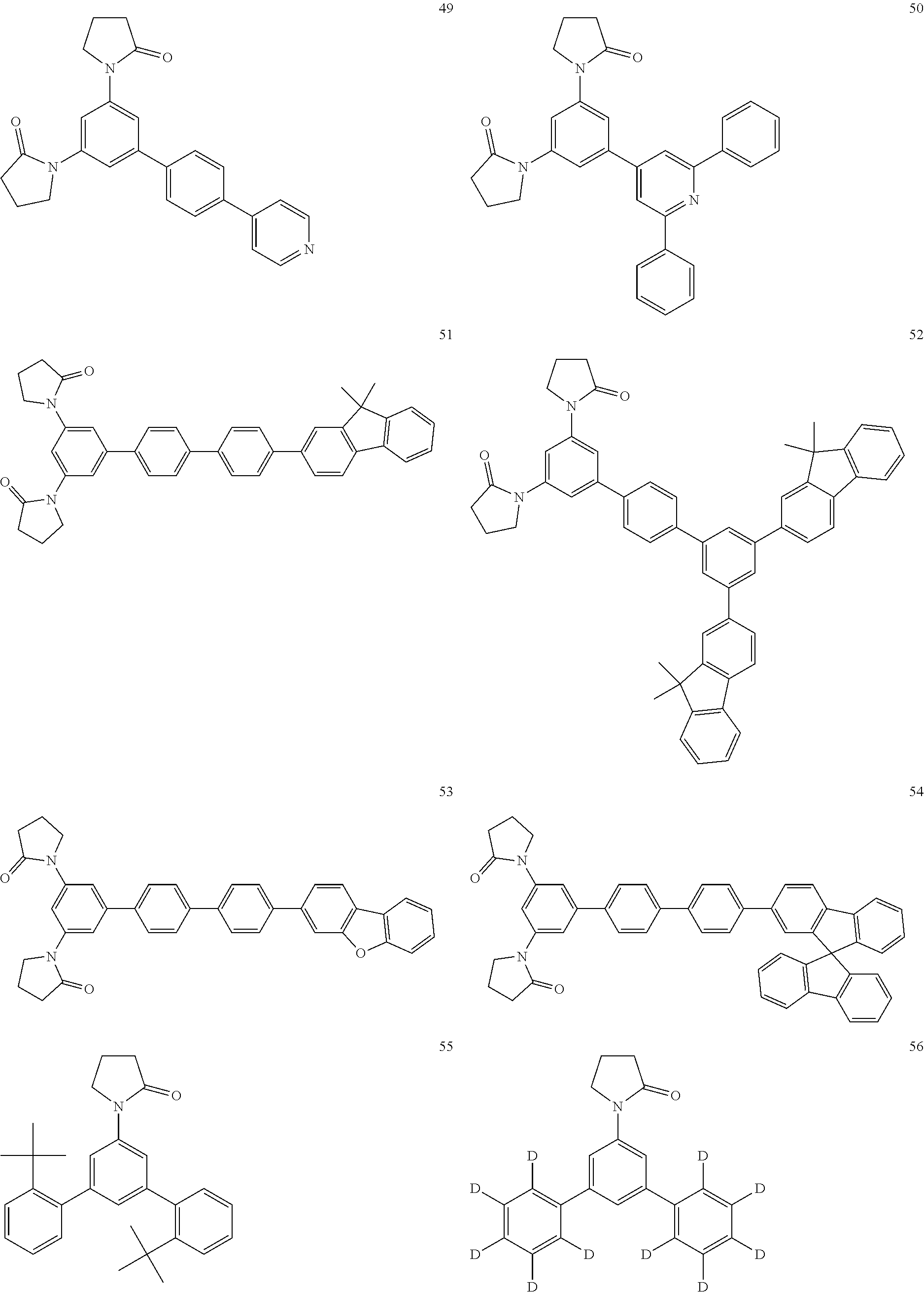

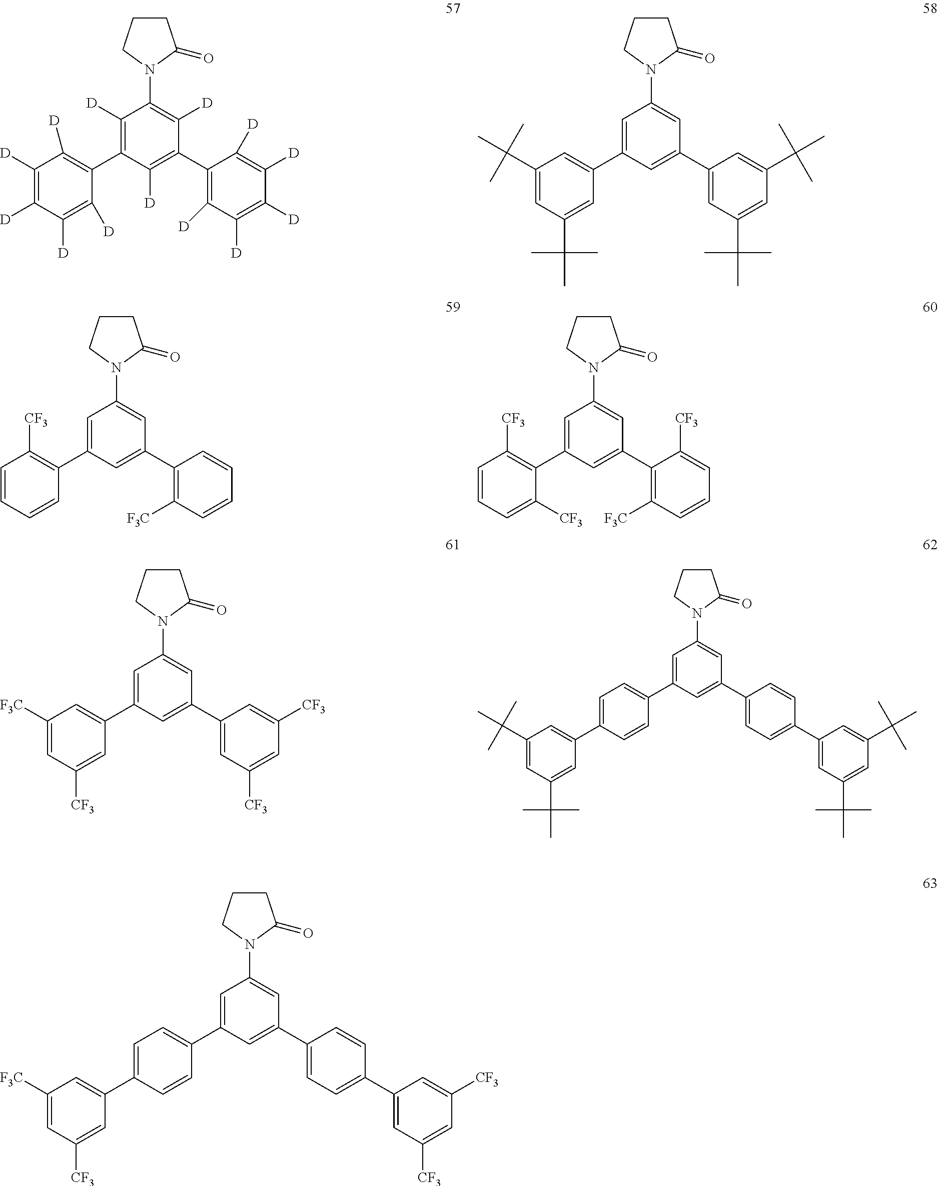

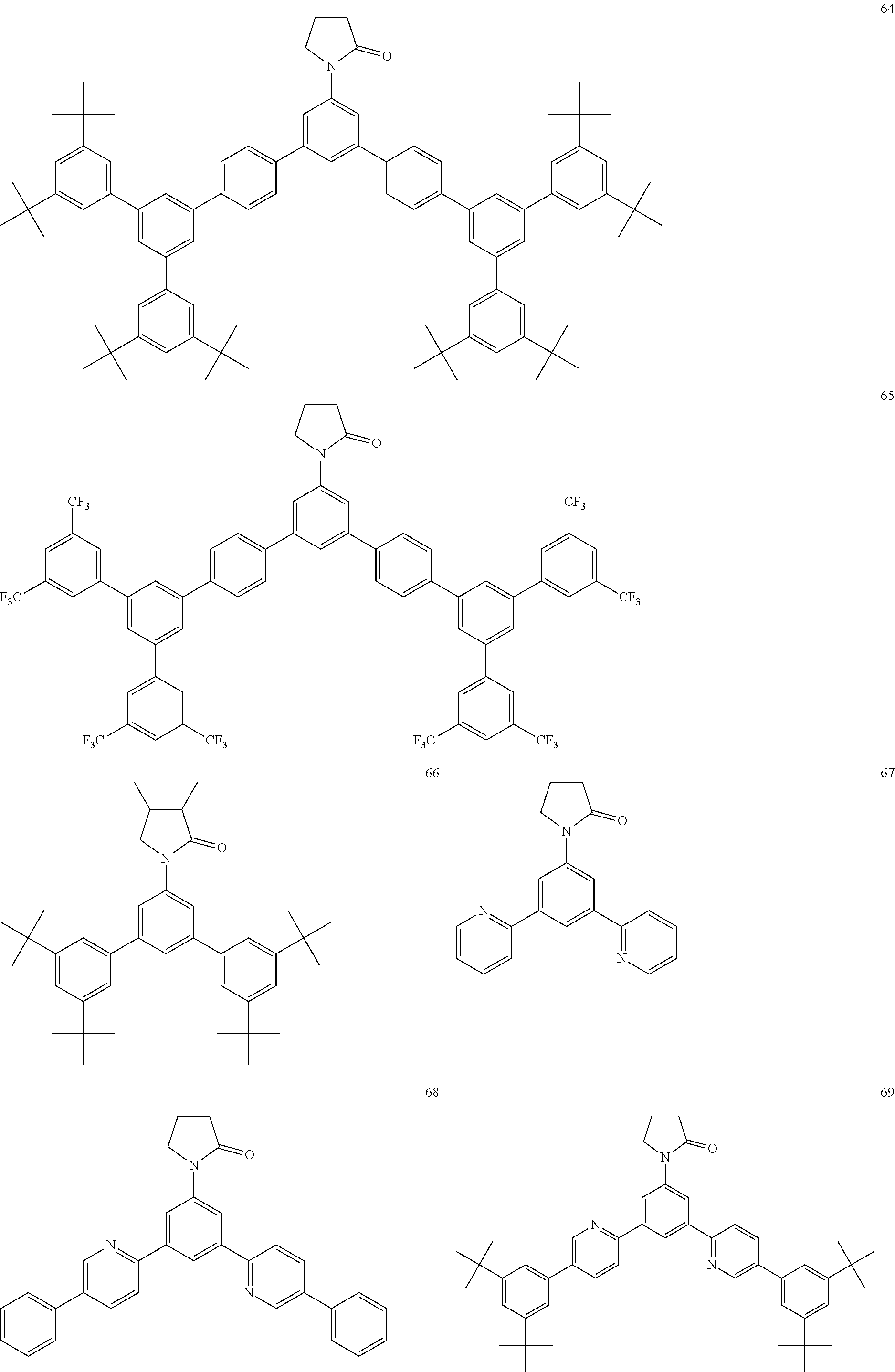

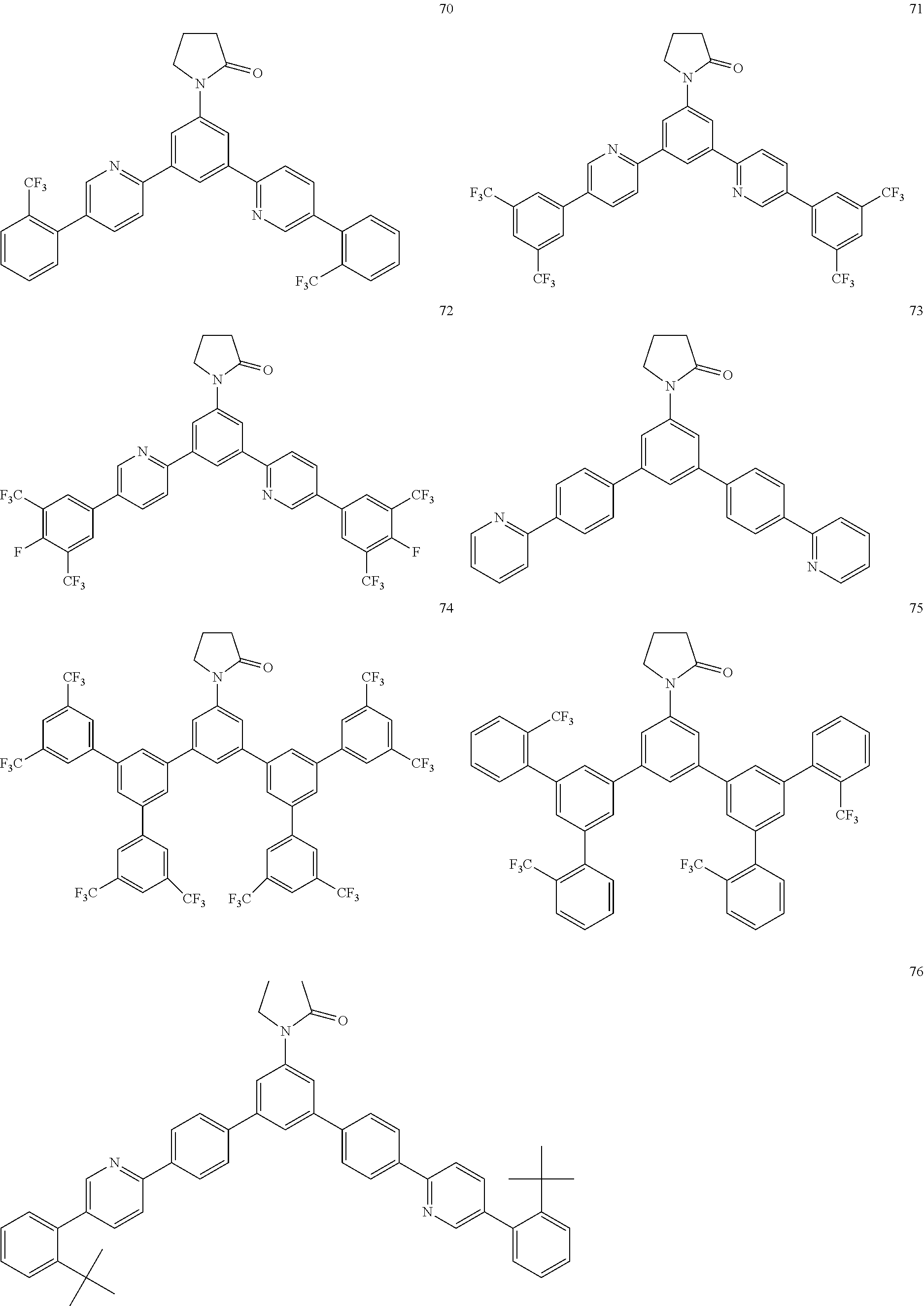

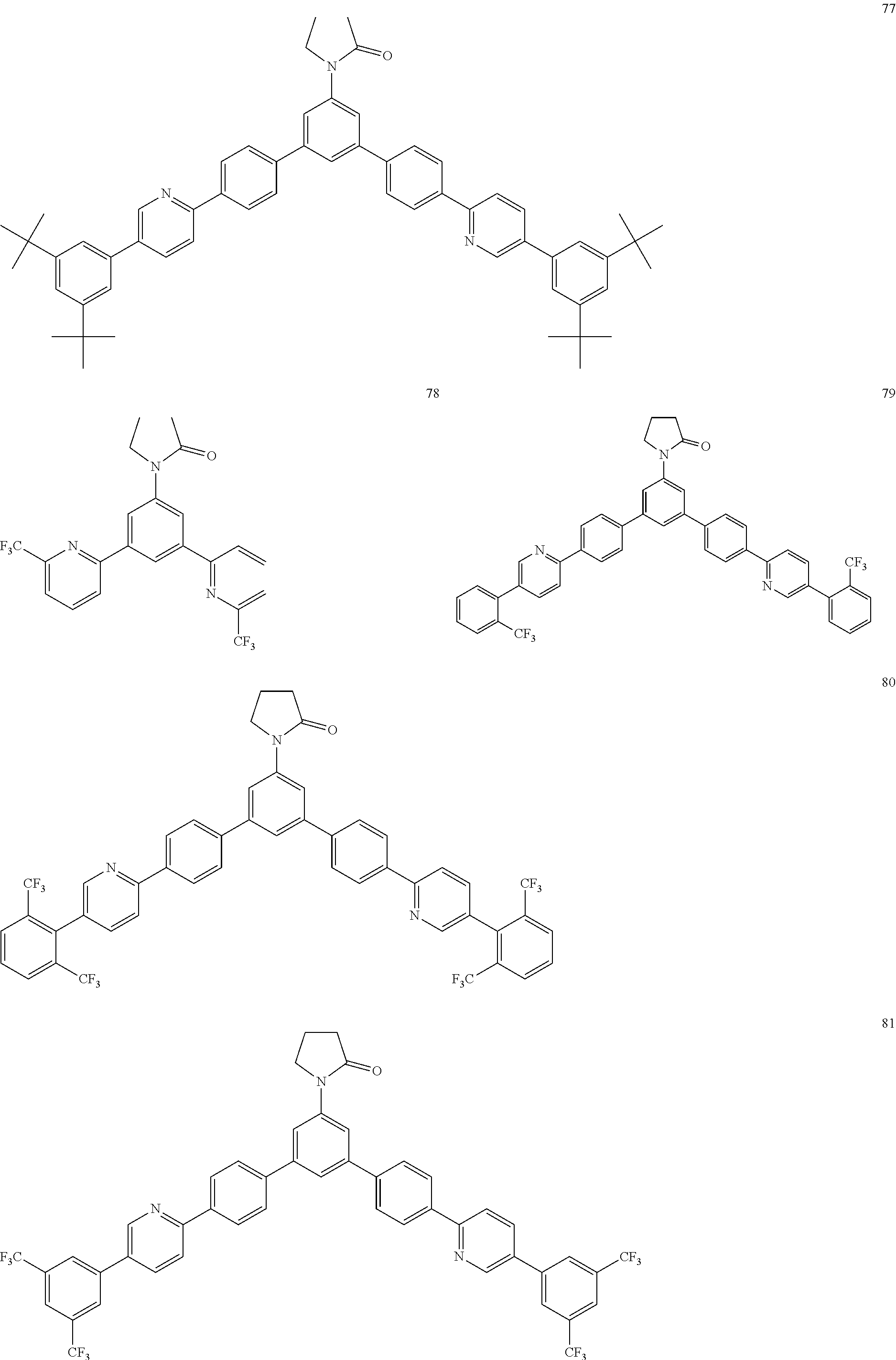

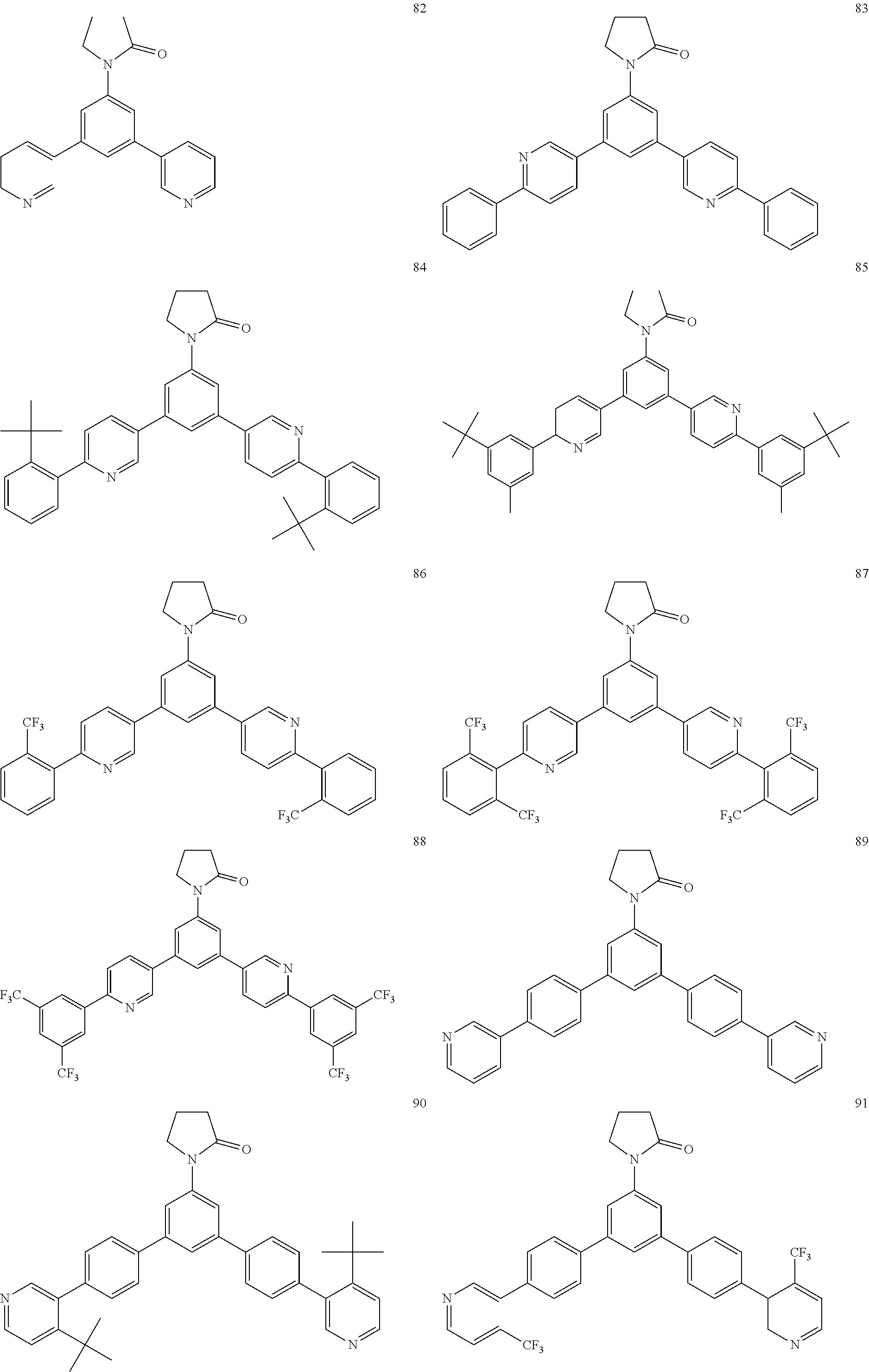

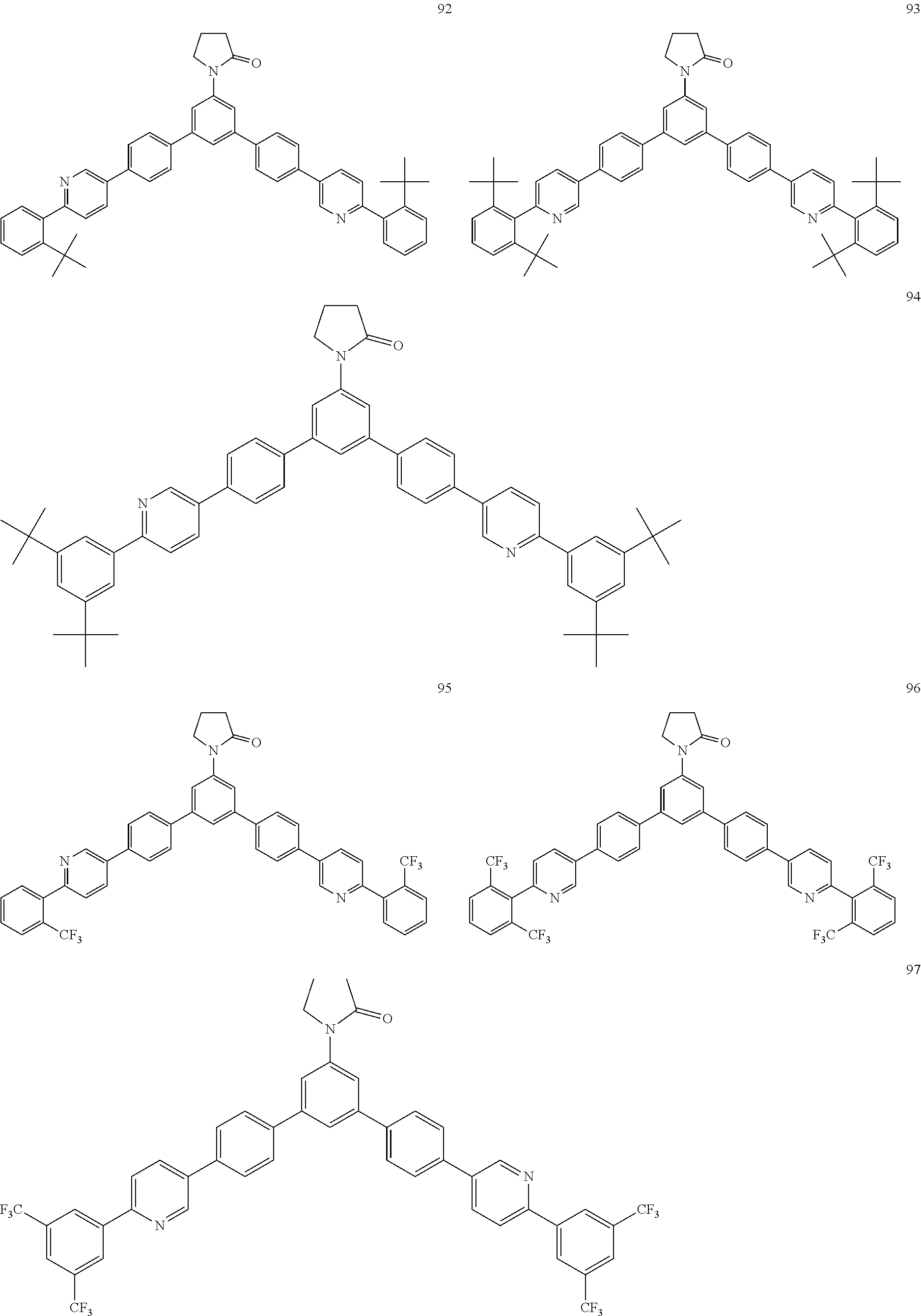

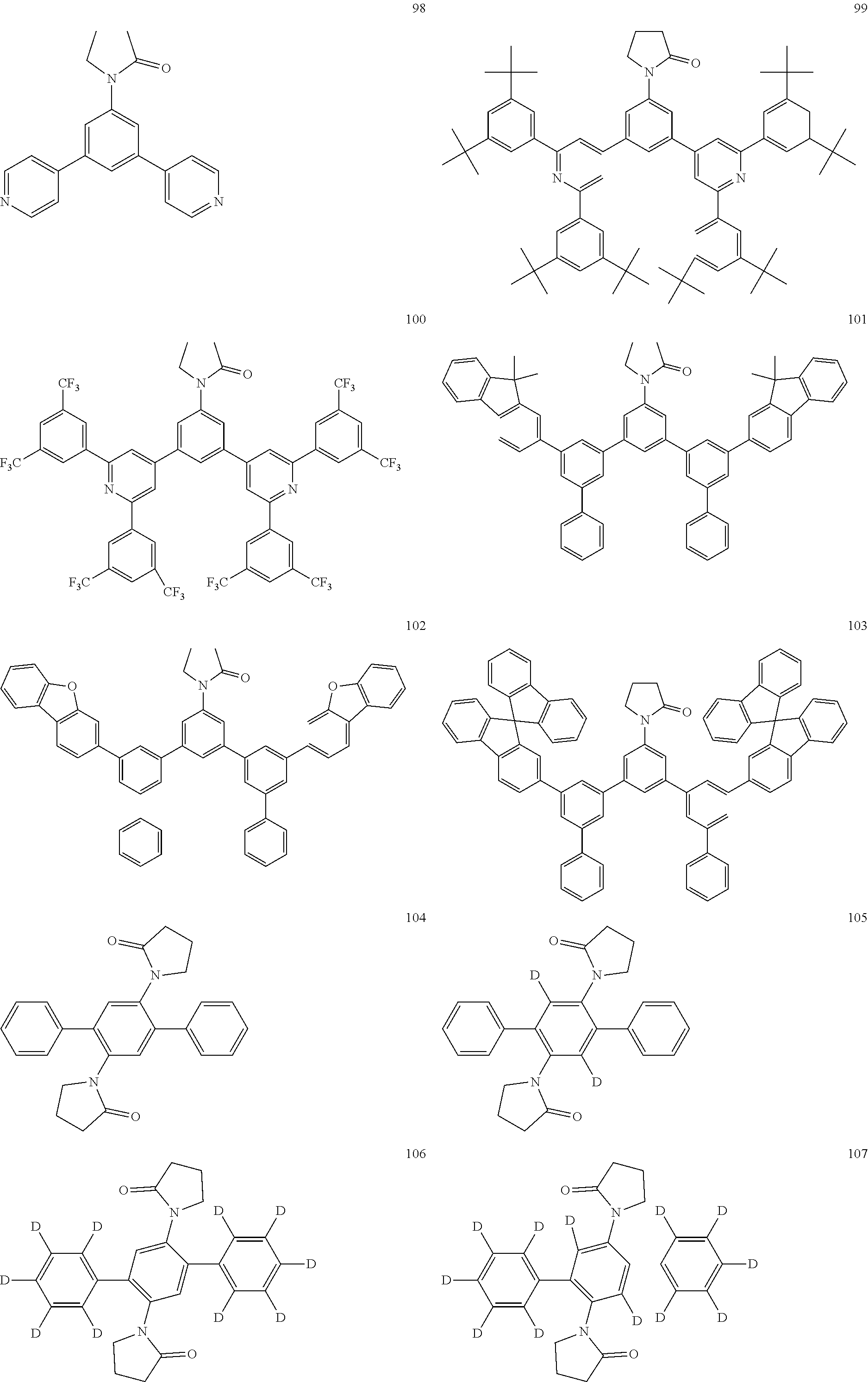

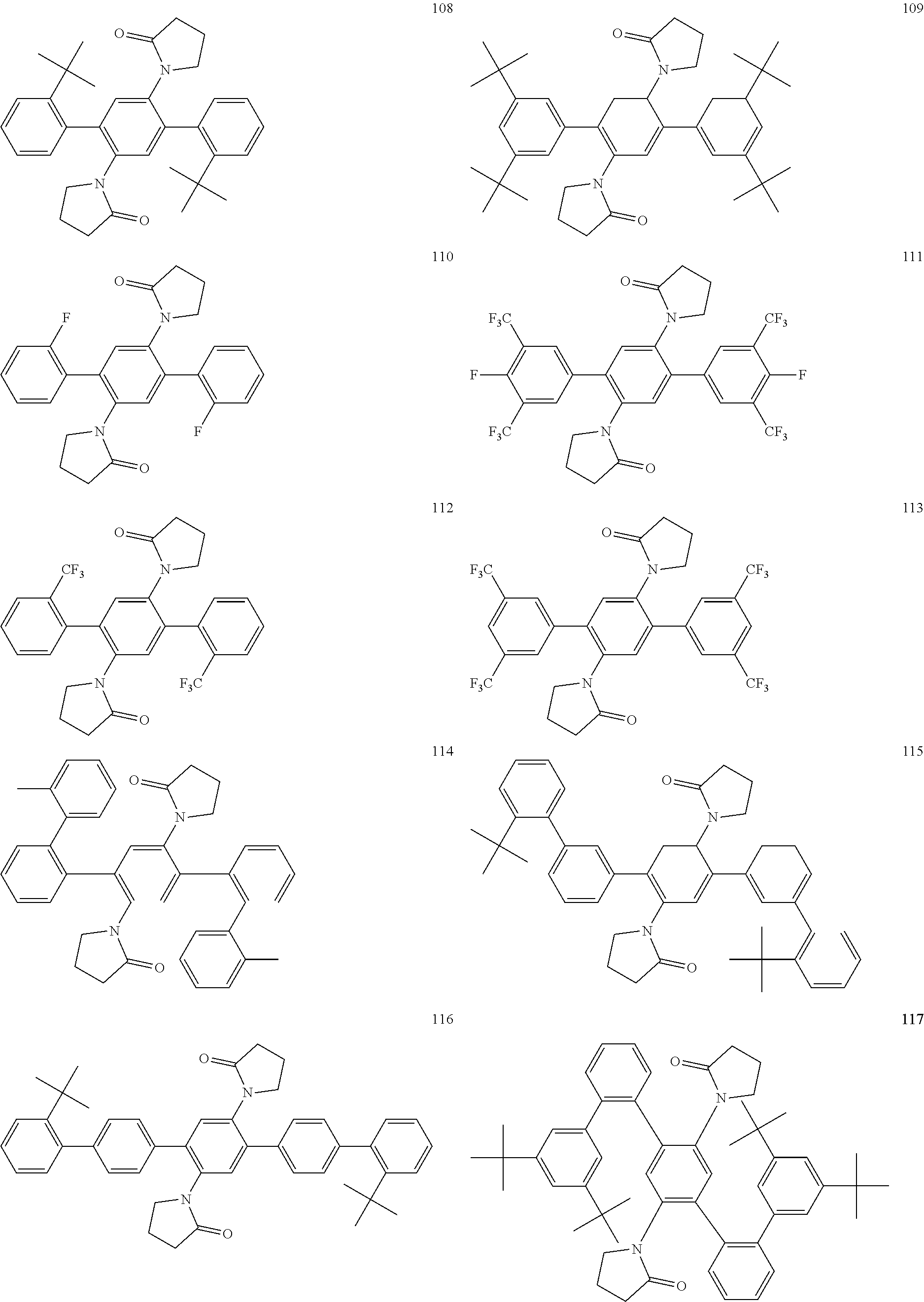

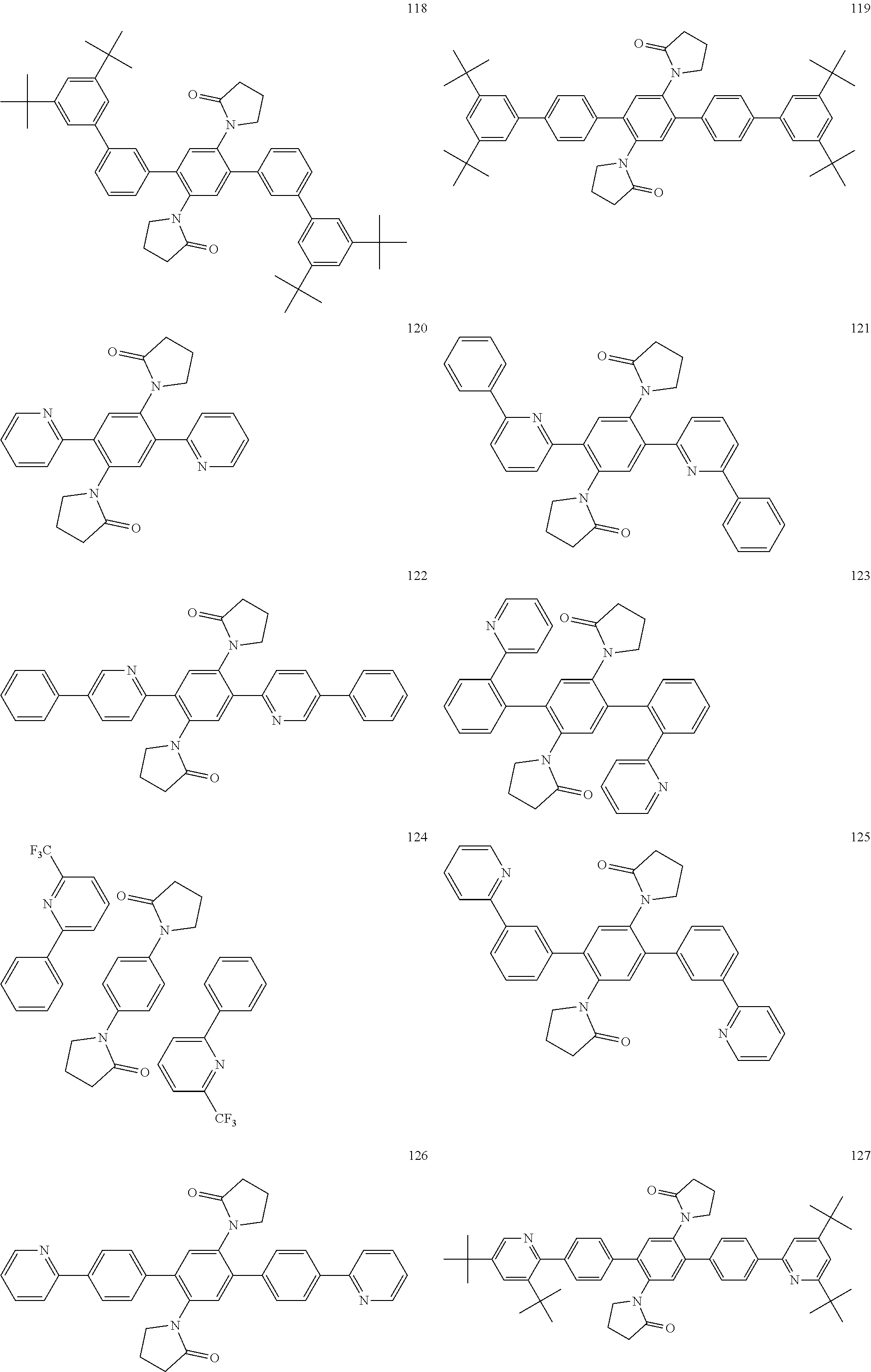

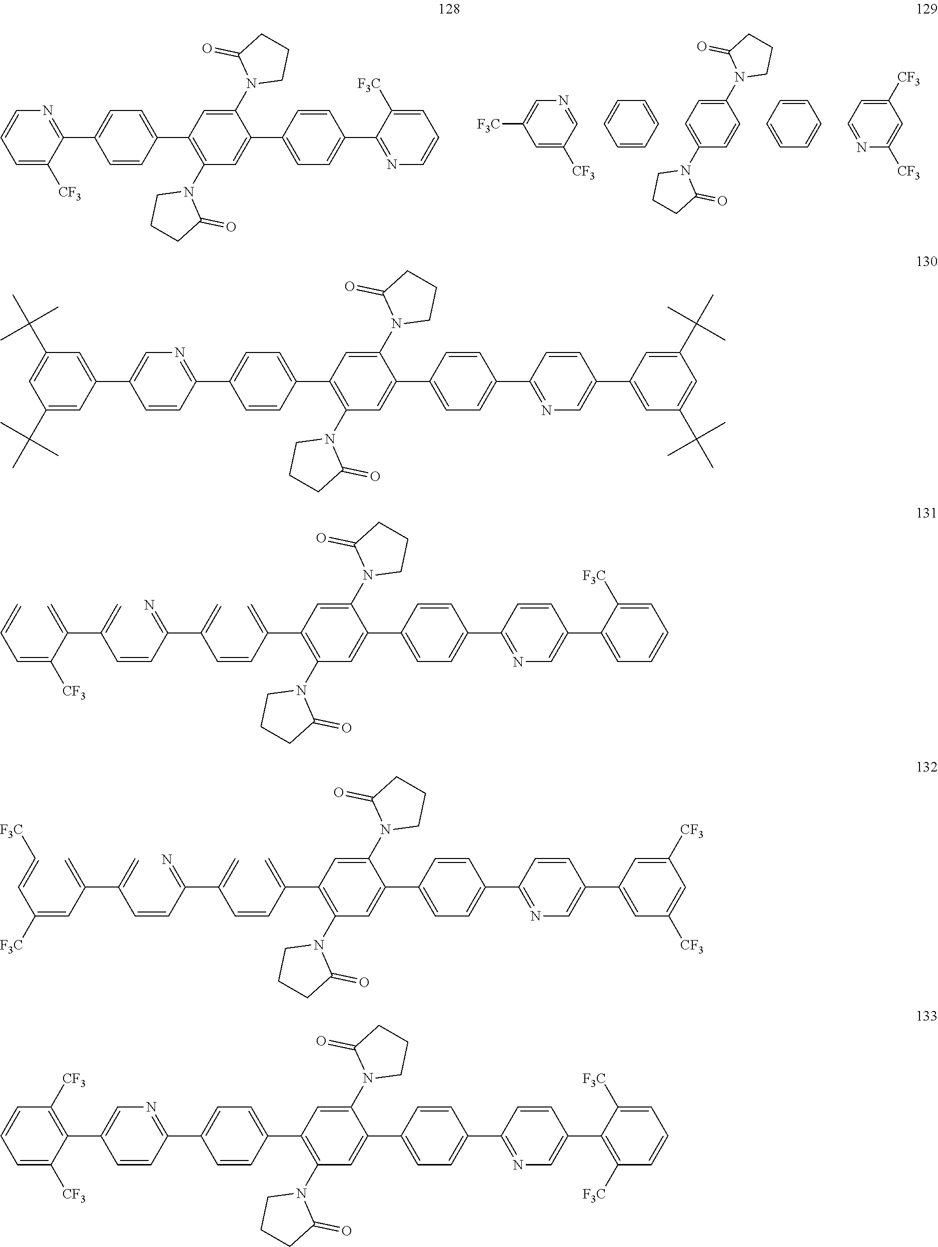

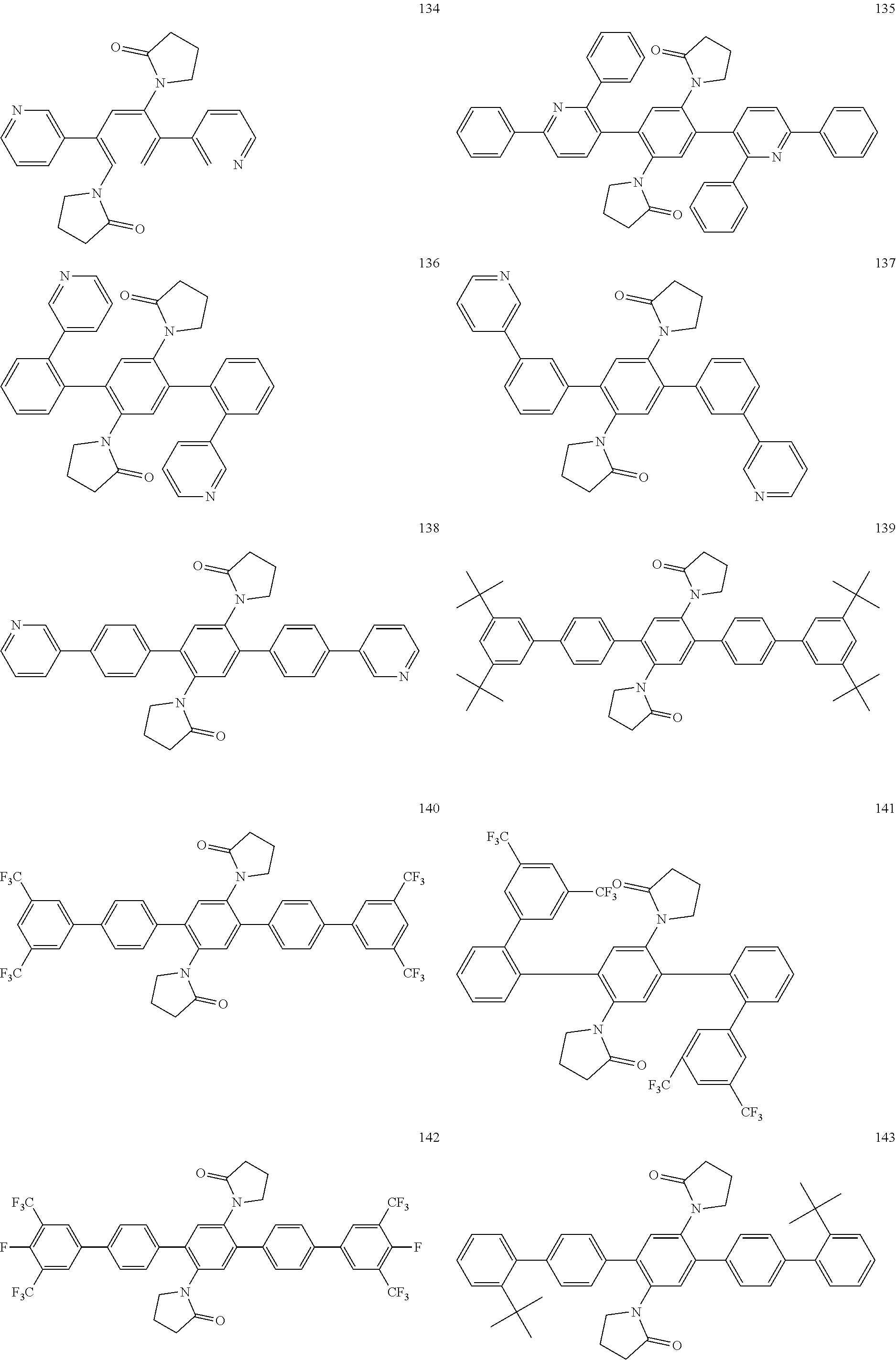

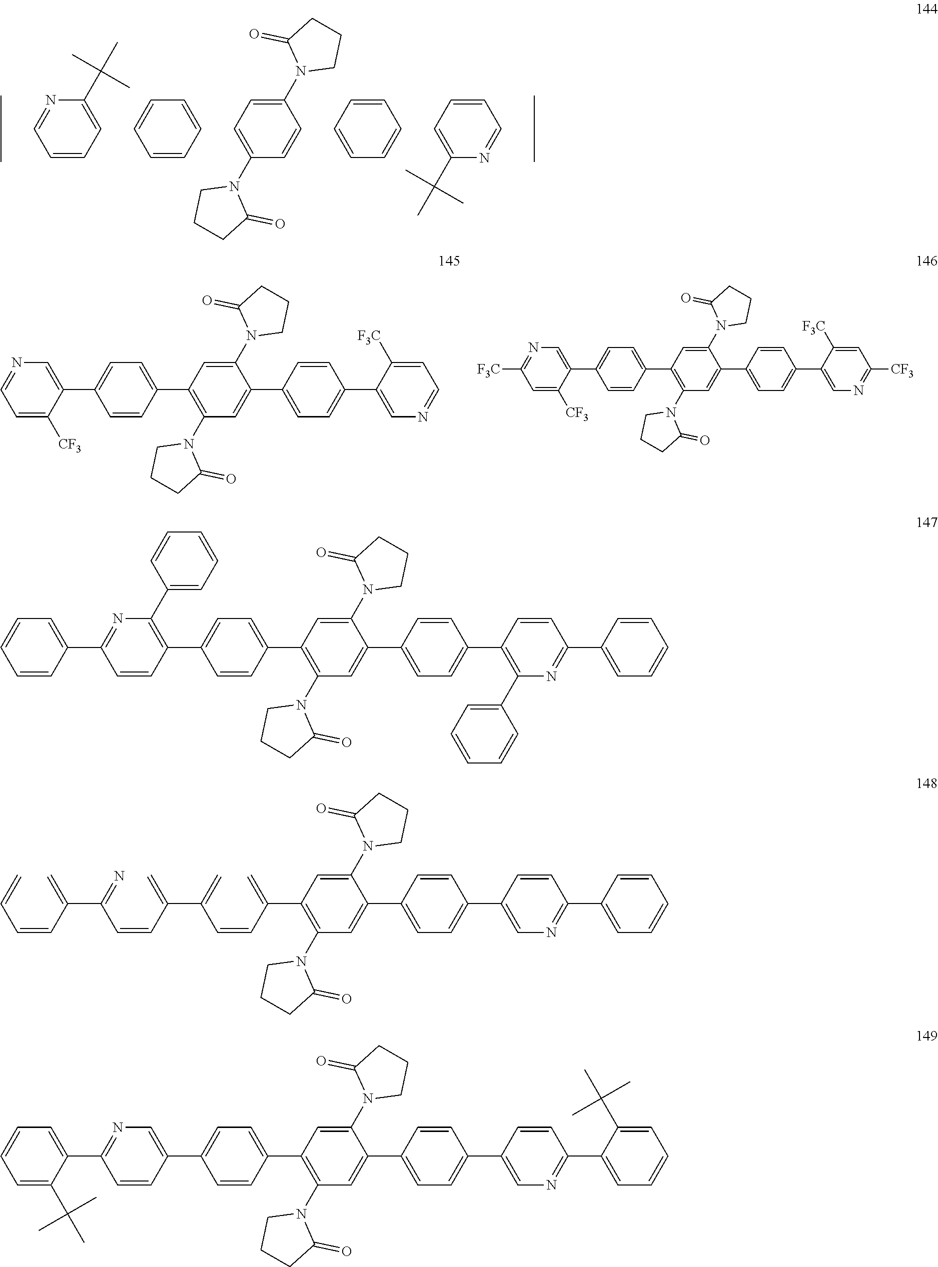

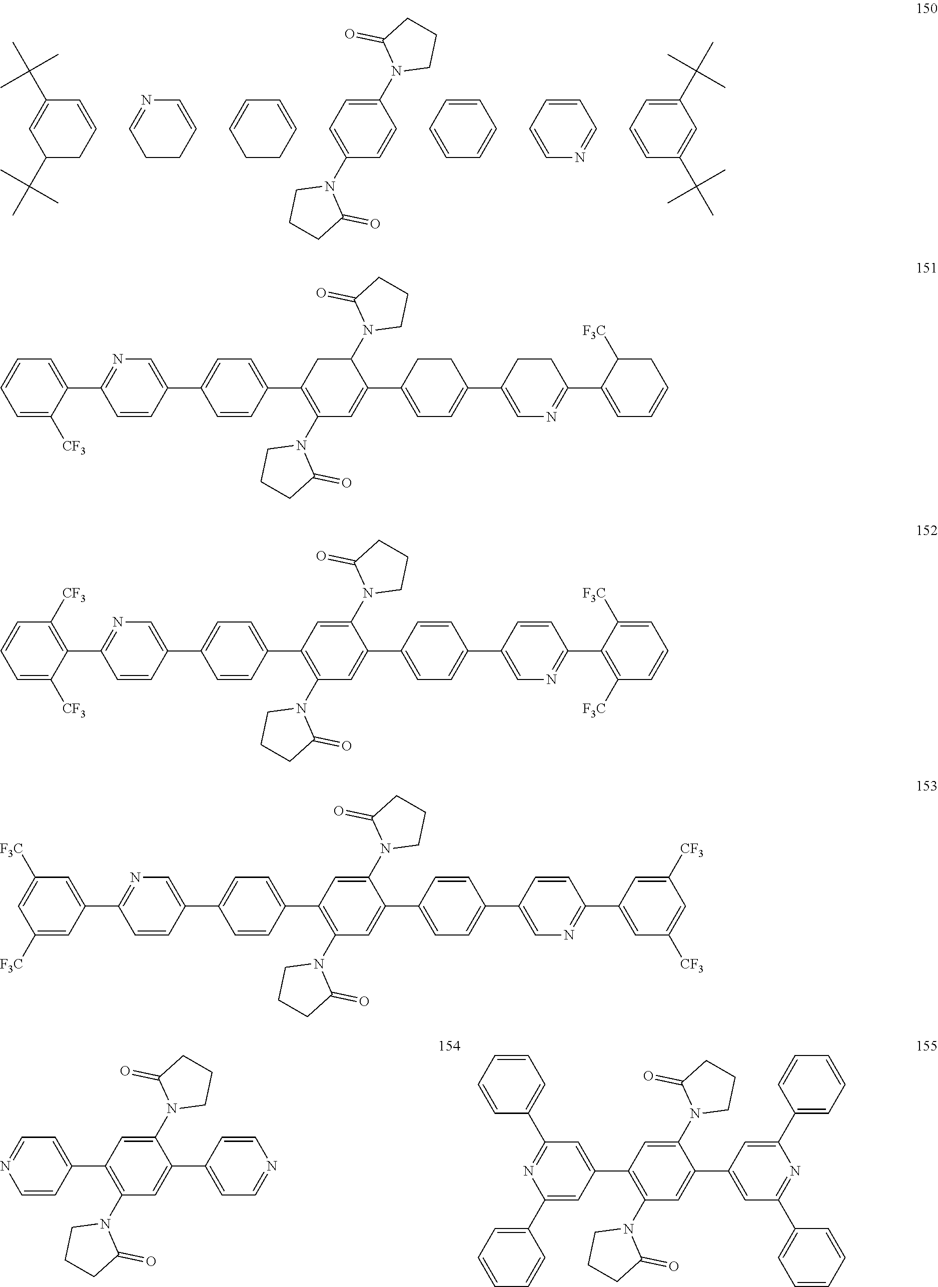

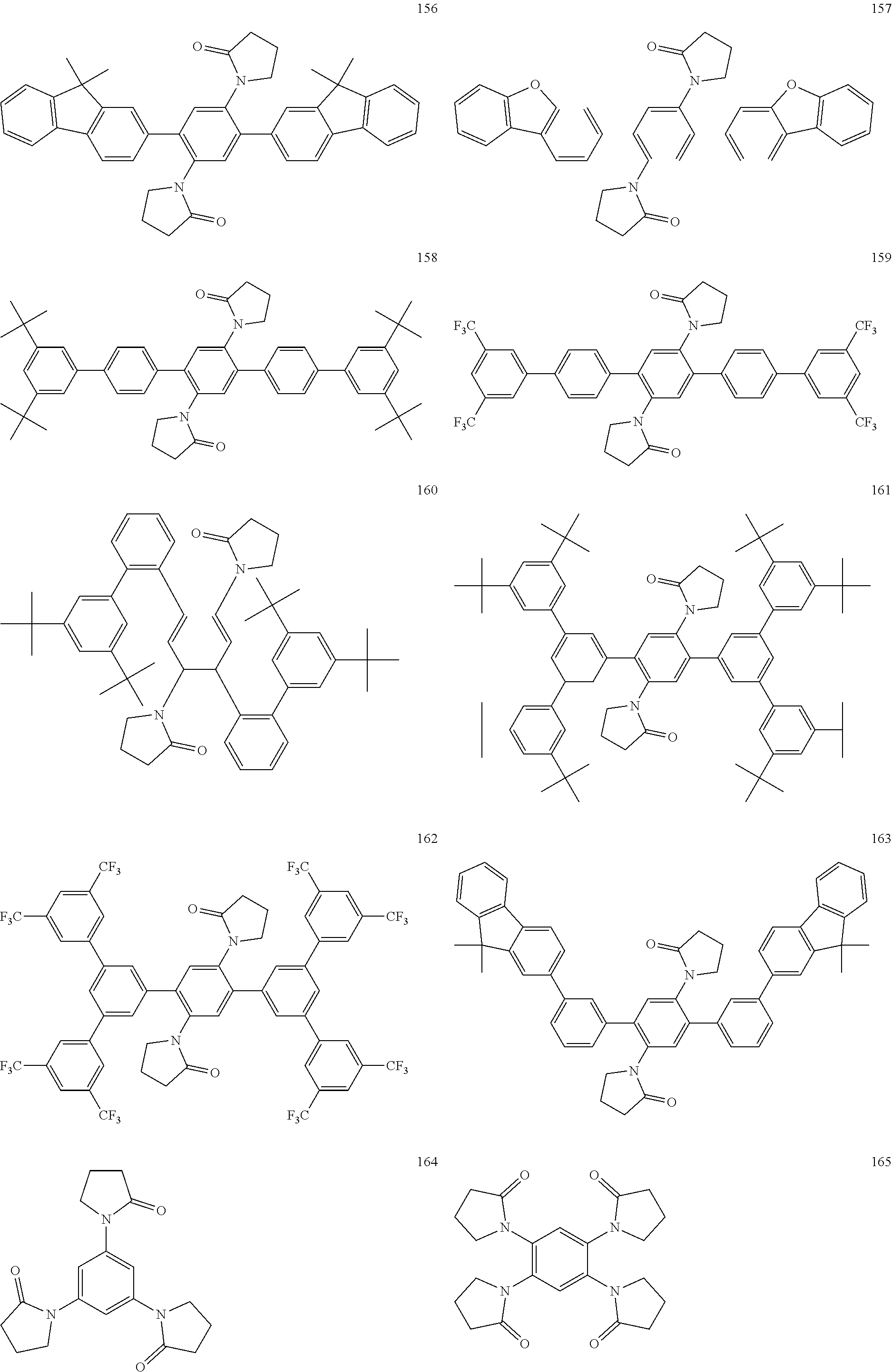

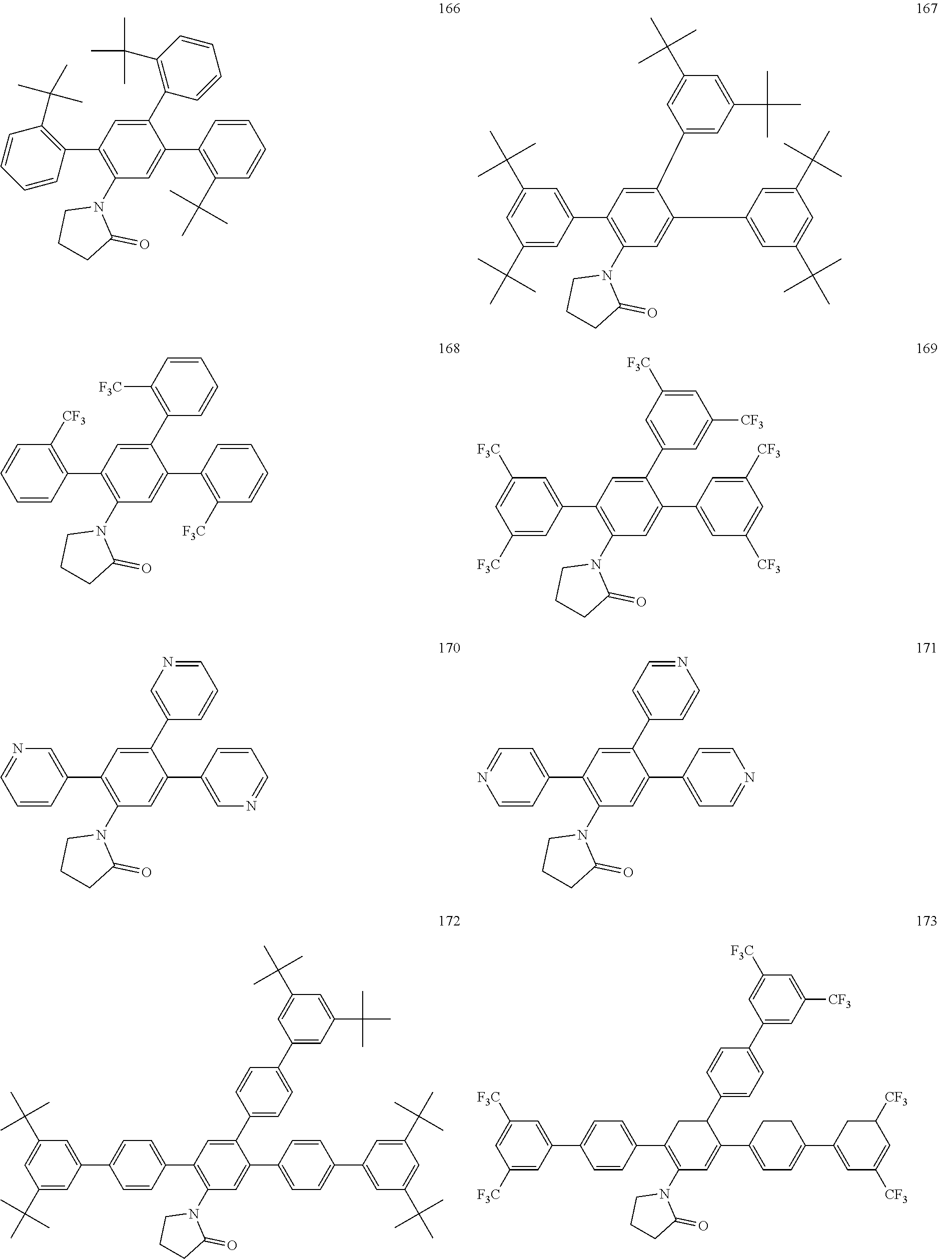

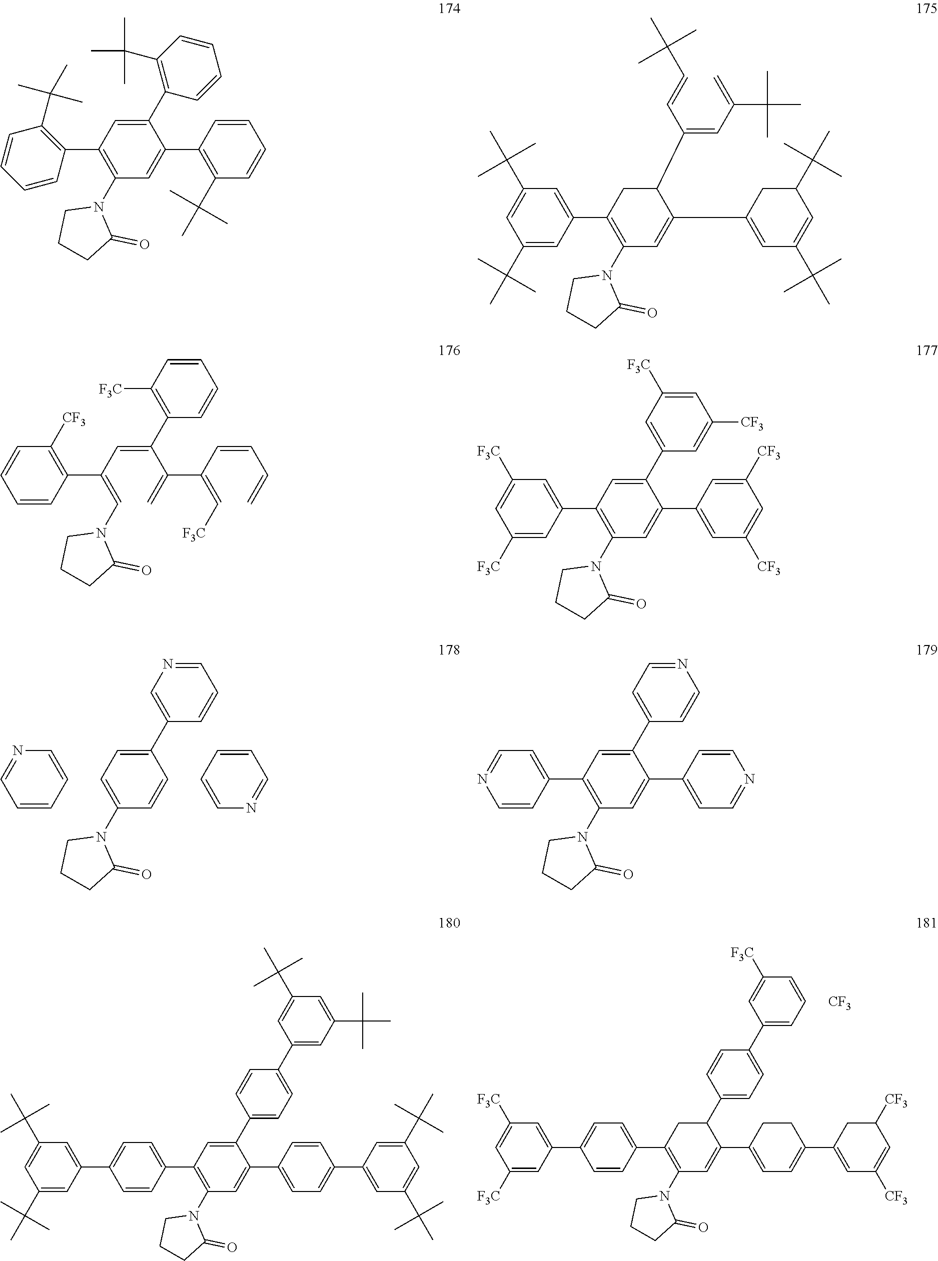

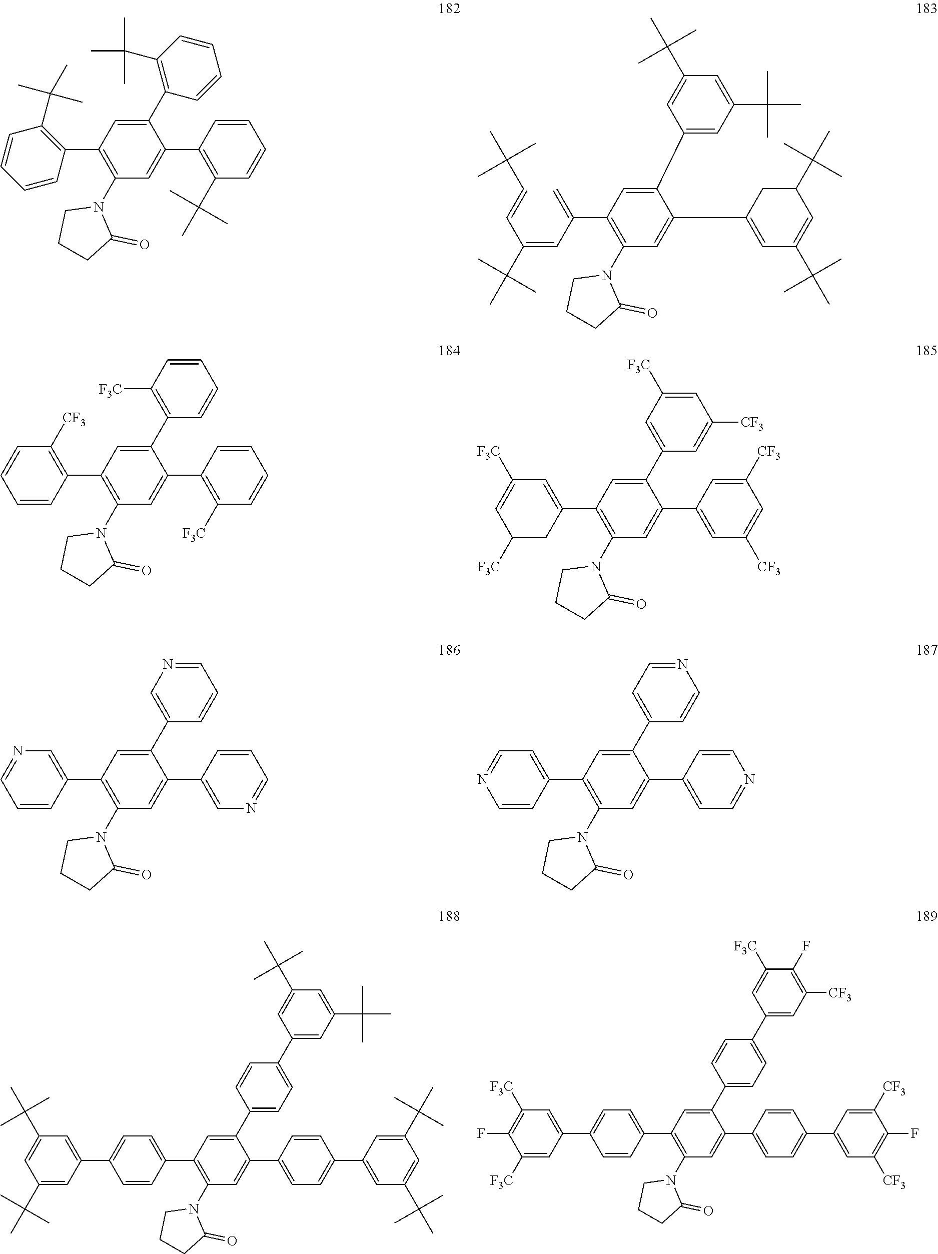

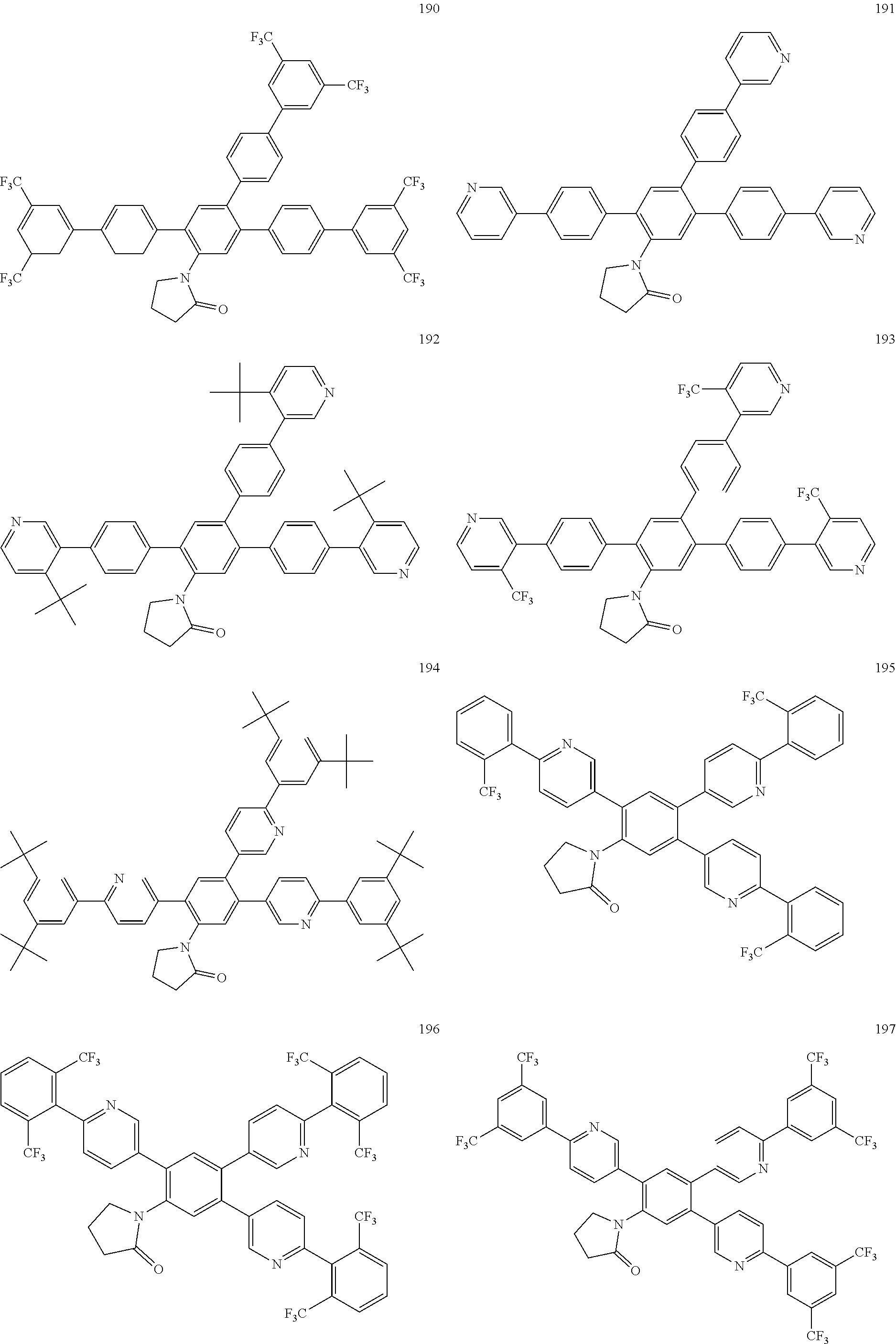

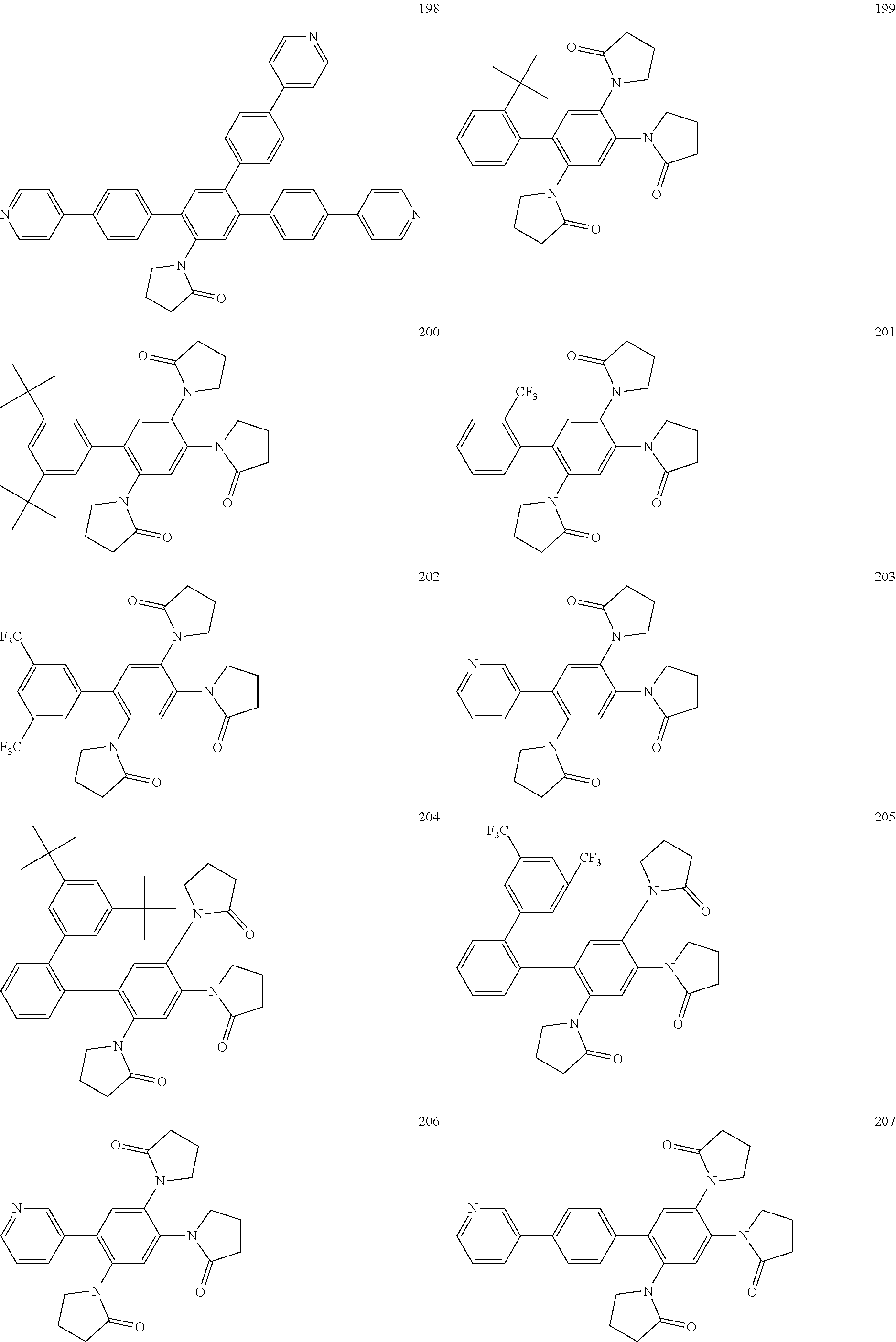

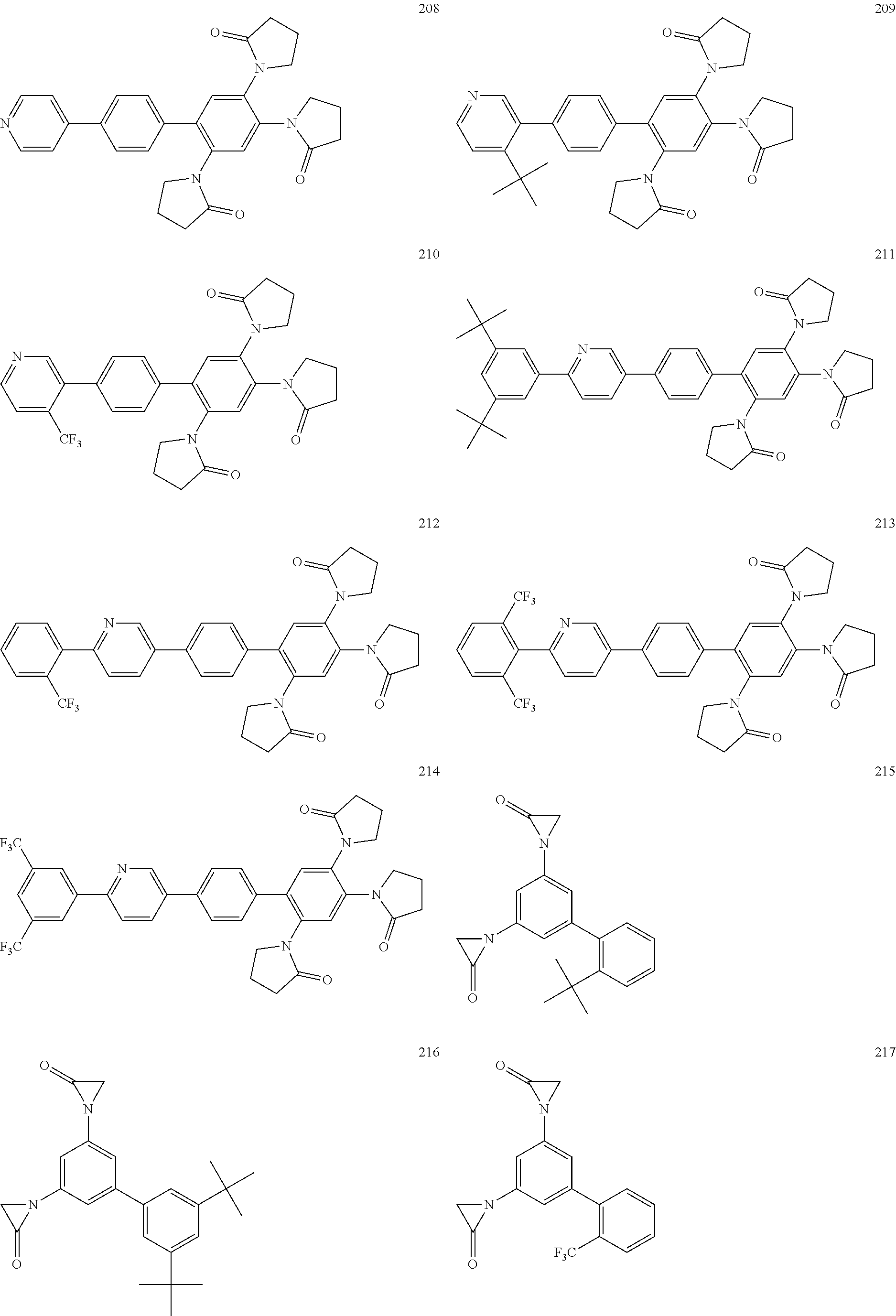

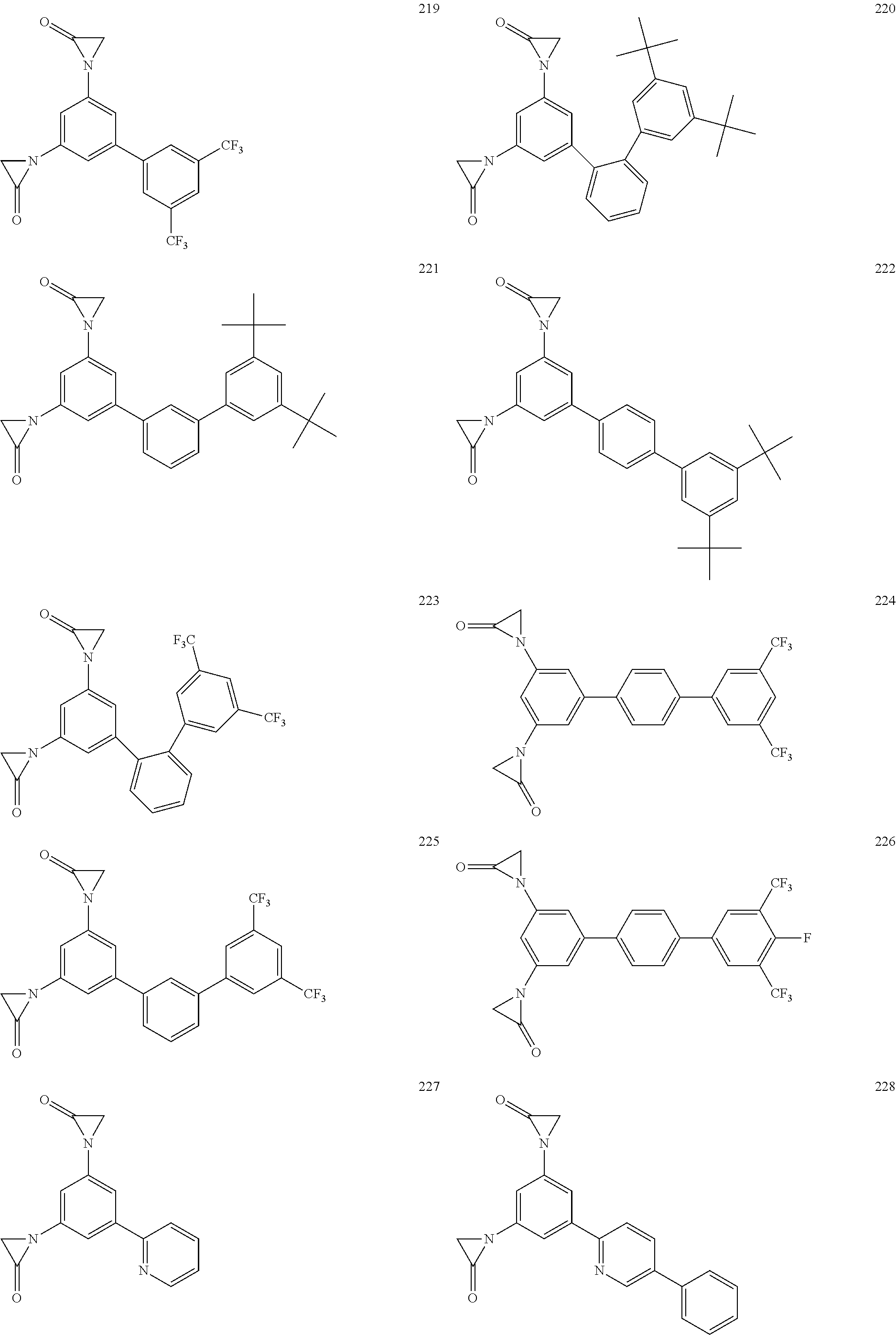

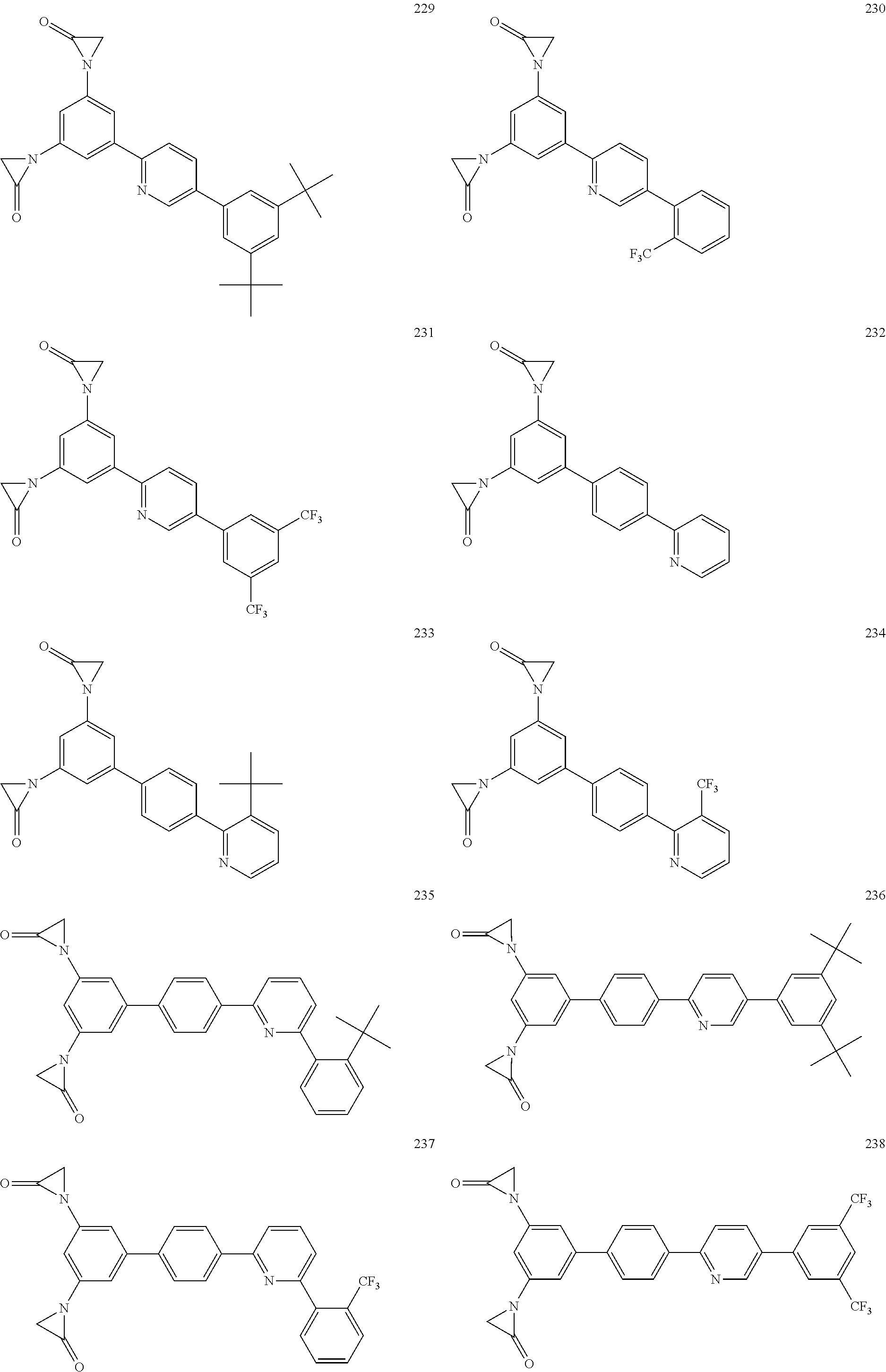

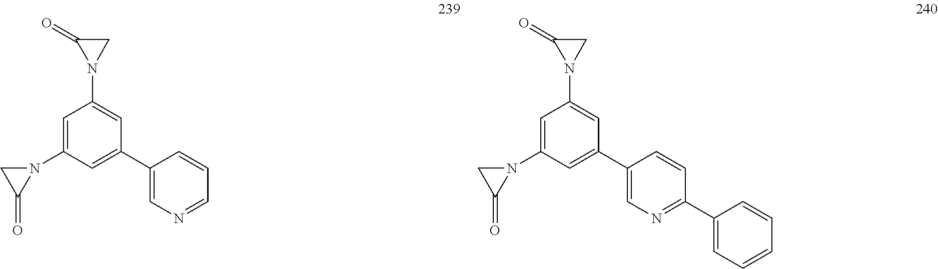 [SMILES: O=C1CN1c1cc(-c2cccnc2)cc(N2CC2=O)c1 ; O=C1CN1c1cc(-c2ccc(-c3ccccc3)nc2)cc(N2CC2=O)c1]

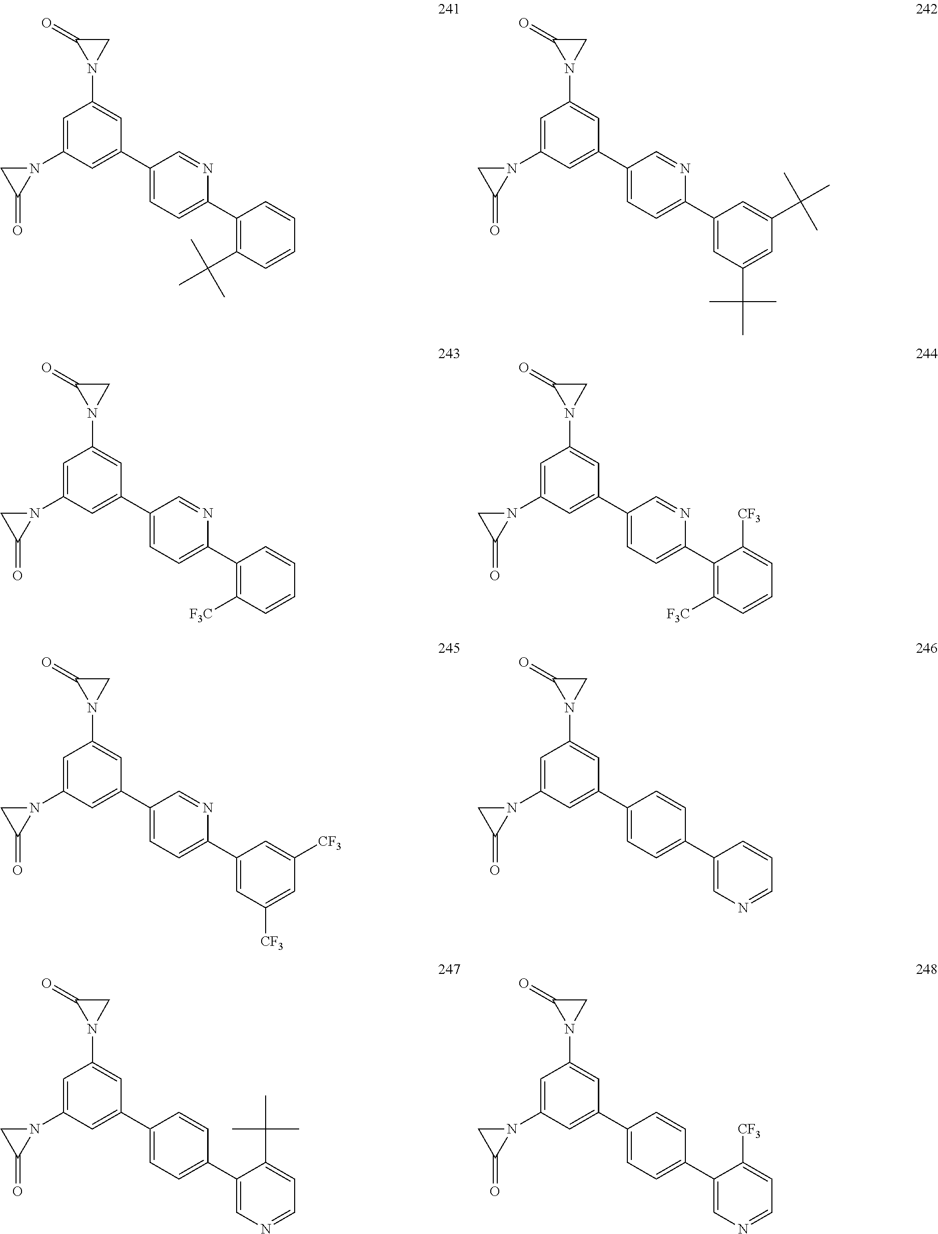

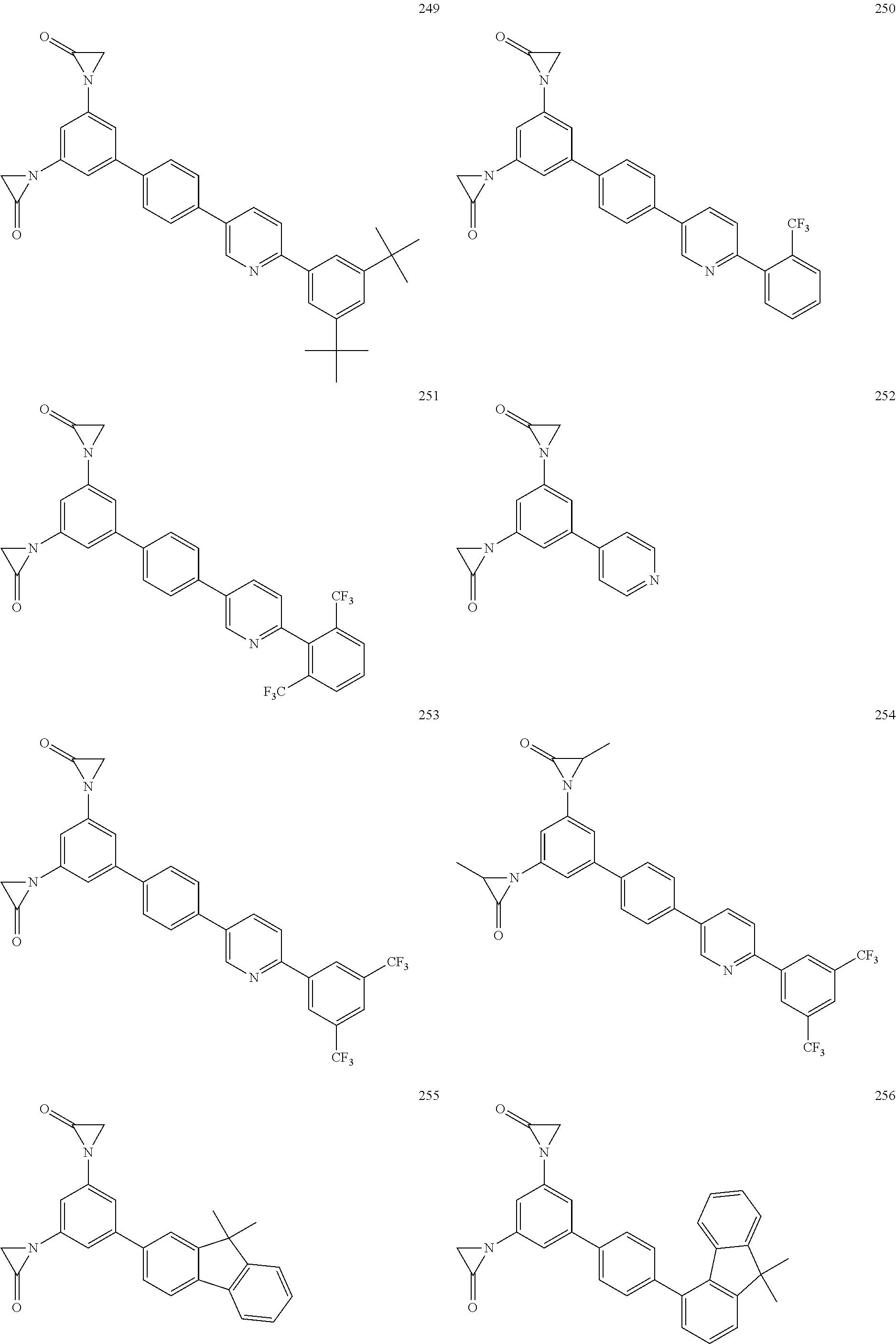

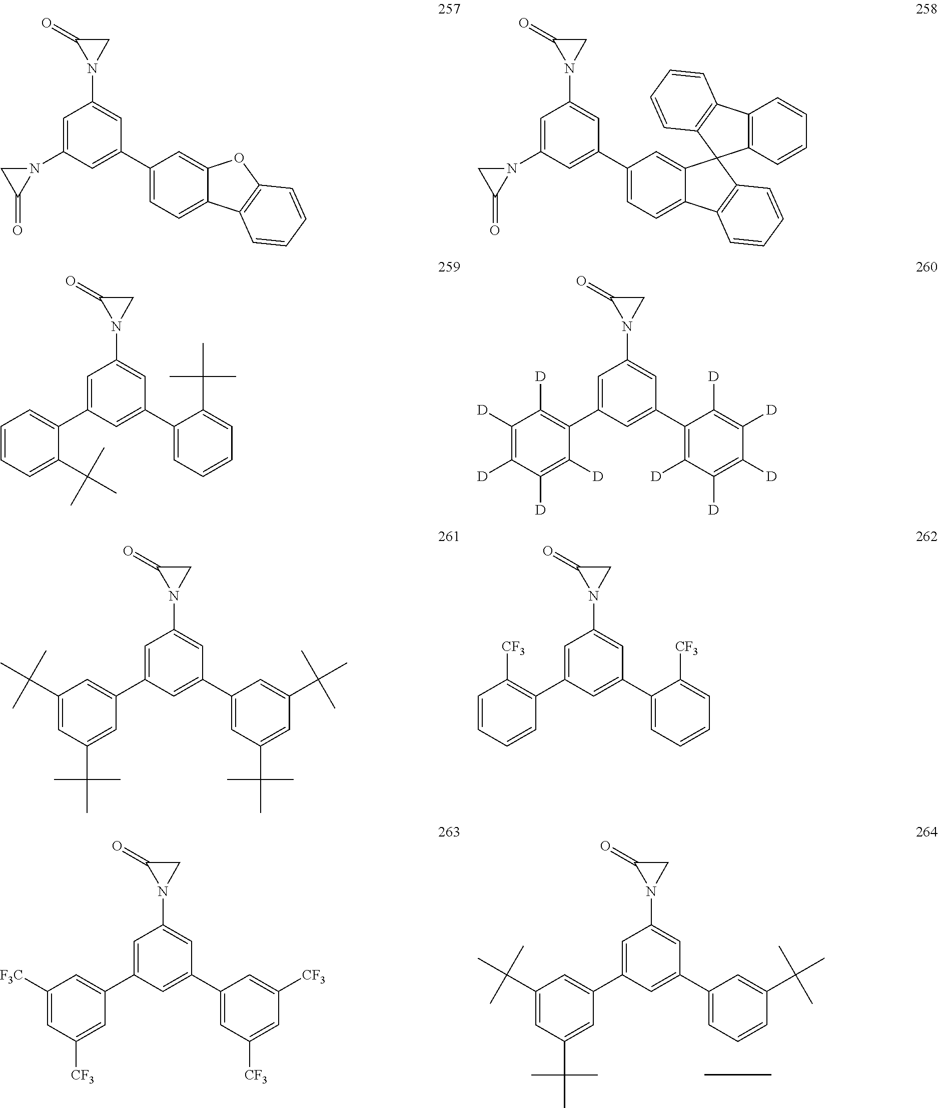

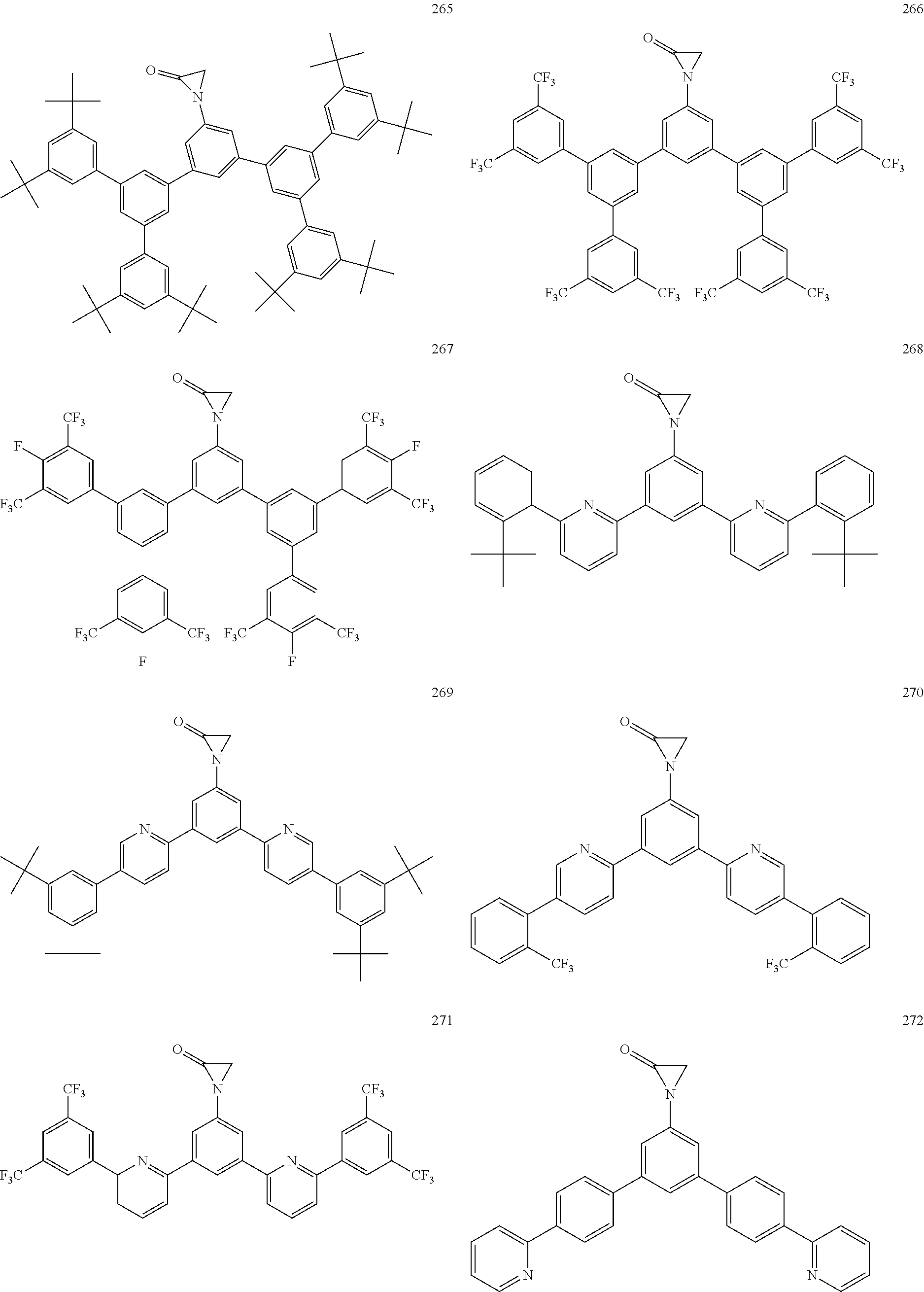

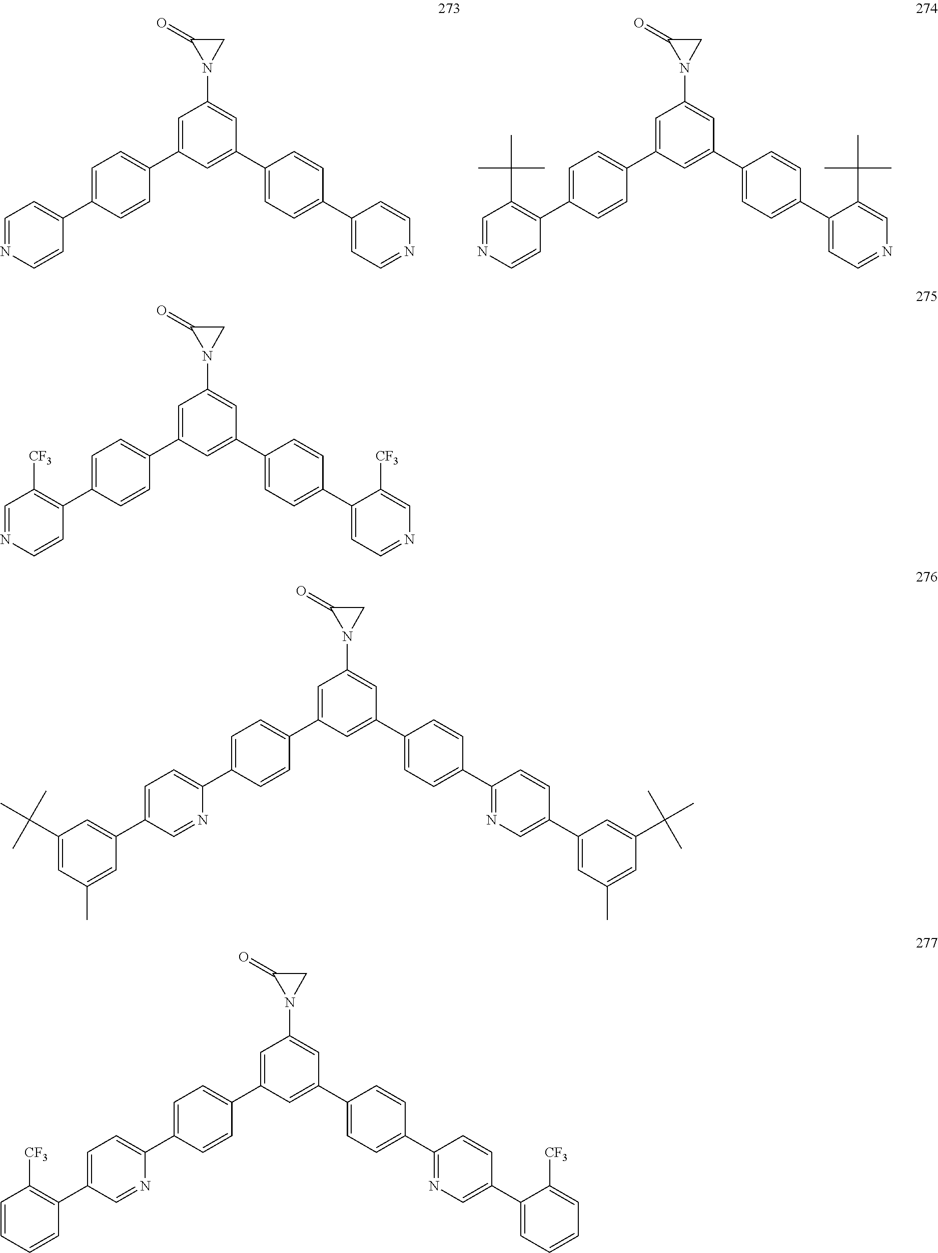 [SMILES: O=C1CN1c1cc(-c2ccc(-c3ccncc3)cc2)cc(-c2ccc(-c3ccncc3)cc2)c1 ; CC(C)(C)c1cnccc1-c1ccc(-c2cc(-c3ccc(-c4ccncc4C(C)(C)C)cc3)cc(N3CC3=O)c2)cc1 ; O=C1CN1c1cc(-c2ccc(-c3ccncc3C(F)(F)F)cc2)cc(-c2ccc(-c3ccncc3C(F)(F)F)cc2)c1 ; Cc1cc(-c2ccc(-c3ccc(-c4cc(-c5ccc(-c6ccc(-c7cc(C)cc(C(C)(C)C)c7)cn6)cc5)cc(N5CC5=O)c4)cc3)nc2)cc(C(C)(C)C)c1 ; O=C1CN1c1cc(-c2ccc(-c3ccc(-c4ccccc4C(F)(F)F)cn3)cc2)cc(-c2ccc(-c3ccc(-c4ccccc4C(F)(F)F)cn3)cc2)c1]

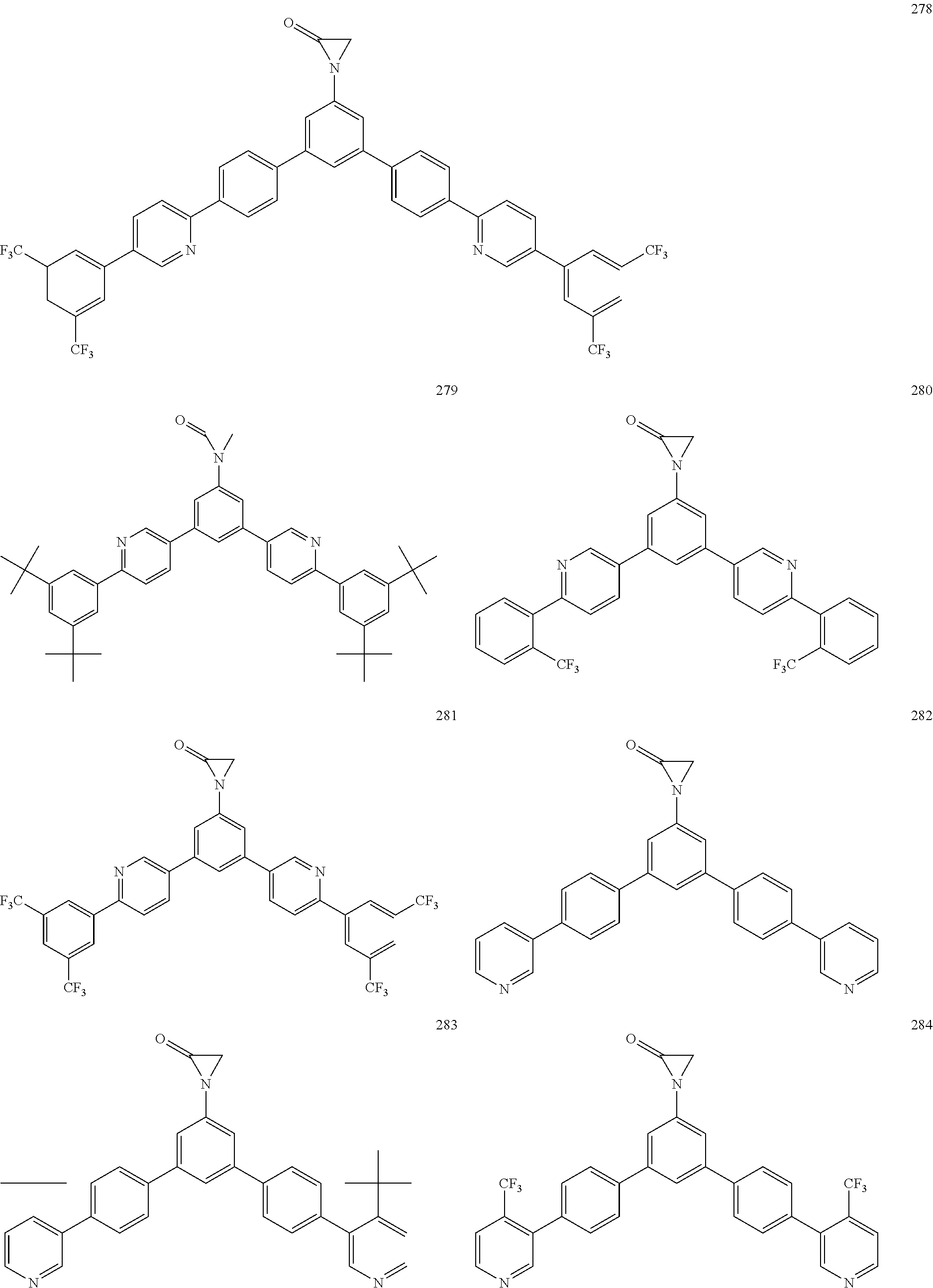

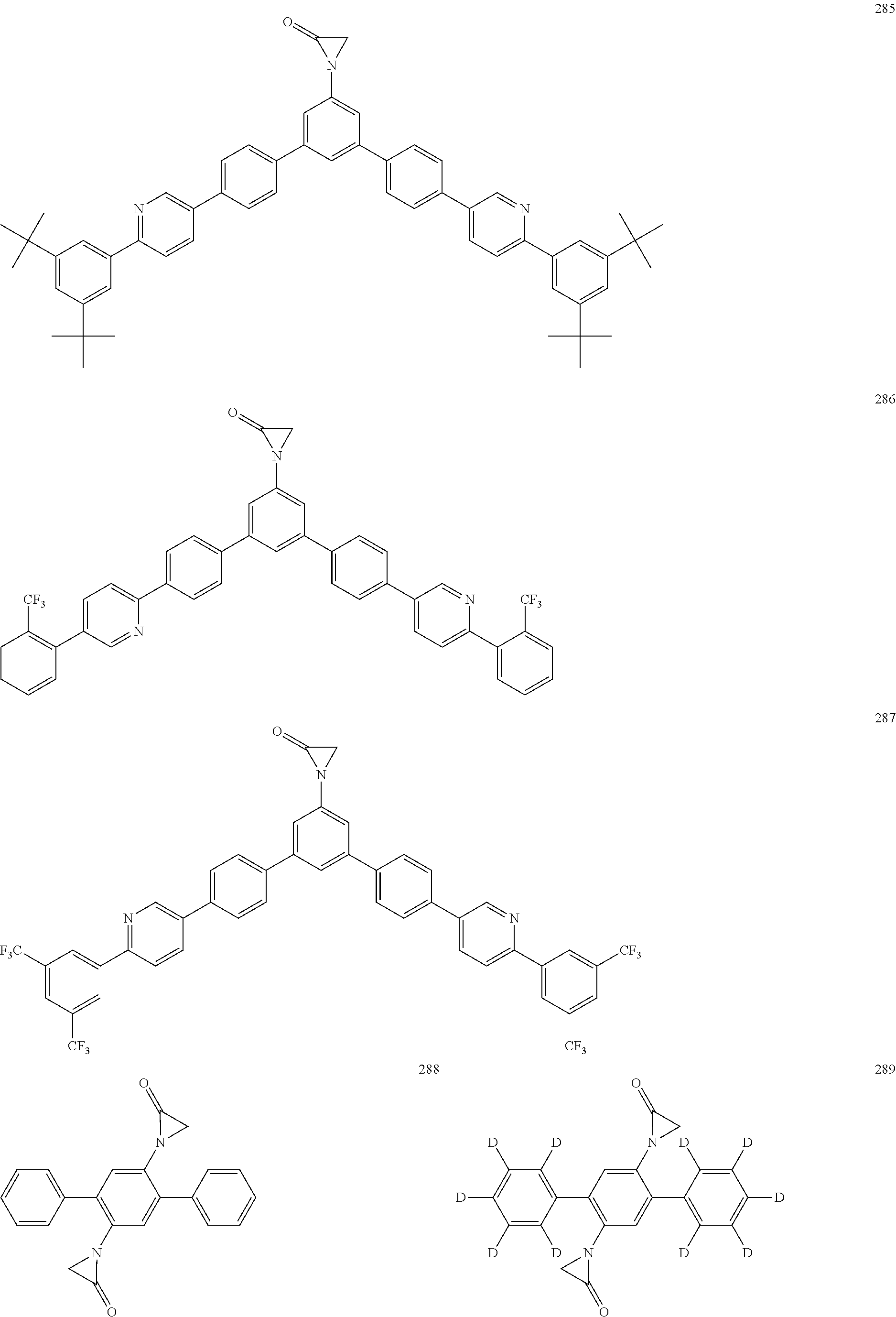

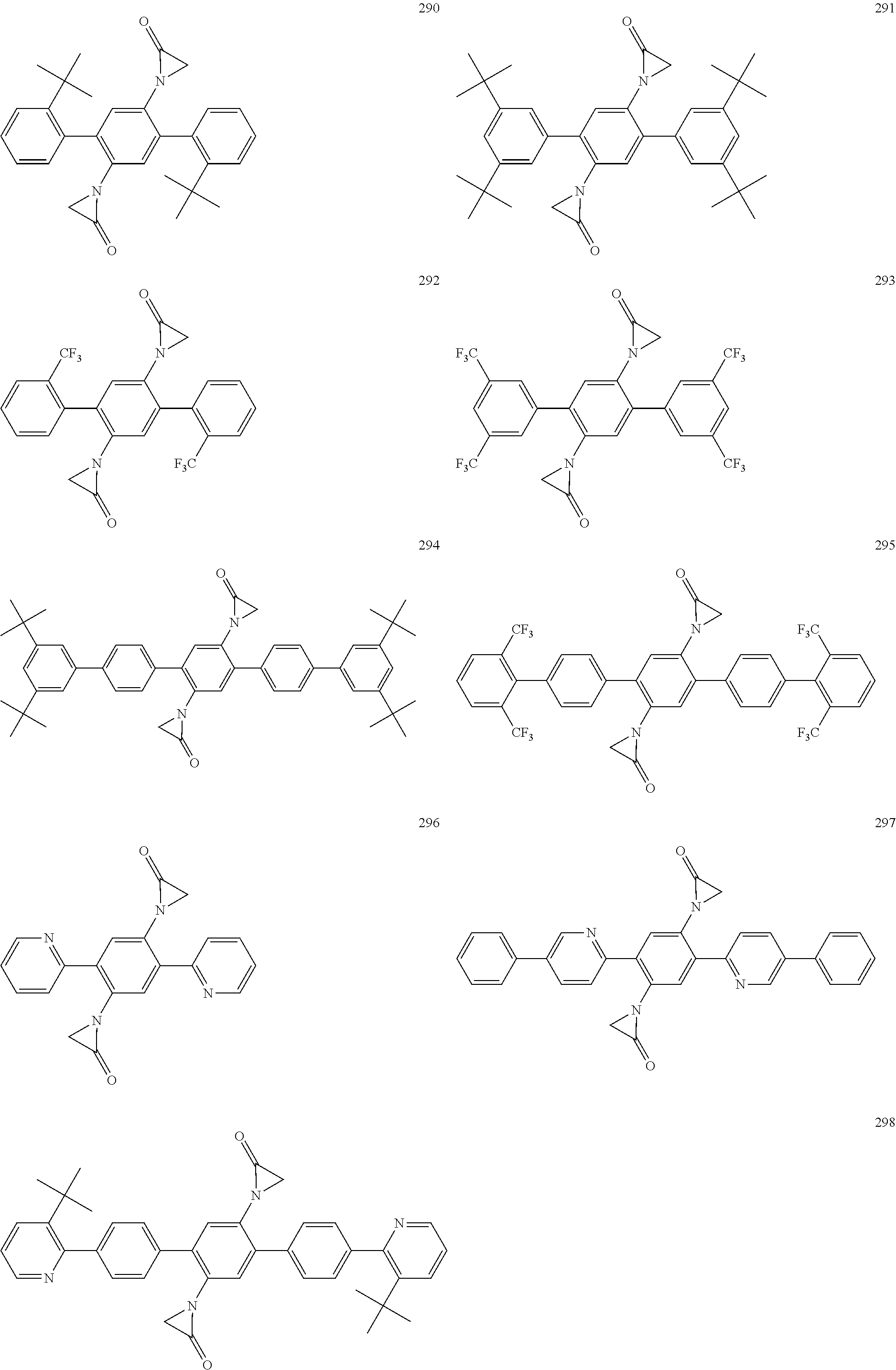

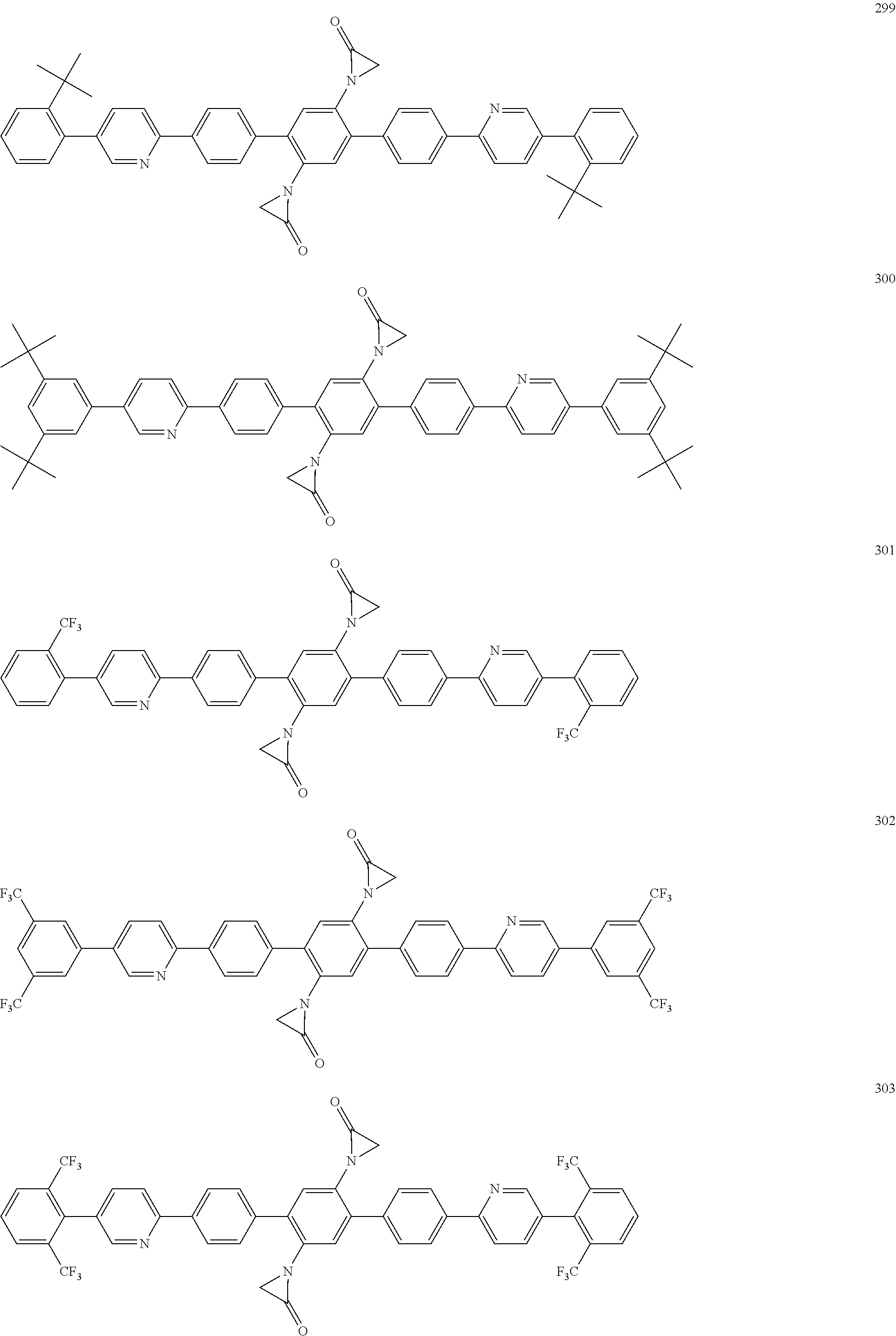

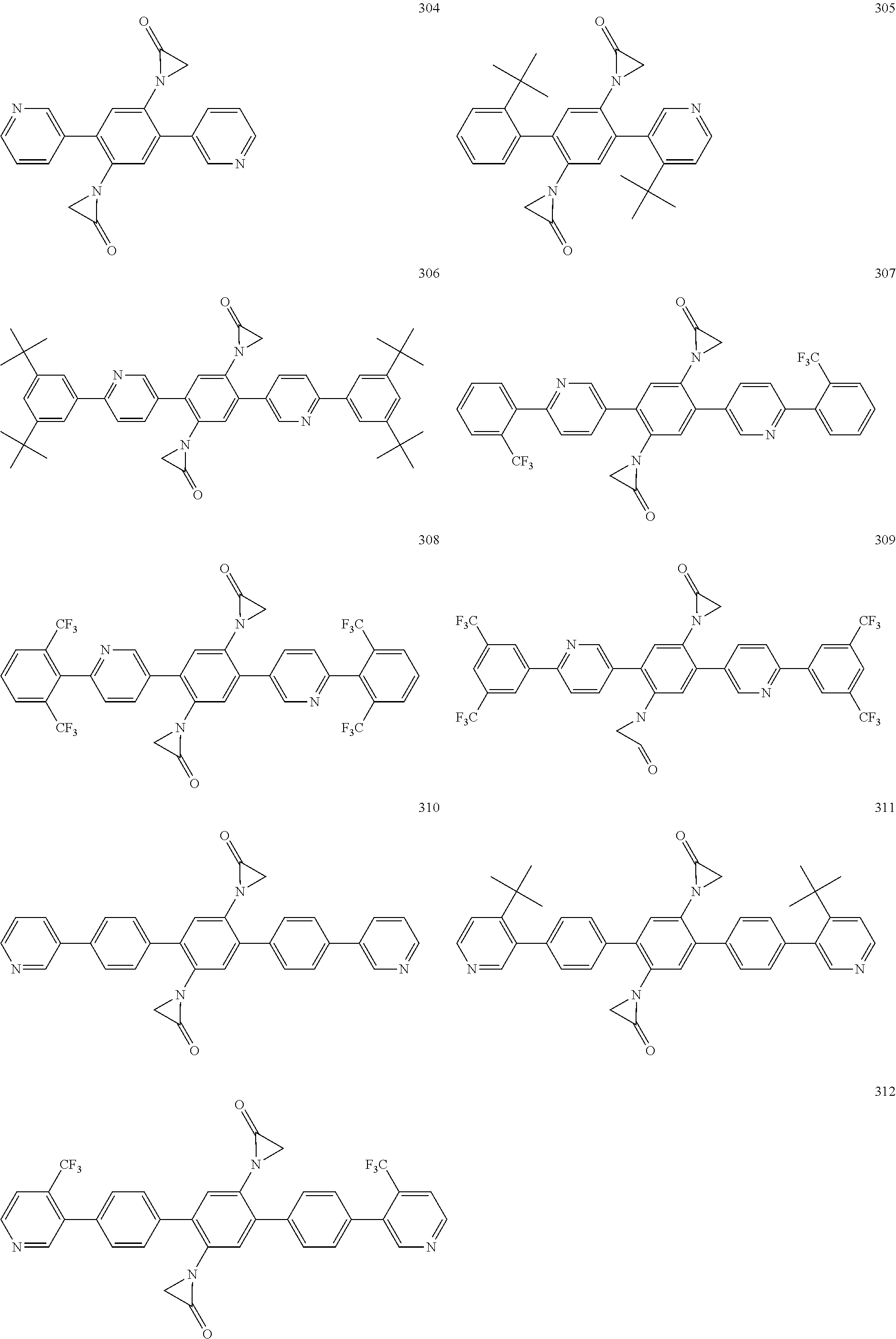

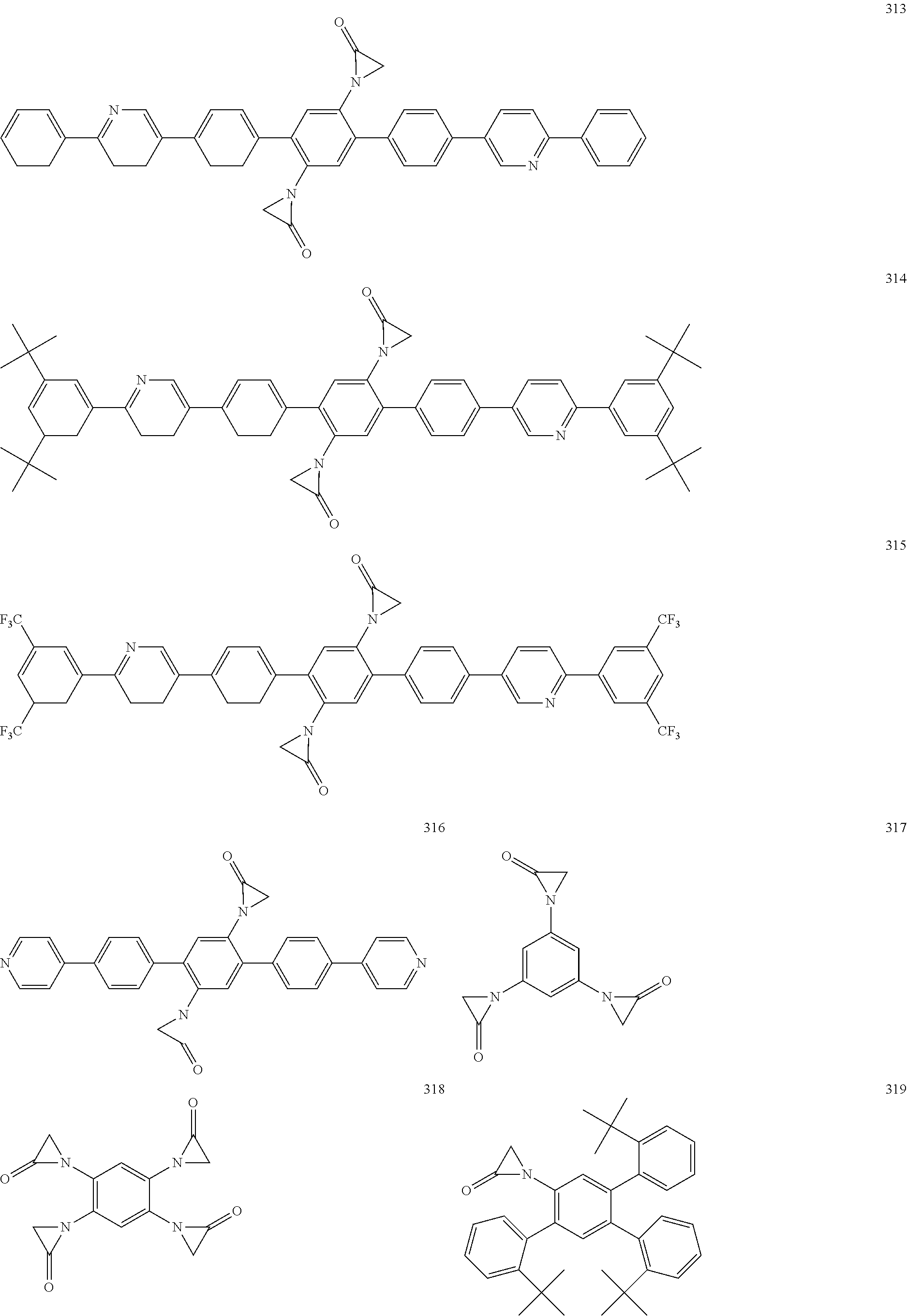

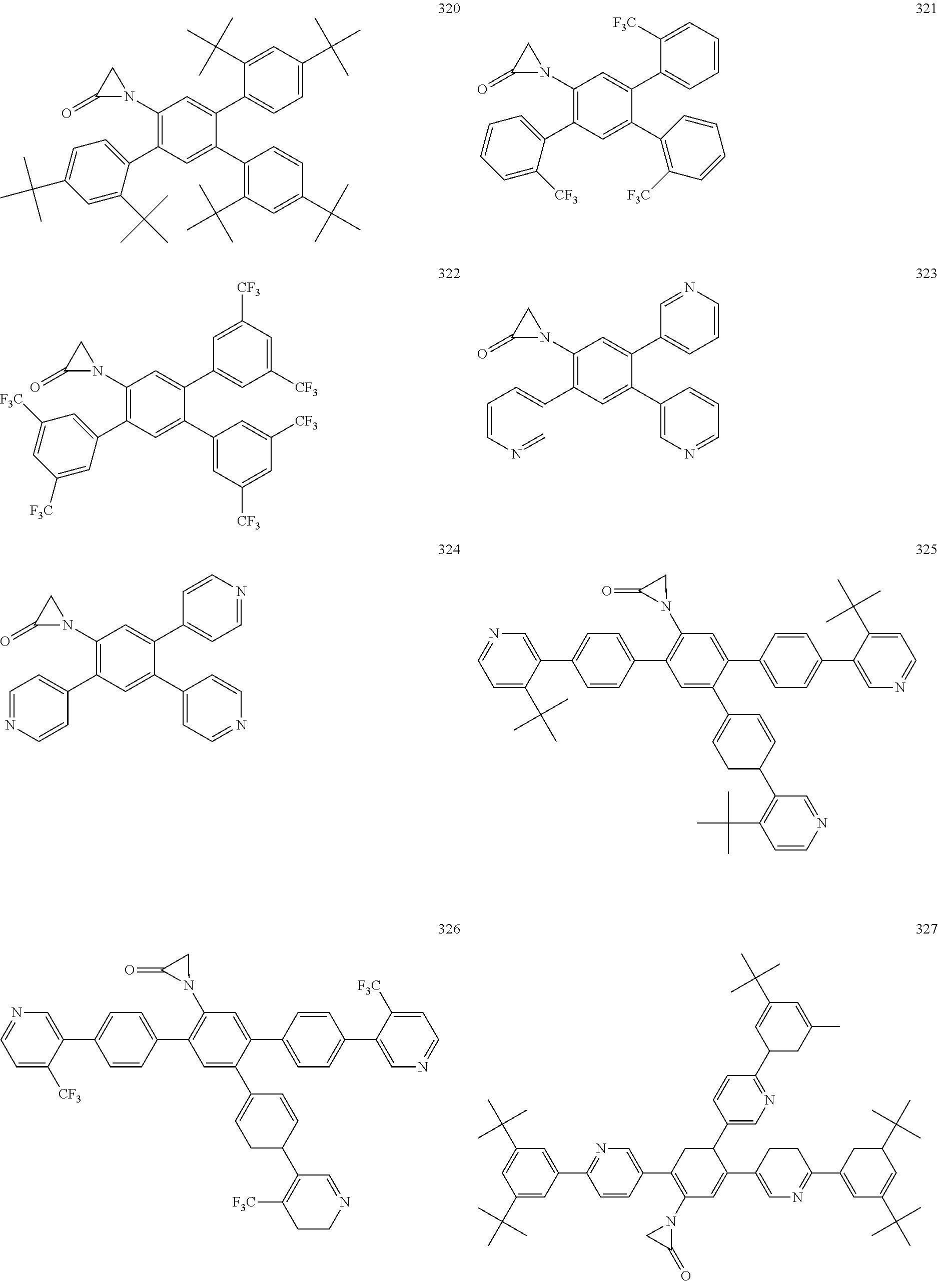

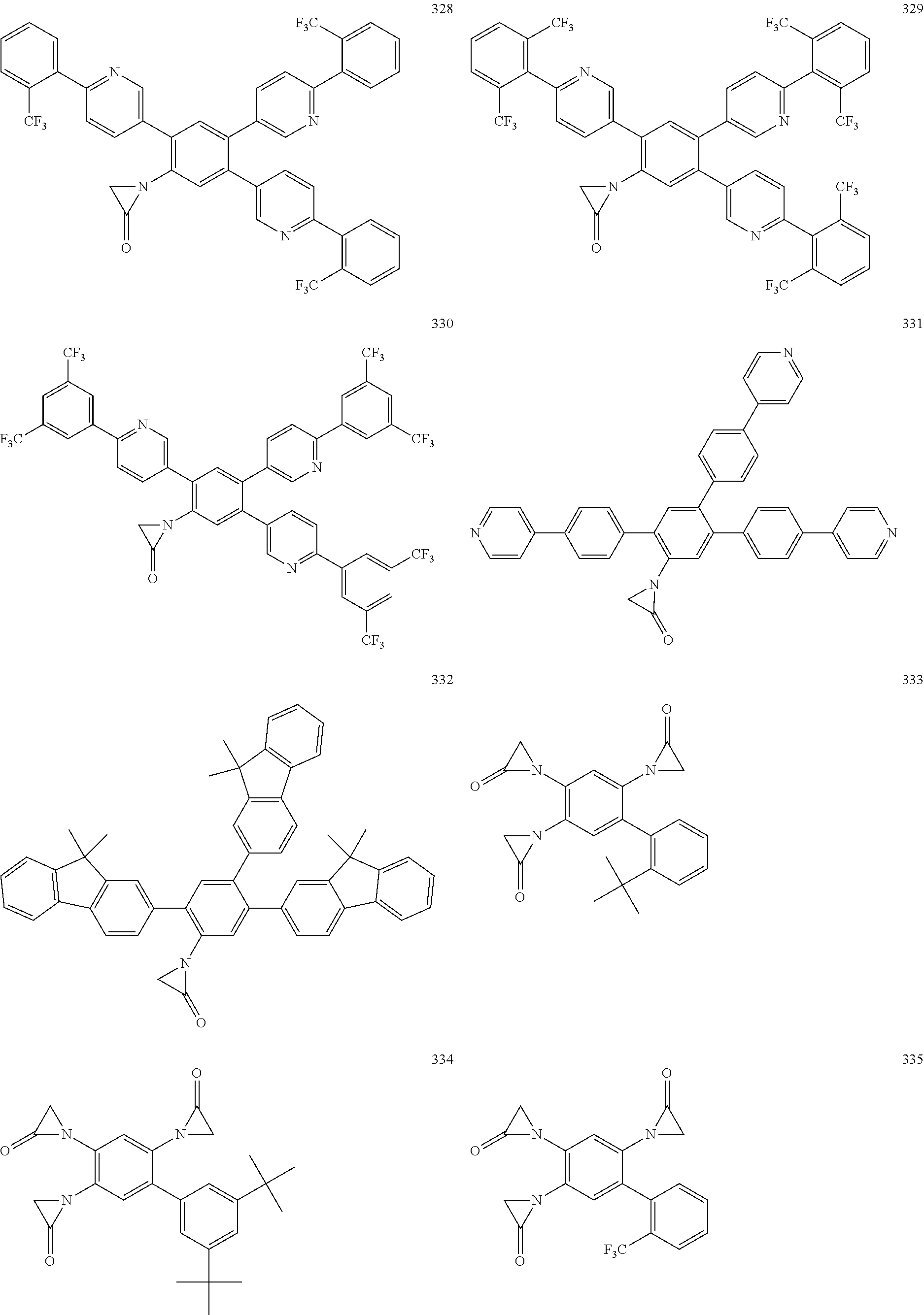

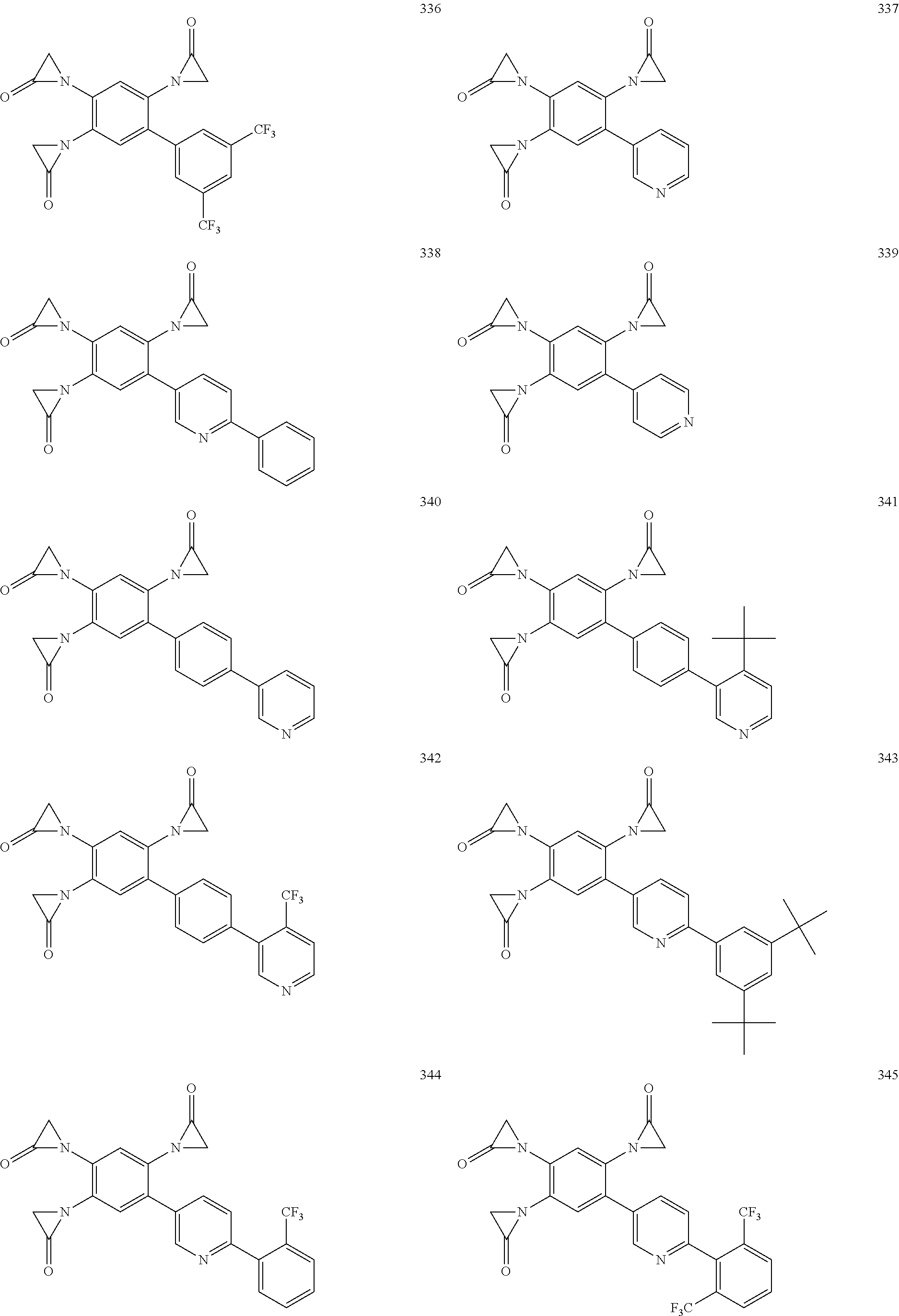

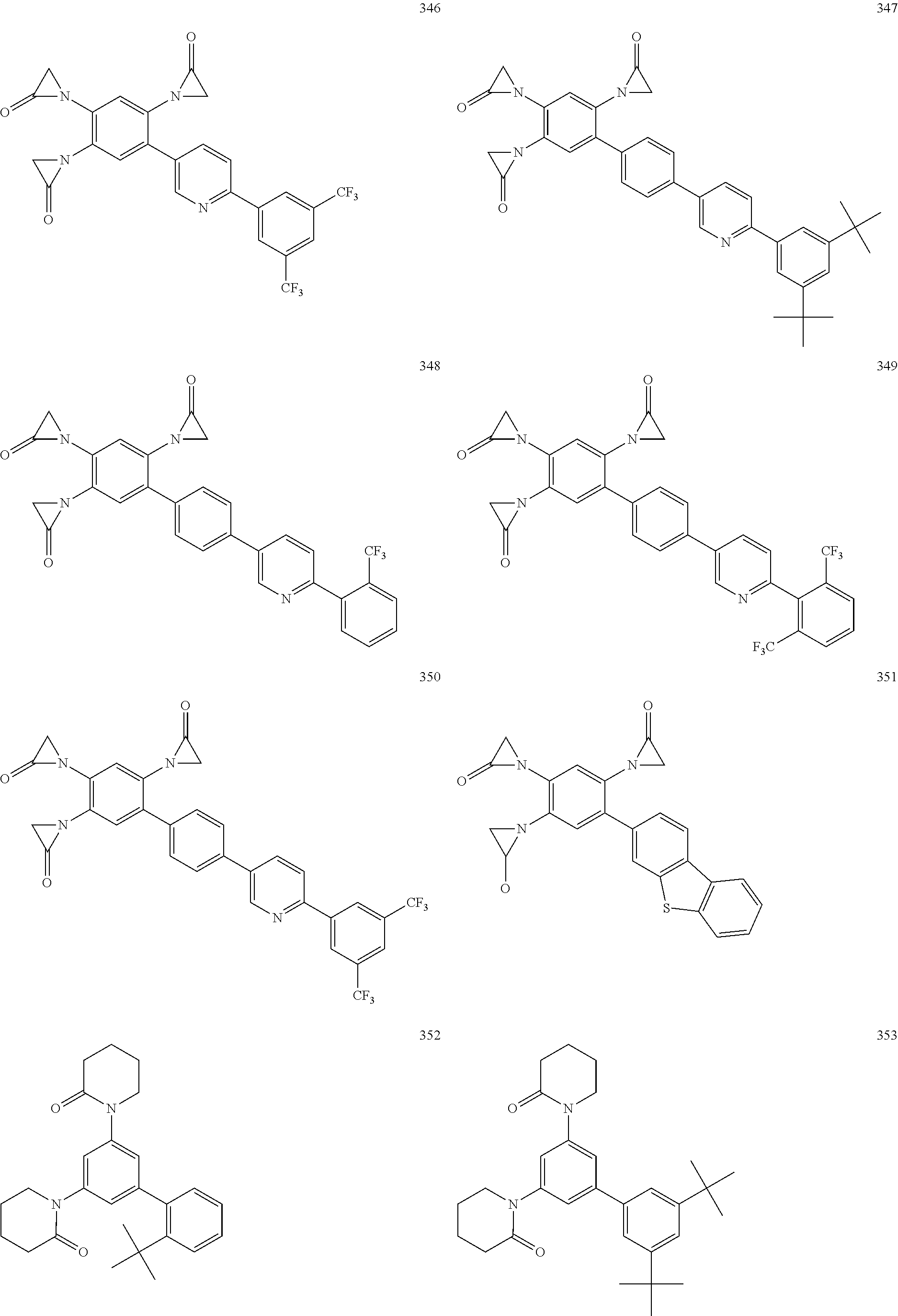

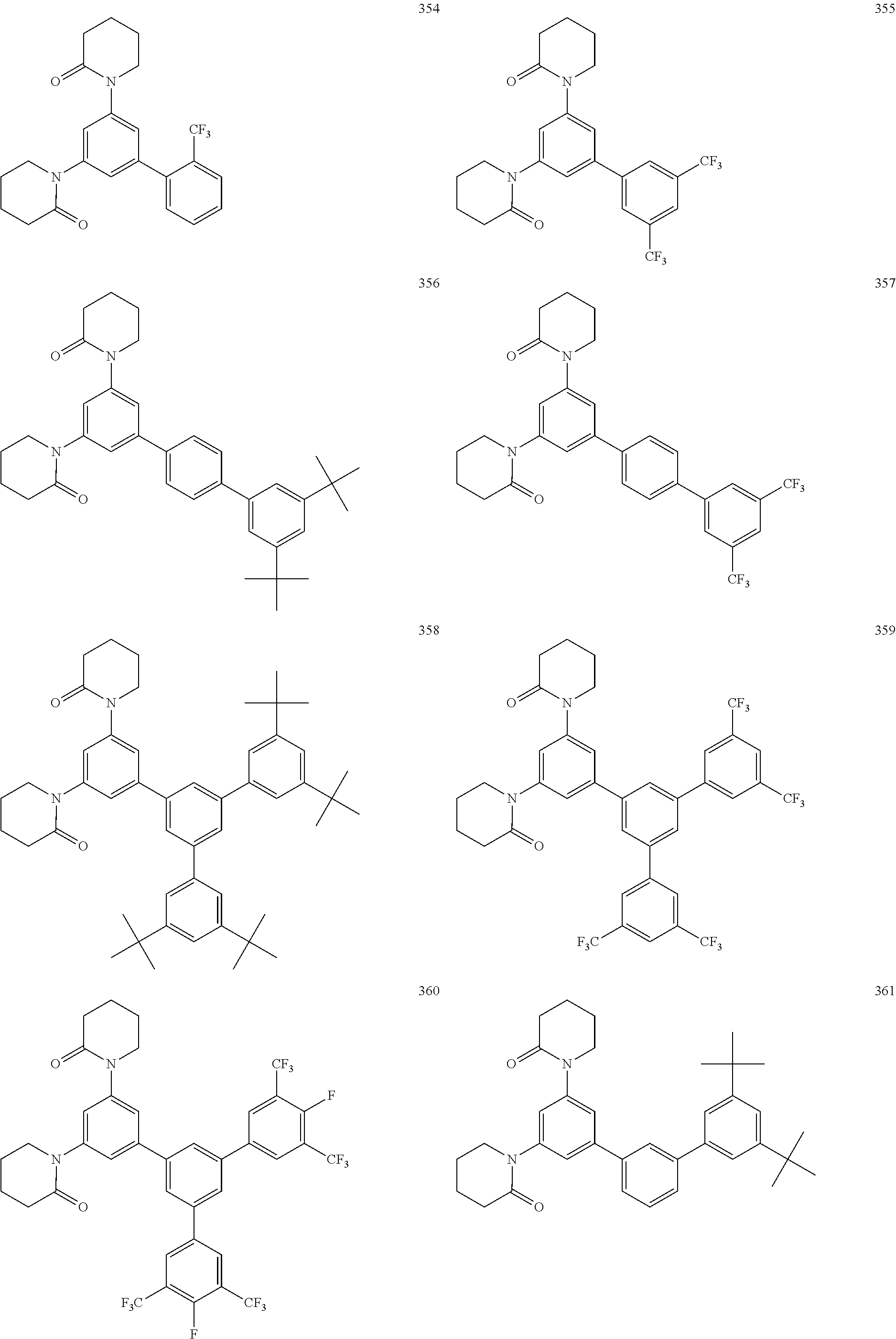

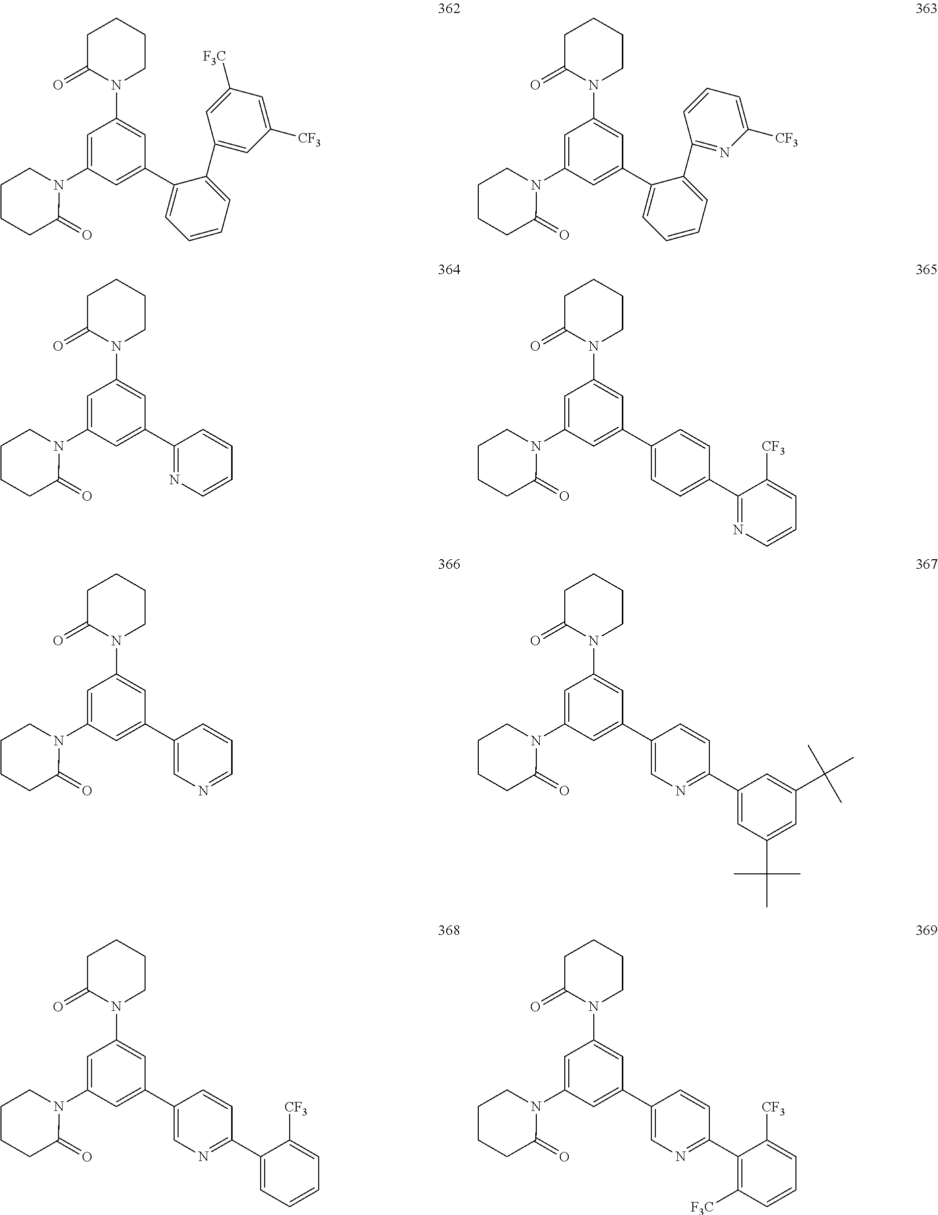

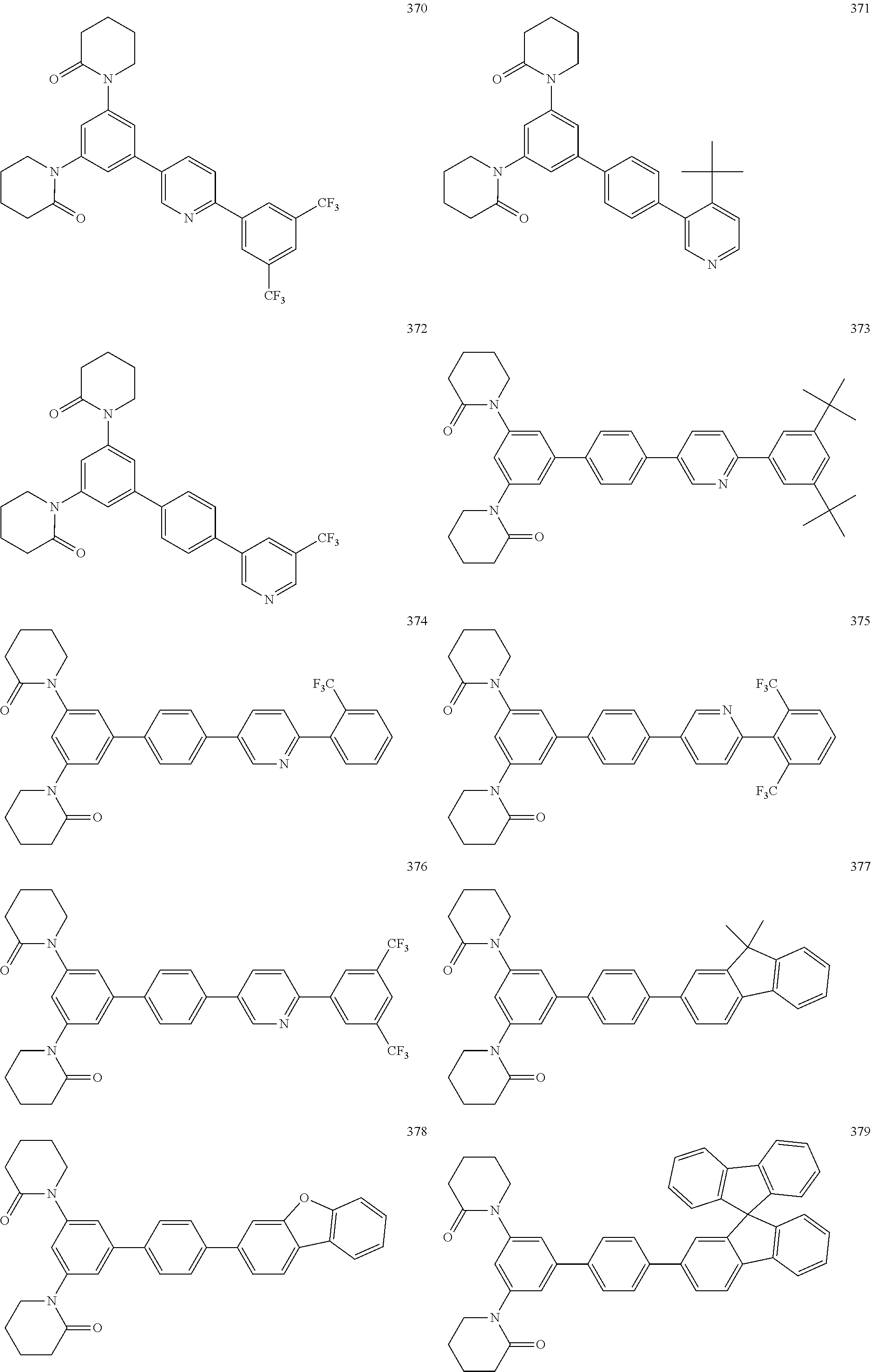

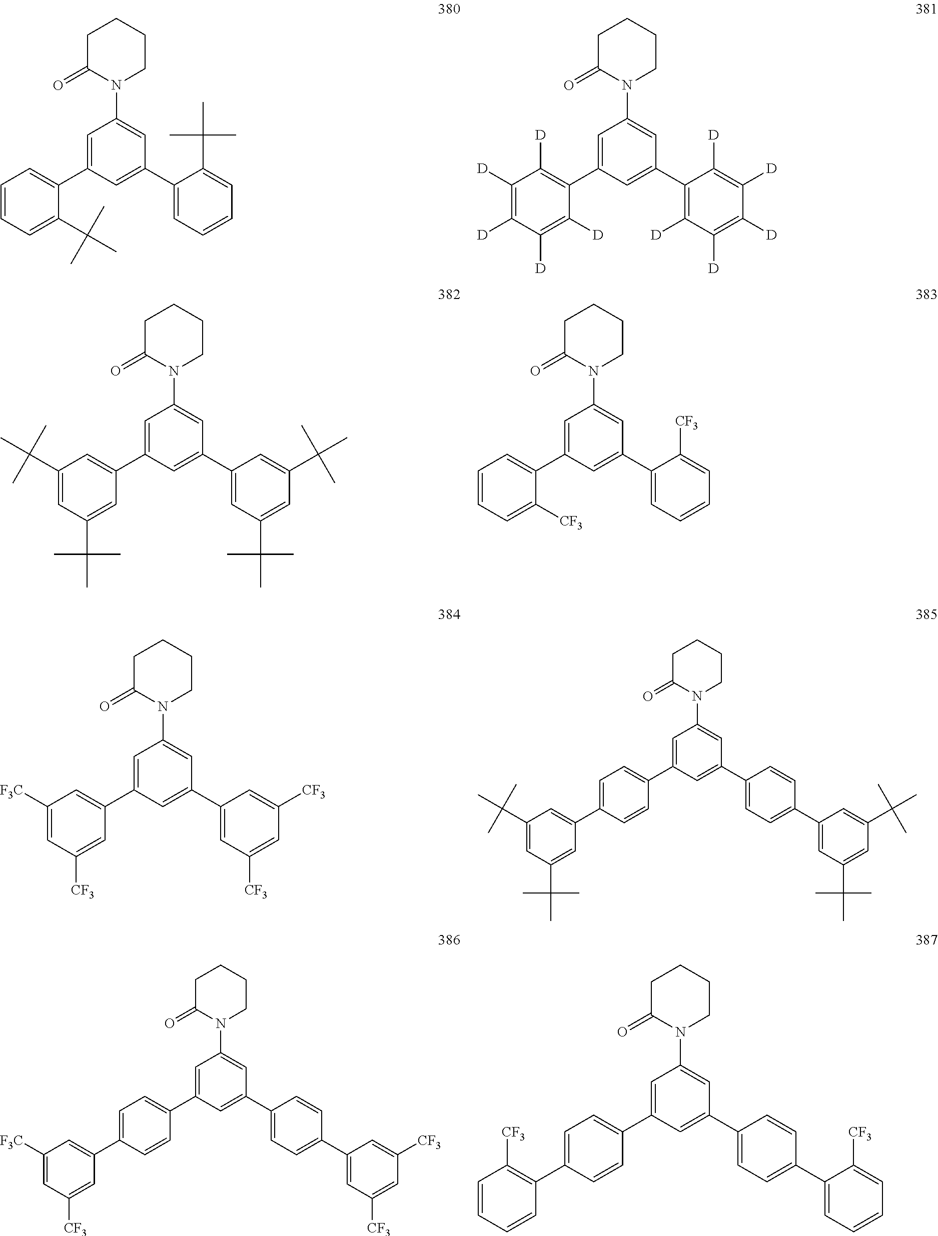

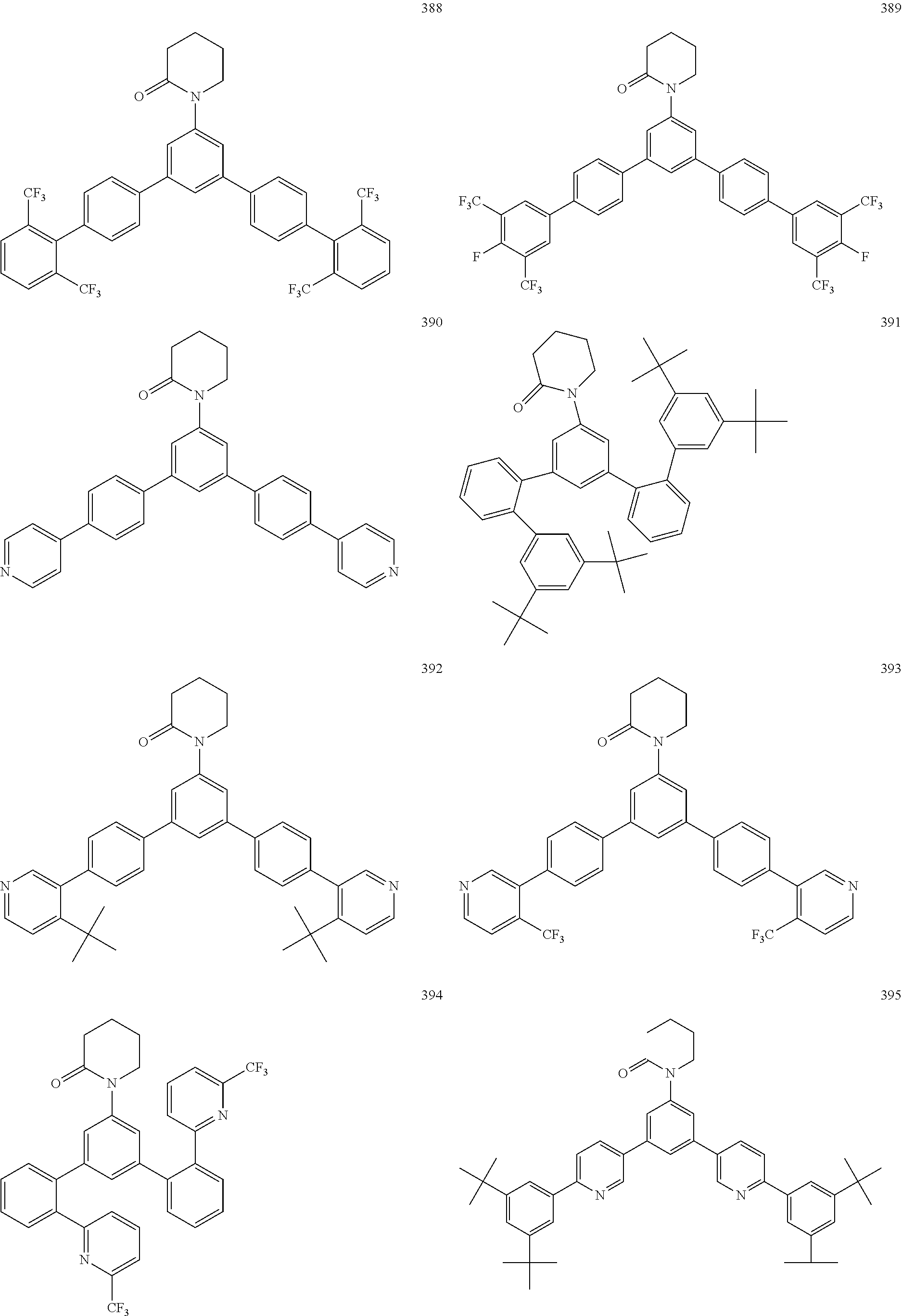

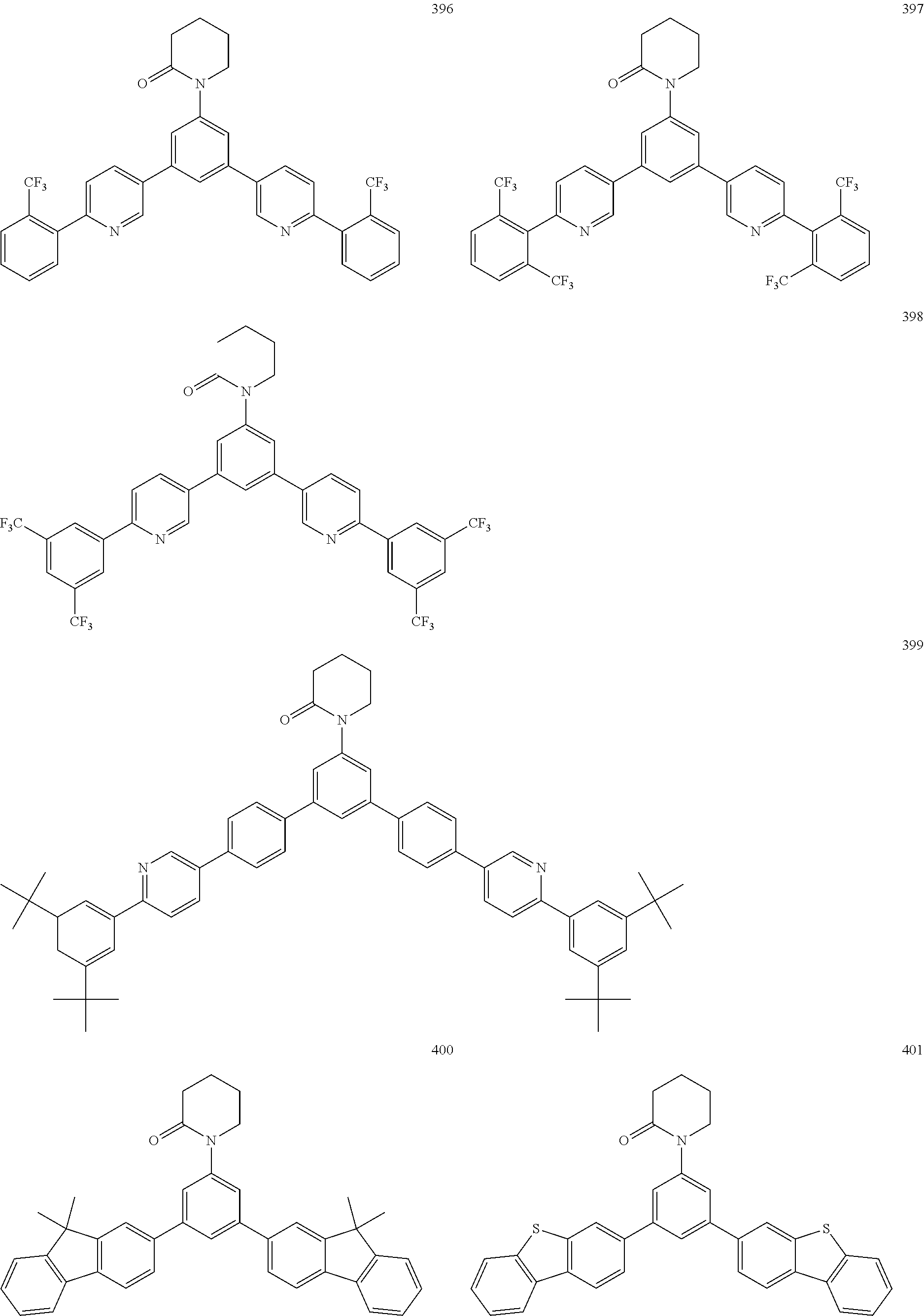

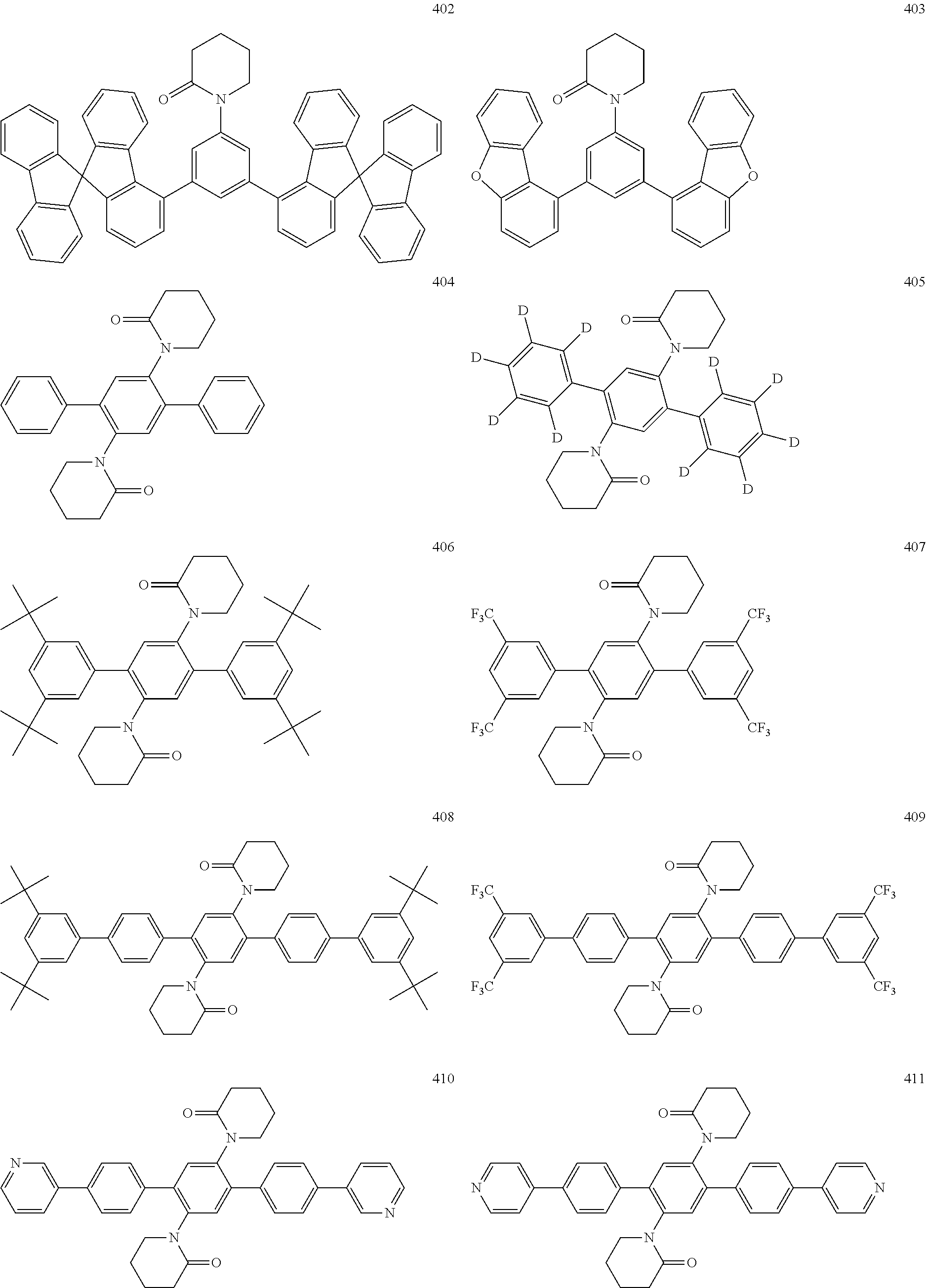

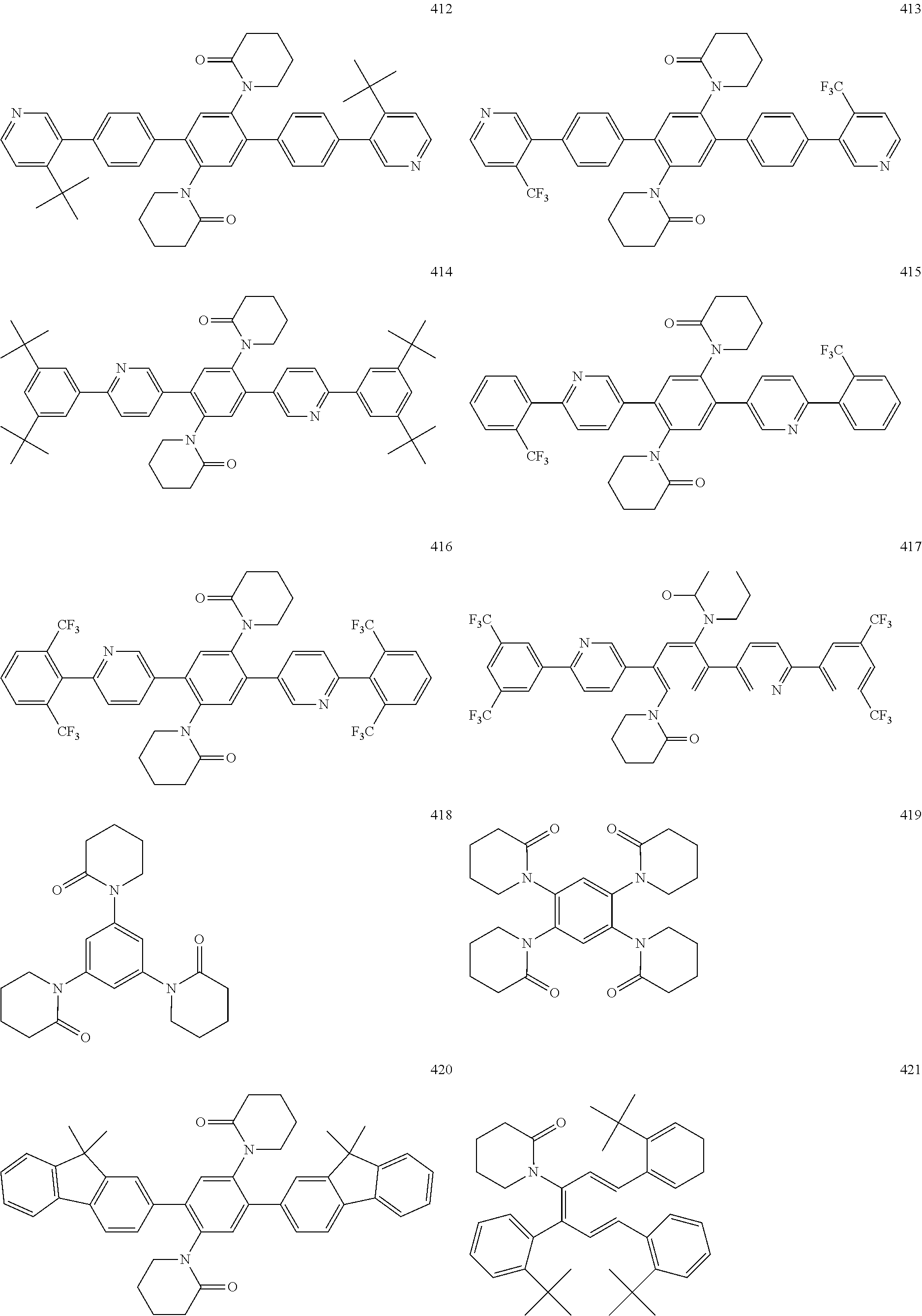

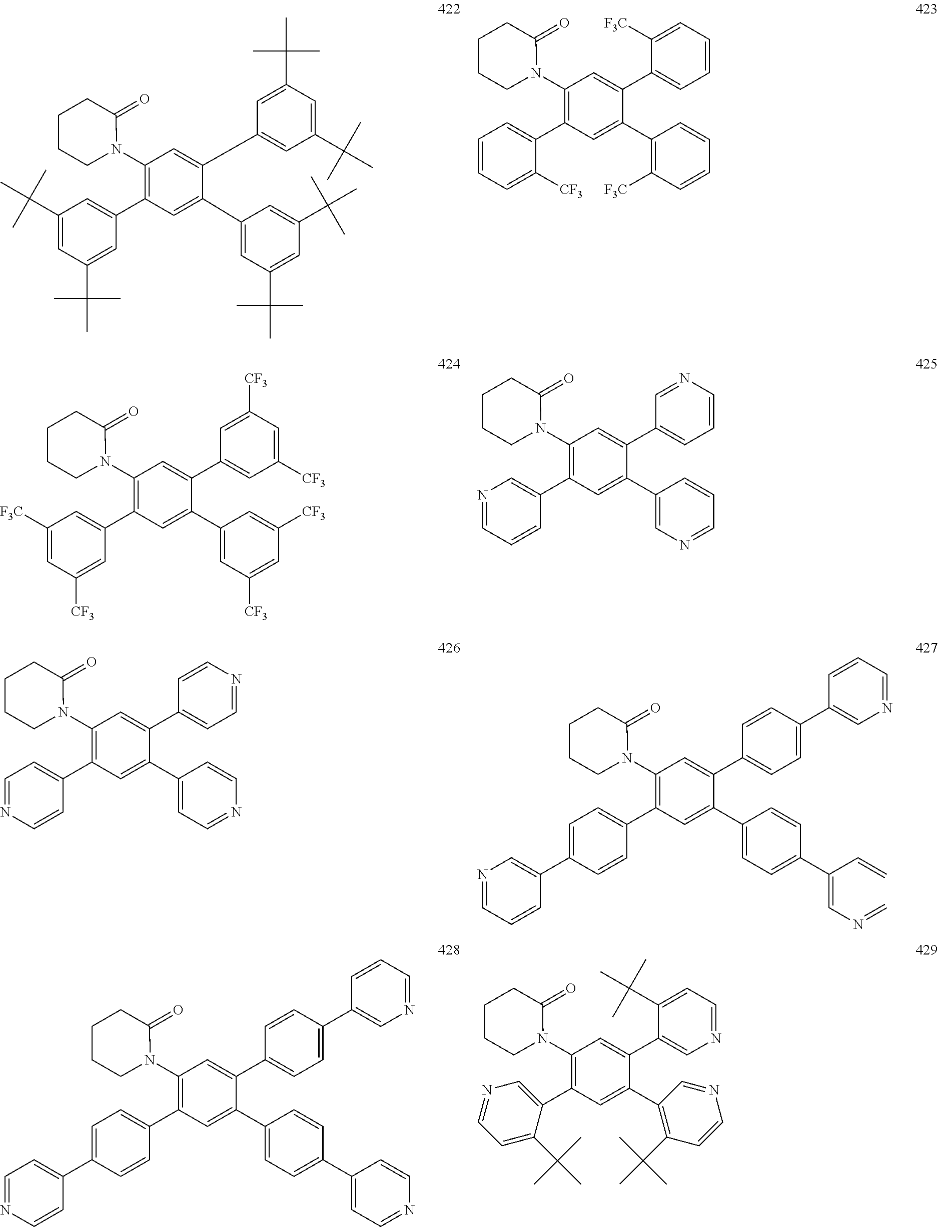

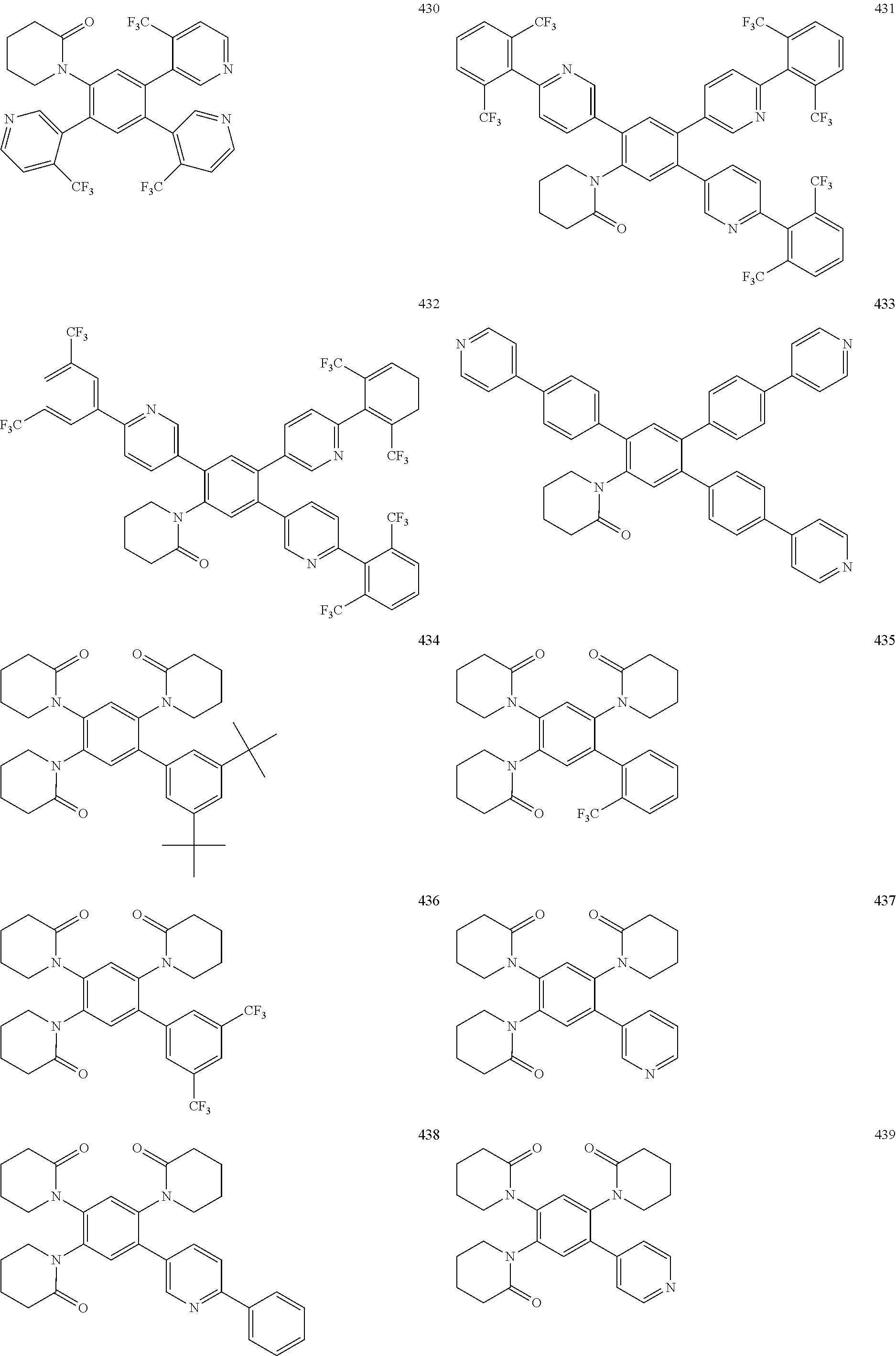

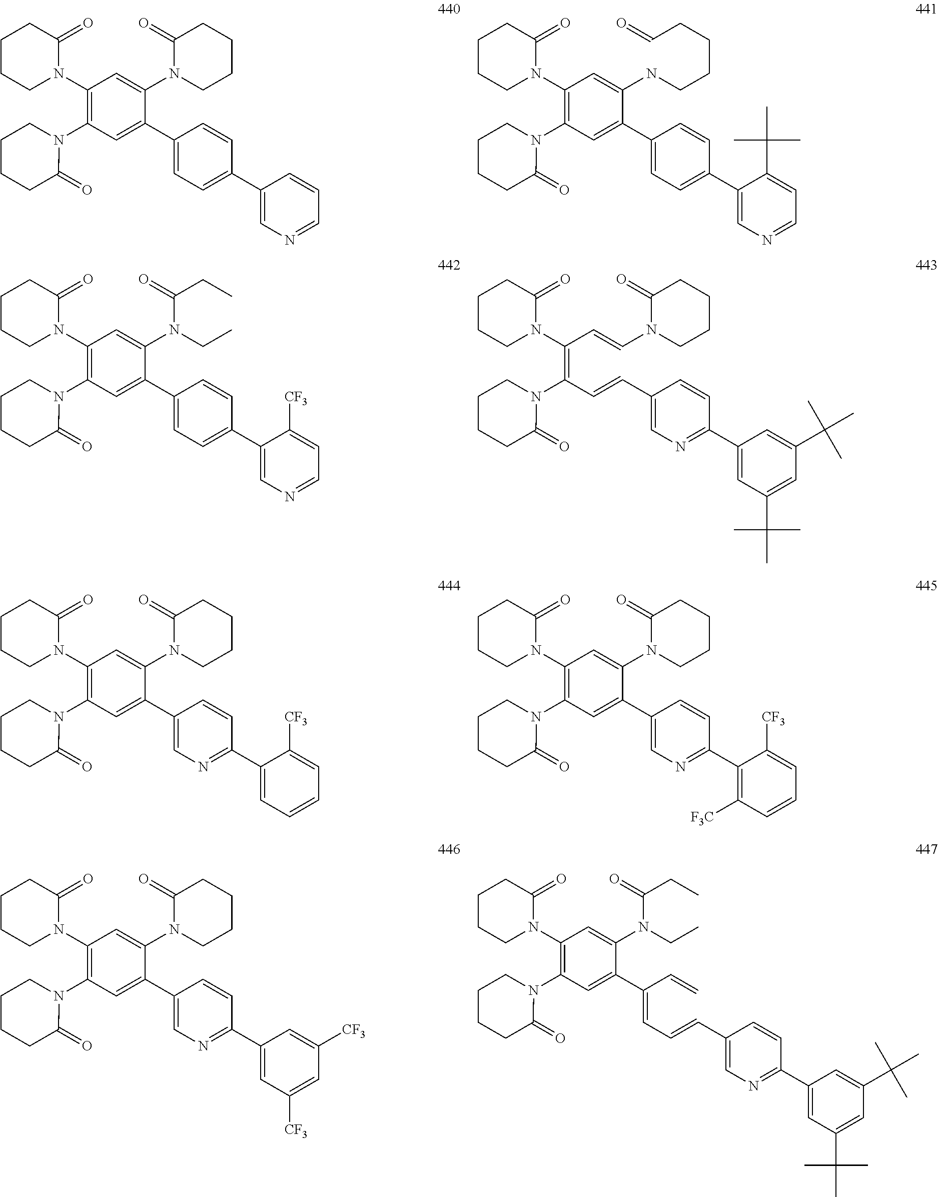

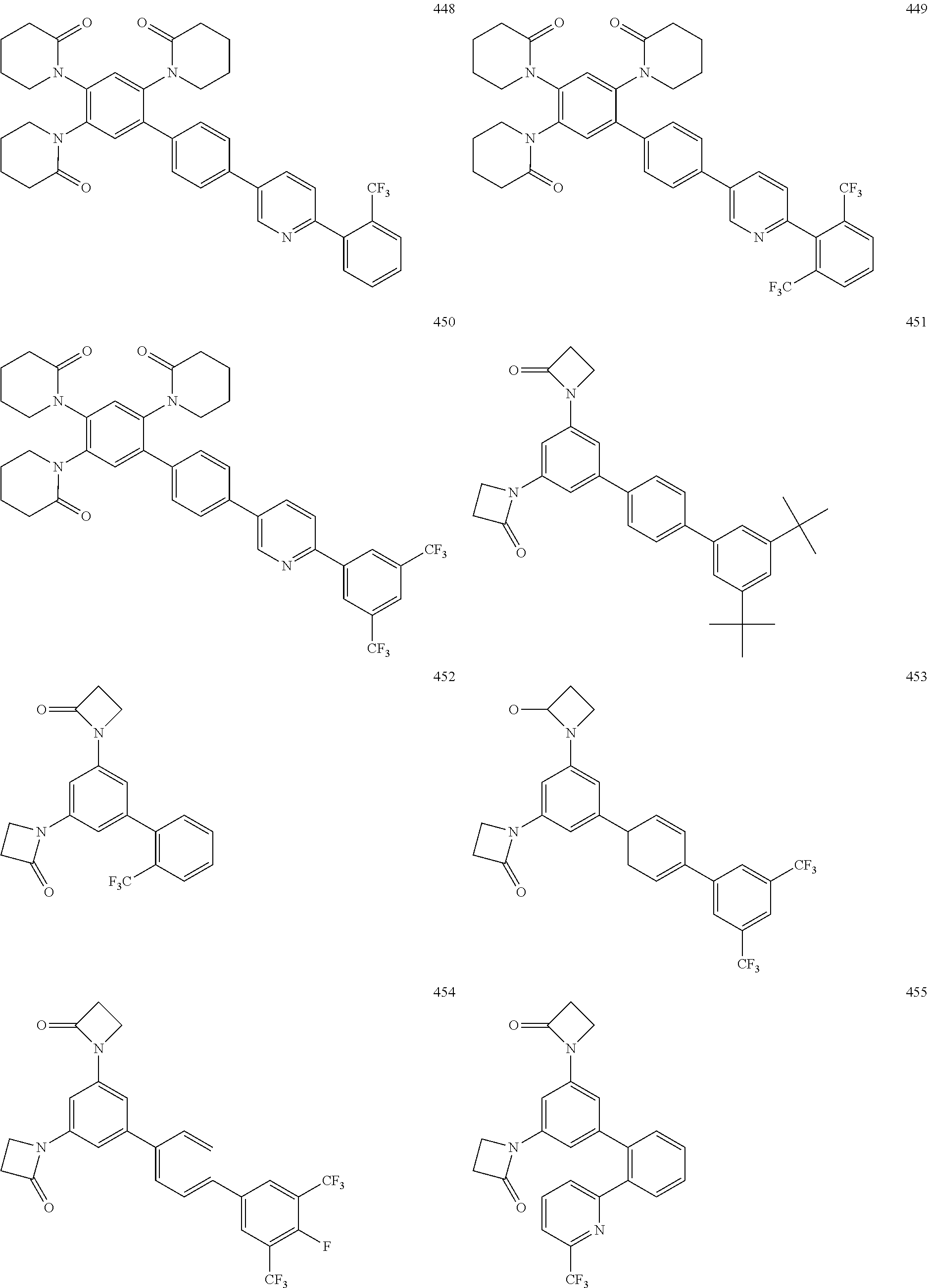

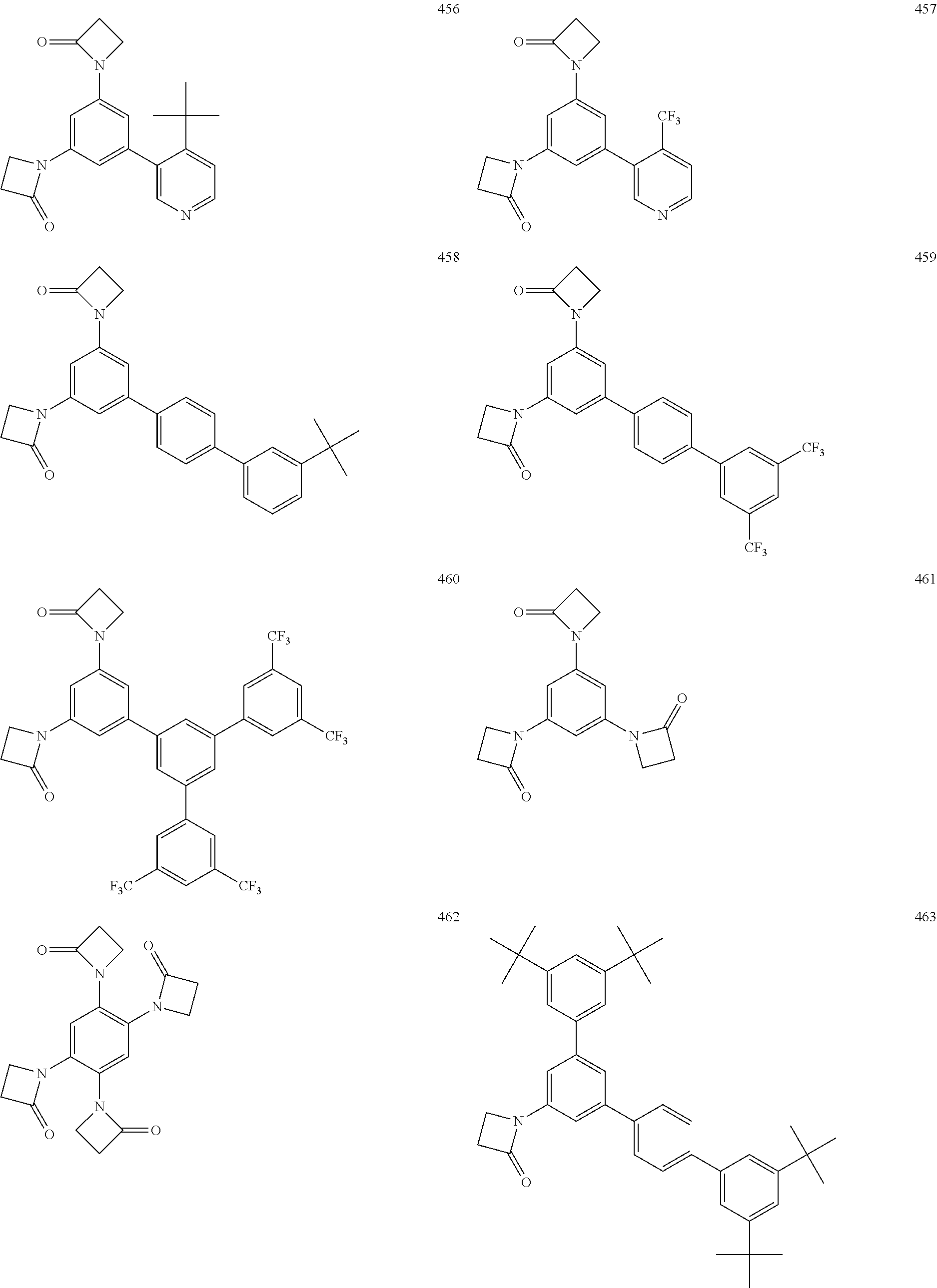

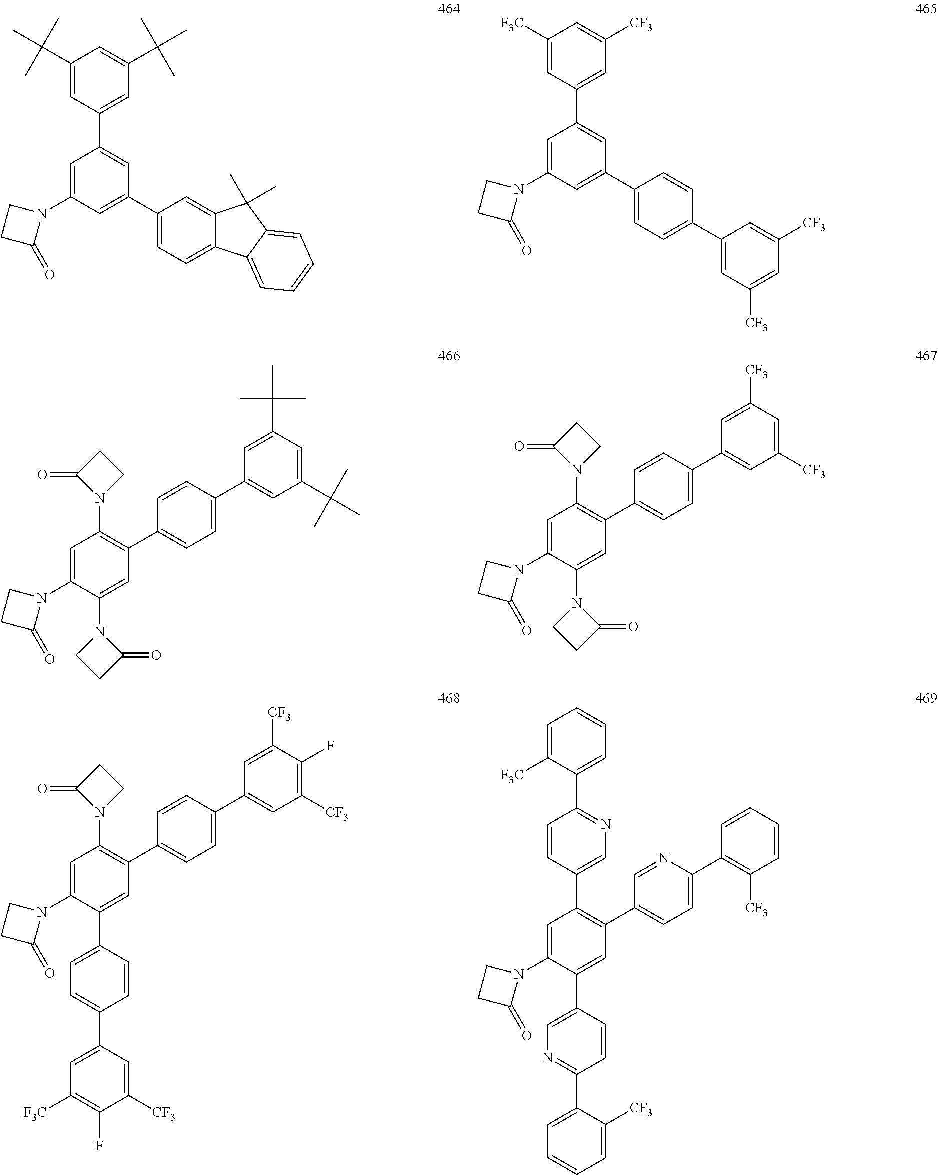

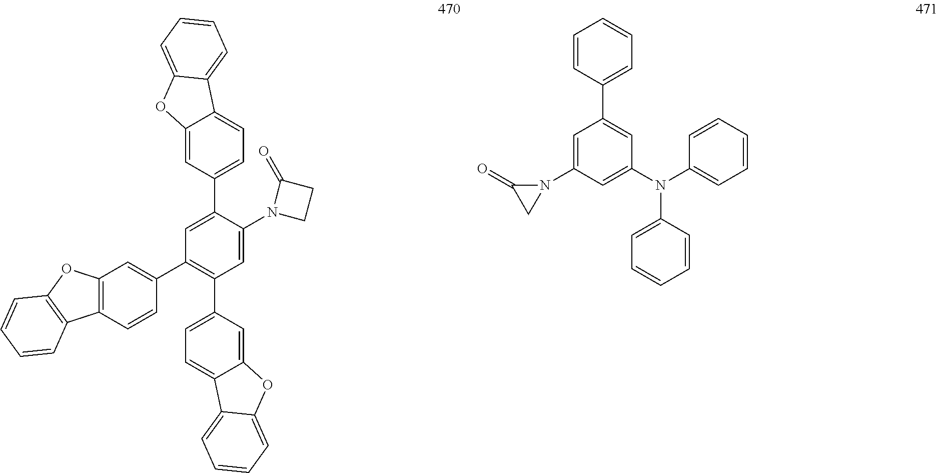 [SMILES: O=C1CCN1c1cc(-c2ccc3c(c2)oc2ccccc23)c(-c2ccc3c(c2)oc2ccccc23)cc1-c1ccc2c(c1)oc1ccccc12 ; O=C1CN1c1cc(-c2ccccc2)cc(N(c2ccccc2)c2ccccc2)c1]

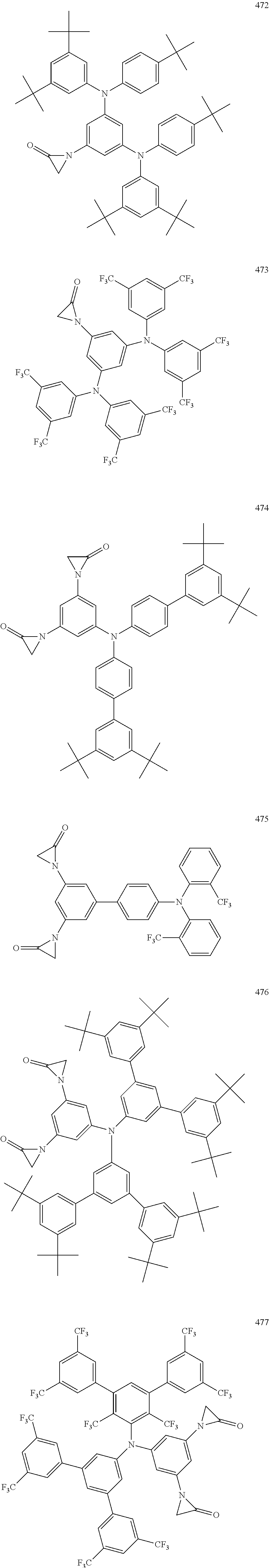

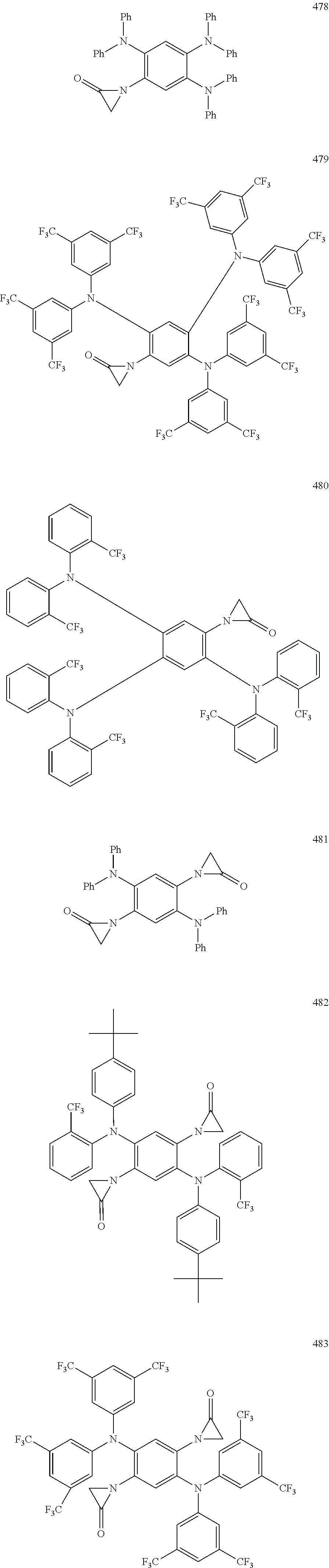

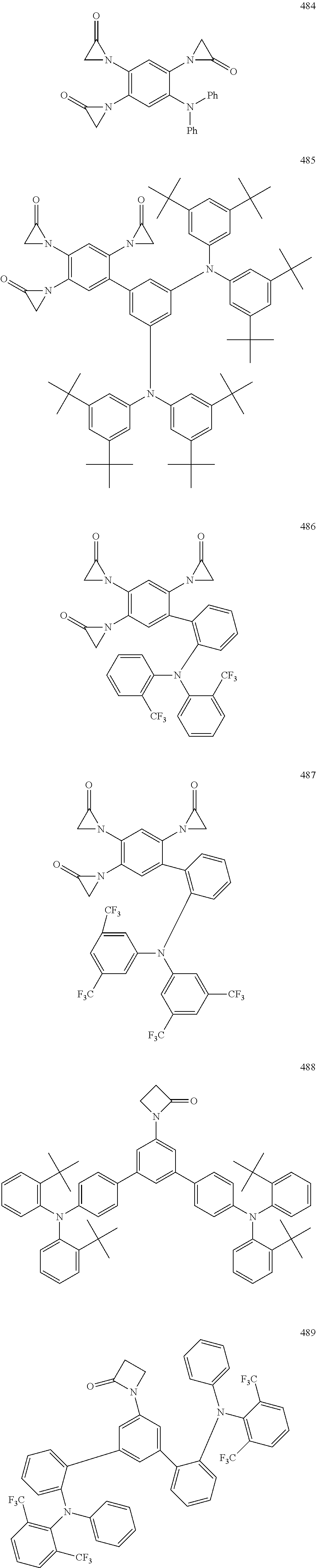

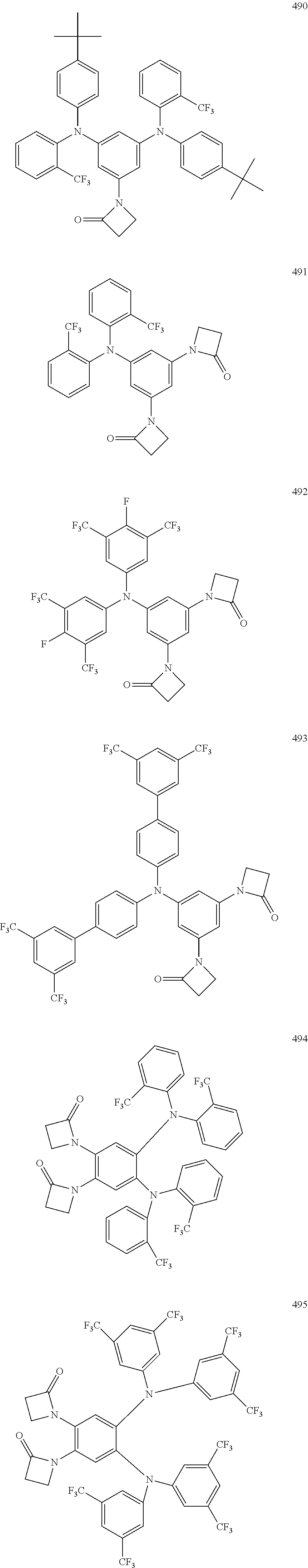

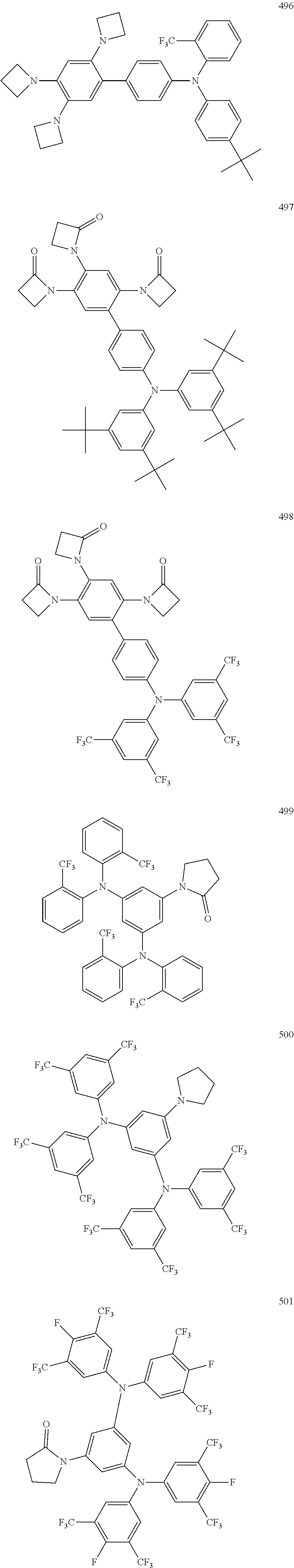

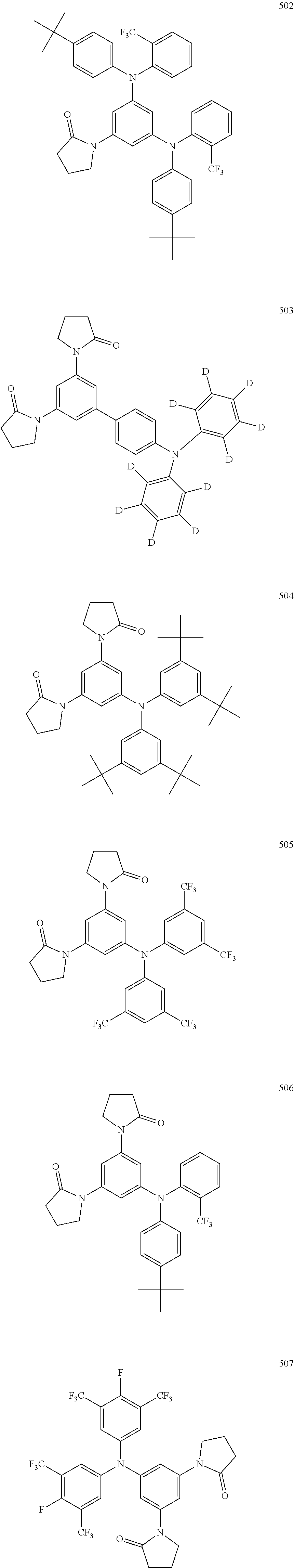

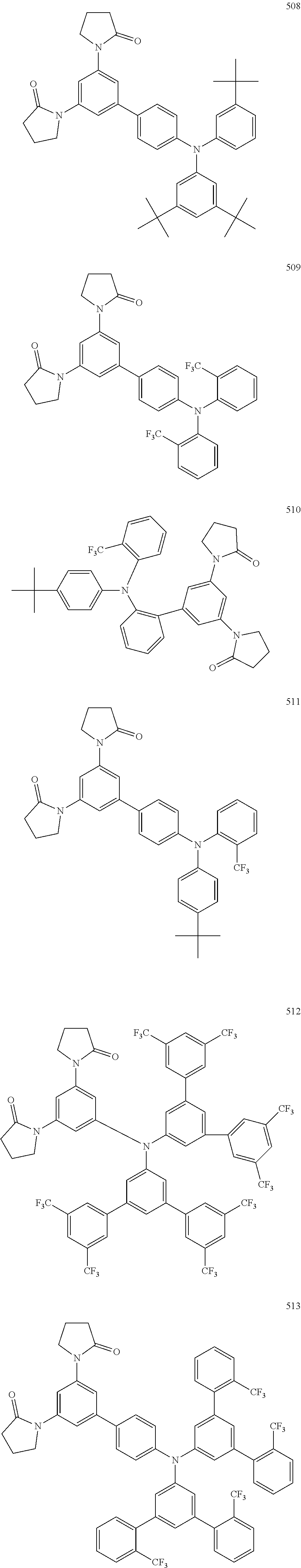

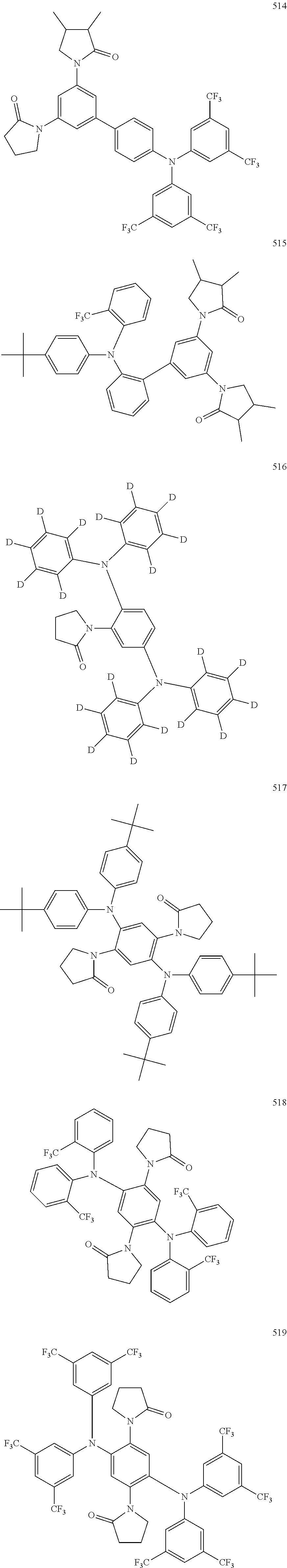

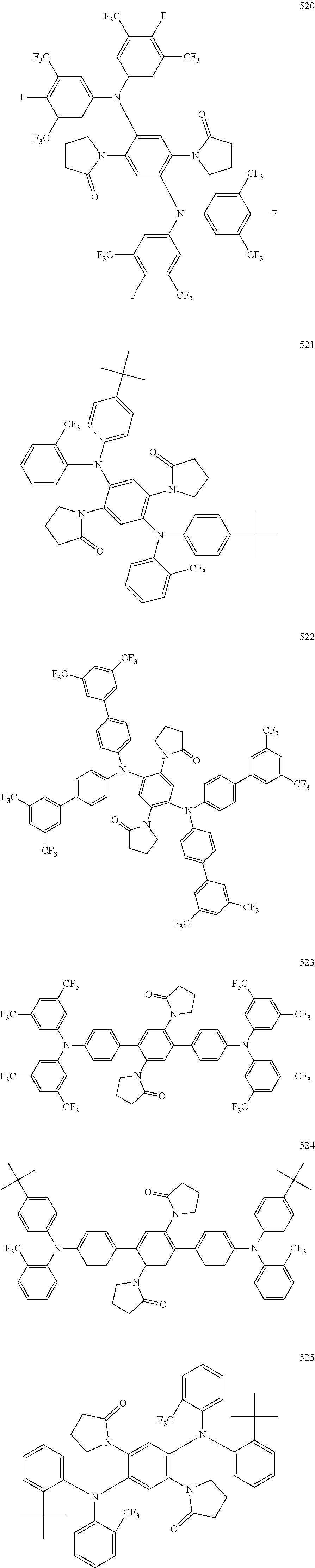

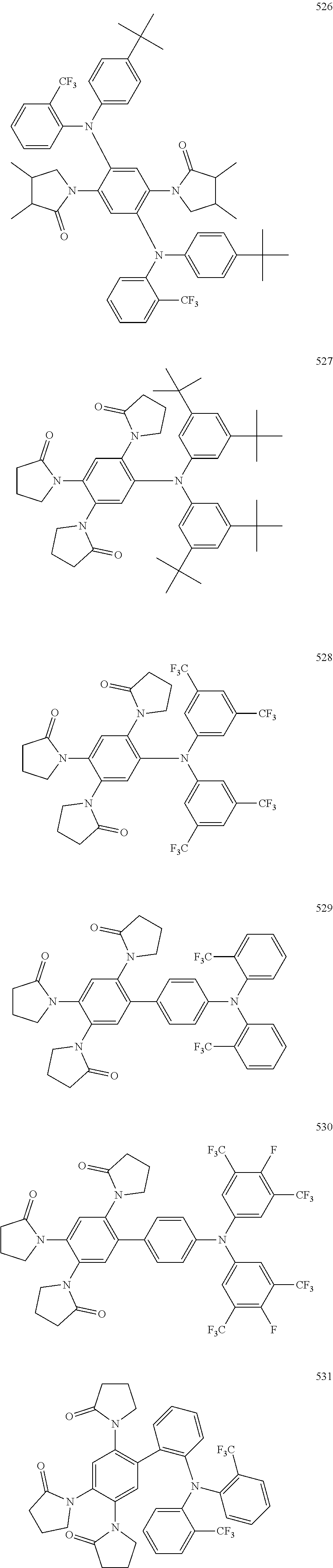

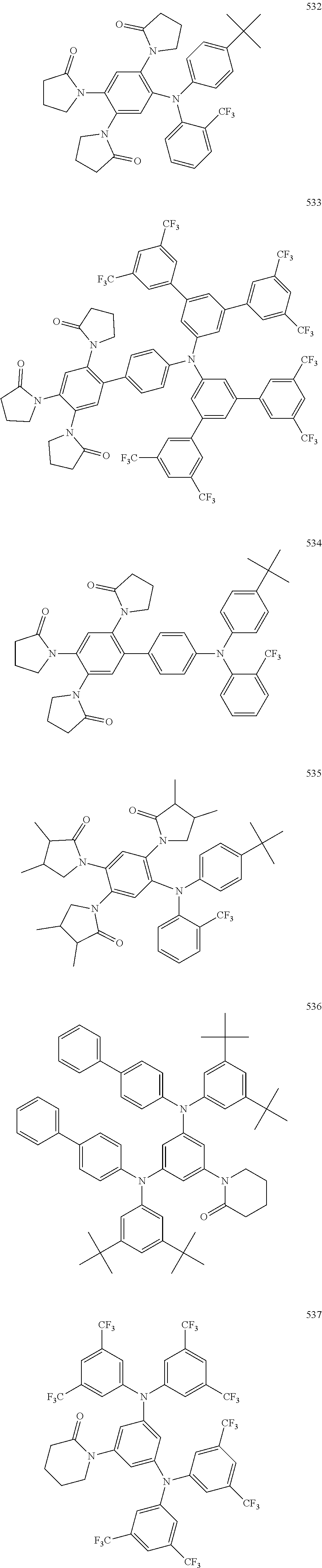

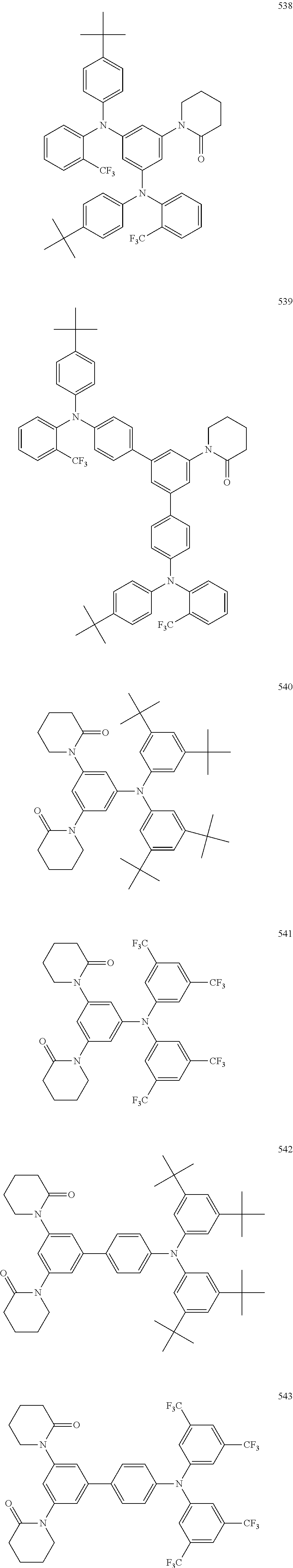

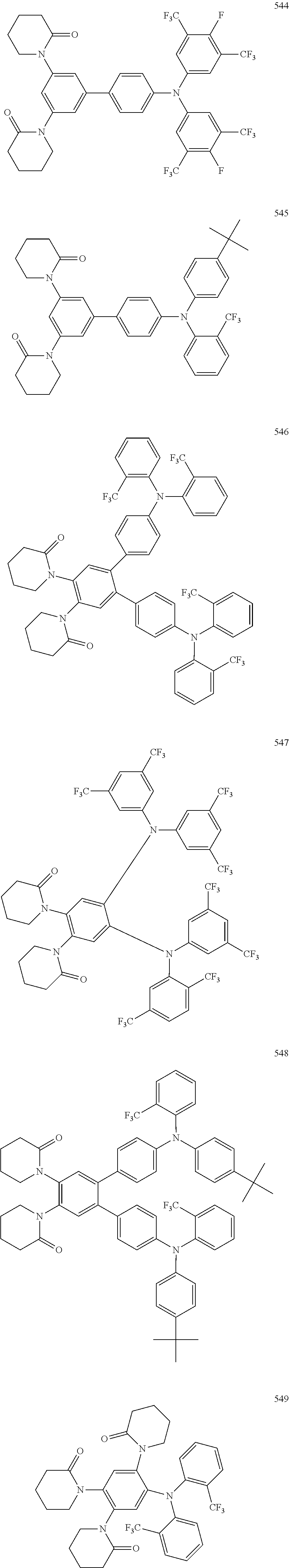

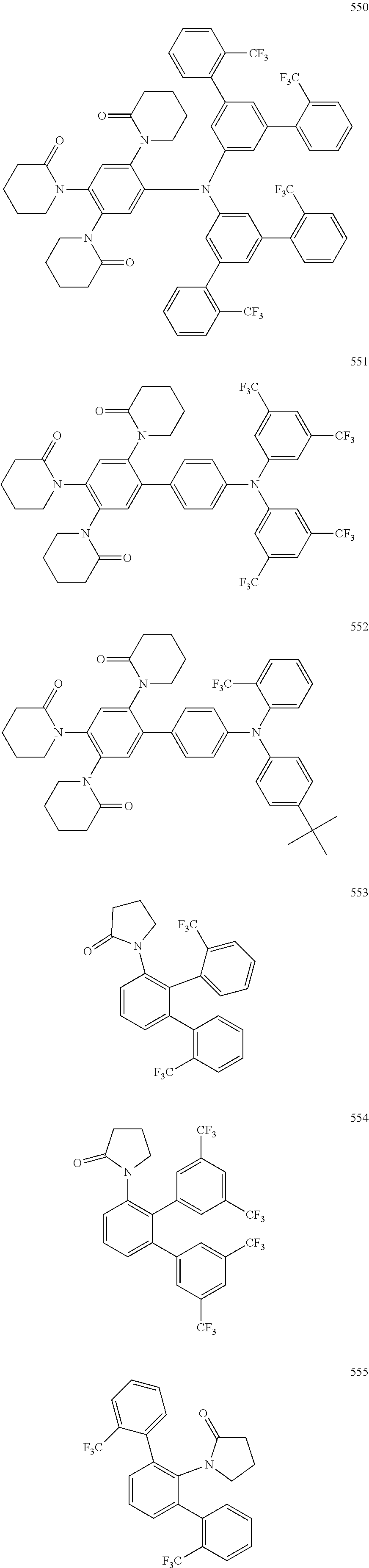

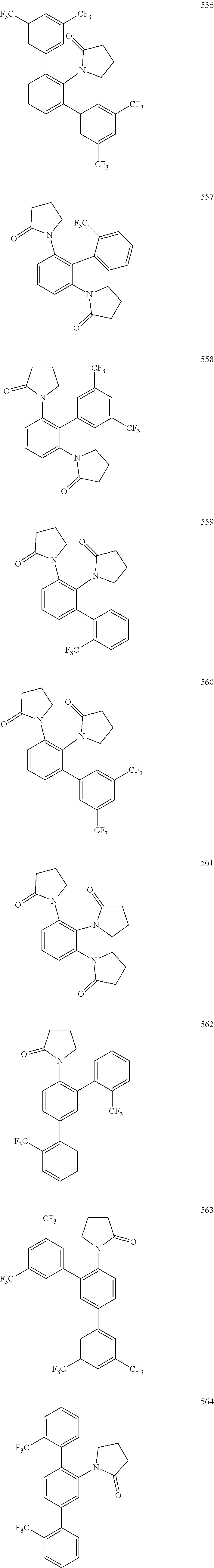

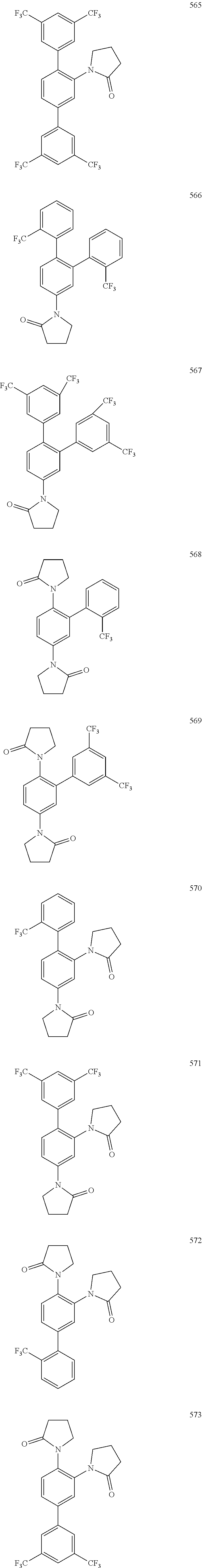

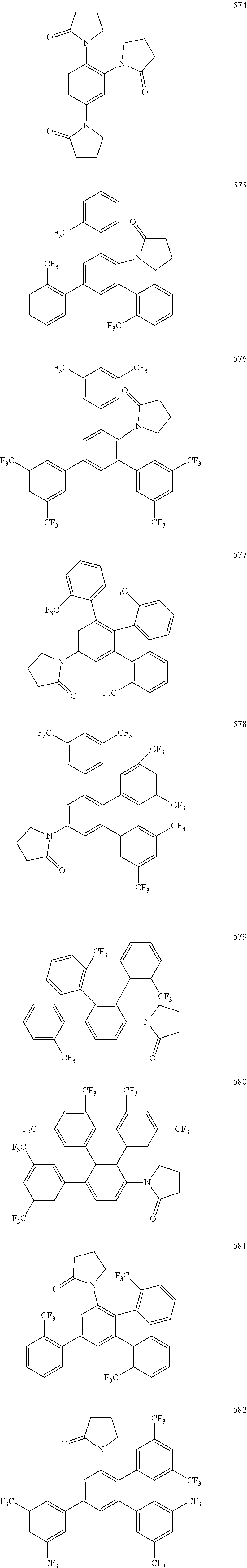

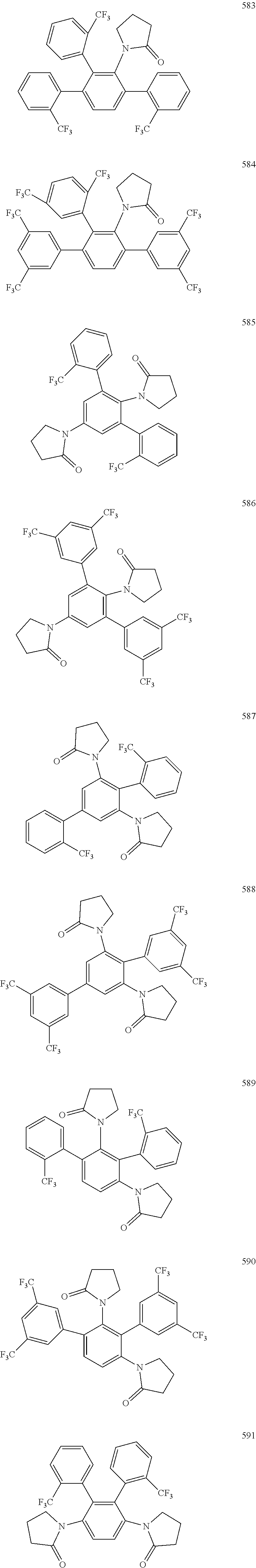

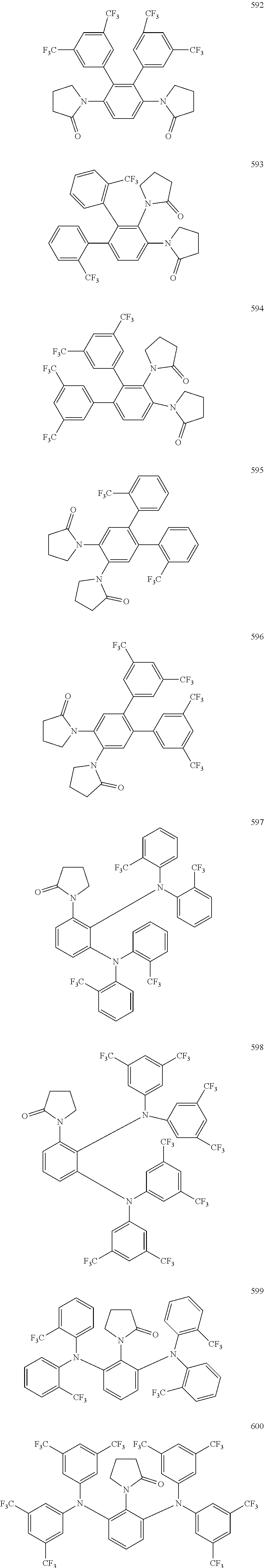

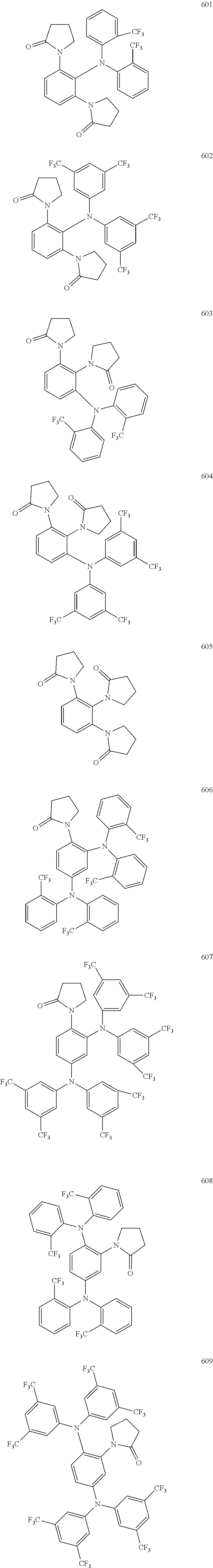

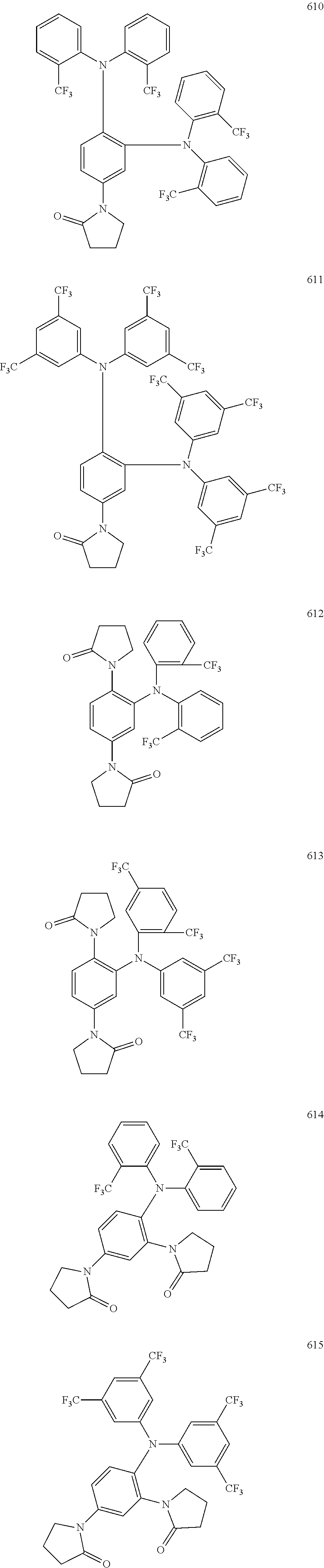

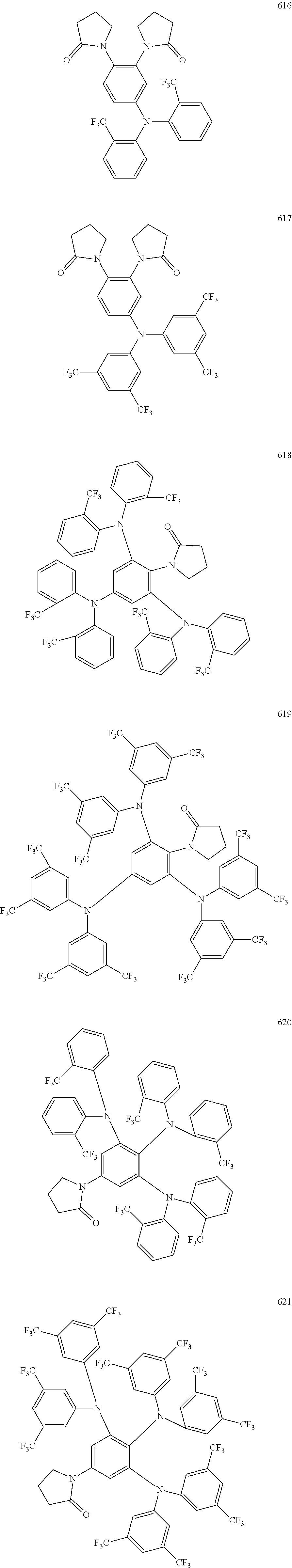

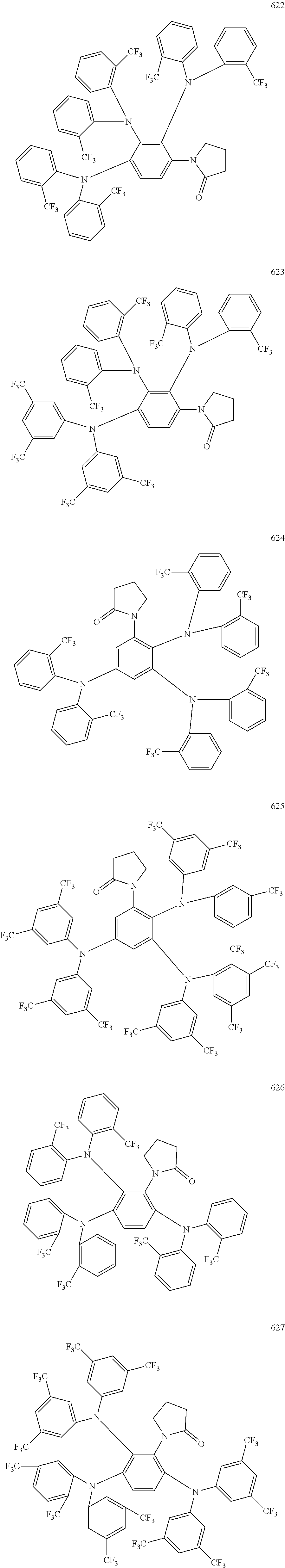

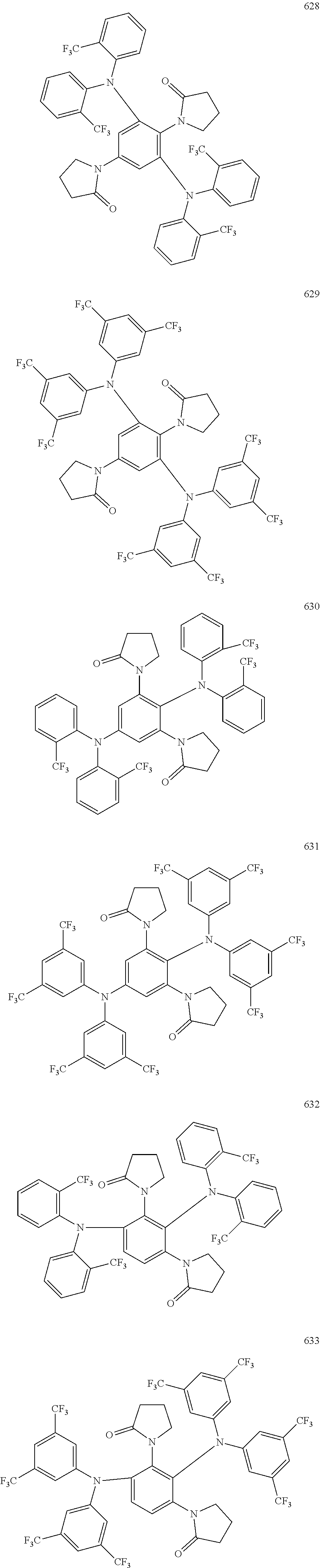

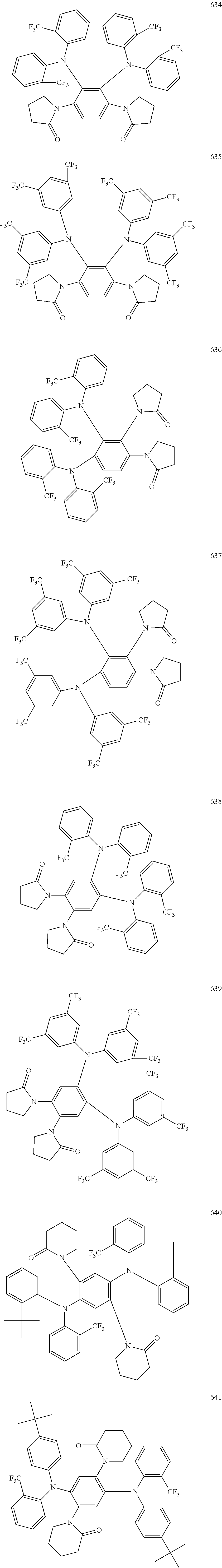

4. An organic light emitting device comprising a first electrode, a second electrode, and one or more organic layers arranged between the first and second electrodes, the organic light emitting device further comprising:
a light efficiency improving layer (capping layer) formed on at least one side opposite to the organic layer among upper or lower portions of the first electrode and the second electrode,
wherein the light efficiency improving layer includes a compound represented by Formula I above.

5. The organic light emitting device of claim 4, wherein the light efficiency improving layer is formed on at least one of a lower portion of the first electrode or an upper portion of the second electrode.
